(19) United States
(12) Patent Application Publication
(10) Pub. No.: US 2016/0368919 A1
(43) Pub. Date: Dec. 22, 2016

(54) NOVEL PYRIDINE PYRAZINONES AS BET-FAMILY BROMODOMAIN INHIBITORS

(71) Applicant: Pfizer Inc., New York, NY (US)

(72) Inventors: Agustin Casimiro-Garcia, Concord, MA (US); Bruce Allen Lefker, Arlington, MA (US); Nikolaos Papaioannou, Boston, MA (US); Arjun Venkat Narayanan, Cambridge, MA (US); Jotham Wadsworth Coe, Niantic, CT (US)

(73) Assignee: Pfizer Inc., New York, NY (US)

(21) Appl. No.: 15/184,097

(22) Filed: Jun. 16, 2016

Related U.S. Application Data
(60) Provisional application No. 62/181,281, filed on Jun. 18, 2015.

(51) Int. Cl. C07D 471/04

(52) U.S. Cl. CPC C07D 471/04

(57) ABSTRACT

Disclosed are novel pyridine pyrazinones or pharmaceutically acceptable salts thereof. Pharmaceutical compositions and the use of the compounds to treat diseases or disorders that are BET family bromodomain-dependent are also disclosed. Methods for preparing and using these compounds are further described.

### CROSS-REFERENCE TO RELATED APPLICATION

This application claims the benefit of Provisional Patent Application Ser. No. 62/181,281, filed Jun. 18, 2015, which is hereby incorporated by reference in its entirety.

### FIELD OF THE INVENTION

The present invention relates to novel pyridine pyrazinone compounds, or pharmaceutically acceptable salts thereof, and pharmaceutical compositions comprising the same. The present invention also relates to methods of treating a subject by administering a therapeutically effective amount of these compounds, or salts thereof, to a subject. In general, these compounds are BET-family bromodomain inhibitors.

### BACKGROUND OF THE INVENTION

Bromodomain-containing proteins are of substantial biological interest, as components of transcription factor complexes and determinants of epigenetic memory. The BET family (BRD2, BRD3, BRD4 and BRDT) shares a common domain architecture featuring two amino-terminal bromodomains that exhibit high levels of sequence conservation, and a more divergent carboxy-terminal recruitment domain (Filippakopoulos, P. et al., Nature 2010, 468, 1067-1073). BRD2 and BRD3 are reported to associate with histones along actively transcribed genes and may be involved in facilitating transcriptional elongation (Leroy et al, Mol. Cell. 2008, 30, 51-60). It has also been reported that BRD4 or BRD3 may fuse with NUT (nuclear protein in testis) forming novel fusion oncogenes, BRD4-NUT or BRD3-NUT, in a highly malignant form of epithelial neoplasia (French et al. Cancer Res., 2003, 63, 304-307 and French et al. J. Clin. Oncol. 2004, 22, 4135-4139). Data suggests that BRD—NUT fusion proteins contribute to carcinogensesis (Oncogene 2008, 27, 2237-2242). To date, BRDT is thought to be uniquely expressed in the testes and ovary. All family members have been reported to have some function in controlling or executing aspects of the cell cycle, and have been shown to remain in complex with chromosomes during cell division—suggesting a role in the maintenance of epigenetic memory. In addition some viruses make use of these proteins to tether their genomes to the host cell chromatin, as part of the process of viral replication (You et al. Cell 2004 117, 349-60). BRD4 appears to be involved in the recruitment of the pTEF-P complex to inducible genes, resulting in phosphorylation of RNA polymerase and increased transcriptional output (Hargreaves et al, Cell 2009 138, 129-145). BRD-4 has also been shown to bind to acetylated lysine-310 of the RelA subunit of NF-κB resulting in enhanced transcriptional activation of NF-κB and the expression of a subset of NF-κB responsive inflammatory genes (Huang et al, Mol Cell Biol 2009 29 1375-1387).

Bromodomain-containing protein 4 (BRD4) is a member of the BET family that, in yeast and animals, contains two tandem bromodomains (BD1 and BD2) and an extraterminal (ET) domain. BRD4 is a double bromodomain-containing protein that binds preferentially to acetylated chromatin and acetylated lysine-310 of the RelA subunit of NF-κB. In humans, four BET proteins (BRD2, BRD3, BRD4 and BRDT) exhibit similar gene arrangements, domain organizations, and some functional properties (Wu, S. et al., J. Biol. Chem. 2007, 282, 13141-13145).

There remains a need for further novel and potent small molecule compounds which act as BET-family bromodomain inhibitors.

### SUMMARY OF THE INVENTION

This invention relates to a compound of Formula I (Embodiment 1):

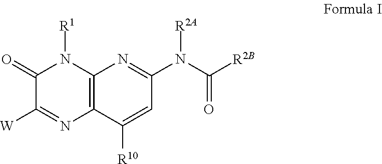

or a pharmaceutically acceptable salt thereof, wherein
R¹ is selected from the group consisting of:
—C₃-C₇cycloalkyl optionally substituted with one, two, three or four E;
(ii) 4 to 7 membered heterocyclyl optionally substituted with one, two, three or four E, which said 4 to 7 membered heterocyclyl comprises one or two heteroatoms independently selected for each occurrence from the group consisting of N, O and S; and
(iii)

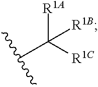

R^{1A} is selected from the group consisting of
   (i) C₁-C₆alkyl optionally substituted with one, two, three, four, five or six E;
   (ii) —C₃-C₇cycloalkyl optionally substituted with one, two, three, four or five E;
   (iii) phenyl optionally substituted with one, two, three, four or five E;
   (iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
   (v) 5 to 6 membered heteroaryl optionally substituted with one, two, three, four or five E, which said 5 to 6 membered heteroaryl comprises one, two or three heteroatoms independently selected for each occurrence from the group consisting of N, O and S;
R^{1B} is selected from the group consisting of
   (i) —H; and
   (ii) —C₁-C₆alkyl optionally substituted with one, two, three, four, five or six E;
R^{1C} is selected from the group consisting of
   (i) —H;
   (ii) —CH₃ optionally substituted with one, two, or three J;
   (iii) —CH₂CH₃ optionally substituted with one, two, three, four or five J;
   (iv) —CH₂CH₂CH₃ optionally substituted with one, two, three, four, five, six or seven J; and
   (v) —CH(CH₃)₂ optionally substituted with one, two, three, four, five, six or seven J;
R^{2A} is selected from the group consisting of
   (i) —H;
   (ii) —CH₃ optionally substituted with one, two or three J;
   (iii) —CH₂CH₃ optionally substituted with one, two, three, four, or five J; and
   (iv) cyclopropyl optionally substituted with one, two, three, four or five J;
R^{2B} is selected from the group consisting of
   (i) —C₁-C₆alkyl optionally substituted with one, two, three or four G;
   (ii) —OC₁-C₆alkyl optionally substituted with one, two, three or four G;
   (iii) —NH₂;
   (iv) —NH(C₁-C₆alkyl), which C₁-C₆alkyl is optionally substituted with one, two, three or four G;
   (v) —N(C₁-C₆alkyl)₂, which C₁-C₆alkyl is, independently for each occurrence, optionally substituted with one, two, three or four G;
   (vi) C₃-C₅cycloalkyl optionally substituted with one, two, three or four G; and
   (vii) 4 to 7 membered heterocyclyl optionally substituted with one, two, three or four G, which said 4 to 7 membered heterocyclyl comprises one or two heteroatoms independently selected for each occurrence from the group consisting of N, O and S;
W is selected from the group consisting of:
   (i)

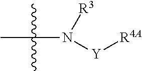

(ii)

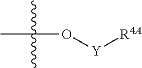

(iii)

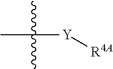

(iv)

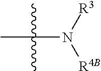

(v)

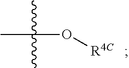

and
(vi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S;
Y is selected from the group consisting of:
   (i) —CH₂— optionally substituted with one or two J;
   (ii) —(CH₂)₂— optionally substituted with one, two, three or four J;
   (iii) —(CH₂)₃— optionally substituted with one, two, three, four, five or six J; and
   (iv) —(CH₂)₄— optionally substituted with one, two, three, four, five, six, seven or eight J;
R³ is selected from the group consisting of:
   (i) —H;
   (ii) —CH₃ optionally substituted with one, two, or three J;
   (iii) —CH₂CH₃ optionally substituted with one, two, three, four or five J;
   (iv) —CH₂CH₂CH₃ optionally substituted with one, two, three, four, five, six or seven J; and
   (v) —CH(CH₃)₂ optionally substituted with one, two, three, four, five, six or seven J;
R^{4A} is selected from the group consisting of
   (i) —H;
   (ii) —C₁-C₆alkyl optionally substituted with one, two, three or four G;
   (iii) —CO₂H;
   (iv) —C(O)C₁-C₆alkyl optionally substituted with one, two, three or four G;
   (v) —C(O)OC₁-C₆alkyl optionally substituted with one, two, three or four G;
   (vi) —C(O)NH₂;
   (vii) —C(O)NH(C₁-C₆alkyl) optionally substituted with one, two, three or four G;
   (viii) —C(O)N(C₁-C₆alkyl)₂ optionally substituted with one, two, three or four G; (ix) C(O)NHSO₂C₁-C₃alkyl optionally substituted with one, two, three or four G;
   (x) —NH(C₁-C₃alkyl) optionally substituted with one, two, three or four G;
   (xi) —N(C₁-C₃alkyl)₂ optionally substituted with one, two, three or four G;
   (xii) —NHC(O)C₁-C₃alkyl optionally substituted with one, two, three or four G;
   (xiii) —N(C₁-C₃alkyl)C(O)C₁-C₃alkyl optionally substituted with one, two, three or four G;
   (xiv) —NHSO₂C₁-C₃alkyl optionally substituted with one, two, three or four G;
   (xv) —N(C₁-C₃alkyl)SO₂C₁-C₃alkyl optionally substituted with one, two, three or four G;
   (xvi) —SO₂NH₂;
   (xvii) —SO₂NH(C₁-C₃alkyl) optionally substituted with one, two, three or four G;
   (xviii) —SO₂N(C₁-C₃alkyl)₂ optionally substituted with one, two, three or four G;
   (xix) —C₃-C₇cycloalkyl optionally substituted with one, two, three or four G;
   (xx) phenyl optionally substituted with one, two, three or four G;
   (xxi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
   (xxii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4B} is selected from the group consisting of
   (i) —H;
   (ii) —C₁-C₆alkyl optionally substituted with one, two, three or four G;
   (iii) —C(O)C₁-C₆alkyl optionally substituted with one, two, three or four G;
   (iv) —C(O)OC₁-C₆alkyl optionally substituted with one, two, three or four G;
   (v) —C(O)NH₂;
   (vi) —C(O)NH(C₁-C₆alkyl) optionally substituted with one, two, three or four G;
   (vii) —C(O)N(C₁-C₆alkyl)₂ optionally substituted with one, two, three or four G;
   (viii) —C(O)NHSO₂C₁-C₃alkyl optionally substituted with one, two, three or four G;
   (ix) —C₃-C₇cycloalkyl optionally substituted with one, two, three or four G;
   (x) phenyl optionally substituted with one, two, three or four G;
   (xi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
   (xii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4C} is selected from the group consisting of
   (i) —H;
   (ii) —C₁-C₆alkyl optionally substituted with one, two, three or four G;
   (iii) —C₃-C₇cycloalkyl optionally substituted with one, two, three or four G;
   (iv) phenyl optionally substituted with one, two, three or four G;
   (v) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
   (vi) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R¹⁰ is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂;
E is independently selected for each occurrence from the group consisting of:
(i) —OH;
(ii) —CN;
(iii) —CO₂H;
(iv) —C(O)H;
(v) halo;
(vi) —C₁-C₃alkyl optionally substituted with one, two, three or four J;
(vii) —C₁-C₃alkylCO₂H which —C₁-C₃alkyl is optionally substituted with one, two, three or four J;
(viii) —C₃-C₇cycloalkyl optionally substituted with one, two, three, four, five or six J;
(ix) —C₁-C₃alkylC₃-C₆cycloalkyl optionally substituted with one, two, three, four, five or six J;
(x) —OC₁-C₃alkyl, optionally substituted with one, two, three or four J;
(xi) —OC₃-C₇cycloalkyl optionally substituted with one, two, three, four, five or six J;
(xii) —OC₁-C₃alkylC₃-C₇cycloalkyl optionally substituted with one, two, three, four, five or six J;
(xiii) —SC₁-C₃alkyl, optionally substituted with one, two, three or four J;
(xiv) —SC₃-C₇cycloalkyl optionally substituted with one, two, three, four, five or six J;
(xv) —SC₁-C₃alkylC₃-C₇cycloalkyl optionally substituted with one, two, three, four, five or six J;
(xvi) —C(O)C₁-C₃alkyl, optionally substituted with one, two, three or four J;
(xvii) —C(O)OC₁-C₃alkyl, optionally substituted with one, two, three or four J;
(xviii) —NH₂;
(xix) —NH(C₁-C₃alkyl) optionally substituted with one, two, three or four J;
(xx) —N(C₁-C₃alkyl)₂ which —C₁-C₃alkyl is, independently for each occurrence, optionally substituted with one, two, three or four J;
(xxi) —C(O)NH₂;
(xxii) —C(O)NHC₁-C₃alkyl, optionally substituted with one, two, three or four J;
(xxiii) —C(O)N(C₁-C₃alkyl)₂, which C₁-C₃alkyl is, independently for each occurrence, optionally substituted with one, two, three or four J;
(xxiv) —NHC(O)C₁-C₃alkyl, optionally substituted with one, two, three or four J;
(x) —SO₂(C₁-C₃alkyl), optionally substituted with one, two, three or four J;
(xxvi) —SO₂NH(C₁-C₃alkyl), optionally substituted with one, two, three or four J;
(xxvii) —NHSO₂(C₁-C₃alkyl), optionally substituted with one, two, three or four J; and
(xxviii) phenyl optionally substituted with one, two, three, or four J;
G is independently selected for each occurrence from the group consisting of
(i) —OH;
(ii) —CN;
(iii) —CO₂H;
(iv) —C(O)H;
(v) halo;
(vi) —C₁-C₃alkyl, optionally substituted with one, two, three or four J;
(vii) —C₁-C₃alkylCO₂H, which —C₁-C₃alkyl is optionally substituted with one, two, three or four J;
(viii) —C₁-C₃alkylC₃-C₆cycloalkyl optionally substituted with one, two, three, four, five or six J;
(ix) —OC₁-C₃alkyl, optionally substituted with one, two, three or four J;
(x) —OC₁-C₃alkylC₃-C₆cycloalkyl optionally substituted with one, two, three, four, five or six J;
(xi) —SC₁-C₃alkyl, optionally substituted with one, two, three or four J;
(xii) —SC₁-C₃alkylC₃-C₆cycloalkyl optionally substituted with one, two, three, four, five or six J;
(xiii) —C(O)C₁-C₃alkyl, optionally substituted with one, two, three or four J;
(xiv) —C(O)OC₁-C₃alkyl, optionally substituted with one, two, three or four J;
(xv) —NH₂;
(xvi) —NH(C₁-C₃alkyl), optionally substituted with one, two, three or four J;
(xvii) —N(C₁-C₃alkyl)₂, which —C₁-C₃alkyl is, independently for each occurrence, optionally substituted with one, two, three or four J;
(xviii) —C(O)NH₂;
(xix) —C(O)NHC₁-C₃alkyl, optionally substituted with one, two, three or four J;
(xx) —C(O)N(C₁-C₃alkyl)₂, which —C₁-C₃alkyl is, independently for each occurrence, optionally substituted with one, two, three or four J;
(x) —NHC(O)C₁-C₃alkyl, optionally substituted with one, two, three or four J;
(xxii) —SO₂(C₁-C₃alkyl), optionally substituted with one, two, three or four J;
(xxiii) —SO₂NH(C₁-C₃alkyl), optionally substituted with one, two, three or four J; and
(xxiv) —NHSO₂(C₁-C₃alkyl) optionally substituted with one, two, three or four J; and
J is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂.

In another Embodiment (1.1), the invention provides a compound of Formula (I′):

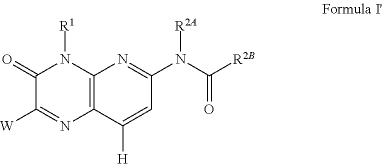

or a pharmaceutically acceptable salt thereof, and wherein R¹, R^{1A}, R^{1B}, R^{1C}, R^{2A}, R^{2B}, W, Y, R³, R^{4A}, R^{4B}, R^{4C}, E, G, and J are all defined as for a compound of Formula (I).

In another Embodiment (2), the invention provides a compound of Formula (I), or Embodiment (1.1), or a pharmaceutically acceptable salt thereof, wherein R¹ is —C₃-C₇cycloalkyl optionally substituted as defined for a compound of Formula (I).

In another Embodiment (2.1), the invention provides a compound of Formula (I), Embodiment (1.1), or Embodiment (2), or a pharmaceutically acceptable salt thereof, wherein R¹ is —C₃-C₇cycloalkyl selected from the group consisting of cyclopropyl, cyclobutyl, cyclopentyl, and cyclohexyl, preferably cyclopropyl, cyclobutyl and cyclopentyl, which cyclopropyl, cyclobutyl, cyclopentyl or cyclohexyl is optionally substituted as defined for a compound of Formula (I).

In another Embodiment (2.2), the invention provides a compound of Formula (I), Embodiment (1.1), Embodiment (2), or Embodiment (2.1), or a pharmaceutically acceptable salt thereof, wherein R¹ is selected from the group consisting of cyclopropyl, cyclobutyl, cyclopentyl and cyclohexyl, preferably cyclopropyl, cyclobutyl and cyclopentyl, which cyclopropyl, cyclobutyl, cyclopentyl or cyclohexyl is unsubstituted or substituted with one, two, three of four E, which E is independently selected for each occurrence from the group consisting of halo, for example —F or —Cl; —C₁-C₃alkyl, for example methyl or ethyl; —OC₁-C₃alkyl; —C₃-C₇cycloalkyl, for example cyclopentyl; and phenyl, and which E is optionally further substituted as defined for a compound of Formula (I), for example E is —C₁-C₃alkyl substituted with one, two, three or four J, to form, for example, —CF₃.

In another Embodiment (2.3), the invention provides a compound of Formula (I), Embodiment (1.1), Embodiment (2), Embodiment (2.1), or Embodiment (2.2), or a pharmaceutically acceptable salt thereof, wherein R¹ is selected from the group consisting of cyclopropyl, cyclobutyl, cyclopentyl and cyclohexyl, preferably cyclopropyl, cyclobutyl and cyclopentyl, which cyclopropyl, cyclobutyl, cyclopentyl or cyclohexyl is unsubstituted or substituted with one or two E, which E is independently selected for each occurrence from the group consisting of —C₁-C₃alkyl, for example ethyl; —C₃-C₇cycloalkyl, for example cyclopentyl; and phenyl, and which E is optionally further substituted as defined for a compound of Formula (I).

In another Embodiment (2.4), the invention provides a compound of Formula (I), Embodiment (1.1), Embodiment (2), Embodiment (2.1), Embodiment (2.2), or Embodiment (2.3), or a pharmaceutically acceptable salt thereof, wherein R¹ is selected from the group consisting of cyclopropyl, which cyclopropyl is unsubstituted or substituted with one E, which E is cyclopentyl; cyclobutyl, which cyclobutyl is unsubstituted or substituted with one E, which E is phenyl; and cyclopentyl, which cyclopentyl is unsubstituted or substituted with two E, which E are both ethyl.

In another Embodiment (3), the invention provides a compound of Formula (I), or Embodiment (1.1), or a pharmaceutically acceptable salt thereof, wherein R¹ is 4 to 7 membered heterocyclyl optionally substituted as defined for a compound of Formula (I), which said 4 to 7 membered heterocyclyl comprises one or two heteroatoms independently selected for each occurrence from the group consisting of N, O and S.

In another Embodiment (3.1), the invention provides a compound of Formula (I), or Embodiment (1.1), or a pharmaceutically acceptable salt thereof, wherein R¹ is selected from the group consisting of tetrahydrofuranyl, pyrrolidinyl, tetrahydrothiophenyl, pyrazolidinyl, imidazolidinyl, dioxolanyl, thiazolidinyl, isoxazolidinyl, tetrahydropyranyl, piperidinyl, piperizinyl and morpholinyl, which tetrahydrofuranyl, pyrrolidinyl, tetrahydrothiophenyl, pyrazolidinyl, imidazolidinyl, dioxolanyl, thiazolidinyl, isoxazolidinyl, tetrahydropyranyl, piperidinyl, piperizinyl and morpholinyl is optionally substituted as defined for a compound of Formula (I).

In another Embodiment (3.2), the invention provides a compound of Formula (I), or Embodiment (1.1), Embodiment (3) or Embodiment (3.1), or a pharmaceutically acceptable salt thereof, wherein R¹ is selected from the group consisting of tetrahydrofuranyl and tetrahydropyranyl, preferably tetrahydrofuranyl, which tetrahydrofuranyl or tetrahydropyranyl is optionally substituted as defined for a compound of Formula (I).

In another Embodiment (3.3), the invention provides a compound of Formula (I), or Embodiment (1.1), Embodiment (3), Embodiment (3.1) or Embodiment (3.2), or a pharmaceutically acceptable salt thereof, wherein R¹ is selected from the group consisting of tetrahydrofuranyl and tetrahydropyranyl, preferably tetrahydrofuranyl, which tetrahydrofuranyl or tetrahydropyranyl is unsubstituted or substituted with one, two, three of four E, which E is independently selected for each occurrence from the group consisting of halo, for example F or —Cl; —C₁-C₃alkyl, for example methyl or ethyl; —OC₁-C₃alkyl; —C₃-C₇cycloalkyl, for example cyclopentyl; and phenyl, and which E is optionally further substituted as defined for a compound of Formula (I), for example E is —C₁-C₃alkyl substituted with one, two, three or four J, to form, for example, —CF₃.

In another Embodiment (3.4), the invention provides a compound of Formula (I), or Embodiment (1.1), Embodiment (3), Embodiment (3.1), Embodiment (3.2), or Embodiment (3.3), or a pharmaceutically acceptable salt thereof, wherein R¹ is tetrahydrofuranyl which tetrahydrofuranyl is unsubstituted or substituted with one E, which E is phenyl.

In an another Embodiment (4), the invention provides a compound of Formula (I), or Embodiment (1.1), or a pharmaceutically acceptable salt thereof, wherein R¹ is

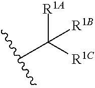

optionally substituted as defined for a compound of Formula (I).

In another Embodiment (4.1), the invention provides a compound of Formula (I), Embodiment (1.1), or Embodiment (4), or a pharmaceutically acceptable salt thereof, wherein R¹ is

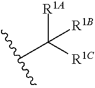

and R^{1A} is selected from the group consisting of —C₁-C₆alkyl, more preferably —C₁-C₄alkyl, for example methyl, ethyl or n-propyl; —C₃-C₇cycloalkyl, for example cyclohexyl; phenyl; and 5 to 6 membered heteroaryl which said 5 to 6 membered heteroaryl comprises one, two or three heteroatoms independently selected for each occurrence from the group consisting of N, O and S, for example pyridyl, pyridazinyl, or pyrimidinyl, which R^{1A} is optionally substituted as defined for a compound of Formula (I).

In another Embodiment (4.2), the invention provides a compound of Formula (I), Embodiment (1.1), Embodiment (4), or Embodiment (4.1), or a pharmaceutically acceptable salt thereof, wherein R¹ is

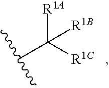

and R^{1A} is selected from the group consisting of methyl, ethyl, n-propyl, cyclohexyl, phenyl, pyridyl, pyridazinyl and pyrimidinyl which R^{1A} is optionally substituted as defined for a compound of Formula (I).

In another Embodiment (4.2a), the invention provides a compound of Formula (I), Embodiment (1.1), Embodiment (4), Embodiment (4.1), or Embodiment (4.2), or a pharmaceutically acceptable salt thereof, wherein R¹ is

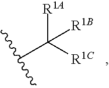

and R^{1A} is selected from the group consisting of methyl, ethyl, n-propyl, cyclohexyl, phenyl, pyridyl, pyridazinyl and pyrimidinyl which R^{1A} is optionally substituted with E as defined for a compound of Formula (I), which E is independently selected for each occurrence from the group consisting of —OH; —F; —Cl; —CH₃; —OCH₃; and —CF₃.

In another Embodiment (4.3), the invention provides a compound of Formula (I), Embodiment (1.1), Embodiment (4), Embodiment (4.1), Embodiment (4.2) or Embodiment (4.2a), or a pharmaceutically acceptable salt thereof, wherein R¹ is

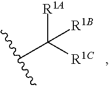

and R^{1A} is selected from the group consisting of methyl, ethyl, n-propyl, phenyl, and pyridyl, which R^{1A} is optionally substituted as defined for a compound of Formula (I).

In another Embodiment (4.4), the invention provides a compound of Formula (I), Embodiment 1.1), Embodiment (4), Embodiment (4.1), Embodiment (4.2), Embodiment (4.2a) or Embodiment (4.3), or a pharmaceutically acceptable salt thereof, wherein R¹ is

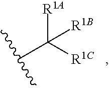

and R^{1A} is selected from the group consisting of methyl, phenyl, and pyridyl, which R^{1A} is optionally substituted as defined for a compound of Formula (I).

In another Embodiment (4.5), the invention provides for a compound of Formula (I), Embodiment (1.1), Embodiment (4), Embodiment (4.1), Embodiment (4.2), Embodiment (4.2a) Embodiment (4.3), or Embodiment (4.4), or a pharmaceutically acceptable salt thereof, wherein R¹ is

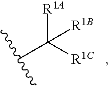

and R^{1A} is selected from the group consisting of methyl, phenyl and pyridyl, and which R^{1A} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (I), for example E is —C₁-C₃alkyl substituted with one, two, three or four J, to form, for example, —CF₃.

In another Embodiment (4.6), the invention provides for a compound of Formula (I), Embodiment (1.1), Embodiment (4), Embodiment (4.1), Embodiment (4.2), Embodiment (4.2a) Embodiment (4.3), Embodiment (4.4), or Embodiment (4.5), or a pharmaceutically acceptable salt thereof, wherein R¹ is

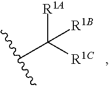

and R^{1A} is selected from the group consisting of methyl, phenyl, and pyridyl, which methyl, phenyl, and pyridyl is unsubstituted or substituted with one or two E, which E is independently selected from the group consisting of —OH, to form, for example, CH₂OH; —F, to form, for example, —CF₃ or fluorophenyl; —C₁-C₃alkyl, for example methyl, to form, for example, methylphenyl or methylpyridyl; —OC₁-C₃alkyl, for example methoxy or ethoxy, to form, for example, —CH₂OCH₃, —CH₂OCH₂CH₃, methoxyphenyl, or methoxypyridyl.

In another Embodiment (4.7), the invention provides a compound of Formula (I), Embodiment (1.1), Embodiment (4), Embodiment (4.1), Embodiment (4.2), Embodiment (4.2a) Embodiment (4.3), Embodiment (4.4), Embodiment (4.5), or Embodiment (4.6), or a pharmaceutically acceptable salt thereof, wherein R¹ is

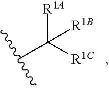

and R^{1A} is selected from the group consisting of —CH₂OCH₃; phenyl; methoxyphenyl; and pyridyl.

In another Embodiment (4.8), the invention provides a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), or a pharmaceutically acceptable salt thereof, wherein R¹ is

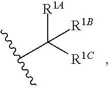

and R^{1B} is selected from the group consisting of —H; and —C₁-C₆alkyl, more preferably —C₁-C₄alkyl, for example methyl, ethyl, n-propyl or i-propyl, which R^{1B} is optionally substituted as defined for a compound of Formula (I).

In another Embodiment (4.9), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), or Embodiment (4.8), or a pharmaceutically acceptable salt thereof, wherein R¹ is

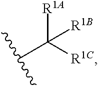

and R^{1B} is selected from the group consisting of —H, methyl, ethyl, n-propyl and i-propyl, which R^{1B} is optionally substituted as defined for a compound of Formula (I).

In another Embodiment (4.9a), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), Embodiment (4.8), Embodiment (4.9), or a pharmaceutically acceptable salt thereof, wherein R¹ is

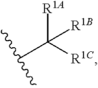

and R^{1B} is selected from the group consisting of —H, methyl, ethyl, n-propyl and i-propyl, which R^{1B} is optionally substituted with E as defined for a compound of Formula (I), which E is independently selected for each occurrence from the group consisting of —OH; —F; —Cl; —CH₃; —OCH₃; and —CF₃.

In another Embodiment (4.10), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), Embodiment (4.8), Embodiment (4.9), or Embodiment (4.9a), or a pharmaceutically acceptable salt thereof, wherein R¹ is

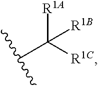

and R^{1B} is selected from the group consisting of methyl, ethyl, n-propyl and i-propyl, which R^{1B} is unsubstituted or substituted with one or two E, which E is independently selected for each occurrence from the group consisting of —CN; —OH; halo, for example —F or Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (I), for example E is —C₁-C₃alkyl substituted with one, two, three or four J, to form, for example, —CF₃.

In another Embodiment (4.11), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), Embodiment (4.8), Embodiment (4.9), Embodiment (4.9a), or Embodiment (4.10), or a pharmaceutically acceptable salt thereof, wherein R¹ is

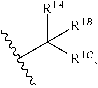

and R^{1B} is selected from the group consisting of methyl, ethyl, n-propyl and i-propyl, which R^{1B} is unsubstituted or substituted with one or two E, which E is independently selected for each occurrence from the group consisting of —OC₁-C₃alkyl, for example methoxy, to form, for example —CH₂OCH₃.

In another Embodiment (4.12), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), Embodiment (4.8), Embodiment (4.9), Embodiment (4.9a), Embodiment (4.10), or Embodiment (4.11), or a pharmaceutically acceptable salt thereof, wherein R¹ is

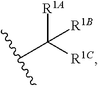

and R^{1B} is selected from the group consisting of H, methyl, ethyl, n-propyl, i-propyl, and —CH₂OCH₃.

In another Embodiment (4.13), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), Embodiment (4.8), Embodiment (4.9), Embodiment (4.9a), Embodiment (4.10), Embodiment (4.11), or Embodiment (4.12), or a pharmaceutically acceptable salt thereof, wherein R¹ is

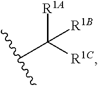

and R^{1C} is selected from the group consisting of —CH₃; and —H.

In another Embodiment (4.14), the invention provides for a compound of Formula (I), Embodiment (1.1), Embodiment (4), Embodiment (4.1), Embodiment (4.2), Embodiment (4.2a) Embodiment (4.3), Embodiment (4.4), Embodiment (4.5), Embodiment (4.6), Embodiment (4.7), Embodiment (4.8), Embodiment (4.9), Embodiment (4.9a), Embodiment (4.10), Embodiment (4.11), Embodiment (4.12), or Embodiment (14.13) or a pharmaceutically acceptable salt thereof, wherein R¹ is

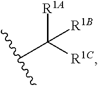

### and R^{1C} is —H.

In an another Embodiment (5), the invention provides a compound of Formula (Ia),

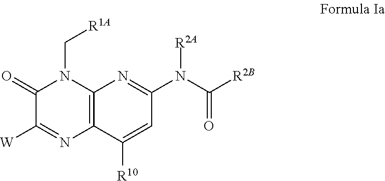

or a pharmaceutically acceptable salt thereof, wherein:

R^{1A} is selected from the group consisting of
(i) —C₁-C₆alkyl optionally substituted with one, two, three, four, five or six E;
(ii) —C₃-C₇cycloalkyl optionally substituted with one, two, three, four or five E;
(iii) phenyl optionally substituted with one, two, three, four or five E;
(iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
(v) 5 to 6 membered heteroaryl optionally substituted with one, two, three, four or five E, which said 5 to 6 membered heteroaryl comprises one, two or three heteroatoms independently selected for each occurrence from the group consisting of N, O and S;
   and wherein R^{2A}, R^{2B}, W, Y, R³, R^{4A}, R^{4B}, R^{4C}, R¹⁰, E, G, and J are all defined as for a compound of Formula (I).

In another Embodiment (5.0), the invention provides a compound of Formula (Ia′):

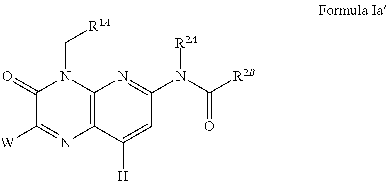

or a pharmaceutically acceptable salt thereof, and wherein R^{1A}, R^{2A}, R^{2B}, W, Y, R³, R^{4A}, R^{4B}, R^{4C}, E, G, and J are all defined as for a compound of Formula (Ia).

In another Embodiment (5.1), the invention provides a compound of Formula (Ia), or Embodiment (5.0), or a pharmaceutically acceptable salt thereof, wherein R^{1A} is selected from the group consisting of —C₃-C₇cycloalkyl; phenyl; 4 to 7 membered heterocyclyl, which said 4 to 7 membered heterocyclyl comprises one or two heteroatoms independently selected for each occurrence from the group consisting of N, O and S; and 5 to 6 membered heteroaryl, which said 5 to 6 membered heteroaryl comprises one, two or three heteroatoms independently selected for each occurrence from the group consisting of N, O and S, which R^{1A} is optionally substituted as defined for a compound of Formula (Ia).

In another Embodiment (5.1a), the invention provides a compound of Formula (Ia), or Embodiment (5.0), or a pharmaceutically acceptable salt thereof, wherein R^{1A} is selected from the group consisting of —C₃-C₇cycloalkyl, for example cyclohexyl; phenyl; and 5 to 6 membered heteroaryl which said 5 to 6 membered heteroaryl comprises one, two or three heteroatoms independently selected for each occurrence from the group consisting of N, O and S, for example pyridyl, pyridazinyl, or pyrimidinyl, which R^{1A} is optionally substituted as defined for a compound of Formula (Ia).

In another Embodiment (5.2), the invention provides a compound of Formula (Ia), Embodiment (5.0), or Embodiment (5.1), or a pharmaceutically acceptable salt thereof, wherein R^{1A} is selected from the group consisting of cyclohexyl, phenyl, pyridyl, pyridazinyl and pyrimidinyl which R^{1A} is optionally substituted as defined for a compound of Formula (Ia).

In another Embodiment (5.3), the invention provides a compound of Formula (Ia), Embodiment (5.0), Embodiment (5.1), or Embodiment (5.2), or a pharmaceutically acceptable salt thereof, wherein R¹ is

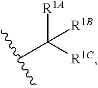

and R^{1A} is selected from the group consisting of cyclohexyl, phenyl, pyridyl, pyridazinyl and pyrimidinyl which R^{1A} is optionally substituted with E as defined for a compound of Formula (Ia), which E is independently selected for each occurrence from the group consisting of —OH; —F; —Cl; —CH₃; —OCH₃; and —CF₃.

In another Embodiment (5.4), the invention provides a compound of Formula (1a), Embodiment (5.0), Embodiment (5.1), Embodiment (5.2), or Embodiment (5.3), or a pharmaceutically acceptable salt thereof, wherein R^{1A} is selected from the group consisting of phenyl, and pyridyl which R^{1A} is optionally substituted as defined for a compound of Formula (Ia).

In another Embodiment (5.5), the invention provides for a compound of Formula (Ia), Embodiment (5.0), Embodiment (5.1), Embodiment (5.2), Embodiment (5.2a), Embodiment (5.3), or Embodiment (5.4), or a pharmaceutically acceptable salt thereof, wherein R^{1A} is selected from the group consisting of phenyl and pyridyl, and which R^{1A} is unsubstituted or substituted with one, or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (Ia), for example E is —C₁-C₃alkyl substituted with one, two, three or four J, to form, for example, —CF₃.

In another Embodiment (5.6), the invention provides for a compound of Formula (Ia), Embodiment (5.0), Embodiment (5.1), Embodiment (5.2), Embodiment (5.2a), Embodiment (5.3), Embodiment (5.4), or Embodiment (5.5), or a pharmaceutically acceptable salt thereof, wherein R^{1A} is selected from the group consisting of phenyl, and pyridyl, which phenyl and pyridyl is unsubstituted or substituted with one or two E, which E is independently selected for each occurrence from the group consisting of —OH, to form, for example, phenol; —F, to form, for fluorophenyl; —C₁-C₃alkyl, for example methyl, to form, for example, methylphenyl or methylpyridyl; —OC₁-C₃alkyl, for example methoxy or ethoxy, to form, for example, methoxyphenyl, or methoxypyridyl.

In another Embodiment (5.7), the invention provides a compound of Formula (Ia), Embodiment (5.0), Embodiment (5.1), Embodiment (5.2), Embodiment (5.2a), Embodiment (5.3), Embodiment (5.4), Embodiment (5.5), or Embodiment (5.6), or a pharmaceutically acceptable salt thereof, wherein R^{1A} is selected from the group consisting of phenyl; methoxyphenyl; and pyridyl.

In another Embodiment (6), this invention relates to a compound of Formula (Ib):

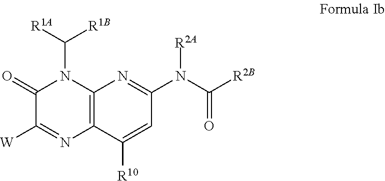

or a pharmaceutically acceptable salt thereof, wherein:

R^{1A} is selected from the group consisting of
(i) —C₁-C₆alkyl optionally substituted with one, two, three, four, five or six E;
(ii) —C₃-C₇cycloalkyl optionally substituted with one, two, three, four or five E;
(iii) phenyl optionally substituted with one, two, three, four or five E;
(iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
(v) 5 to 6 membered heteroaryl optionally substituted with one, two, three, four or five E, which said 5 to 6 membered heteroaryl comprises one, two or three heteroatoms independently selected for each occurrence from the group consisting of N, O and S;

R^{1B} is —C₁-C₆alkyl optionally substituted with one, two, three, four, five or six E;

and wherein R^{2A}, R^{2B}, W, Y, R³, R^{4A}, R^{4B}, R^{4C}, R¹⁰, E, G, and J are all defined as for a compound of Formula (I).

In another Embodiment (6.0), the invention provides a compound of Formula (Ib′):

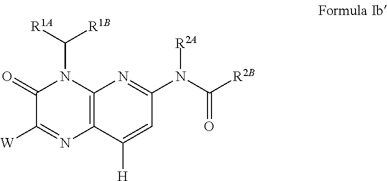

or a pharmaceutically acceptable salt thereof, according to any preceding Embodiment, and wherein R^{1A}, R^{2A}, R^{2B}, W, Y, R³, R^{4A}, R^{4B}, R^{4C}, E, G, and J are all defined as for a compound of Formula (Ib).

In another Embodiment (6.1), the invention provides a compound of Formula (Ib), or Embodiment (6.0), or a pharmaceutically acceptable salt thereof, wherein R^{1A} is selected from the group consisting of —C₁-C₆alkyl, more preferably —C₁-C₄alkyl, for example methyl, ethyl, or n-propyl; —C₃-C₇cycloalkyl, for example cyclohexyl; phenyl; and 5 to 6 membered heteroaryl which said 5 to 6 membered heteroaryl comprises one, two or three heteroatoms independently selected for each occurrence from the group consisting of N, O and S, for example pyridyl, pyridazinyl, or pyrimidinyl, which R^{1A} is optionally substituted as defined for a compound of Formula (Ib).

In another Embodiment (6.2), the invention provides a compound of Formula (Ib), Embodiment (6.0), or Embodiment (6.1), or a pharmaceutically acceptable salt thereof, wherein R^{1A} is selected from the group consisting of methyl, ethyl, n-propyl, cyclohexyl, phenyl, pyridyl, pyridazinyl and pyrimidinyl which R^{1A} is optionally substituted as defined for a compound of Formula (Ib).

In another Embodiment (6.2a), the invention provides a compound of Formula (Ib), Embodiment (6.0), Embodiment (6.1), or Embodiment (6.2), or a pharmaceutically acceptable salt thereof, wherein R¹ is

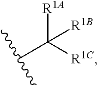

and R^{1A} is selected from the group consisting of methyl, ethyl, n-propyl, cyclohexyl, phenyl, pyridyl, pyridazinyl and pyrimidinyl which R^{1A} is optionally substituted with E as defined for a compound of Formula (Ib), which E is independently selected for each occurrence from the group consisting of —OH; —F; —Cl; —CH₃; —OCH₃; and —CF₃.

In another Embodiment (6.3), the invention provides a compound of Formula (Ib), Embodiment (6.0), Embodiment (6.1), Embodiment (6.2), or Embodiment (6.2a), or a pharmaceutically acceptable salt thereof, wherein R^{1A} is selected from the group consisting of methyl, ethyl, n-propyl, phenyl, and pyridyl, which R^{1A} is optionally substituted as defined for a compound of Formula (Ib).

In another Embodiment (6.4), the invention provides a compound of Formula (Ib), Embodiment (6.0), Embodiment (6.1), Embodiment (6.2), Embodiment (6.2a), or Embodiment (6.3), or a pharmaceutically acceptable salt thereof, wherein R^{1A} is selected from the group consisting of methyl, phenyl, and pyridyl, which R^{1A} is optionally substituted as defined for a compound of Formula (Ib).

In another Embodiment (6.5), the invention provides for a compound of Formula (Ib), Embodiment (6.0), Embodiment (6.1), Embodiment (6.2), Embodiment (6.2a), Embodiment (6.3), or Embodiment (6.4), or a pharmaceutically acceptable salt thereof, wherein R^{1A} is selected from the group consisting of methyl, phenyl and pyridyl, and which R^{1A} is unsubstituted or substituted with one, or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (Ib), for example E is —C₁-C₃alkyl substituted with one, two, three or four J, to form, for example, —CF₃.

In another Embodiment (6.6), the invention provides for a compound of Formula (Ib), Embodiment (6.0), Embodiment (6.1), Embodiment (6.2), Embodiment (6.2a), Embodiment (6.3), Embodiment (6.4), or Embodiment (6.5), or a pharmaceutically acceptable salt thereof, wherein R^{1A} is selected from the group consisting of methyl, phenyl, and pyridyl, which methyl, phenyl, and pyridyl is unsubstituted or substituted with one or two E, which E is independently selected for each occurrence from the group consisting of —OH, to form, for example, CH₂OH; —F, to form, for example, CF₃ or fluorophenyl; —C₁-C₃alkyl, for example methyl, to form, for example, methylphenyl or methylpyridyl; —OC₁-C₃alkyl, for example methoxy or ethoxy, to form, for example, —CH₂OCH₃, —CH₂OCH₂CH₃, methoxyphenyl, or methoxypyridyl.

In another Embodiment (6.7), the invention provides a compound of Formula (Ib), Embodiment (6.0), Embodiment (6.1), Embodiment (6.2), Embodiment (6.2a), Embodiment (6.3), Embodiment (6.4), Embodiment (6.5), or Embodiment (6.6), or a pharmaceutically acceptable salt thereof, wherein R^{1A} is selected from the group consisting of —CH₂OCH₃; phenyl; methoxyphenyl; and pyridyl.

In another Embodiment (6.8), the invention provides a compound of Formula (Ib), Embodiment (6.0), Embodiment (6.1), Embodiment (6.2), Embodiment (6.2a), Embodiment (6.3), Embodiment (6.4), Embodiment (6.5), Embodiment (6.6), or Embodiment (6.7), or a pharmaceutically acceptable salt thereof, wherein R^{1B} is selected from the group consisting of —C₁-C₆alkyl, more preferably —C₁-C₄alkyl, for example methyl, ethyl, n-propyl or i-propyl, which R^{1B} is optionally substituted as defined for a compound of Formula (Ib).

In another Embodiment (6.9), the invention provides a compound of Formula (Ib), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), or Embodiment (6.8), or a pharmaceutically acceptable salt thereof, wherein R^{1B} is selected from the group consisting of methyl, ethyl, n-propyl and i-propyl, which R^{1B} is optionally substituted as defined for a compound of Formula (Ib).

In another Embodiment (6.9a), the invention provides for a compound of Formula (Ib), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), Embodiment (6.8), or Embodiment (6.9), or a pharmaceutically acceptable salt thereof, wherein R¹ is

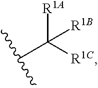

and R^{1B} is selected from the group consisting of methyl, ethyl, n-propyl and i-propyl, which R^{1B} is optionally substituted with E as defined for a compound of Formula (Ib), which E is independently selected for each occurrence from the group consisting of —OH; —F; —Cl; —CH₃; —OCH₃; and —CF₃.

In another Embodiment (6.10), the invention provides a compound of Formula (Ib), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), Embodiment (6.8), Embodiment (6.9), or Embodiment (6.9a), or a pharmaceutically acceptable salt thereof, wherein R^{1B} is selected from the group consisting of methyl, ethyl, n-propyl and i-propyl, which R^{1B} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (Ib), for example E is —C₁-C₃alkyl substituted with one, two, three or four J, to form, for example, —CF₃.

In another Embodiment (6.11), the invention provides a compound of Formula (Ib), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), Embodiment (6.8), Embodiment (6.9), Embodiment (6.9a), or Embodiment (6.10), or a pharmaceutically acceptable salt thereof, wherein R^{1B} is selected from the group consisting of methyl, ethyl, n-propyl and i-propyl, which R^{1B} is unsubstituted or substituted with one or two E, which E is independently selected for each occurrence from the group consisting of —OC₁-C₃alkyl, for example methoxy, to form, for example —CH₂OCH₃.

In another Embodiment (6.12), the invention provides a compound of Formula (Ib), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), Embodiment (6.8), Embodiment (6.9), Embodiment (6.9a), Embodiment (6.10), or Embodiment (6.11), or a pharmaceutically acceptable salt thereof, wherein R^{1B} is selected from the group consisting of methyl, ethyl, n-propyl, i-propyl, and —CH₂OCH₃.

In another Embodiment (7), the invention provides a compound of Formula (I), Formula (Ia), or Formula (Ib), or a pharmaceutically acceptable salt thereof, according to any preceding Embodiment, wherein R^{2A} is selected from the group consisting of —CH₃, —CH₂CH₃, and cyclopropyl, which R^{2A} is optionally substituted as defined for a compound of Formula (I), Formula (Ia), or Formula (Ib) respectively.

In another Embodiment (7.1), the invention provides a compound of Formula (I), Formula (Ia), or Formula (Ib), or a pharmaceutically acceptable salt thereof, according to any preceding Embodiment, wherein R^{2A} is selected from the group consisting of —CH₃, and —CH₂CH₃, which R^{2A} is optionally substituted as defined for a compound of Formula (I), Formula (Ia), or Formula (Ib) respectively.

In another Embodiment (7.2), the invention provides a compound of Formula (I), Formula (Ia), or Formula (Ib), or a pharmaceutically acceptable salt thereof, according to any preceding Embodiment, wherein R^{2A} is selected from the group consisting of —CH₃, which —CH₃ is unsubstituted or substituted with one, two or three J; and —CH₂CH₃, which —CH₂CH₃ is unsubstituted or substituted with one, two, three, four, or five J.

In another Embodiment (7.2a), the invention provides a compound of Formula (I), Formula (Ia), or Formula (Ib), or a pharmaceutically acceptable salt thereof, according to any preceding Embodiment, wherein R^{2A} is selected from the group consisting of —CH₃, which —CH₃ is unsubstituted or substituted with one, two or three J; and —CH₂CH₃, which —CH₂CH₃ is unsubstituted or substituted with one, two, three, four, or five J, which J is independently for each occurrence selected from the group consisting of —H, —F, —Cl, —CH₃; —CF₃; —OCH₃; and —OCF₃.

In another Embodiment (7.2b), the invention provides a compound of Formula (I), Formula (Ia), or Formula (Ib), or a pharmaceutically acceptable salt thereof, according to any preceding Embodiment, wherein R^{2A} is selected from the group consisting of —CH₃, which —CH₃ is unsubstituted or substituted with one, two or three J; and —CH₂CH₃, which —CH₂CH₃ is unsubstituted or substituted with one, two, three, four, or five J, which J is selected independently for each occurrence from the group consisting of —F to form, for example, —CF₃ or —CF₂CF₃; —Cl; —CH₃; —CF₃; and —OCH₃.

In another Embodiment (7.3), the invention provides a compound of Formula (I), Formula (Ia), or Formula (Ib), or a pharmaceutically acceptable salt thereof, according to any preceding Embodiment, wherein R^{2A} is selected from the group consisting of —CH₃ which —CH₃ is optionally substituted with one, two or three —F; and —CH₂CH₃ which —CH₂CH₃ is optionally substituted with one, two, three, four of five —F.

In another Embodiment (7.4), the invention provides a compound of Formula (I), Formula (Ia), or Formula (Ib), or a pharmaceutically acceptable salt thereof, according to any preceding Embodiment, wherein R^{2A} is —CH₃.

In another Embodiment (8), the invention provides a compound of Formula (I), Formula (Ia), or Formula (Ib), or a pharmaceutically acceptable salt thereof, according to any preceding Embodiment, wherein R^{2B} is selected from the group consisting of —C₁-C₆alkyl, more preferably —C₁-C₄alkyl, for example methyl, ethyl, n-propyl, or i-propyl; —OC₁-C₆alkyl, for example —OCH₃; —NH(C₁-C₆alkyl), for example NH(CH₃); —N(C₁-C₆alkyl)₂, for example N(CH₃)₂; C₃₋₅cycloalkyl, for example cyclopropyl or cyclobutyl; and a 4 to 7 membered heterocyclyl, which 4 to 7 membered heterocyclyl comprises one or two heteroatoms independently selected for each occurrence from the group consisting of N, O and S, for example oxetanyl, tetrahydrofuranyl, pyrrolidinyl, tetrahydrothiophenyl, pyrazolidinyl, imidazolidinyl, dioxolanyl, thiazolidinyl, isoxazolidinyl, tetrahydropyranyl, piperidinyl, piperizinyl or morpholinyl, and which R^{2B} is optionally substituted as defined for a compound of Formula (I), Formula (Ia), or Formula (Ib) respectively.

In another Embodiment (8.1), the invention provides a compound of Formula (I), Formula (Ia), or Formula (Ib), or a pharmaceutically acceptable salt thereof, according to any preceding Embodiment, wherein R^{2B} is selected from the group consisting of methyl, ethyl, n-propyl, i-propyl, —NH(CH₃), —N(CH₃)₂, cyclobutyl, and oxetanyl, and which R^{2B} is optionally substituted as defined for a compound of Formula (I), Formula (Ia), or Formula (Ib) respectively.

In another Embodiment (8.2), the invention provides a compound of Formula (I), Formula (Ia), or Formula (Ib), or a pharmaceutically acceptable salt thereof, according to any preceding Embodiment, wherein R^{2B} is selected from the group consisting of methyl, ethyl, n-propyl, i-propyl, —NH(CH₃), —N(CH₃)₂, cyclobutyl, and oxetanyl, and which R^{2B} is unsubstituted or substituted with one or two G, which G is independently selected for each occurrence from the group consisting of —OH to form, for example, —CH₂OH; halo, for example —F to form, for example 2,2-difluorocyclobutyl, or —Cl; —C₁-C₃alkyl, for example methyl to form, for example, 2,2-dimethylcyclobutyl; and —OC₁-C₃alkyl, for example —OCH₃, and which G is optionally further substituted as defined for a compound of Formula (I), for example G is —C₁-C₃alkyl substituted with one, two, three or four J, to form, for example, —CF₃.

In another Embodiment (8.3), the invention provides a compound of Formula (I), Formula (Ia), or Formula (Ib), or a pharmaceutically acceptable salt thereof, according to any preceding Embodiment, wherein R^{2B} is selected from the group consisting of methyl, —CH₂OH, ethyl, n-propyl, i-propyl, —NH(CH₃), —N(CH₃)₂, cyclobutyl, 2,2-difluorocyclobutyl, 2,2-dimethylcyclobutyl and oxetanyl.

In another Embodiment (8.4), the invention provides a compound of Formula (I), Formula (Ia), or Formula (Ib), or a pharmaceutically acceptable salt thereof, according to any preceding Embodiment, wherein R^{2B} is selected from the group consisting of methyl, ethyl, n-propyl, i-propyl, and —NH(CH₃).

In another Embodiment (9), the invention provides a compound of Formula (I), Formula (Ia), or Formula (Ib), or a pharmaceutically acceptable salt thereof, according to any preceding Embodiment, R^{2A} is —CH₃; and R^{2B} is selected from the group consisting of —C₁-C₆alkyl, more preferably —C₁-C₄alkyl, for example methyl, ethyl, n-propyl, or i-propyl; —OC₁-C₆alkyl, for example —OCH₃; —NH(C₁-C₆alkyl), for example —NH(CH₃); —N(C₁-C₆alkyl)₂, for example —N(CH₃)₂; —C₃₋₅cycloalkyl, for example cyclopropyl or cyclobutyl; and a 4 to 7 membered heterocyclyl, which 4 to 7 membered heterocyclyl comprises one or two heteroatoms independently selected for each occurrence from the group consisting of N, O and S, for example oxetanyl, tetrahydrofuranyl, pyrrolidinyl, tetrahydrothiophenyl, pyrazolidinyl, imidazolidinyl, dioxolanyl, thiazolidinyl, isoxazolidinyl, tetrahydropyranyl, piperidinyl, piperizinyl or morpholinyl, and which R^{2B} is optionally further substituted as defined for a compound of Formula (I), Formula (Ia), or Formula (Ib) respectively.

In another Embodiment (9.1), the invention provides a compound of Formula (I), Formula (Ia), or Formula (Ib), or a pharmaceutically acceptable salt thereof, according to any preceding Embodiment, R^{2A} is —CH₃; and wherein R^{2B} is selected from the group consisting of methyl, ethyl, n-propyl, i-propyl, —NH(CH₃), —N(CH₃)₂, cyclobutyl, and oxetanyl, and which R^{2B} is optionally substituted as defined for a compound of Formula (I), Formula (Ia), or Formula (Ib) respectively.

In another Embodiment (9.2), the invention provides a compound of Formula (I), Formula (Ia), or Formula (Ib), or a pharmaceutically acceptable salt thereof, according to any preceding Embodiment, R^{2A} is —CH₃; and wherein R^{2B} is selected from the group consisting of methyl, —CH₂OH, ethyl, n-propyl, i-propyl, —NH(CH₃), —N(CH₃)₂, cyclobutyl, 2,2-difluorocyclobutyl, 2,2-dimethylcyclobutyl and oxetanyl.

In another Embodiment (10), the invention provides a compound of Formula (I), Formula (Ia), or Formula (Ib), or a pharmaceutically acceptable salt thereof, according to any preceding Embodiment, wherein W is

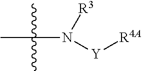

and where R³, Y and R^{4A} are as defined for a compound of Formula (I), Formula (Ia) or Formula (Ib) respectively.

In another Embodiment (10.1), the invention provides a compound of Formula (I), Formula (Ia), or Formula (Ib), or a pharmaceutically acceptable salt thereof, according to any preceding Embodiment, wherein W is

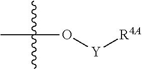

Y and where Y and R^{4A} are as defined for a compound of Formula (I), Formula (Ia) or Formula (Ib) respectively.

In another Embodiment (10.2), the invention provides a compound of Formula (I), Formula (Ia), or Formula (Ib), or a pharmaceutically acceptable salt thereof, according to any preceding Embodiment, wherein W is

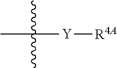

and where Y and R^{4A} are as defined for a compound of Formula (I), Formula (Ia) or Formula (Ib) respectively.

In another Embodiment (10.3), the invention provides a compound of Formula (I), Formula (Ia), or Formula (Ib), or a pharmaceutically acceptable salt thereof, according to any preceding Embodiment, wherein W is

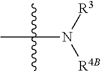

and where R³ and R^{4B} are as defined for a compound of Formula (I), Formula (Ia) or Formula (Ib) respectively.

In another Embodiment (10.4), the invention provides a compound of Formula (1), Formula (1a) or Formula (Ib), or a pharmaceutically acceptable salt thereof, according to any preceding Embodiment, wherein W is

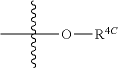

and where R^{4C} is defined for a compound of Formula (I), Formula (Ia) or Formula (Ib) respectively.

In another Embodiment (10.5), the invention provides a compound of Formula (1), Formula (1a) or Formula (Ib), or a pharmaceutically acceptable salt thereof, according to any preceding Embodiment, wherein W is a 4 to 7 membered heterocyclyl which said 4 to 7 membered heterocyclyl comprises one, two, three or four heteroatoms independently selected for each occurrence from the group consisting of N, O and S, and which W is optionally further substituted as defined for a compound of Formula (I), Formula (Ia) or Formula (Ib) respectively.

In another Embodiment (11), the invention provides a compound of Formula (I), Formula (Ia), or Formula (Ib), or a pharmaceutically acceptable salt thereof, according to any preceding Embodiment, wherein Y is selected from the group consisting of —CH₂— and —CH₂CH₂—, which Y is unsubstituted or optionally further substituted as defined for a compound of Formula (I), Formula (Ia) or Formula (Ib) respectively.

In another Embodiment (11.0), the invention provides a compound of Formula (I), Formula (Ia), or Formula (Ib), or a pharmaceutically acceptable salt thereof, according to any preceding Embodiment, wherein Y is selected from the group consisting of —CH₂— and —CH₂CH₂—, which Y is unsubstituted or optionally further substituted as defined for a compound of Formula (I), Formula (Ia) or Formula (Ib) respectively, which J is independently for each occurrence selected from the group consisting of —H, —F, —Cl, —CH₃; —CF₃; —OCH₃; and —OCF₃.

In another Embodiment (11.1), the invention provides a compound of Formula (I), Formula (Ia), or Formula (Ib), or a pharmaceutically acceptable salt thereof, according to any preceding Embodiment, wherein Y is —CH₂—.

In another Embodiment (11.2), the invention provides a compound of Formula (I), Formula (Ia), or Formula (Ib), or a pharmaceutically acceptable salt thereof, according to any preceding Embodiment, wherein Y is —CH₂CH₂—.

In another Embodiment (11.3), the invention provides a compound of Formula (I), Formula (Ia), or Formula (Ib), or a pharmaceutically acceptable salt thereof, according to any preceding Embodiment, wherein Y is —CH(CH₃)—.

In another Embodiment (11.4), the invention provides a compound of Formula (I), Formula (Ia), or Formula (Ib), or a pharmaceutically acceptable salt thereof, according to any preceding Embodiment, wherein Y is —CH(CF₃)—.

In another Embodiment (11.5), the invention provides a compound of Formula (I), Formula (Ia), or Formula (Ib), or a pharmaceutically acceptable salt thereof, according to any preceding Embodiment, wherein Y is —CH(CH₃)CH₂—.

In another Embodiment (11.6), the invention provides a compound of Formula (I), Formula (Ia), or Formula (Ib), or a pharmaceutically acceptable salt thereof, according to any preceding Embodiment, wherein Y is —CH(CF₃)CH₂—.

In another Embodiment (11.7), the invention provides a compound of Formula (I), Formula (Ia), or Formula (Ib), or a pharmaceutically acceptable salt thereof, according to any preceding Embodiment, wherein Y is —CH₂CH(CH₃)—.

In another Embodiment (11.8), the invention provides a compound of Formula (I), Formula (Ia), or Formula (Ib), or a pharmaceutically acceptable salt thereof, according to any preceding Embodiment, wherein Y is —CH₂CH(CF₃)—.

In another Embodiment (12), the invention provides a compound of Formula (I), Formula (Ia), or Formula (Ib), or a pharmaceutically acceptable salt thereof, according to any preceding Embodiment, wherein R³ is —H.

In another Embodiment (12.1), the invention provides a compound of Formula (I), Formula (Ia), or Formula (Ib), or a pharmaceutically acceptable salt thereof, according to any preceding Embodiment, wherein R³ is —CH₃, which R³ unsubstituted or optionally further substituted as defined for a compound of Formula (I), Formula (Ia) or Formula (Ib) respectively.

In another Embodiment (12.2), the invention provides a compound of Formula (I), Formula (Ia), or Formula (Ib), or a pharmaceutically acceptable salt thereof, according to any preceding Embodiment, wherein R³ is —CH₃, which R³ is unsubstituted or optionally substituted with one, two or three J, which J is independently selected from the group consisting of —H, —F, —CH₃; and —CF₃.

In another Embodiment (12.3), the invention provides a compound of Formula (I), Formula (Ia), or Formula (Ib), or a pharmaceutically acceptable salt thereof, according to any preceding Embodiment, wherein R³ is —CH₃.

In another Embodiment (13), the invention provides a compound of Formula (I), Formula (Ia), or Formula (Ib), or a pharmaceutically acceptable salt thereof, according to any preceding Embodiment, wherein R^{4A} is selected from the group consisting of —C₁-C₆alkyl, preferably —C₁-C₄alkyl, for example —CH₃; —CO₂H; —C(O)OC₁-C₆alkyl; —C(O)NH₂; —C(O)NH(C₁-C₆alkyl), for example —C(O)NHCH₃; —C(O)N(C₁-C₆alkyl)₂; —C(O)NHSO₂C₁-C₃alkyl; 4 to 7 membered heterocyclyl which said 4 to 7 membered heterocyclyl comprises one, two, three or four heteroatoms independently selected for each occurrence from the group consisting of N, O and S; and 5 to 6 membered heteroaryl, which said 5 to 6 membered heteroaryl ring comprises one, two, three or four heteroatoms independently selected for each occurrence from the group consisting of N, O and S; and which R^{4A} is optionally further substituted as defined for a compound of Formula (I), Formula (Ia) or Formula (Ib) respectively.

In another Embodiment (13.1), the invention provides a compound of Formula (I), Formula (Ia), or Formula (Ib), or a pharmaceutically acceptable salt thereof, according to any preceding Embodiment, wherein R^{4A} is selected from the group consisting of —C₁-C₆alkyl, preferably —C₁-C₄alkyl, for example —CH₃; —CO₂H; —C(O)OC₁-C₆alkyl for example —C(O)OCH₃ and —C(O)OCH₂CH₃; —C(O)NH(C₁-C₆alkyl), for example —C(O)NHCH₃; —C(O)NHSO₂C₁-C₃alkyl for example —C(O)NHSO₂CH₃; 4 to 7 membered heterocyclyl which said 4 to 7 membered heterocyclyl comprises one, two, three or four heteroatoms independently selected for each occurrence from the group consisting of N, O and S, for example morpholinyl, pyranyl, piperidinyl, or piperazinyl; and 5 to 6 membered heteroaryl, which said 5 to 6 membered heteroaryl ring comprises one, two, three or four heteroatoms independently selected for each occurrence from the group consisting of N, O and S, for example tetrazolyl; and which R^{4A} is optionally further substituted as defined for a compound of Formula (I), Formula (Ia) or Formula (Ib) respectively.

In another Embodiment (13.2), the invention provides a compound of Formula (I), Formula (Ia), or Formula (Ib), or a pharmaceutically acceptable salt thereof, according to any preceding Embodiment, wherein R^{4A} is selected from the group consisting of —CH₃; —CO₂H; —C(O)OCH₃; —C(O)OCH₂CH₃; —C(O)NHCH₃; —C(O)NHSO₂CH₃; morpholinyl; and tetrazolyl and which R^{4A} is optionally further substituted as defined for a compound of Formula (I), Formula (Ia) or Formula (Ib) respectively.

In another Embodiment (13.3), the invention provides a compound of Formula (I), Formula (Ia), or Formula (Ib), or a pharmaceutically acceptable salt thereof, according to any preceding Embodiment, wherein R^{4A} is selected from the group consisting of —CH₃; —CO₂H; —C(O)OCH₃; —C(O)OCH₂CH₃; —C(O)NHCH₃; —C(O)NHSO₂CH₃; morpholinyl; and tetrazolyl and which R^{4A} is optionally further substituted with G as defined for a compound of Formula (I), Formula (Ia) or Formula (Ib) respectively, which G is selected, independently for each occurrence, from the group consisting of —OH; —F; —Cl; —CH₃; —OCH₃; and —CF₃.

In another Embodiment (13.4), the invention provides a compound of Formula (I), Formula (Ia), or Formula (Ib), or a pharmaceutically acceptable salt thereof, according to any preceding Embodiment, wherein R^{4A} is selected from the group consisting of —CH₃; —CO₂H; —C(O)OCH₃; —C(O)OCH₂CH₃; and —C(O)NHCH₃.

In another Embodiment (13.5), the invention provides a compound of Formula (I), Formula (Ia), or Formula (Ib), or a pharmaceutically acceptable salt thereof, according to any preceding Embodiment, wherein R^{4A} is selected from the group consisting of —CH₃; —CO₂H; and —C(O)NHCH₃.

In another Embodiment (14), the invention provides a compound of Formula (I), Formula (Ia), or Formula (Ib), or a pharmaceutically acceptable salt thereof, according to any preceding Embodiment, wherein R^{4B} is selected from the group consisting of —C₁-C₆alkyl, preferably —C₁-C₄alkyl; —C(O)C₁-C₆alkyl; —C(O)OC₁-C₆alkyl; —C(O)NH₂; —C(O)NH(C₁-C₆alkyl); —C(O)N(C₁-C₆alkyl)₂; —C(O)NHSO₂C₁-C₃alkyl; 4 to 7 membered heterocyclyl which said 4 to 7 membered heterocyclyl comprises one, two, three or four heteroatoms independently selected for each occurrence from the group consisting of N, O and S; and 5 to 6 membered heteroaryl, which said 5 to 6 membered heteroaryl ring comprises one, two, three or four heteroatoms independently selected for each occurrence from the group consisting of N, O and S; and which R^{4B} is optionally further substituted as defined for a compound of Formula (I), Formula (Ia) or Formula (Ib) respectively.

In another Embodiment (14.1), the invention provides a compound of Formula (I), Formula (Ia), or Formula (Ib), or a pharmaceutically acceptable salt thereof, according to any preceding Embodiment, wherein R^{4B} is selected from the group consisting of —C₁-C₆alkyl, preferably —C₁-C₄alkyl, for example CH₃; —C(O)C₁-C₆alkyl, for example —C(O)CH₃; —C(O)NH(C₁-C₆alkyl), for example —C(O)NHCH₃; —C(O)NHSO₂C₁-C₃alkyl for example —C(O)NHSO₂CH₃; 4 to 7 membered heterocyclyl which said 4 to 7 membered heterocyclyl comprises one, two, three or four heteroatoms independently selected for each occurrence from the group consisting of N, O and S, for example morpholinyl, pyranyl, piperidinyl, or piperazinyl; and 5 to 6 membered heteroaryl, which said 5 to 6 membered heteroaryl ring comprises one, two, three or four heteroatoms independently selected for each occurrence from the group consisting of N, O and S, for example tetrazolyl; and which R^{4B} is optionally further substituted as defined for a compound of Formula (I), Formula (Ia) or Formula (Ib) respectively.

In another Embodiment (14.2), the invention provides a compound of Formula (I), Formula (Ia), or Formula (Ib), or a pharmaceutically acceptable salt thereof, according to any preceding Embodiment, wherein R^{4B} is selected from the group consisting of —CH₃; —C(O)CH₃; —C(O)NHCH₃; —C(O)NHSO₂CH₃; morpholinyl; and tetrazolyl; and which R^{4B} is optionally further substituted as defined for a compound of Formula (I), Formula (Ia) or Formula (Ib) respectively.

In another Embodiment (14.3), the invention provides a compound of Formula (I), Formula (Ia), or Formula (Ib), or a pharmaceutically acceptable salt thereof, according to any preceding Embodiment, wherein R^{4B} is selected from the group consisting of —CH₃; —C(O)CH₃; —C(O)NHCH₃; —C(O)NHSO₂CH₃; morpholinyl; and tetrazolyl; and which R^{4B} is optionally further substituted with G as defined for a compound of Formula (I), Formula (Ia) or Formula (Ib) respectively, which G is selected, independently for each occurrence, from the group consisting of —OH; —F; —Cl; —CH₃; —OCH₃; and —CF₃.

In another Embodiment (14.4), the invention provides a compound of Formula (I), Formula (Ia), or Formula (Ib), or a pharmaceutically acceptable salt thereof, according to any preceding Embodiment, wherein R^{4B} is selected from the group consisting of —CH₃; —C(O)CH₃; —C(O)NHCH₃; —C(O)NHSO₂CH₃; morpholinyl; and tetrazolyl.

In another Embodiment (15), the invention provides a compound of Formula (I), Formula (Ia), or Formula (Ib), or a pharmaceutically acceptable salt thereof, according to any preceding Embodiment, wherein R^{4C} is selected from the group consisting of —C₁-C₆alkyl, preferably —C₁-C₄alkyl; 4 to 7 membered heterocyclyl which said 4 to 7 membered heterocyclyl comprises one, two, three or four heteroatoms independently selected for each occurrence from the group consisting of N, O and S; and 5 to 6 membered heteroaryl, which said 5 to 6 membered heteroaryl ring comprises one, two, three or four heteroatoms independently selected for each occurrence from the group consisting of N, O and S; and which R^{4C} is optionally further substituted as defined for a compound of Formula (I), Formula (Ia) or Formula (Ib) respectively.

In another Embodiment (15.1), the invention provides a compound of Formula (I), Formula (Ia), or Formula (Ib), or a pharmaceutically acceptable salt thereof, according to any preceding Embodiment, wherein R^{4C} is selected from the group consisting of —C₁-C₆alkyl, preferably —C₁-C₄alkyl, for example CH₃; 4 to 7 membered heterocyclyl which said 4 to 7 membered heterocyclyl comprises one, two, three or four heteroatoms independently selected for each occurrence from the group consisting of N, O and S, for example morpholinyl, pyranyl, piperidinyl, or piperazinyl; and 5 to 6 membered heteroaryl, which said 5 to 6 membered heteroaryl ring comprises one, two, three or four heteroatoms independently selected for each occurrence from the group consisting of N, O and S, for example tetrazolyl; and which R^{4C} is optionally further substituted as defined for a compound of Formula (I), Formula (Ia) or Formula (Ib) respectively.

In another Embodiment (15.2), the invention provides a compound of Formula (I), Formula (Ia), or Formula (Ib), or a pharmaceutically acceptable salt thereof, according to any preceding Embodiment, wherein R^{4C} is selected from the group consisting of —CH₃; morpholinyl; and tetrazolyl; and which R^{4C} is optionally further substituted as defined for a compound of Formula (I), Formula (Ia) or Formula (Ib) respectively.

In another Embodiment (15.3), the invention provides a compound of Formula (I), Formula (Ia), or Formula (Ib), or a pharmaceutically acceptable salt thereof, according to any preceding Embodiment, wherein R^{4C} is selected from the group consisting of —CH₃; morpholinyl; and tetrazolyl; and which R^{4C} is optionally further substituted with G as defined for a compound of Formula (I), Formula (Ia) or Formula (Ib) respectively, which G is selected, independently for each occurrence, from the group consisting of —OH; —F; —Cl; —CH₃; —OCH₃; and —CF₃.

In another Embodiment (15.4), the invention provides a compound of Formula (I), Formula (Ia), or Formula (Ib), or a pharmaceutically acceptable salt thereof, according to any preceding Embodiment, wherein R^{4C} is selected from the group consisting of —CH₃; morpholinyl; and tetrazolyl.

In another Embodiment (15.5), the invention provides a compound of Formula (I), Formula (Ia), or Formula (Ib), or a pharmaceutically acceptable salt thereof, according to any preceding Embodiment, wherein R^{4C} is —CH₃.

In another Embodiment (16), the invention provides a compound of Formula (I), Formula (Ia), or Formula (Ib), or a pharmaceutically acceptable salt thereof, according to any preceding Embodiment, wherein W is

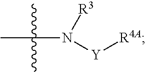

R³ is —H; and where Y and R^{4A} are as defined for a compound of Formula (I), Formula (Ia) or Formula (Ib) respectively.

In another Embodiment (16.1), the invention provides a compound of Formula (I), Formula (Ia), or Formula (Ib), or a pharmaceutically acceptable salt thereof, according to any preceding Embodiment, wherein W is

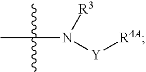

R³ is —H; Y is —CH₂—, which Y is optionally further substituted as defined for a compound Formula (I), Formula (Ia) or Formula (Ib) respectively; and R^{4A} is as defined for a compound of Formula (I), Formula (Ia) or Formula (Ib) respectively.

In another Embodiment (16.2), the invention provides a compound of Formula (I), Formula (Ia), or Formula (Ib), or a pharmaceutically acceptable salt thereof, according to any preceding Embodiment, wherein W is

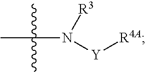

R³ is —H; Y is —CH₂CH₂—, which Y is optionally further substituted as defined for a compound Formula (I), Formula (Ia) or Formula (Ib) respectively; and R^{4A} is as defined for a compound of Formula (I), Formula (Ia) or Formula (Ib) respectively.

In another Embodiment (16.3), the invention provides a compound of Formula (I), Formula (Ia), or Formula (Ib), or a pharmaceutically acceptable salt thereof, according to any preceding Embodiment, wherein W is

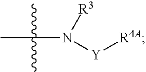

R³ is —H; Y is —CH₂—, which Y is optionally further substituted as defined for a compound Formula (I), Formula (Ia) or Formula (Ib) respectively; and R^{4A} is —CH₃; —CO₂H; —C(O)NHCH₃; —C(O)NHSO₂CH₃; morpholinyl; and tetrazolyl.

In another Embodiment (16.4), the invention provides a compound of Formula (I), Formula (Ia), or Formula (Ib), or a pharmaceutically acceptable salt thereof, according to any preceding Embodiment, wherein W is

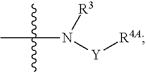

R³ is —H; Y is —CH₂CH₂—, which Y is optionally further substituted as defined for a compound Formula (I), Formula (Ia) or Formula (Ib) respectively; and R^{4A} is —CH₃; —CO₂H; —C(O)NHCH₃; —C(O)NHSO₂CH₃; morpholinyl; and tetrazolyl.

In another Embodiment (17), the invention provides a compound of Formula (I), Formula (Ia), or Formula (Ib), or a pharmaceutically acceptable salt thereof, according to any preceding Embodiment, wherein E is independently selected for each occurrence from the group consisting of —OH; halo; —C₁-C₃alkyl; —OC₁-C₃alkyl; —C(O)C₁-C₃alkyl; —C(O)OC₁-C₃alkyl; —NH₂; —NH(C₁-C₃alkyl); —N(C₁-C₃alkyl)₂; —C₃-C₇cycloalkyl; and phenyl, which E is optionally further substituted as defined for a compound of Formula (I), Formula (Ia) or Formula (Ib) respectively.

In another Embodiment (17.1), the invention provides a compound of Formula (I), Formula (Ia), or Formula (Ib), or a pharmaceutically acceptable salt thereof, according to any preceding Embodiment, wherein E is independently selected for each occurrence from the group consisting of —OH; —F; —Cl; —CH₃; —OCH₃; and —CF₃.

In another Embodiment (18), the invention provides a compound of Formula (I), Formula (Ia), or Formula (Ib), or a pharmaceutically acceptable salt thereof, according to any preceding Embodiment, wherein G is independently selected for each occurrence from the group consisting of —OH; halo; —C₁-C₃alkyl; —OC₁-C₃alkyl; —C(O)C₁-C₃alkyl; —C(O)OC₁-C₃alkyl; —NH₂; —NH(C₁-C₃alkyl); and —N(C₁-C₃alkyl)₂, which G is optionally further substituted as defined for a compound of Formula (I), Formula (Ia) or Formula (Ib) respectively.

In another Embodiment (18.1), the invention provides a compound of Formula (I), Formula (Ia), or Formula (Ib), or a pharmaceutically acceptable salt thereof, according to any preceding Embodiment, wherein G is independently selected for each occurrence from the group consisting of —OH; —F; —Cl; —CH₃; —OCH₃; and —CF₃.

In another Embodiment (19), the invention provides a compound of Formula (I), Formula (Ia), or Formula (Ib), or a pharmaceutically acceptable salt thereof, according to any preceding Embodiment, wherein J is independently selected for each occurrence from the group consisting of —H, —OH, —F, —Cl, —CH₃, —CF₃, —CH₂OH, —OCH₃, —OCF₃, and —OCF₂H.

In another Embodiment (19.1), the invention provides a compound of Formula (I), Formula (Ia), or Formula (Ib), or a pharmaceutically acceptable salt thereof, according to any preceding Embodiment, wherein J is independently selected for each occurrence from the group consisting of —H; —F; —Cl; —CH₃; —CF₃; —OCH₃; and —OCF₃.

In another Embodiment (19.1a), the invention provides a compound of Formula (I), Formula (Ia), or Formula (Ib), or a pharmaceutically acceptable salt thereof, according to any preceding Embodiment, wherein J is independently selected for each occurrence from the group consisting of —H; —F; —CH₃; and —CF₃.

In another Embodiment (19.2), the invention provides a compound of Formula (I), Formula (Ia), or Formula (Ib), or a pharmaceutically acceptable salt thereof, according to any preceding Embodiment, wherein R¹⁰ is independently selected for each occurrence from the group consisting of —H, —OH, —F, —Cl, —CH₃, —CF₃, —CH₂OH, —OCH₃, —OCF₃, and —OCF₂H.

In another Embodiment (19.3), the invention provides a compound of Formula (I), Formula (Ia), or Formula (Ib), or a pharmaceutically acceptable salt thereof, according to any preceding Embodiment, wherein R¹⁰ is independently selected for each occurrence from the group consisting of —H, —F, —Cl, —CH₃, —CF₃, and —OCF₃.

In another Embodiment (19.4), the invention provides a compound of Formula (I), Formula (Ia), or Formula (Ib), or a pharmaceutically acceptable salt thereof, according to any preceding Embodiment, wherein R¹⁰ is —H.

In another Embodiment (50), the invention provides a compound of Formula (I), or a pharmaceutically acceptable salt thereof, according to any preceding Embodiment, wherein

R¹ is

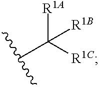

R^{1A} is selected from the group consisting of methyl, ethyl, n-propyl, phenyl, and pyridyl, which R^{1A} is optionally substituted as defined for a compound of Formula (I);

R^{1B} is selected from the group consisting of —H, methyl, ethyl, n-propyl and i-propyl, which R^{1B} is optionally substituted as defined for a compound of Formula (I);

R^{1C} is —H;

R^{2A} is selected from the group consisting of —CH₃, and —CH₂CH₃, which R^{2A} is optionally substituted as defined for a compound of Formula (I);

R^{2B} is selected from the group consisting of methyl, ethyl, n-propyl, i-propyl, and —NH(CH₃);

W is

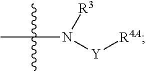

Y is selected from the group consisting of —CH₂— and —CH₂CH₂—, which Y is unsubstituted or optionally further substituted as defined for a compound of Formula (I);

R³ is —H;

R^{4A} is selected from the group consisting of —CH₃; —CO₂H; and —C(O)NHCH₃;

R¹⁰ is —H;

and where E, G and J are all defined as for a compound of Formula (I).

In another Embodiment (50.1), the invention provides a compound of Embodiment (50), or a pharmaceutically acceptable salt thereof, wherein

E is independently selected for each occurrence from the group consisting of —OH; —F; —Cl; —CH₃; —OCH₃; and —CF₃;

G is independently selected for each occurrence from the group consisting of —OH; —F; —Cl; —CH₃; —OCH₃; and —CF₃; and

J is independently selected for each occurrence from the group consisting of —H, —F, —Cl, —CH₃; —CF₃; —OCH₃; and —OCF₃.

In another Embodiment (50.2), the invention provides a compound of Formula (I), or a pharmaceutically acceptable salt thereof, according to any preceding Embodiment, wherein

R¹ is

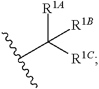

R^{1A} is selected from the group consisting of —CH₂OCH₃; phenyl; methoxyphenyl; and pyridyl;

R^{1B} is selected from the group consisting of methyl, ethyl, n-propyl, i-propyl, and —CH₂OCH₃;

R^{1C} is —H;

R^{2A} is selected from the group consisting of —CH₃;

R^{2B} is selected from the group consisting of methyl, ethyl, n-propyl, i-propyl, and —NH(CH₃);

W is

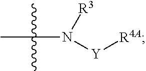

Y is selected from the group consisting of —CH₂— and —CH₂CH₂—;

R³ is —H;

R^{4A} is selected from the group consisting of —CH₃; —CO₂H; and —C(O)NHCH₃; and

R¹⁰ is —H.

The present invention also relates to a pharmaceutical composition comprising a compound of Formula (I), or a pharmaceutically acceptable salt thereof, and a pharmaceutically acceptable excipient.

The present invention also relates to a method of treating a disease or a disorder in a patient, comprising administering to a patient in need thereof a therapeutically effective amount of a compound of Formula (I), or a pharmaceutically acceptable salt thereof or a pharmaceutical composition comprising a therapeutically effective amount of a compound of Formula (I), or a pharmaceutically acceptable salt thereof.

In another Embodiment, the present invention further provides a method of inhibiting a BET family bromodomain in a cell, comprising contacting the cell with a therapeutically effective amount of a compound of Formula (I), or a pharmaceutically acceptable salt thereof, or a pharmaceutical composition comprising a therapeutically effective amount of a compound of Formula (I), or a pharmaceutically acceptable salt thereof.

This invention also relates to a compound of Formula II:

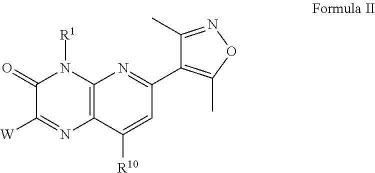

or a pharmaceutically acceptable salt thereof, wherein
R¹ is selected from the group consisting of:
(i) —C₃-C₇cycloalkyl optionally substituted with one, two, three or four E;
(ii) 4 to 7 membered heterocyclyl optionally substituted with one, two, three or four E, which said 4 to 7 membered heterocyclyl comprises one or two heteroatoms independently selected for each occurrence from the group consisting of N, O and S; and
(iii)

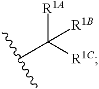

R^{1A} is selected from the group consisting of
   (i) —C₁-C₆alkyl optionally substituted with one, two, three, four, five or six E;
   (ii) —C₃-C₇cycloalkyl optionally substituted with one, two, three, four or five E;
   (iii) phenyl optionally substituted with one, two, three, four or five E;
   (iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
   (v) 5 to 6 membered heteroaryl optionally substituted with one, two, three, four or five E, which said 5 to 6 membered heteroaryl comprises one, two or three heteroatoms independently selected for each occurrence from the group consisting of N, O and S;
R^{1B} is selected from the group consisting of
   (i) —H; and
   (ii) —C₁-C₆alkyl optionally substituted with one, two, three, four, five or six E;
R^{1C} is selected from the group consisting of
   (i) —H;
   (ii) —CH₃ optionally substituted with one, two, or three J;
   (iii) —CH₂CH₃ optionally substituted with one, two, three, four or five J;
   (iv) —CH₂CH₂CH₃ optionally substituted with one, two, three, four, five, six or seven J; and
   (v) —CH(CH₃)₂ optionally substituted with one, two, three, four, five, six or seven J;
W is selected from the group consisting of:
   (i)

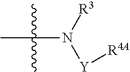

(ii)

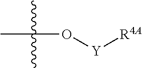

(iii)

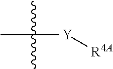

(iv)

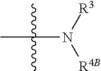

(v)

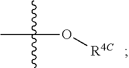

and
   (vi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S;
Y is selected from the group consisting of:
   (i) —CH₂— optionally substituted with one or two J;
   (ii) —(CH₂)₂— optionally substituted with one, two, three or four J;
   (iii) —(CH₂)₃— optionally substituted with one, two, three, four, five or six J; and
   (iv) —(CH₂)₄— optionally substituted with one, two, three, four, five, six, seven or eight J;
R³ is selected from the group consisting of:
   (i) —H;
   (ii) —CH₃ optionally substituted with one, two, or three J;
   (iii) —CH₂CH₃ optionally substituted with one, two, three, four or five J;
   (iv) —CH₂CH₂CH₃ optionally substituted with one, two, three, four, five, six or seven J; and
   (v) CH(CH₃)₂ optionally substituted with one, two, three, four, five, six or seven J;
R^{4A} is selected from the group consisting of
   (i) —H;
   (ii) —C₁-C₆alkyl optionally substituted with one, two, three or four G;
   (iii) —CO₂H;
   (iv) —C(O)C₁-C₆alkyl optionally substituted with one, two, three or four G;
   (v) —C(O)OC₁-C₆alkyl optionally substituted with one, two, three or four G;
   (vi) —C(O)NH₂;
   (vii) —C(O)NH(C₁-C₆alkyl) optionally substituted with one, two, three or four G;
   (viii) —C(O)N(C₁-C₆alkyl)₂ optionally substituted with one, two, three or four G;
   (ix) —C(O)NHSO₂C₁-C₃alkyl optionally substituted with one, two, three or four G;
   (x) —NH(C₁-C₃alkyl) optionally substituted with one, two, three or four G;
   (xi) —N(C₁-C₃alkyl)₂ optionally substituted with one, two, three or four G;
   (xii) —NHC(O)C₁-C₃alkyl optionally substituted with one, two, three or four G;
   (xiii) —N(C₁-C₃alkyl)C(O)C₁-C₃alkyl optionally substituted with one, two, three or four G;
   (xiv) —NHSO₂C₁-C₃alkyl optionally substituted with one, two, three or four G;
   (xv) —N(C₁-C₃alkyl)SO₂C₁-C₃alkyl optionally substituted with one, two, three or four G;
   (xvi) —SO₂NH₂;
   (xvii) —SO₂NH(C₁-C₃alkyl) optionally substituted with one, two, three or four G;
   (xviii) —SO₂N(C₁-C₃alkyl)₂ optionally substituted with one, two, three or four G;
   (xix) —C₃-C₇cycloalkyl optionally substituted with one, two, three or four G;
   (xx) phenyl optionally substituted with one, two, three or four G;
   (xxi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
   (xxii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4B} is selected from the group consisting of
   (i) —H;
   (ii) —C₁-C₆alkyl optionally substituted with one, two, three or four G;
   (iii) —C(O)C₁-C₆alkyl optionally substituted with one, two, three or four G;
   (iv) —C(O)OC₁-C₆alkyl optionally substituted with one, two, three or four G;
   (v) —C(O)NH₂;
   (vi) —C(O)NH(C₁-C₆alkyl) optionally substituted with one, two, three or four G;
   (vii) —C(O)N(C₁-C₆alkyl)₂ optionally substituted with one, two, three or four G;
   (viii) —C(O)NHSO₂C₁-C₃alkyl optionally substituted with one, two, three or four G;
   (ix) —C₃-C₇cycloalkyl optionally substituted with one, two, three or four G;
   (x) phenyl optionally substituted with one, two, three or four G;
   (xi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
   (xii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4C} is selected from the group consisting of
   (i) —H;
   (ii) —C₁-C₆alkyl optionally substituted with one, two, three or four G;
   (iii) —C₃-C₇cycloalkyl optionally substituted with one, two, three or four G;
   (iv) phenyl optionally substituted with one, two, three or four G;
   (v) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
   (vi) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R¹⁰ is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂;
E is independently selected for each occurrence from the group consisting of:
(i) —OH;
(ii) —CN;
(iii) —CO₂H;
(iv) —C(O)H;
(v) halo;
(vi) —C₁-C₃alkyl optionally substituted with one, two, three or four J;
(vii) —C₁-C₃alkylCO₂H which —C₁-C₃alkyl is optionally substituted with one, two, three or four J;
(viii) —C₃-C₇cycloalkyl optionally substituted with one, two, three, four, five or six J;
(ix) —C₁-C₃alkylC₃-C₆cycloalkyl optionally substituted with one, two, three, four, five or six J;
(x) —OC₁-C₃alkyl, optionally substituted with one, two, three or four J;
(xi) —OC₃-C₇cycloalkyl optionally substituted with one, two, three, four, five or six J;
(xii) —OC₁-C₃alkylC₃-C₇cycloalkyl optionally substituted with one, two, three, four, five or six J;
(xiii) —SC₁-C₃alkyl, optionally substituted with one, two, three or four J;
(xiv) —SC₃-C₇cycloalkyl optionally substituted with one, two, three, four, five or six J;
(xv) —SC₁-C₃alkylC₃-C₇cycloalkyl optionally substituted with one, two, three, four, five or six J;
(xvi) —C(O)C₁-C₃alkyl, optionally substituted with one, two, three or four J;
(xvii) —C(O)OC₁-C₃alkyl, optionally substituted with one, two, three or four J;
(xviii) —NH₂;
(xix) —NH(C₁-C₃alkyl) optionally substituted with one, two, three or four J;
(xx) —N(C₁-C₃alkyl)₂ which —C₁-C₃alkyl is, independently for each occurrence, optionally substituted with one, two, three or four J;
(xxi) —C(O)NH₂;
(xxii) —C(O)NHC₁-C₃alkyl, optionally substituted with one, two, three or four J;
(xxiii) —C(O)N(C₁-C₃alkyl)₂, which —C₁-C₃alkyl is, independently for each occurrence, optionally substituted with one, two, three or four J;
(xxiv) —NHC(O)C₁-C₃alkyl, optionally substituted with one, two, three or four J;
(xxv) —SO₂(C₁-C₃alkyl), optionally substituted with one, two, three or four J;
(xxvi) —SO₂NH(C₁-C₃alkyl), optionally substituted with one, two, three or four J;
(xxvii) —NHSO₂(C₁-C₃alkyl), optionally substituted with one, two, three or four J; and
(xxviii) phenyl optionally substituted with one, two, three, or four J;
G is independently selected for each occurrence from the group consisting of
(i) —OH
(ii) —CN;
(iii) —CO₂H;
(iv) —C(O)H;
(v) halo;
(vi) —C₁-C₃alkyl, optionally substituted with one, two, three or four J;
(vii) —C₁-C₃alkylCO₂H, which —C₁-C₃alkyl is optionally substituted with one, two, three or four J;
(viii) —C₁-C₃alkylC₃-C₆cycloalkyl optionally substituted with one, two, three, four, five or six J;
(ix) —OC₁-C₃alkyl, optionally substituted with one, two, three or four J;
(x) —OC₁-C₃alkylC₃-C₆cycloalkyl optionally substituted with one, two, three, four, five or six J;
(xi) —SC₁-C₃alkyl, optionally substituted with one, two, three or four J;
(xii) —SC₁-C₃alkylC₃-C₆cycloalkyl optionally substituted with one, two, three, four, five or six J;
(xiii) —C(O)C₁-C₃alkyl, optionally substituted with one, two, three or four J;
(xiv) —C(O)OC₁-C₃alkyl, optionally substituted with one, two, three or four J;
(xv) —NH₂;
(xvi) —NH(C₁-C₃alkyl), optionally substituted with one, two, three or four J;
(xvii) —N(C₁-C₃alkyl)₂, which —C₁-C₃alkyl is, independently for each occurrence, optionally substituted with one, two, three or four J;
(xviii) —C(O)NH₂;
(xix) —C(O)NHC₁-C₃alkyl, optionally substituted with one, two, three or four J;
(xx) —C(O)N(C₁-C₃alkyl)₂, which —C₁-C₃alkyl is, independently for each occurrence, optionally substituted with one, two, three or four J;
(xxi) —NHC(O)C₁-C₃alkyl, optionally substituted with one, two, three or four J;
(xxii) —SO₂(C₁-C₃alkyl), optionally substituted with one, two, three or four J;
(xxiii) —SO₂NH(C₁-C₃alkyl), optionally substituted with one, two, three or four J; and
(xxiv) —NHSO₂(C₁-C₃alkyl) optionally substituted with one, two, three or four J; and
J is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂.

In another Embodiment (20.0), the invention provides a compound of Formula (II′):

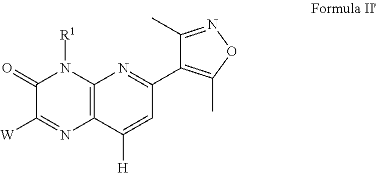

or a pharmaceutically acceptable salt thereof, and wherein R¹, R^{1A}, R^{1B}, R^{1C}, W, Y, R³, R^{4A}, R^{4B}, R^{4C}, E, G, and J are all defined as for a compound of Formula (II).

In one Embodiment (20), the invention provides a compound of Formula (II), or Embodiment (20.0), or a pharmaceutically acceptable salt thereof, wherein R¹ is —C₃-C₇cycloalkyl optionally substituted as defined for a compound of Formula (II).

In another Embodiment (20.1), the invention provides a compound of Formula (II), Embodiment (20.0), or Embodiment (20), or a pharmaceutically acceptable salt thereof, wherein R¹ is —C₃-C₇cycloalkyl selected from the group consisting of cyclopropyl, cyclobutyl, cyclopentyl, and cyclohexyl, preferably cyclopropyl, cyclobutyl and cyclopentyl, which cyclopropyl, cyclobutyl, cyclopentyl or cyclohexyl is optionally substituted as defined for a compound of Formula (II).

In another Embodiment (20.2), the invention provides a compound of Formula (II), Embodiment (20.0), Embodiment (20), or Embodiment (20.1), or a pharmaceutically acceptable salt thereof, wherein R¹ is selected from the group consisting of cyclopropyl, cyclobutyl, cyclopentyl and cyclohexyl, preferably cyclopropyl, cyclobutyl and cyclopentyl, which cyclopropyl, cyclobutyl, cyclopentyl or cyclohexyl is unsubstituted or substituted with one, two, three of four E, which E is independently selected for each occurrence from the group consisting of halo, for example —F or —Cl; —C₁-C₃alkyl, for example methyl or ethyl; —OC₁-C₃alkyl; —C₃-C₇cycloalkyl, for example cyclopentyl; and phenyl, and which E is optionally further substituted as defined for a compound of Formula (II), for example E is —C₁-C₃alkyl substituted with one, two, three or four J, to form, for example, —CF₃.

In another Embodiment (20.3), the invention provides a compound of Formula (II), Embodiment (20.0), Embodiment (20), Embodiment (20.1), or Embodiment (20.2), or a pharmaceutically acceptable salt thereof, wherein R¹ is selected from the group consisting of cyclopropyl, cyclobutyl, cyclopentyl and cyclohexyl, preferably cyclopropyl, cyclobutyl and cyclopentyl, which cyclopropyl, cyclobutyl, cyclopentyl or cyclohexyl is unsubstituted or substituted with one or two E, which E is independently selected for each occurrence from the group consisting of —C₁-C₃alkyl, for example ethyl; —C₃-C₇cycloalkyl, for example cyclopentyl; and phenyl, and which E is optionally further substituted as defined for a compound of Formula (II).

In another Embodiment (20.4), the invention provides a compound of Formula (II), Embodiment (20.0), Embodiment (20), Embodiment (20.1), Embodiment (20.2), or Embodiment (20.3), or a pharmaceutically acceptable salt thereof, wherein R¹ is selected from the group consisting of cyclopropyl, which cyclopropyl is unsubstituted or substituted with one E, which E is cyclopentyl; cyclobutyl, which cyclobutyl is unsubstituted or substituted with one E, which E is phenyl; and cyclopentyl, which cyclopentyl is unsubstituted or substituted with two E, which E are both ethyl.

In another Embodiment (21), the invention provides a compound of Formula (II), or Embodiment (20.0), or a pharmaceutically acceptable salt thereof, wherein R¹ is 4 to 7 membered heterocyclyl optionally substituted as defined for a compound of Formula (II), which said 4 to 7 membered heterocyclyl comprises one or two heteroatoms independently selected for each occurrence from the group consisting of N, O and S.

In another Embodiment (21.1), the invention provides a compound of Formula (II), Embodiment (20.0), or Embodiment (21), or a pharmaceutically acceptable salt thereof, wherein R¹ is selected from the group consisting of tetrahydrofuranyl, pyrrolidinyl, tetrahydrothiophenyl, pyrazolidinyl, imidazolidinyl, dioxolanyl, thiazolidinyl, isoxazolidinyl, tetrahydropyranyl, piperidinyl, piperizinyl and morpholinyl, which tetrahydrofuranyl, pyrrolidinyl, tetrahydrothiophenyl, pyrazolidinyl, imidazolidinyl, dioxolanyl, thiazolidinyl, isoxazolidinyl, tetrahydropyranyl, piperidinyl, piperizinyl and morpholinyl is optionally substituted as defined for a compound of Formula (II).

In another Embodiment (21.2), the invention provides a compound of Formula (II), Embodiment (20.0), Embodiment (21) or Embodiment (21.1), or a pharmaceutically acceptable salt thereof, wherein R¹ is selected from the group consisting of tetrahydrofuranyl and tetrahydropyranyl, preferably tetrahydrofuranyl, which tetrahydrofuranyl or tetrahydropyranyl is optionally substituted as defined for a compound of Formula (II).

In another Embodiment (21.3), the invention provides a compound of Formula (II), Embodiment (20.0), Embodiment (21), Embodiment (21.1) or Embodiment (21.2), or a pharmaceutically acceptable salt thereof, wherein R¹ is selected from the group consisting of tetrahydrofuranyl and tetrahydropyranyl, preferably tetrahydrofuranyl, which tetrahydrofuranyl or tetrahydropyranyl is unsubstituted or substituted with one, two, three of four E, which E is independently selected for each occurrence from the group consisting of halo, for example —F or —Cl; —C₁-C₃alkyl, for example methyl or ethyl; —OC₁-C₃alkyl; —C₃-C₇cycloalkyl, for example cyclopentyl; and phenyl, and which E is optionally further substituted as defined for a compound of Formula (II), for example E is —C₁-C₃alkyl substituted with one, two, three or four J, to form, for example, —CF₃.

In another Embodiment (21.4), the invention provides a compound of Formula (II), Embodiment (20.0), Embodiment (21), Embodiment (21.1), Embodiment (21.2), or Embodiment (21.3), or a pharmaceutically acceptable salt thereof, wherein R¹ is tetrahyrdopyranyl.

In another Embodiment (22), the invention provides a compound of Formula (II), or Embodiment (20.0), or a pharmaceutically acceptable salt thereof, wherein R¹ is

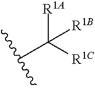

optionally substituted as defined for a compound of Formula (II).

In another Embodiment (22.1), the invention provides a compound of Formula (II), Embodiment (20.0), or Embodiment (22), or a pharmaceutically acceptable salt thereof, with the proviso that the compound is not:

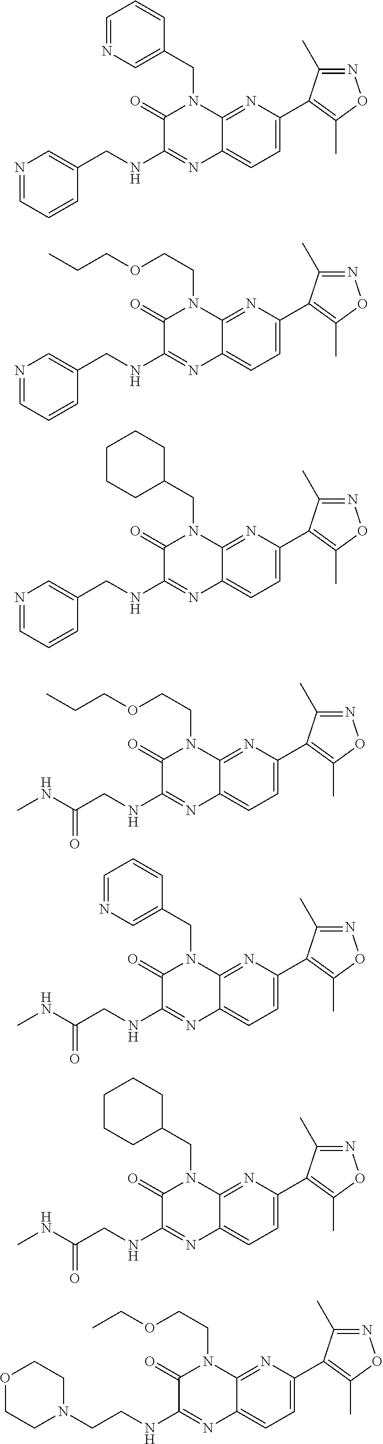

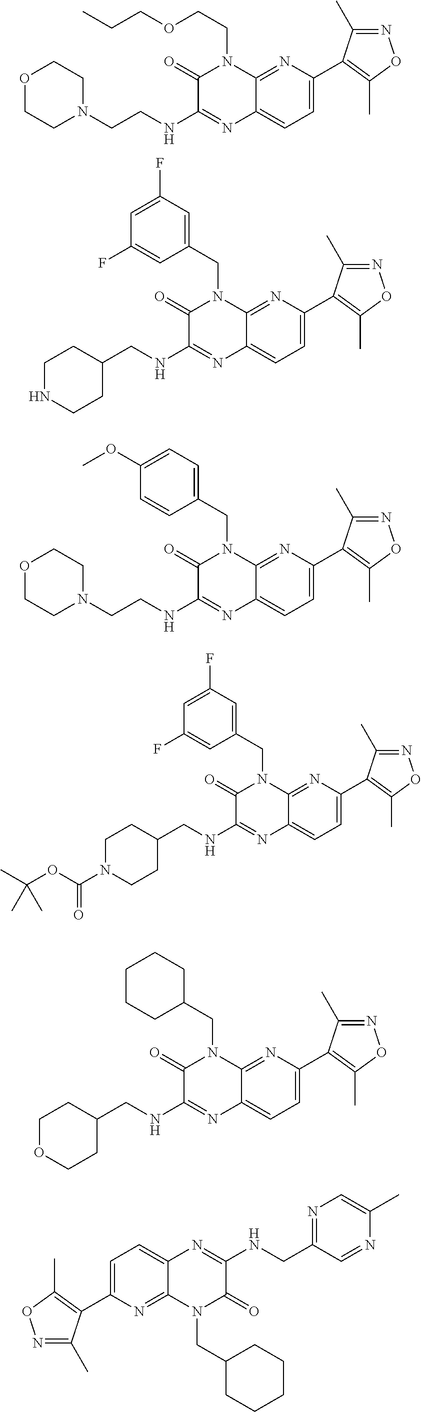

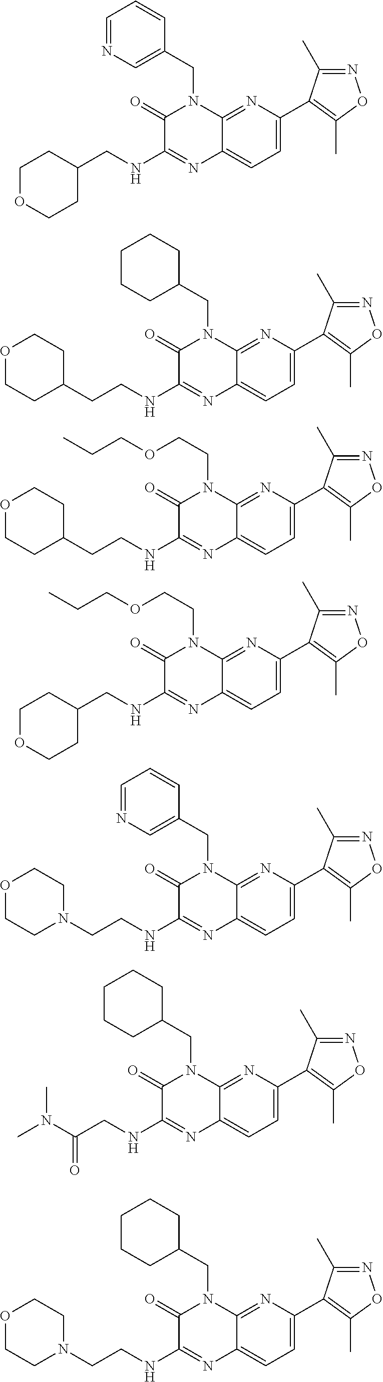

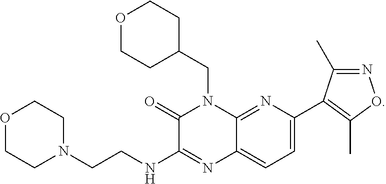 [SMILES: Cc1noc(C)c1-c1ccc2nc(NCCN3CCOCC3)c(=O)n(CC3CCOCC3)c2n1]

In another Embodiment (22.2), the invention provides a compound of Formula (II), Embodiment (20.0), Embodiment (22), or Embodiment (22.1), or a pharmaceutically acceptable salt thereof, wherein R¹ is

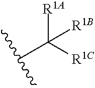

and R^{1A} is selected from the group consisting of —C₁-C₆alkyl, more preferably —C₁-C₄alkyl, for example methyl, ethyl or n-propyl; —C₃-C₇cycloalkyl, for example cyclohexyl; phenyl; and 5 to 6 membered heteroaryl which said 5 to 6 membered heteroaryl comprises one, two or three heteroatoms independently selected for each occurrence from the group consisting of N, O and S, for example pyridyl, pyridazinyl, or pyrimidinyl, which R^{1A} is optionally substituted as defined for a compound of Formula (II).

In another Embodiment (22.3), the invention provides a compound of Formula (II), Embodiment (20.0), Embodiment (22), Embodiment (22.1), or Embodiment (22.2), or a pharmaceutically acceptable salt thereof, wherein R¹ is

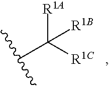

and R^{1A} is selected from the group consisting of methyl, ethyl, n-propyl, cyclohexyl, phenyl, pyridyl, pyridazinyl and pyrimidinyl which R^{1A} is optionally substituted as defined for a compound of Formula (II).

In another Embodiment (22.3a), the invention provides a compound of Formula (II), Embodiment (20.0), Embodiment (22), Embodiment (22.1), Embodiment (22.2), or Embodiment (22.3), or a pharmaceutically acceptable salt thereof, wherein R¹ is

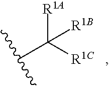

and R^{1A} is selected from the group consisting of methyl, ethyl, n-propyl, cyclohexyl, phenyl, pyridyl, pyridazinyl and pyrimidinyl which R^{1A} is optionally substituted with E as defined for a compound of Formula (II), which E is independently selected for each occurrence from the group consisting of —OH; —F; —Cl; —CH₃; —OCH₃; and —CF₃.

In another Embodiment (22.4), the invention provides a compound of Formula (II), Embodiment (20.0), Embodiment (22), Embodiment (22.1), Embodiment (22.2), Embodiment (22.3), or Embodiment (22.3a), or a pharmaceutically acceptable salt thereof, wherein R¹ is

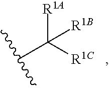

and R^{1A} is selected from the group consisting of methyl, ethyl, n-propyl, phenyl, and pyridyl, which R^{1A} is optionally substituted as defined for a compound of Formula (II).

In another Embodiment (22.5), the invention provides a compound of Formula (II), Embodiment (20.0), Embodiment (22), Embodiment (22.1), Embodiment (22.2), Embodiment (22.3), Embodiment (22.3a), or Embodiment (22.4), or a pharmaceutically acceptable salt thereof, wherein R¹ is

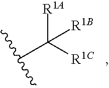

and R^{1A} is selected from the group consisting of methyl, phenyl, and pyridyl, which R^{1A} is optionally substituted as defined for a compound of Formula (II).

In another Embodiment (22.6), the invention provides a compound of Formula (II), Embodiment (20.0), Embodiment (22), Embodiment (22.1), Embodiment (22.2), Embodiment (22.3), Embodiment (22.3a), Embodiment (22.4), or Embodiment (22.5), or a pharmaceutically acceptable salt thereof, wherein R¹ is

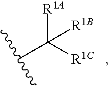

and R^{1A} is selected from the group consisting of methyl, phenyl and pyridyl, and which R^{1A} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (II), for example E is —C₁-C₃alkyl substituted with one, two, three or four J, to form, for example, —CF₃.

In another Embodiment (22.7), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), or Embodiment (22.6), or a pharmaceutically acceptable salt thereof, wherein R¹ is

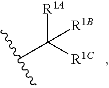

and R^{1A} is selected from the group consisting of methyl, phenyl, and pyridyl, which methyl, phenyl, and pyridyl is unsubstituted or substituted with one or two E, which E is independently selected from the group consisting of —OH, to form, for example, CH₂OH; —F, to form, for example, —CF₃ or fluorophenyl; —C₁-C₃alkyl, for example methyl, to form, for example, methylphenyl or methylpyridyl; —OC₁-C₃alkyl, for example methoxy or ethoxy, to form, for example, —CH₂OCH₃, —CH₂OCH₂CH₃, methoxyphenyl, or methoxypyridyl.

In another Embodiment (22.8), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), or Embodiment (22.7), or a pharmaceutically acceptable salt thereof, wherein R¹ is

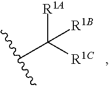

and R^{1A} is selected from the group consisting of ethyl; —CH₂OCH₃; phenyl; methoxyphenyl; ethoxyphenyl; pyridyl; and pyridinyl.

In another Embodiment (22.9), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), or Embodiment (22.8), or a pharmaceutically acceptable salt thereof, wherein R¹ is

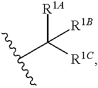

and R^{1B} is selected from the group consisting of —H; and —C₁-C₆alkyl, more preferably —C₁-C₄alkyl, for example methyl, ethyl, n-propyl or i-propyl, which R^{1B} is optionally substituted as defined for a compound of Formula (II).

In another Embodiment (22.10), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), or Embodiment (22.9a), or a pharmaceutically acceptable salt thereof, wherein R¹ is

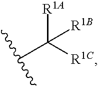

and R^{1B} is selected from the group consisting of —H, methyl, ethyl, n-propyl and i-propyl, which R^{1B} is optionally substituted as defined for a compound of Formula (II).

In another Embodiment (22.10a), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), or Embodiment (22.9a), or a pharmaceutically acceptable salt thereof, wherein R¹ is

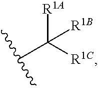

and R^{1B} is selected from the group consisting of —H, methyl, ethyl, n-propyl and i-propyl, which R^{1B} is optionally substituted with E as defined for a compound of Formula (I), which E is independently selected for each occurrence from the group consisting of —OH; —F; —Cl; —CH₃; —OCH₃; and —CF₃.

In another Embodiment (22.11), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), or Embodiment (22.10a), or a pharmaceutically acceptable salt thereof, wherein R¹ is

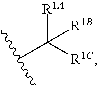

and R^{1B} is selected from the group consisting of —H, methyl, ethyl, n-propyl and i-propyl, which R^{1B} is unsubstituted or substituted with one or two E, which E is independently selected for each occurrence from the group consisting of —CN; —OH; halo, for example —F or —Cl; —C₁-C₃alkyl, for example methyl; —OC₁-C₃alkyl, for example methoxy; —NH₂; —NH(C₁-C₃alkyl); and —N(C₁-C₃alkyl)₂, which substituent E is optionally further substituted as defined for a compound of Formula (II).

In another Embodiment (22.12), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), Embodiment (22.10a), or Embodiment (22.11), or a pharmaceutically acceptable salt thereof, wherein R¹ is

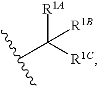

and R^{1B} is selected from the group consisting of —H, methyl, ethyl, n-propyl and i-propyl, which R^{1B} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (II), for example E is —C₁-C₃alkyl substituted with one, two, three or four J, to form, for example, —CF₃.

In another Embodiment (22.13), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), Embodiment (22.10a), Embodiment (22.11), or Embodiment (22.12), or a pharmaceutically acceptable salt thereof, wherein R¹ is

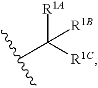

and R^{1B} is selected from the group consisting of —H, methyl, ethyl, n-propyl, i-propyl, and —CH₂OCH₃.

In another Embodiment (22.14), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), Embodiment (22.10a), Embodiment (22.11), Embodiment (22.12), or Embodiment (22.13), or a pharmaceutically acceptable salt thereof, wherein R¹ is

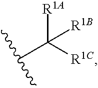

and R^{1C} is selected from the group consisting of —CH₃; and —H.

In another Embodiment (22.15), the invention provides a compound of Formula (II), Embodiment (20.0), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), Embodiment (22.10a), Embodiment (22.11), Embodiment (22.12), Embodiment (22.13), or Embodiment (22.14) or a pharmaceutically acceptable salt thereof, wherein R¹ is

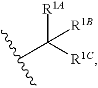

### and R^{1C} is —H.

In an another Embodiment (23), the invention provides a compound of Formula (IIa),

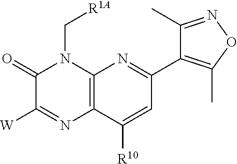

Formula IIa
   or a pharmaceutically acceptable salt thereof, wherein:

R^{1A} is selected from the group consisting of
(i) —C₁-C₆alkyl optionally substituted with one, two, three, four, five or six E;
(ii) —C₃-C₇cycloalkyl optionally substituted with one, two, three, four or five E;
(iii) phenyl optionally substituted with one, two, three, four or five E;
(iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
(v) 5 to 6 membered heteroaryl optionally substituted with one, two, three, four or five E, which said 5 to 6 membered heteroaryl comprises one, two or three heteroatoms independently selected for each occurrence from the group consisting of N, O and S;
   and wherein W, Y, R³, R^{4A}, R^{4B}, R^{4C}, R¹⁰, E, G, and J are all defined as for a compound of Formula (II).

In another Embodiment (23.0), the invention provides a compound of Formula (IIa′):

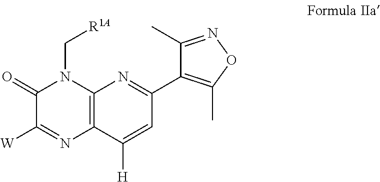

or a pharmaceutically acceptable salt thereof, and wherein R^{1A}, W, Y, R³, R^{4A}, R^{4B}, R^{4C}, E, G, and J are all defined as for a compound of Formula (IIa).

In another Embodiment (23.1), the invention provides a compound of Formula (IIa), or Embodiment (23.0), or a pharmaceutically acceptable salt thereof, with the proviso that the compound is not:

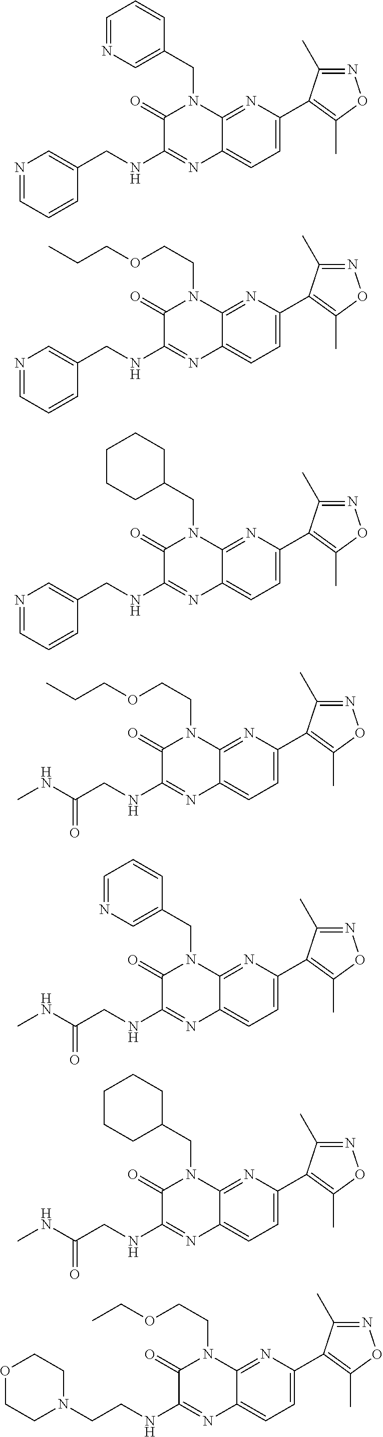

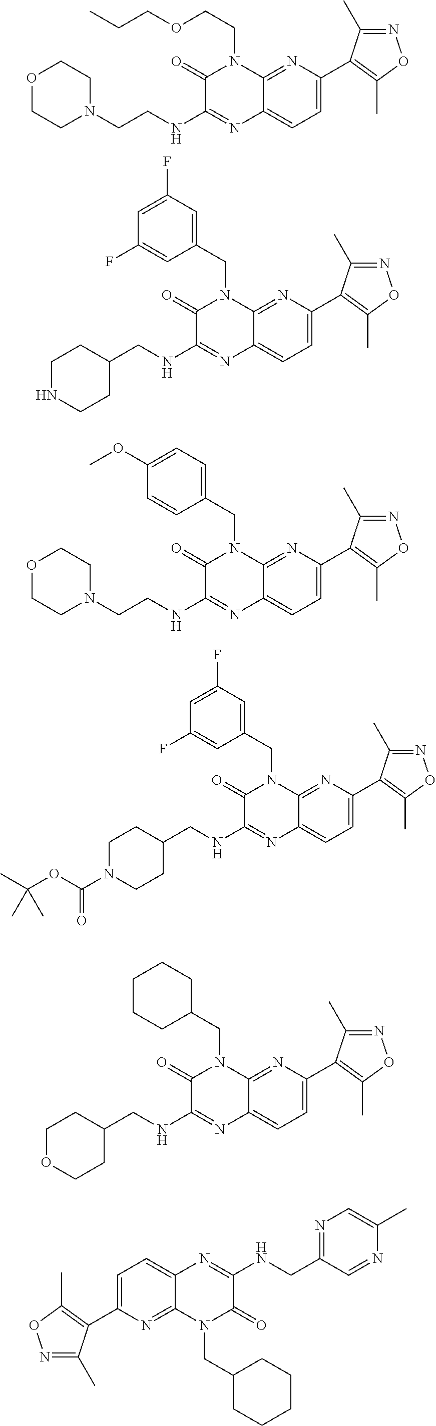

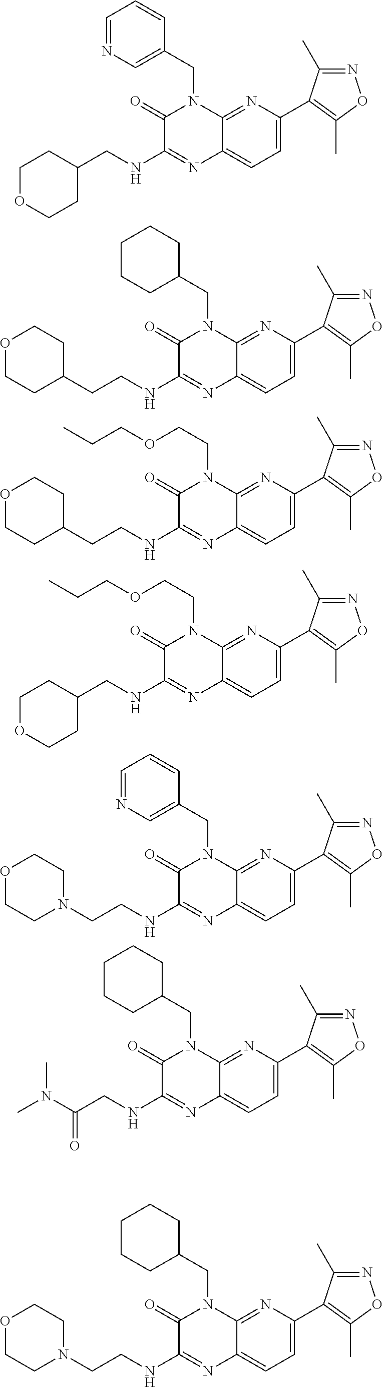

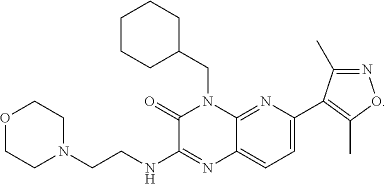 [SMILES: Cc1noc(C)c1-c1ccc2nc(NCCN3CCOCC3)c(=O)n(CC3CCCCC3)c2n1]

In another Embodiment (23.2), the invention provides a compound of Formula (IIa), Embodiment (23.0), Embodiment (23), or Embodiment (23.1), or a pharmaceutically acceptable salt thereof, wherein R^{1A} is selected from the group consisting of —C₃-C₇cycloalkyl; phenyl; 4 to 7 membered heterocyclyl, which said 4 to 7 membered heterocyclyl comprises one or two heteroatoms independently selected for each occurrence from the group consisting of N, O and S; and 5 to 6 membered heteroaryl, which said 5 to 6 membered heteroaryl comprises one, two or three heteroatoms independently selected for each occurrence from the group consisting of N, O and S, which R^{1A} is optionally substituted as defined for a compound of Formula (IIa).

In another Embodiment (23.2a), the invention provides a compound of Formula (IIa), Embodiment (23.0), Embodiment (23), Embodiment (23.1), or Embodiment (23.2), or a pharmaceutically acceptable salt thereof, wherein R^{1A} is selected from the group consisting of —C₃-C₇cycloalkyl, for example cyclohexyl; phenyl; and 5 to 6 membered heteroaryl which said 5 to 6 membered heteroaryl comprises one, two or three heteroatoms independently selected for each occurrence from the group consisting of N, O and S, for example pyridyl, pyridazinyl, or pyrimidinyl, which R^{1A} is optionally substituted as defined for a compound of Formula (IIa).

In another Embodiment (23.3), the invention provides a compound of Formula (IIa), Embodiment (23.0), Embodiment (23), Embodiment (23.1), or Embodiment (23.2), or a pharmaceutically acceptable salt thereof, wherein R^{1A} is selected from the group consisting of cyclohexyl, phenyl, pyridyl, pyridazinyl and pyrimidinyl which R^{1A} is optionally substituted as defined for a compound of Formula (IIa).

In another Embodiment (23.4), the invention provides a compound of Formula (IIa), Embodiment (23.0), Embodiment (23), Embodiment (23.1), Embodiment (23.2), or Embodiment (23.3a), or a pharmaceutically acceptable salt thereof, wherein R^{1A} is selected from the group consisting of cyclohexyl, phenyl, pyridyl, pyridazinyl and pyrimidinyl which R^{1A} is optionally substituted with E as defined for a compound of Formula (IIa), which E is independently selected for each occurrence from the group consisting of —OH; —F; —Cl; —CH₃; —OCH₃; and —CF₃.

In another Embodiment (23.5), the invention provides a compound of Formula (IIa), Embodiment (23.0), Embodiment (23), Embodiment (23.1), Embodiment (23.2), Embodiment (23.3), or Embodiment (23.3a), or a pharmaceutically acceptable salt thereof, wherein R^{1A} is selected from the group consisting of phenyl, and pyridyl, which R^{1A} is optionally substituted as defined for a compound of Formula (IIa).

In another Embodiment (23.6), the invention provides a compound of Formula (IIa), Embodiment (23.0), Embodiment (23), Embodiment (23.1), Embodiment (23.2), Embodiment (23.3), Embodiment (23.3a), Embodiment (23.4), or Embodiment (23.5), or a pharmaceutically acceptable salt thereof, wherein R^{1A} is selected from the group consisting of phenyl and pyridyl, and which R^{1A} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (IIa), for example E is —C₁-C₃alkyl substituted with one, two, three or four J, to form, for example, —CF₃.

In another Embodiment (23.7), the invention provides a compound of Formula (IIa), Embodiment (23.0), Embodiment (23), Embodiment (23.1), Embodiment (23.2), Embodiment (23.3), Embodiment (23.3a), Embodiment (23.4), Embodiment (23.5), or Embodiment (23.6), or a pharmaceutically acceptable salt thereof, wherein R^{1A} is selected from the group consisting of phenyl, and pyridyl, which methyl, phenyl, and pyridyl is unsubstituted or substituted with one or two E, which E is independently selected from the group consisting of —OH, to form, for example, CH₂OH; —F, to form, for example, —CF₃ or fluorophenyl; —C₁-C₃alkyl, for example methyl, to form, for example, methylphenyl or methylpyridyl; —OC₁-C₃alkyl, for example methoxy or ethoxy, to form, for example, —CH₂OCH₃, —CH₂OCH₂CH₃, methoxyphenyl, or methoxypyridyl.

In another Embodiment (23.8), the invention provides a compound of Formula (IIa), Embodiment (23.0), Embodiment (23), Embodiment (23.1), Embodiment (23.2), Embodiment (23.3), Embodiment (23.3a), Embodiment (23.4), Embodiment (23.5), Embodiment (23.6), or Embodiment (23.7), or a pharmaceutically acceptable salt thereof, wherein R^{1A} is selected from the group consisting of phenyl; methoxyphenyl; ethoxyphenyl; pyridyl; and pyridinyl.

In another Embodiment (24), this invention relates to a compound of Formula (IIb):

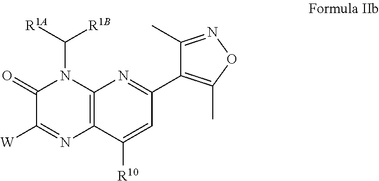

or a pharmaceutically acceptable salt thereof, wherein: R^{1A} is selected from the group consisting of
(i) —C₁-C₆alkyl optionally substituted with one, two, three, four, five or six E;
(ii) —C₃-C₇cycloalkyl optionally substituted with one, two, three, four or five E;
(iii) phenyl optionally substituted with one, two, three, four or five E;
(iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
(v) 5 to 6 membered heteroaryl optionally substituted with one, two, three, four or five E, which said 5 to 6 membered heteroaryl comprises one, two or three heteroatoms independently selected for each occurrence from the group consisting of N, O and S;
R^{1B} is —C₁-C₆ alkyl optionally substituted with one, two, three or four E;
   and wherein W, Y, R³, R^{4A}, R^{4B}, R^{4C}, R¹⁰, E, G, and J are all defined as for a compound of Formula (II).

In another Embodiment (24.0), the invention provides a compound of Formula (IIb′):

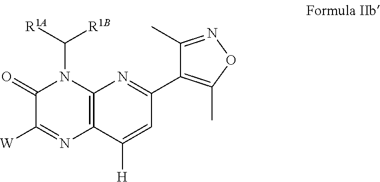

or a pharmaceutically acceptable salt thereof, according to any preceding Embodiment, and wherein R^{1A}, R^{1B}, W, Y, R³, R^{4A}, R^{4B}, R^{4C}, E, G, and J are all defined as for a compound of Formula (IIb).

In another Embodiment (24.1), the invention provides a compound of Formula (IIb), Embodiment (24), or Embodiment (24.0), or a pharmaceutically acceptable salt thereof, wherein R^{1A} is selected from the group consisting of —C₁-C₆alkyl, more preferably —C₁-C₄alkyl, for example methyl, ethyl or n-propyl; —C₃-C₇cycloalkyl, for example cyclohexyl; phenyl; and 5 to 6 membered heteroaryl which said 5 to 6 membered heteroaryl comprises one, two or three heteroatoms independently selected for each occurrence from the group consisting of N, O and S, for example pyridyl, pyridazinyl, or pyrimidinyl, which R^{1A} is optionally substituted as defined for a compound of Formula (IIb).

In another Embodiment (24.2), the invention provides a compound of Formula (IIb), Embodiment (24), Embodiment (24.0), Embodiment (24.1), or a pharmaceutically acceptable salt thereof, wherein R^{1A} is selected from the group consisting of methyl, ethyl, n-propyl, cyclohexyl, phenyl, pyridyl, pyridazinyl and pyrimidinyl which R^{1A} is optionally substituted as defined for a compound of Formula (IIb).

In another Embodiment (24.2a), the invention provides a compound of Formula (IIb), Embodiment (24), Embodiment (24.0), Embodiment (24.1), or Embodiment (24.2), or a pharmaceutically acceptable salt thereof, wherein R^{1A} is selected from the group consisting of methyl, ethyl, n-propyl, cyclohexyl, phenyl, pyridyl, pyridazinyl and pyrimidinyl which R^{1A} is optionally substituted with E as defined for a compound of Formula (IIb), which E is independently selected for each occurrence from the group consisting of —OH; —F; —Cl; —CH₃; —OCH₃; and —CF₃.

In another Embodiment (24.3), the invention provides a compound of Formula (IIb), Embodiment (24), Embodiment (24.0), Embodiment (24.1), Embodiment (24.1), or Embodiment (24.2a), or a pharmaceutically acceptable salt thereof, wherein R^{1A} is selected from the group consisting of methyl, ethyl, n-propyl, phenyl, and pyridyl, which R^{1A} is optionally substituted as defined for a compound of Formula (IIb).

In another Embodiment (24.4), the invention provides a compound of Formula (IIb), Embodiment (24), Embodiment (24.0), Embodiment (24.1), Embodiment (24.2), Embodiment (24.2a), or Embodiment (24.3), or a pharmaceutically acceptable salt thereof, wherein R^{1A} is selected from the group consisting of methyl, phenyl, and pyridyl, which R^{1A} is optionally substituted as defined for a compound of Formula (IIb).

In another Embodiment (24.5), the invention provides a compound of Formula (IIb), Embodiment (24), Embodiment (24.0), Embodiment (24.1), Embodiment (24.2), Embodiment (24.2a), Embodiment (24.3), or Embodiment (24.4), or a pharmaceutically acceptable salt thereof, wherein R^{1A} is selected from the group consisting of methyl, phenyl and pyridyl, and which R^{1A} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (IIb), for example E is —C₁-C₃alkyl substituted with one, two, three or four J, to form, for example, —CF₃.

In another Embodiment (24.6), the invention provides a compound of Formula (IIb), Embodiment (24), Embodiment (24.0), Embodiment (24.1), Embodiment (24.2), Embodiment (24.2a), Embodiment (24.3), Embodiment (24.4), or Embodiment (24.5), or a pharmaceutically acceptable salt thereof, wherein R^{1A} is selected from the group consisting of methyl, phenyl, and pyridyl, which methyl, phenyl, and pyridyl is unsubstituted or substituted with one or two E, which E is independently selected from the group consisting of —OH, to form, for example, CH₂OH; —F, to form, for example, —CF₃ or fluorophenyl; —C₁-C₃alkyl, for example methyl, to form, for example, methylphenyl or methylpyridyl; —OC₁-C₃alkyl, for example methoxy or ethoxy, to form, for example, —CH₂OCH₃, —CH₂OCH₂CH₃, methoxyphenyl, or methoxypyridyl.

In another Embodiment (24.7), the invention provides a compound of Formula (IIb), Embodiment (24), Embodiment (24.0), Embodiment (24.1), Embodiment (24.2), Embodiment (24.2a), Embodiment (24.3), Embodiment (24.4), Embodiment (24.5), or Embodiment (24.6), or a pharmaceutically acceptable salt thereof, wherein R^{1A} is selected from the group consisting of ethyl; —CH₂OCH₃; phenyl; methoxyphenyl; ethoxyphenyl; pyridyl; and pyridinyl.

In another Embodiment (24.8), the invention provides a compound of Formula (IIb), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), or Embodiment (24.7), or a pharmaceutically acceptable salt thereof, wherein R^{1B} is selected from the group consisting of —C₁-C₆alkyl, more preferably —C₁-C₄alkyl, for example methyl, ethyl, n-propyl or i-propyl, which R^{1B} is optionally substituted as defined for a compound of Formula (IIb).

In another Embodiment (24.9), the invention provides a compound of Formula (IIb), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), or Embodiment (24.8), or a pharmaceutically acceptable salt thereof, wherein R^{1B} is selected from the group consisting of methyl, ethyl, n-propyl and i-propyl, which R^{1B} is optionally substituted as defined for a compound of Formula (IIb).

In another Embodiment (24.9a), the invention provides a compound of Formula (IIb), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), Embodiment (24.8), or Embodiment (24.9), or a pharmaceutically acceptable salt thereof, wherein R^{1B} is selected from the group consisting of methyl, ethyl, n-propyl and i-propyl, which R^{1B} is optionally substituted with E as defined for a compound of Formula (IIb), which E is independently selected for each occurrence from the group consisting of —OH; —F; —Cl; —CH₃; —OCH₃; and —CF₃.

In another Embodiment (24.10), the invention provides a compound of Formula (IIb), Embodiment (24), Embodiment (24.0), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), Embodiment (24.8), Embodiment (24.9), or Embodiment (24.9a), or a pharmaceutically acceptable salt thereof, wherein R^{1B} is selected from the group consisting of methyl, ethyl, n-propyl and i-propyl, which R^{1B} is unsubstituted or substituted with one or two E, which E is independently selected for each occurrence from the group consisting of —CN; —OH; halo, for example —F or —Cl; —C₁-C₃alkyl, for example methyl or ethyl; —OC₁-C₃alkyl, for example methoxy or ethoxy; —C₃-C₇cycloalkyl, for example cyclopentyl; —NH₂; —NH(C₁-C₃alkyl); and —N(C₁-C₃alkyl)₂, which substituent E is optionally further substituted as defined for a compound of Formula (IIb), for example E is —C₁-C₃alkyl substituted with one, two, three or four J, to form, for example, —CF₃.

In another Embodiment (24.11), the invention provides a compound of Formula (IIb), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), Embodiment (24.8), Embodiment (24.9), Embodiment (24.9a), or Embodiment (24.10), or a pharmaceutically acceptable salt thereof, wherein R^{1B} is selected from the group consisting of methyl, ethyl, n-propyl and i-propyl, which R^{1B} is unsubstituted or substituted with one or two E, which E is independently selected for each occurrence from the group consisting of —OC₁-C₃alkyl, for example methoxy, to form, for example —CH₂OCH₃.

In another Embodiment (24.12), the invention provides a compound of Formula (IIb), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), Embodiment (24.8), Embodiment (24.9), Embodiment (24.9a), Embodiment (24.10), or Embodiment (24.11), or a pharmaceutically acceptable salt thereof, wherein R^{1B} is selected from the group consisting of methyl, ethyl, n-propyl, i-propyl, and —CH₂OCH₃.

In another Embodiment (25), the invention provides a compound of Formula (II), Formula (IIa), or Formula (IIb), or a pharmaceutically acceptable salt thereof, according to any preceding Embodiment of Formula (II), Formula (IIa), or Formula (IIb), wherein W is

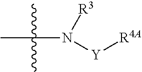

and where R³, Y and R^{4A} are as defined for a compound of Formula (II), Formula (IIa) or Formula (IIb) respectively.

In another Embodiment (25.1), the invention provides a compound of Formula (II), Formula (IIa), or Formula (IIb), or a pharmaceutically acceptable salt thereof, according to any preceding Embodiment of Formula (II), Formula (IIa), or Formula (IIb), wherein W is

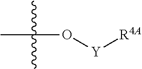

and where Y and R^{4A} are as defined for a compound of Formula (II), Formula (IIa) or Formula (IIb) respectively.

In another Embodiment (25.2), the invention provides a compound of Formula (II), Formula (IIa), or Formula (IIb), or a pharmaceutically acceptable salt thereof, according to any preceding Embodiment of Formula (II), Formula (IIa), or Formula (IIb), wherein W is

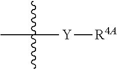

and where Y and R^{4A} are as defined for a compound of Formula (II), Formula (IIa) or Formula (IIb) respectively.

In another Embodiment (25.3), the invention provides a compound of Formula (II), Formula (IIa), or Formula (IIb), or a pharmaceutically acceptable salt thereof, according to any preceding Embodiment of Formula (II), Formula (IIa), or Formula (IIb), wherein W is

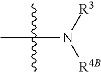

and where R³ and R^{4A} are as defined for a compound of Formula (II), Formula (IIa) or Formula (IIb) respectively.

In another Embodiment (25.4), the invention provides a compound of Formula (II), Formula (IIa), or Formula (IIb), or a pharmaceutically acceptable salt thereof, according to any preceding Embodiment of Formula (II), Formula (IIa), or Formula (IIb), wherein W is

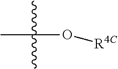

and where R^{4C} is as defined for a compound of Formula (II), Formula (IIa) or Formula (IIb) respectively.

In another Embodiment (25.5), the invention provides a compound of Formula (II), Formula (IIa), or Formula (IIb), or a pharmaceutically acceptable salt thereof, according to any preceding Embodiment of Formula (II), Formula (IIa), or Formula (IIb), wherein W is a 4 to 7 membered heterocyclyl which said 4 to 7 membered heterocyclyl comprises one, two, three or four heteroatoms independently selected for each occurrence from the group consisting of N, O and S, and which W is optionally further substituted as defined for a compound of Formula (II), Formula (IIa) or Formula (IIb) respectively.

In another Embodiment (26), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is selected from the group consisting of —CH₂— and —CH₂CH₂—, which Y is unsubstituted or optionally further substituted as defined for a compound of Formula (II), Formula (IIa) or Formula (IIb) respectively.

In another Embodiment (26.0), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is selected from the group consisting of —CH₂— and —CH₂CH₂—, which Y is unsubstituted or optionally further substituted as defined for a compound of Formula (II), Formula (IIa) or Formula (IIb) respectively, which J is independently for each occurrence selected from the group consisting of —H, —F, —Cl, —CH₃; —CF₃; —OCH₃; and —OCF₃.

In another Embodiment (26.1), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH₂—.

In another Embodiment (26.2), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH₂CH₂—.

In another Embodiment (26.3), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH(CH₃)—.

In another Embodiment (26.4), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH(CF₃)—.

In another Embodiment (26.5), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH(CH₃)CH₂—.

In another Embodiment (26.6), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH(CF₃)CH₂—.

In another Embodiment (26.7), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH₂CH(CH₃)—.

In another Embodiment (26.8), the invention provides a compound of Formula (II), Formula (IIa), or Formula (IIb), or a pharmaceutically acceptable salt thereof, according to any preceding Embodiment Formula (II), Formula (IIa) or Formula (IIb), wherein Y is —CH₂CH(CF₃)—.

In another Embodiment (27), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R³ is —H.

In another Embodiment (27.1), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R³ is —CH₃, which R³ unsubstituted or optionally further substituted as defined for a compound of Formula (II), Formula (IIa) or Formula (IIb) respectively.

In another Embodiment (27.2), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R³ is —CH₃, which R³ is unsubstituted or optionally substituted with one, two or three J, which J is independently selected from the group consisting of —H, —F, —CH₃; and —CF₃.

In another Embodiment (27.3), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R³ is —CH₃.

In another Embodiment (28), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4A} is selected from the group consisting of —C₁-C₆alkyl, preferably —C₁-C₄alkyl; —CO₂H; —C(O)OC₁-C₆alkyl; —C(O)NH₂; —C(O)NH(C₁-C₆alkyl); —C(O)N(C₁-C₆alkyl)₂; —C(O)NHSO₂C₁-C₃alkyl; 4 to 7 membered heterocyclyl which said 4 to 7 membered heterocyclyl comprises one, two, three or four heteroatoms independently selected for each occurrence from the group consisting of N, O and S; and 5 to 6 membered heteroaryl, which said 5 to 6 membered heteroaryl ring comprises one, two, three or four heteroatoms independently selected for each occurrence from the group consisting of N, O and S; and which R^{4A} is optionally further substituted as defined for a compound of Formula (II), Formula (IIa) or Formula (IIb) respectively.

In another Embodiment (28.1), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4A} is selected from the group consisting of —C₁-C₆alkyl, preferably —C₁-C₄alkyl, for example —CH₃; —CO₂H; —C(O)OC₁-C₆alkyl for example —C(O)OCH₃ and —C(O)OCH₂CH₃; —C(O)NH(C₁-C₆alkyl), for example —C(O)NHCH₃; —C(O)NHSO₂C₁-C₃alkyl for example —C(O)NHSO₂CH₃; 4 to 7 membered heterocyclyl which said 4 to 7 membered heterocyclyl comprises one, two, three or four heteroatoms independently selected for each occurrence from the group consisting of N, O and S, for example morpholinyl, pyranyl, piperidinyl, or piperazinyl; and 5 to 6 membered heteroaryl, which said 5 to 6 membered heteroaryl ring comprises one, two, three or four heteroatoms independently selected for each occurrence from the group consisting of N, O and S, for example tetrazolyl; and which R^{4A} is optionally further substituted as defined for a compound of Formula (II), Formula (IIa) or Formula (IIb) respectively.

In another Embodiment (28.2), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4A} is selected from the group consisting of —CH₃; —CO₂H; —C(O)OCH₃; —C(O)OCH₂CH₃; —C(O)NHCH₃; —C(O)NHSO₂CH₃; morpholinyl; and tetrazolyl; and which R^{4A} is optionally further substituted as defined for a compound of Formula (II), Formula (IIa) or Formula (IIb) respectively.

In another Embodiment (28.3), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4A} is selected from the group consisting of —CH₃; —CO₂H; —C(O)OCH₃; —C(O)OCH₂CH₃; —C(O)NHCH₃; —C(O)NHSO₂CH₃; morpholinyl; and tetrazolyl; and which R^{4A} is optionally further substituted with G as defined for a compound of Formula (II), Formula (IIa) or Formula (IIb) respectively, which G is selected, independently for each occurrence, from the group consisting of —OH; —F; —Cl; —CH₃; —OCH₃; and —CF₃.

In another Embodiment (28.4), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4A} is selected from the group consisting of —CO₂H; —C(O)OCH₃; —C(O)OCH₂CH₃; —C(O)NHCH₃; —C(O)NHSO₂CH₃; morpholinyl; and tetrazolyl.

In another Embodiment (28.5), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4A} is selected from the group consisting of —CO₂H; —C(O)NHCH₃; —C(O)NHSO₂CH₃; morpholinyl; and tetrazolyl.

In another Embodiment (29), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4B} is selected from the group consisting of —C₁-C₆alkyl, preferably —C₁-C₄alkyl; —C(O)C₁-C₆alkyl; —C(O)OC₁-C₆alkyl; —C(O)NH₂; —C(O)NH(C₁-C₆alkyl); —C(O)N(C₁-C₆alkyl)₂; —C(O)NHSO₂C₁-C₃alkyl; 4 to 7 membered heterocyclyl which said 4 to 7 membered heterocyclyl comprises one, two, three or four heteroatoms independently selected for each occurrence from the group consisting of N, O and S; and 5 to 6 membered heteroaryl, which said 5 to 6 membered heteroaryl ring comprises one, two, three or four heteroatoms independently selected for each occurrence from the group consisting of N, O and S; and which R^{4B} is optionally further substituted as defined for a compound of Formula (II), Formula (IIa) or Formula (IIb) respectively.

In another Embodiment (29.1), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4B} is selected from the group consisting of —C₁-C₆alkyl, preferably —C₁-C₄alkyl, for example CH₃; —C(O)C₁-C₆alkyl, for example —C(O)CH₃; —C(O)NH(C₁-C₆alkyl), for example —C(O)NHCH₃; —C(O)NHSO₂C₁-C₃alkyl for example —C(O)NHSO₂CH₃; 4 to 7 membered heterocyclyl which said 4 to 7 membered heterocyclyl comprises one, two, three or four heteroatoms independently selected for each occurrence from the group consisting of N, O and S, for example morpholinyl, pyranyl, piperidinyl, or piperazinyl; and 5 to 6 membered heteroaryl, which said 5 to 6 membered heteroaryl ring comprises one, two, three or four heteroatoms independently selected for each occurrence from the group consisting of N, O and S, for example tetrazolyl; and which R^{4B} is optionally further substituted as defined for a compound of Formula (II), Formula (IIa) or Formula (IIb) respectively.

In another Embodiment (29.2), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4B} is selected from the group consisting of —CH₃; —C(O)CH₃; —C(O)NHCH₃; —C(O)NHSO₂CH₃; morpholinyl; and tetrazolyl; and which R^{4B} is optionally further substituted as defined for a compound of Formula (II), Formula (IIa) or Formula (IIb) respectively.

In another Embodiment (29.3), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4B} is selected from the group consisting of —CH₃; —C(O)CH₃; —C(O)NHCH₃; —C(O)NHSO₂CH₃; morpholinyl; and tetrazolyl; and which R^{4B} is optionally further substituted with G as defined for a compound of Formula (II), Formula (IIa) or Formula (IIb) respectively, which G is selected, independently for each occurrence, from the group consisting of —OH; —F; —Cl; —CH₃; —OCH₃; and —CF₃.

In another Embodiment (29.4), the invention provides a compound of Formula (II), Formula (IIa), or Formula (IIb), or a pharmaceutically acceptable salt thereof, according to any preceding Embodiment, wherein R^{4B} is selected from the group consisting of —CH₃; —C(O)CH₃; —C(O)NHCH₃; —C(O)NHSO₂CH₃; morpholinyl; and tetrazolyl.

In another Embodiment (30), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4C} is selected from the group consisting of —C₁-C₆alkyl, preferably —C₁-C₄alkyl; 4 to 7 membered heterocyclyl which said 4 to 7 membered heterocyclyl comprises one, two, three or four heteroatoms independently selected for each occurrence from the group consisting of N, O and S; and 5 to 6 membered heteroaryl, which said 5 to 6 membered heteroaryl ring comprises one, two, three or four heteroatoms independently selected for each occurrence from the group consisting of N, O and S; and which R^{4C} is optionally further substituted as defined for a compound of Formula (II), Formula (IIa) or Formula (IIb) respectively.

In another Embodiment (30.1), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4C} is selected from the group consisting of —C₁-C₆alkyl, preferably —C₁-C₄alkyl, for example CH₃; 4 to 7 membered heterocyclyl which said 4 to 7 membered heterocyclyl comprises one, two, three or four heteroatoms independently selected for each occurrence from the group consisting of N, O and S, for example morpholinyl, pyranyl, piperidinyl, or piperazinyl; and 5 to 6 membered heteroaryl, which said 5 to 6 membered heteroaryl ring comprises one, two, three or four heteroatoms independently selected for each occurrence from the group consisting of N, O and S, for example tetrazolyl; and which R^{4C} is optionally further substituted as defined for a compound of Formula (II), Formula (IIa) or Formula (IIb) respectively.

In another Embodiment (30.2), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4C} is selected from the group consisting of —CH₃; morpholinyl; and tetrazolyl; and which R^{4C} is optionally further substituted as defined for a compound of Formula (II), Formula (IIa) or Formula (IIb) respectively.

In another Embodiment (30.3), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4C} is selected from the group consisting of —CH₃; morpholinyl; and tetrazolyl; and which R^{4C} is optionally further substituted with G as defined for a compound of Formula (II), Formula (IIa) or Formula (IIb) respectively, which G is selected, independently for each occurrence, from the group consisting of —OH; —F; —Cl; —CH₃; —OCH₃; and —CF₃.

In another Embodiment (30.4), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4C} is selected from the group consisting of —CH₃; morpholinyl; and tetrazolyl

In another Embodiment (30.5), the invention provides a compound of Formula (II), Formula (IIa), or Formula (IIb), or a pharmaceutically acceptable salt thereof, according to any preceding Embodiment Formula (II), Formula (IIa) or Formula (IIb), wherein R^{4C} is —CH₃.

In another Embodiment (31), the invention provides a compound of Formula (II), Formula (IIa), or Formula (IIb), or a pharmaceutically acceptable salt thereof, according to any preceding Embodiment Formula (II), Formula (IIa) or Formula (IIb), wherein W is

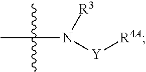

R³ is —H; and where Y and R^{4A} are as defined for a compound of Formula (II), Formula (IIa) or Formula (IIb) respectively.

In another Embodiment (31.1), the invention provides a compound of Formula (II), Formula (IIa), or Formula (IIb), or a pharmaceutically acceptable salt thereof, according to any preceding Embodiment Formula (II), Formula (IIa) or Formula (IIb), wherein W is

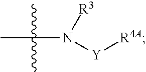

R³ is —H; Y is —CH₂—, which Y is optionally further substituted as defined for a compound Formula (II), Formula (IIa) or Formula (IIb) respectively; and R^{4A} is as defined for a compound of Formula (II), Formula (IIa) or Formula (IIb) respectively.

In another Embodiment (31.2), the invention provides a compound of Formula (II), Formula (IIa), or Formula (IIb), or a pharmaceutically acceptable salt thereof, according to any preceding Embodiment Formula (II), Formula (IIa) or Formula (IIb), wherein W is

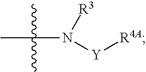

R³ is —H; Y is —CH₂CH₂—, which Y is optionally further substituted as defined for a compound Formula (II), Formula (IIa) or Formula (IIb) respectively; and R^{4A} is as defined for a compound of Formula (II), Formula (IIa) or Formula (IIb) respectively.

In another Embodiment (31.3), the invention provides a compound of Formula (II), Formula (IIa), or Formula (IIb), or a pharmaceutically acceptable salt thereof, according to any preceding Embodiment Formula (II), Formula (IIa) or Formula (IIb), wherein W is

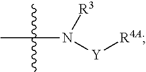

R³ is —H; Y is —CH₂—, which Y is optionally further substituted as defined for a compound Formula (II), Formula (IIa) or Formula (IIb) respectively; and R^{4A} is —CH₃; —CO₂H; —C(O)NHCH₃; —C(O)NHSO₂CH₃; morpholinyl; and tetrazolyl.

In another Embodiment (31.4), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein W is

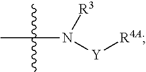

R³ is —H; Y is —CH₂CH₂—, which Y is optionally further substituted as defined for a compound Formula (II), Formula (IIa) or Formula (IIb) respectively; and R^{4A} is —CH₃; —CO₂H; —C(O)NHCH₃; —C(O)NHSO₂CH₃; morpholinyl; and tetrazolyl.

In another Embodiment (32), the invention provides a compound of Formula (II), Formula (IIa), or Formula (IIb), or a pharmaceutically acceptable salt thereof, according to any preceding Embodiment Formula (II), Formula (IIa) or Formula (IIb), wherein E is selected independently for each occurrence from the group consisting of —OH; halo; —C₁-C₃alkyl; —OC₁-C₃alkyl; —C(O)C₁-C₃alkyl; —C(O)OC₁-C₃alkyl; —NH₂; —NH(C₁-C₃alkyl); —N(C₁-C₃alkyl)₂; —C₃-C₇cycloalkyl; and phenyl, which E is optionally further substituted as defined for a compound of Formula (II), Formula (IIa) or Formula (IIb) respectively.

In another Embodiment (32.1), the invention provides a compound of Formula (II), Formula (IIa), or Formula (IIb), or a pharmaceutically acceptable salt thereof, according to any preceding Embodiment, wherein E is independently selected for each occurrence from the group consisting of —OH; —F; —Cl; —CH₃; —OCH₃ and —CF₃.

In another Embodiment (33), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein G is independently selected for each occurrence from the group consisting of —OH; halo; —C₁-C₃alkyl; —OC₁-C₃alkyl; —C(O)C₁-C₃alkyl; —C(O)OC₁-C₃alkyl; —NH₂; —NH(C₁-C₃alkyl); and —N(C₁-C₃alkyl)₂, which G is optionally further substituted as defined for a compound of Formula (II), Formula (IIa) or Formula (IIb) respectively.

In another Embodiment (33.1), the invention provides a compound of Formula (II), Formula (IIa), or Formula (IIb), or a pharmaceutically acceptable salt thereof, according to any preceding Embodiment, wherein G is independently selected for each occurrence from the group consisting of wherein G is independently selected for each occurrence from the group consisting —OH; —F; —Cl; —CH₃; —OCH₃; and —CF₃.

In another Embodiment (34), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein J is independently selected for each occurrence from the group consisting of —H, —OH, —F, —Cl, —CH₃, —CH₂OH, —OCH₃, —OCF₃, and —OCF₂H.

In another Embodiment (34.1), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein J is independently selected for each occurrence from the group consisting of —H, —F, —Cl, —CH₃; —CF₃; —OCH₃; and —OCF₃.

In another Embodiment (34.2), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein J is independently selected for each occurrence from the group consisting of —H, —F, —CH₃; and —CF₃.

In another Embodiment (35), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein R¹⁰ is independently selected for each occurrence from the group consisting of —H, —OH, —F, —C₁, —CH₃, —CF₃, —CH₂OH, —OCH₃, —OCF₃, and —OCF₂H.

In another Embodiment (35.1), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein R¹⁰ is independently selected for each occurrence from the group consisting of —H, —F, —C₁, —CH₃, —CF₃, and —OCF₃.

In another Embodiment (35.2), the invention provides a compound of Formula (II), Formula (IIa), or Formula (IIb), or a pharmaceutically acceptable salt thereof, according to any preceding Embodiment of Formula (II), Formula (IIa) or Formula (IIb), wherein R¹⁰ is —H

In another Embodiment (36), the invention provides a compound of Formula (II), or a pharmaceutically acceptable salt thereof, according to any preceding Embodiment of Formula (II), wherein

R¹ is

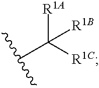

R^{1A} is selected from the group consisting of methyl, ethyl, n-propyl, phenyl, and pyridyl, which R^{1A} is optionally substituted as defined for a compound of Formula (I);

R^{1B} is selected from the group consisting of —H, methyl, ethyl, n-propyl and i-propyl, which R^{1B} is optionally substituted as defined for a compound of Formula (I);

R^{1C} is —H;

W is

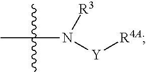

Y is selected from the group consisting of —CH₂— and —CH₂CH₂—, which Y is unsubstituted or optionally further substituted as defined for a compound of Formula (I);

R³ is —H;

R^{4A} is selected from the group consisting of —CH₃; —CO₂H; and —C(O)NHCH₃;

R¹⁰ is —H;

and where E, G and J are all defined as for a compound of Formula (II).

In another Embodiment (36.1), the invention provides a compound of Embodiment (36), or a pharmaceutically acceptable salt thereof, wherein

E is independently selected for each occurrence from the group consisting of OH; —F; —Cl; —CH₃; —OCH₃; and —CF₃;

G is independently selected for each occurrence from the group consisting of —OH; —F; —Cl; —CH₃; —OCH₃; and —CF₃; and

J is independently selected for each occurrence from the group consisting of —H, —F, —Cl, —CH₃; —CF₃; —OCH₃; and —OCF₃.

In another Embodiment (36.2), the invention provides a compound of Formula (II), or a pharmaceutically acceptable salt thereof, according to any preceding Embodiment of Formula (II), wherein

R¹ is

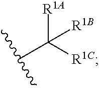

R^{1A} is selected from the group consisting of —CH₂OCH₃; phenyl; methoxyphenyl; and pyridyl;

R^{1B} is selected from the group consisting of methyl, ethyl, n-propyl, i-propyl, and —CH₂OCH₃;

R^{1C} is —H;

W is

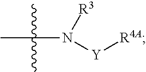

Y is selected from the group consisting of —CH₂— and —CH₂CH₂—;

R³ is —H;

R^{4A} is selected from the group consisting of —CH₃; —CO₂H; and —C(O)NHCH₃; and

R¹⁰ is —H.

The present invention also relates to a pharmaceutical composition comprising a compound of Formula (II), or a pharmaceutically acceptable salt thereof, and a pharmaceutically acceptable excipient.

The present invention also relates to a method of treating a disease or a disorder in a patient, comprising administering to a patient in need thereof a therapeutically effective amount of a compound of Formula (II), or a pharmaceutically acceptable salt thereof or a pharmaceutical composition comprising a therapeutically effective amount of a compound of Formula (II), or a pharmaceutically acceptable salt thereof.

In another embodiment, the present invention further provides a method of inhibiting a BET family bromodomain in a cell, comprising contacting the cell with a therapeutically effective amount of a compound of Formula (II), or a pharmaceutically acceptable salt thereof, or a pharmaceutical composition comprising a therapeutically effective amount of a compound of Formula (II), or a pharmaceutically acceptable salt thereof.

### BRIEF DESCRIPTION OF THE DRAWINGS

FIG. 1 shows the effect of BET inhibitors on cell proliferation in MM1.S (A) and OPM-2 (B) multiple myeloma cell lines.

FIG. 2 shows that Example 10 down-regulated c-MYC and MYB mRNA expression over 2 to 24 hours (A) and protein expression by Western blot (B) in MM1.S cells.

FIG. 3 shows the effect of BET inhibitors on cell proliferation in HCC2429 NMC cell line.

FIG. 4 shows that Example 10 down-regulated c-MYC and SOX2 protein expression at 72 hours by Western blot in HCC2429 cells.

FIG. 5 shows that BET inhibitors induced squamous cell differentiation in HCC2429 cells as indicated by dose dependent increase in mRNA expression of involucrin (A) and Keratin 14 (B) at 72 hours.

### DETAILED DESCRIPTION

The present invention relates to novel heterocyclic compounds of the invention which, in general, inhibit BET family bromodomains.

Compounds reported as BET family bromodomain binding agents include those disclosed in WO 2009/084693, WO 2011/054841, WO 2011/054843, WO 2011/054844, WO 2011/054845, WO 2011/054846, WO 2011/054848, WO 2011/143669, WO 2011/161031, WO 2012/143413, WO 2012/143415, WO 21012/143416, WO 2012/150234, WO 2013/027168, US 2014/142102, US 2014/140956, WO 2014/095775, WO 2014/154760, WO 2014/154762, WO 2014/160873, WO 2014/170350, and WO 2014/173241.

Throughout this application, it should be noted that, as used in this specification and the appended claims, the singular form “a”, “an” and “the” include plural references unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention is related. The following terms are defined for purposes of the invention as described herein.

As used herein, unless otherwise noted, “alkyl” whether used alone or as part of a substituent group refers to a saturated straight or branched hydrocarbon chain (ie a substituent obtained from a hydrocarbon by removal of a hydrogen) having from one to twenty carbon atoms or any number within this range, for example, from one to six carbon atoms, from one to four carbon atoms or from one to three carbon atoms. Designated numbers of carbon atoms (e.g. C₁₋₆) shall refer independently to the number of carbon atoms in an alkyl moiety or to the alkyl portion of a larger alkyl-containing substituent. Examples of alkyl groups include, but are not limited to, methyl, ethyl, n-propyl, iso-propyl, n-butyl, sec-butyl, iso-butyl, tert-butyl, pentyl, isoamyl, hexyl and the like. Where so indicated, alkyl groups can be optionally substituted. In substituent groups with multiple alkyl groups such as N(C₁-C₆alkyl)₂, the alkyl groups may be the same or different.

As used herein, unless otherwise noted, “alkoxy” refers to groups of formula —Oalkyl, wherein “alkyl” is as defined herein. Designated numbers of carbon atoms (e.g. —OC₁-C₆) shall refer independently to the number of carbon atoms in the alkyl moiety of the alkoxy group, for example, but not limited to, from one to six carbon atoms or from one to three carbon atoms. Examples of alkoxy groups include, but are not limited to, methoxy, ethoxy, n-propoxy, iso-propoxy, n-butoxy, sec-butoxy, iso-butoxy, tert-butoxy, and the like. Where so indicated, alkoxy groups can be optionally substituted.

As used herein, unless otherwise noted, “aryl” whether used alone or part of another group refers to a carbocyclic fully unsaturated or partially unsaturated single or fused ring system. If the rings are fused, one of the rings must be fully unsaturated or partially unsaturated and the fused ring(s) may be fully saturated, partially unsaturated or fully unsaturated. The aryl group may be optionally substituted as defined herein. The term “aryl” embraces aromatic radicals such as phenyl, naphthyl, tetrahydronaphthyl, indanyl, biphenyl, benzo[b][1,4]oxazin-3(4H)-onyl, 2,3-dihydro-1H indenyl and 1,2,3,4-tetrahydronaphthalenyl.

As used herein, unless otherwise noted, “cycloalkyl” whether used alone or as part of another group, refers to a fully saturated hydrocarbon ring having from three to fourteen ring carbon atoms, for example, from four to seven; or from three to seven; or from three to six; or from three to five ring carbon atoms. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Where so indicated, cycloalkyl rings can be optionally substituted. Examples of cycloalkyl groups include, but are not limited to, cyclopropyl, cyclobutyl, cyclopentyl, cyclohexyl, cycloheptyl, cyclooctanyl, decalinyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, unless otherwise noted, the terms “haloalkyl” and “haloalkoxy” are intended to include both branched and straight-chain saturated aliphatic “alkyl” or “alkoxy” groups respectively, wherein “alkyl” and “alkoxy” are as defined herein, having the specified number of carbon atoms and in which at least one hydrogen is replaced with a halogen atom. As used herein, the term “halogen atom” refers to F, Cl, Br and I. Haloalkyl groups include perhaloalkyl groups, wherein all hydrogens of an alkyl group have been replaced with halogens (e.g., —CF₃, —CF₂CF₃). In certain embodiments in which two or more hydrogen atoms are replaced by halogen atoms, the halogen atoms can be the same (e.g., CHF₂, —CF₃) or different (e.g., CF₂Cl). Where so indicated, haloalkyl or haloalkoxy groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

As used herein, unless otherwise noted, the terms “heterocyclyl” and “heterocycloalkyl” are used interchangeably and, whether used alone or as part of another group, are defined herein as referring to a group having one or more rings (e.g., 1, 2 or 3 rings) and having from 3 to 11 ring atoms (e.g. 3 to 6 ring atoms, 4 to 7 ring atoms, 4 to 5 ring atoms) wherein at least one ring atom, alternatively 1 to 5 ring atoms, alternatively 1 to 4 ring atoms, alternatively 1 to 3 ring atoms, alternatively one ring atom, alternatively two ring atoms, is a heteroatom, independently selected, unless indicated otherwise, from the group consisting of nitrogen (N), oxygen (O), and sulfur (S), and wherein the ring that includes the heteroatom is fully saturated. Exemplary heterocyclyl groups have from 3 to 11 ring atoms, alternatively 4 to 7 ring atoms, alternatively 4 to 5 ring atoms, alternatively 3 to 6 ring atoms, of which, where chemically possible, from 1 to 5, alternatively 1 to 4, alternatively 1 to 3, alternatively 4, alternatively 3, alternatively 2, alternatively 1 ring atom, is a heteroatoms independently selected in each instance from, unless indicated otherwise, the group consisting of nitrogen (N), oxygen (O), or sulfur (S). In a group that has a heterocyclyl substituent, unless otherwise stated, the ring atom of the heterocyclyl substituent that is bound to the group may be one of the heteroatoms, or it may be a ring carbon atom, where the ring carbon atom may be in the same ring as the heteroatom(s), or the ring carbon may be in a different ring from the heteroatom(s). Where so indicated, the heterocyclyl substituent can be optionally further substituted with one or more group(s) or substituent(s), which group(s) or substituent(s) may be bound to the heteroatom(s) or may be bound to the ring carbon atom, where the ring carbon atom may be in the same ring as the at least one heteroatom or where the ring carbon atom may be in a different ring rom the heteroatom(s). Examples of monocyclic heterocyclyl groups include, but are not limited to, oxetanyl, diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline.

As used herein, unless otherwise noted, the term “heteroaryl” whether used alone or as part of another group, is defined herein as a single or fused ring system having from five to eleven ring atoms (e.g. from five to ten ring atoms of from five to six ring atoms) wherein at least one ring atom, alternatively 2 ring atoms, alternatively 3 ring atoms, alternatively 4 ring atoms, in at least one ring is a heteroatom independently selected in each instance from, unless otherwise indicated, the group consisting of nitrogen (N), oxygen (O), and sulfur (S), and wherein further at least one of the rings comprising a heteroatom is fully unsaturated or partially unsaturated. In heteroaryl groups that include 2 or more fused rings, additional rings may bear one or more heteroatoms, may be a carbocycle (e.g., 6,7-Dihydro-5H-cyclopentapyrimidine) or may be aryl (e.g., benzofuranyl, benzo-thiophenyl, indolyl, indolinyl, tetrahydroquinolinyl, chromanyl, 1,4-dioxochromanyl). In a group that has a heteroaryl substituent, unless otherwise indicated, the ring atom of the heteroaryl substituent that is bound to the group may be the at least one heteroatom, or it may be a ring carbon atom, where the ring carbon atom may be in the same ring as the at least one heteroatom or where the ring carbon may be in a different ring from the at least one heteroatom. Where so indicated, heteroaryl groups can be substituted. If the heteroaryl substituent is substituted with a group or substituent, the group or substituent may be bound to the heteroatom, or it may be bound to a ring carbon atom, where the ring carbon atom may be in the same ring as the heteroatom(s), or where the ring carbon atom may be in a different ring from the heteroatom(s). Examples of monocyclic heteroaryl rings include, but are not limited to, 1,2,3,4-tetrazolyl, [1,2,3]triazolyl, [1,2,4]triazolyl, triazinyl, thiazol-2-yl, thiazol-4-yl, imidazol-1-yl, 1H-imidazol-2-yl, 1H-imidazol-4-yl, oxazolyl, isoxazolin-5-yl, furan-2-yl, furan-3-yl, thiophen-2-yl, thiophen-4-yl, pyrimidin-2-yl, pyrimidin-4-yl, pyrimidin-5-yl, pyridazinyl, pyrazinyl, pyridin-2-yl, pyridin-3-yl, and pyridin-4-yl pyridinyl. Examples of heteroaryl rings containing 2 or more fused rings include, but are not limited to, benzofuranyl, benzothiophenyl, benzoxazolyl, benzthiazolyl, benztriazolyl, cinnolinyl, naphthyridinyl, benzimidazolyl, aza-indolyl, aza-benzimidazolyl, phenanthridinyl, 7H-purinyl, 9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, and isoquinolinyl. The term “heteroaryl” also includes pyridyl N-oxides and groups containing a pyridine N-oxide ring.

As used herein, unless otherwise stated, the term “amino” refers to —NH₂.

As used herein, unless otherwise stated, the term “alkylamino” refers to —N(H)alkyl, the term “alkyl” having already been defined herein. Examples of alkylamino substituents include, but are not limited to, methylamino, ethylamino, and propylamino.

As used herein, unless otherwise stated, the term “dialkylamino” refers to —N(alkyl)₂ where the two alkyls may be the same or different and where the term “alkyl” has already been defined herein. Examples of dialkylamino substituents include, but are not limited to, dimethylamino, diethylamino, ethylmethylamino, and dipropylamino.

As used herein, unless otherwise stated, the term “amido” refers to —C(═O)NH₂.

As used herein, unless otherwise stated, the term “halogen” or “halogen atom” refers to the group consisting of fluorine (which may be depicted as —F), chlorine (which may be depicted as —Cl), bromine (which may be depicted as —Br), or iodine (which may be depicted as —I).

As used herein, unless otherwise stated, the terms “hydroxy” and “hydroxyl” are used interchangeably and as used herein mean an —OH group. As used herein, unless otherwise noted, the terms “hydroxyalkyl” and “hydroxyalkoxy” are intended to include both branched and straight-chain saturated aliphatic “alkyl” or “alkoxy” groups respectively, wherein “alkyl” and “alkoxy” are as defined herein, having the specified number of carbon atoms and in which at least one hydrogen is replaced with a —OH group. Where so indicated, hydroxyalkyl and hydroxyalkoxy groups can optionally be substituted with one or more substituents in addition to —OH. Examples of hydroxyalkyl groups include, but are not limited to, CH₂OH, CH₂CH₂OH, CH₂(OH)CH₂OH.

As used herein, unless otherwise stated, the term “oxo” ═O.

As used herein, unless otherwise stated, the term “carbonyl” refers to C═O.

As used herein, unless otherwise stated, the term “carboxy” refers to —CO₂H.

As used herein, unless otherwise stated, the term sulfonyl refers to —SO₂—.

As used herein, the term “substituted” is used throughout the specification. The term “substituted” is defined herein as a moiety, whether acyclic or cyclic, which has one or more (e.g. 1-10) hydrogen atoms replaced by a substituent as defined herein below. Substituents include those that are capable of replacing one or two hydrogen atoms of a single moiety at a time, and also those that can replace two hydrogen atoms on two adjacent carbons to form said substituent. For example, substituents that replace single hydrogen atoms include, but are not limited to, halogen, hydroxy, and the like. A two hydrogen atom replacement includes, but is not limited to, carbonyl, oximino, and the like. Substituents that replace two hydrogen atoms from adjacent carbon atoms include, but are not limited to, epoxy, and the like. When a moiety is described as “substituted” any number of its hydrogen atoms can be replaced, as described above. For example, difluoromethyl is a substituted C₁ alkyl; trifluoromethyl is a substituted C₁ alkyl; 4-hydroxyphenyl is a substituted aryl ring; (N,N-dimethyl-5-amino)octanyl is a substituted C₈ alkyl; 3-guanidinopropyl is a substituted C₃ alkyl; and 2-carboxy-3-fluoropyridinyl is a substituted heteroaryl.

A multi-moiety substituent is bound through the atom indicated by “-”. To illustrate this the term “—OC₁-C₃hydroxyalkyl” is an OC₁-C₃alkyl group substituted by a hydroxy group. Further, any carbon number pre-fix attached to a multi-moiety substituent only applies to the moiety it immediately precedes. To illustrate, the term “cycloalkyl(C₁-C₄)alkyl” contains two moieties: alkyl and cycloalkyl. The (C₁-C₄) pre-fix on the cycloalkyl(C₁-C₄)alkyl means that the alkyl moiety of the alkylcycloalkyl contains from 1 to 4 carbon atoms, the (C₁-C₄) pre-fix does not describe the cycloalkyl moiety.

If a group of substituents are collectively described as being optionally substituted by one or more of a list of substituents, the group may include (1) unsubstitutable substituents, (2) substitutable substituents that are not substituted by the optional substituents, and/or (3) substitutable substituents that are substituted by one or more of the optional substituents.

If a substituent is described such that it “may be substituted” or as being “optionally substituted” with up to a particular number of non-hydrogen substituents, that substituent may be either (1) not substituted; or (2) substituted by up to that particular number of non-hydrogen substituents or by up to the maximum number of substitutable positions on the substituents, whichever is less. Thus, for example, if a substituent is described as a heteroaryl optionally substituted with one, two or three substituents, then any heteroaryl with less than three substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent.

At various places in the present specification, substituents of compounds are disclosed in groups or in ranges. It is specifically intended that the description include each and every individual sub-combination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄- C₆, C₄-C₅, and C₅-C₆ alkyl. For example, the term “C₁₋₃ alkyl” is specifically intended to individually disclose C₁, C₂, C₃, C₁-C₃, C₁-C₂, and C₂-C₃ alkyl.

As used herein, the term “compounds of the invention” means, unless otherwise stated, compounds of Formula (I), Formula (Ia), Formula (Ib) Formula (I′), Formula (Ia′), or Formula (Ib′) or compounds of Embodiment (1), Embodiment (1.1), Embodiment (2), Embodiment (2.1), Embodiment (2.2), Embodiment (2.3), Embodiment (2.4), Embodiment (3), Embodiment (3.1), Embodiment (3.2), Embodiment (3.3), Embodiment (3.4), Embodiment (4), Embodiment (4.1), Embodiment (4.2), Embodiment (4.2a), Embodiment (4.3), Embodiment (4.4), Embodiment (4.5), Embodiment (4.6), Embodiment (4.7), Embodiment (4.8), Embodiment (4.9), Embodiment (4.9a), Embodiment (4.10), Embodiment (4.11), Embodiment (4.12), Embodiment (4.13), Embodiment (4.14), Embodiment (5), Embodiment (5.0), Embodiment (5.1), Embodiment (5.1a), Embodiment (5.2), Embodiment (5.3), Embodiment (5.4), Embodiment (5.5), Embodiment (5.6), Embodiment (5.7), Embodiment (6), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), Embodiment (6.8), Embodiment (6.9), Embodiment (6.9a), Embodiment (6.10), Embodiment (6.11), Embodiment (6.12), Embodiment (7), Embodiment (7.1), Embodiment (7.2), Embodiment (7.2a), Embodiment (7.2b), Embodiment (7.3), Embodiment (7.4), Embodiment (8), Embodiment (8.1), Embodiment (8.2), Embodiment (8.3), Embodiment (8.4), Embodiment (9), Embodiment (9.1), Embodiment (9.2), Embodiment (10), Embodiment (10.1), Embodiment (10.2), Embodiment (10.3), Embodiment (10.4), Embodiment (10.5), Embodiment (11), Embodiment (11.0), Embodiment (11.1), Embodiment (11.2), Embodiment (11.3), Embodiment (11.4), Embodiment (11.5), Embodiment (11.6), Embodiment (11.7), Embodiment (11.8), Embodiment (12), Embodiment (12.1), Embodiment (12.2), Embodiment (12.3), Embodiment (13), Embodiment (13.1), Embodiment (13.2), Embodiment (13.3), Embodiment (13.4), Embodiment (13.5), Embodiment (14), Embodiment (14.1), Embodiment (14.2), Embodiment (14.3), Embodiment (14.4), Embodiment (15) Embodiment (15.1), Embodiment (15.2), Embodiment (15.3), Embodiment (15.4), Embodiment (15.5), Embodiment (16), Embodiment (16.1), Embodiment (16.2), Embodiment (16.3), Embodiment (16.4), Embodiment (17), Embodiment (17.1), Embodiment (18), Embodiment (18.1), Embodiment (19), Embodiment (19.1), Embodiment (19.1a), Embodiment (19.2), Embodiment (19.3), Embodiment (19.4), Embodiment (50), Embodiment (50.1), Embodiment (50.2), compounds of Formula (II), Formula (IIa), or Formula (IIb), Formula (II′), Formula (IIa′), or Formula (IIb′), or Embodiment (20), Embodiment (20.0), Embodiment (20.1), Embodiment (20.2), Embodiment (20.3), Embodiment (20.4), Embodiment (21), Embodiment (21.1), Embodiment (21.2), Embodiment (21.3), Embodiment (21.4), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), Embodiment (22.10a), Embodiment (22.11), Embodiment (22.12), Embodiment (22.13), Embodiment (22.14), Embodiment (22.15), Embodiment (23), Embodiment (23.0), Embodiment (23.1), Embodiment (23.2), Embodiment (23.2a), Embodiment (23.3), Embodiment (23.4), Embodiment (23.5), Embodiment (23.6), Embodiment (23.7), Embodiment (23.8), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), Embodiment (24.8), Embodiment (24.9), Embodiment (24.9a), Embodiment (24.10), Embodiment (24.11), Embodiment (24.12), Embodiment (25), Embodiment (25.1), Embodiment (25.2), Embodiment (25.3), Embodiment (25.4), Embodiment (25.5), Embodiment (26), Embodiment (26.0), Embodiment (26.1), Embodiment (26.2), Embodiment (26.3), Embodiment (26.4), Embodiment (26.5), Embodiment (26.6), Embodiment (26.7), Embodiment (26.8), Embodiment (27), Embodiment (27.1), Embodiment (27.2), Embodiment (27.3), Embodiment (28), Embodiment (28.1), Embodiment (28.2), Embodiment (28.3), Embodiment (28.4), Embodiment (28.5), Embodiment (29), Embodiment (29.1), Embodiment (29.2), Embodiment (29.3), Embodiment (29.4), Embodiment (30), Embodiment (30.1), Embodiment (30.2), Embodiment (30.3), Embodiment (30.4), Embodiment (30.5), Embodiment (31), Embodiment (31.1), Embodiment (31.2), Embodiment (31.3), Embodiment (31.4), Embodiment (32), Embodiment (32.1), Embodiment (33), Embodiment (33.1), Embodiment (34), Embodiment (34.1), Embodiment (34.2), Embodiment (35), Embodiment (35.1), Embodiment (35.2), Embodiment (36), or Embodiment (36.1), Embodiment (36.2), or a pharmaceutically acceptable salt of such compounds.

As used herein, the term “compounds of Formula (I)” means, unless otherwise stated, compounds of Formula (I), Formula (Ia), Formula (Ib) Formula (I′), Formula (Ia′), or Formula (Ib′) or compounds of Embodiment (1), Embodiment (1.1), Embodiment (2), Embodiment (2.1), Embodiment (2.2), Embodiment (2.3), Embodiment (2.4), Embodiment (3), Embodiment (3.1), Embodiment (3.2), Embodiment (3.3), Embodiment (3.4), Embodiment (4), Embodiment (4.1), Embodiment (4.2), Embodiment (4.2a), Embodiment (4.3), Embodiment (4.4), Embodiment (4.5), Embodiment (4.6), Embodiment (4.7), Embodiment (4.8), Embodiment (4.9), Embodiment (4.9a), Embodiment (4.10), Embodiment (4.11), Embodiment (4.12), Embodiment (4.13), Embodiment (4.14), Embodiment (5), Embodiment (5.0), Embodiment (5.1), Embodiment (5.1a), Embodiment (5.2), Embodiment (5.3), Embodiment (5.4), Embodiment (5.5), Embodiment (5.6), Embodiment (5.7), Embodiment (6), Embodiment (6.0), Embodiment (6.1), Embodiment (6.2), Embodiment (6.2a), Embodiment (6.3), Embodiment (6.4), Embodiment (6.5), Embodiment (6.6), Embodiment (6.7), Embodiment (6.8), Embodiment (6.9), Embodiment (6.9a), Embodiment (6.10), Embodiment (6.11), Embodiment (6.12), Embodiment (7), Embodiment (7.1), Embodiment (7.2), Embodiment (7.2a), Embodiment (7.2b), Embodiment (7.3), Embodiment (7.4), Embodiment (8), Embodiment (8.1), Embodiment (8.2), Embodiment (8.3), Embodiment (8.4), Embodiment (9), Embodiment (9.1), Embodiment (9.2), Embodiment (10), Embodiment (10.1), Embodiment (10.2), Embodiment (10.3), Embodiment (10.4), Embodiment (10.5), Embodiment (11), Embodiment (11.0), Embodiment (11.1), Embodiment (11.2), Embodiment (11.3), Embodiment (11.4), Embodiment (11.5), Embodiment (11.6), Embodiment (11.7), Embodiment (11.8), Embodiment (12), Embodiment (12.1), Embodiment (12.2), Embodiment (12.3), Embodiment (13), Embodiment (13.1), Embodiment (13.2), Embodiment (13.3), Embodiment (13.4), Embodiment (13.5), Embodiment (14), Embodiment (14.1), Embodiment (14.2), Embodiment (14.3), Embodiment (14.4), Embodiment (15) Embodiment (15.1), Embodiment (15.2), Embodiment (15.3), Embodiment (15.4), Embodiment (15.5), Embodiment (16), Embodiment (16.1), Embodiment (16.2), Embodiment (16.3), Embodiment (16.4), Embodiment (17), Embodiment (17.1), Embodiment (18), Embodiment (18.1), Embodiment (19), Embodiment (19.1), Embodiment (19.1a), Embodiment (19.2), Embodiment (19.3), Embodiment (19.4), Embodiment (50), Embodiment (50.1), Embodiment (50.2), or a pharmaceutically acceptable salt of such compounds.

As used herein, the term “compounds of Formula (II)” means, unless otherwise stated, Formula (II), Formula (IIa), or Formula (IIb), Formula (II′), Formula (IIa′), or Formula (IIb′), or Embodiment (20), Embodiment (20.0), Embodiment (20.1), Embodiment (20.2), Embodiment (20.3), Embodiment (20.4), Embodiment (21), Embodiment (21.1), Embodiment (21.2), Embodiment (21.3), Embodiment (21.4), Embodiment (22), Embodiment (22.1), Embodiment (22.2), Embodiment (22.3), Embodiment (22.3a), Embodiment (22.4), Embodiment (22.5), Embodiment (22.6), Embodiment (22.7), Embodiment (22.8), Embodiment (22.9), Embodiment (22.10), Embodiment (22.10a), Embodiment (22.11), Embodiment (22.12), Embodiment (22.13), Embodiment (22.14), Embodiment (22.15), Embodiment (23), Embodiment (23.0), Embodiment (23.1), Embodiment (23.2), Embodiment (23.2a), Embodiment (23.3), Embodiment (23.4), Embodiment (23.5), Embodiment (23.6), Embodiment (23.7), Embodiment (23.8), Embodiment (24), Embodiment (24.0), Embodiment (24.1), Embodiment (24.2), Embodiment (24.2a), Embodiment (24.3), Embodiment (24.4), Embodiment (24.5), Embodiment (24.6), Embodiment (24.7), Embodiment (24.8), Embodiment (24.9), Embodiment (24.9a), Embodiment (24.10), Embodiment (24.11), Embodiment (24.12), Embodiment (25), Embodiment (25.1), Embodiment (25.2), Embodiment (25.3), Embodiment (25.4), Embodiment (25.5), Embodiment (26), Embodiment (26.0), Embodiment (26.1), Embodiment (26.2), Embodiment (26.3), Embodiment (26.4), Embodiment (26.5), Embodiment (26.6), Embodiment (26.7), Embodiment (26.8), Embodiment (27), Embodiment (27.1), Embodiment (27.2), Embodiment (27.3), Embodiment (28), Embodiment (28.1), Embodiment (28.2), Embodiment (28.3), Embodiment (28.4), Embodiment (28.5), Embodiment (29), Embodiment (29.1), Embodiment (29.2), Embodiment (29.3), Embodiment (29.4), Embodiment (30), Embodiment (30.1), Embodiment (30.2), Embodiment (30.3), Embodiment (30.4), Embodiment (30.5), Embodiment (31), Embodiment (31.1), Embodiment (31.2), Embodiment (31.3), Embodiment (31.4), Embodiment (32), Embodiment (32.1), Embodiment (33), Embodiment (33.1), Embodiment (34), Embodiment (34.1), Embodiment (34.2), Embodiment (35), Embodiment (35.1), Embodiment (35.2), Embodiment (36), or Embodiment (36.1), Embodiment (36.2), or a pharmaceutically acceptable salt of such compounds.

In certain embodiments, the compounds of Formula (I) include:
N-{6-[acetyl(methyl)amino]-4-[(1S)-1-(2-methoxyphenyl)ethyl]-3-oxo-3,4-dihydropyrido[2,3-b]pyrazin-2-yl}-beta-alanine;
N-{6-[acetyl(ethyl)amino]-4-[(1S)-1-(2-methoxyphenyl)ethyl]-3-oxo-3,4-dihydropyrido[2,3-b]pyrazin-2-yl}-beta-alanine;
N-{6-[acetyl(methyl)amino]-3-oxo-4-[(1S)-1-phenylpropyl]-3,4-dihydropyrido[2,3-b]pyrazin-2-yl}-beta-alanine;
N-{6-[(hydroxyacetyl)(methyl)amino]-3-oxo-4-[(1S)-1-phenylpropyl]-3,4-dihydropyrido[2,3-b]pyrazin-2-yl}-beta-alanine;
N-{4-[(1S)-1-(2-methoxyphenyl)ethyl]-2-(methylamino)-3-oxo-3,4-dihydropyrido[2,3-b]pyrazin-6-yl}-N-methylacetamide;
N-{6-[methyl(2-methylpropanoyl)amino]-3-oxo-4-[(1S)-1-phenylpropyl]-3,4-dihydropyrido[2,3-b]pyrazin-2-yl}-beta-alanine;
N-{6-[butanoyl(methyl)amino]-3-oxo-4-[(1S)-1-phenylpropyl]-3,4-dihydropyrido[2,3-b]pyrazin-2-yl}-beta-alanine;
N-{6-[(cyclobutylcarbonyl)(methyl)amino]-3-oxo-4-[(1S)-1-phenylpropyl]-3,4-dihydropyrido[2,3-b]pyrazin-2-yl}-beta-alanine;
N-{6-[methyl(methylcarbamoyl)amino]-3-oxo-4-[(1S)-1-phenylpropyl]-3,4-dihydropyrido[2,3-b]pyrazin-2-yl}-beta-alanine;
N-{6-[methyl(propanoyl)amino]-3-oxo-4-[(1S)-1-phenylpropyl]-3,4-dihydropyrido[2,3-b]pyrazin-2-yl}-beta-alanine;
N-{6-[methyl(propanoyl)amino]-3-oxo-4-[(1S)-1-(pyridin-2-yl)propyl]-3,4-dihydropyrido[2,3-b]pyrazin-2-yl}-beta-alanine;
N-{6-[methyl(propanoyl)amino]-3-oxo-4-(1-phenylcyclobutyl)-3,4-dihydropyrido[2,3-b]pyrazin-2-yl}-beta-alanine;
N-{4-(2, 5-diethylcyclopentyl)-6-[methyl(propanoyl)amino]-3-oxo-3,4-dihydropyrido[2,3-b]pyrazin-2-yl}-beta-alanine;
N-{6-[acetyl(methyl)amino]-4-[(1R)-2-methoxy-1-phenylethyl]-3-oxo-3,4-dihydropyrido[2,3-b]pyrazin-2-yl}-beta-alanine;
N-{6-[methyl(propanoyl)amino]-3-oxo-4-[(3S,4S)-4-phenyltetrahydrofuran-3-yl]-3,4-dihydropyrido[2,3-b]pyrazin-2-yl}-beta-alanine;
N-{6-[acetyl(methyl)amino]-4-[(2R)-1-methoxybutan-2-yl]-3-oxo-3,4-dihydropyrido[2,3-b]pyrazin-2-yl}-beta-alanine;
N-{4-[(2R)-1-methoxypentan-2-yl]-6-[methyl(propanoyl)amino]-3-oxo-3,4-dihydropyrido[2,3-b]pyrazin-2-yl}-beta-alanine;
N-(4-[(2R)-1-methoxybutan-2-yl]-2-{[2-(methylamino)-2-oxoethyl]amino}-3-oxo-3,4-dihydropyrido[2,3-b]pyrazin-6-yl)-N-methylpropanamide;
N-[4-(1,3-dimethoxypropan-2-yl)-2-{[2-(methylamino)-2-oxoethyl]amino}-3-oxo-3,4-dihydropyrido[2,3-b]pyrazin-6-yl]-N-methylpropanamide;
N-[4-(1,3-dimethoxypropan-2-yl)-2-(methylamino)-3-oxo-3,4-dihydropyrido[2,3-b]pyrazin-6-yl]-N-methylpropanamide;
N-{2-(acetylamino)-3-oxo-4-[(1S)-1-phenylpropyl]-3,4-dihydropyrido[2,3-b]pyrazin-6-yl}-N-methylacetamide;
N-{6-[(dimethylcarbamoyl)(methyl)amino]-3-oxo-4-[(1S)-1-phenylpropyl]-3,4-dihydropyrido[2,3-b]pyrazin-2-yl}-beta-alanine;
N-{6-[methyl(propanoyl)amino]-4-[(1S)-2-methyl-1-(pyridin-2-yl) propyl]-3-oxo-3,4-dihydropyrido[2, 3-b]pyrazin-2-yl}-beta-alanine;
N-{4-(1-cyclopentylcyclopropyl)-6-[methyl(propanoyl)amino]-3-oxo-3,4-dihydropyrido[2,3-b]pyrazin-2-yl}-beta-alanine;
N-(6-{[(3,3-dimethylcyclobutyl)carbonyl](methyl)amino}-3-oxo-4-[(1S)-1-phenylpropyl]-3,4-dihydropyrido[2,3-b]pyrazin-2-yl)-beta-alanine;
N-(6-{[(3,3-difluorocyclobutyl)carbonyl](methyl) amino}-3-oxo-4-[(1S)-1-phenylpropyl]-3,4-dihydropyrido[2,3-b]pyrazin-2-yl)-beta-alanine;
N-{6-[methyl(oxetan-3-ylcarbonyl)amino]-3-oxo-4-[(1S)-1-phenylpropyl]-3,4-dihydropyrido[2,3-b]pyrazin-2-yl}-beta-alanine;
N-methyl-N-(2-{[3-(methylamino)-3-oxopropyl]amino}-3-oxo-4-[(1S)-1-phenylpropyl]-3,4-dihydropyrido[2,3-b]pyrazin-6-yl)oxetane-2-carboxamide;
N-methyl-N-(2-{[2-(methylamino)-2-oxoethyl]amino}-3-oxo-4-[(1S)-1-phenylpropyl]-3,4-dihydropyrido[2, 3-b]pyrazin-6-yl) propanamide;
N-methyl-N-(2-{[2-(methylamino)-2-oxoethyl]amino}-4-[(1S)-2-methyl-1-(pyridin-2-yl)propyl]-3-oxo-3,4-dihydropyrido[2,3-b]pyrazin-6-yl)propanamide;
N³-{4-[(1R)-2-methoxy-1-phenylethyl]-6-[methyl(propanoyl)amino]-3-oxo-3,4-dihydropyrido[2,3-b]pyrazin-2-yl}-N-methyl-beta-alaninamide;
N-methyl-N³-{6-[methyl(propanoyl)amino]-3-oxo-4-[(1S)-1-phenylpropyl]-3,4-dihydropyrido[2,3-b]pyrazin-2-yl}-beta-alaninamide;
N-methyl-N³-{6-[methyl(propanoyl)amino]-3-oxo-4-[(1S)-1-(pyridin-2-yl) propyl]-3,4-dihydropyrido[2,3-b]pyrazin-2-yl}-beta-alaninamide; and
N-methyl-N³-{6-[methyl(propanoyl)amino]-4-[(1S)-2-methyl-1-(pyridin-2-yl)propyl]-3-oxo-3,4-dihydropyrido[2,3-b]pyrazin-2-yl}-beta-alaninamide
   or a pharmaceutically acceptable salt thereof.

Preferred compounds of Formula (I) are:
N-{6-[methyl(propanoyl)amino]-3-oxo-4-[(1S)-1-phenylpropyl]-3,4-dihydropyrido[2,3-b]pyrazin-2-yl}-beta-alanine;
N-methyl-N³-{6-[methyl(propanoyl)amino]-3-oxo-4-[(1S)-1-phenylpropyl]-3,4-dihydropyrido[2,3-b]pyrazin-2-yl}-beta-alaninamide;
N-methyl-N-(2-{[2-(methylamino)-2-oxoethyl]amino}-3-oxo-4-[(1S)-1-phenylpropyl]-3,4-dihydropyrido[2,3-b]pyrazin-6-yl)propanamide;
N³-{4-[(1R)-2-methoxy-1-phenylethyl]-6-[methyl(propanoyl)amino]-3-oxo-3,4-dihydropyrido[2,3-b]pyrazin-2-yl}-N-methyl-beta-alaninamide;
N-methyl-N-(2-{[3-(methylamino)-3-oxopropyl]amino}-3-oxo-4-[(1S)-1-phenylpropyl]-3,4-dihydropyrido[2,3-b]pyrazin-6-yl)oxetane-2-carboxamide;
N-{6-[methyl(methylcarbamoyl)amino]-3-oxo-4-[(1S)-1-phenylpropyl]-3,4-dihydropyrido[2,3-b]pyrazin-2-yl}-beta-alanine; and
N-methyl-N³-{6-[methyl(propanoyl)amino]-4-[(1S)-2-methyl-1-(pyridin-2-yl)propyl]-3-oxo-3,4-dihydropyrido[2,3-b]pyrazin-2-yl}-beta-alaninamide
   or a pharmaceutically acceptable salt thereof.

An especially preferred compound of Formula (I) is:
N-{6-[methyl(propanoyl)amino]-3-oxo-4-[(1S)-1-phenylpropyl]-3,4-dihydropyrido[2,3-b]pyrazin-2-yl}-beta-alanine
   or a pharmaceutically acceptable salt thereof.

In certain embodiments, the compounds of Formula (II) include:
6-(3,5-dimethyl-1,2-oxazol-4-yl)-4-[(1S)-1-(2-methoxyphenyl)ethyl]-2-{[2-(morpholin-4-yl)ethyl]amino}pyrido[2,3-b]pyrazin-3(4H)-one;
6-(3,5-dimethyl-1,2-oxazol-4-yl)-4-(2-ethoxybenzyl)-2-{[2-(morpholin-4-yl)ethyl]amino}pyrido[2, 3-b]pyrazin-3(4H)-one;
4-benzyl-6-(3,5-dimethyl-1,2-oxazol-4-yl)-2-{[2-(morpholin-4-yl)ethyl]amino}pyrido[2,3-b]pyrazin
3(4H)-one;
6-(3,5-dimethyl-1,2-oxazol-4-yl)-2-{[2-(morpholin-4-yl)ethyl]amino}-4-[(1R)-1-phenylpropyl]pyrido[2, 3-b]pyrazin-3(4H)-one;
6-(3,5-dimethyl-1,2-oxazol-4-yl)-2-{[2-(morpholin-4-yl)ethyl]amino}-4-[(1S)-1-phenylpropyl]pyrido[2,3-b]pyrazin-3(4H)-one;
6-(3,5-dimethyl-1,2-oxazol-4-yl)-2-{[2-(morpholin-4-yl)ethyl]amino}-4-[(1S)-1-phenylethyl]pyrido[2,3-b]pyrazin-3(4H)-one;
N-{6-(3,5-dimethyl-1,2-oxazol-4-yl)-3-oxo-4-[(1S)-1-phenylethyl]-3,4-dihydropyrido[2,3-b]pyrazin 2-yl}glycine;
N-{6-(3,5-dimethyl-1,2-oxazol-4-yl)-3-oxo-4-[(1S)-1-phenylethyl]-3,4-dihydropyrido[2,3-b]pyrazin 2-yl}-beta-alanine;
N-{6-(3,5-dimethyl-1,2-oxazol-4-yl)-4-[(1S)-1-(2-methoxyphenyl)ethyl]-3-oxo-3,4-dihydropyrido[2,3-b]pyrazin-2-yl}-beta-alanine;
N-{6-(3,5-dimethyl-1,2-oxazol-4-yl)-3-oxo-4-[(1S)-1-phenylpropyl]-3,4-dihydropyrido[2,3-b]pyrazin-2-yl}-beta-alanine;
N-{6-(3,5-dimethyl-1,2-oxazol-4-yl)-3-oxo-4-[(1S)-1-(pyrimidin-2-yl)propyl]-3,4-dihydropyrido[2, 3-b]pyrazin-2-yl}-beta-alanine;
N-{6-(3,5-dimethyl-1,2-oxazol-4-yl)-4-[(2S)-1-methoxybutan-2-yl]-3-oxo-3,4-dihydropyrido[2,3-b]pyrazin-2-yl}-beta-alanine;
N-{6-(3,5-dimethyl-1,2-oxazol-4-yl)-4-[(2R)-1-methoxybutan-2-yl]-3-oxo-3,4-dihydropyrido[2,3-b]pyrazin-2-yl}-beta-alanine;
N-[4-(1,3-dimethoxypropan-2-yl)-6-(3,5-dimethyl-1,2-oxazol-4-yl)-3-oxo-3,4-dihydropyrido[2,3-b]pyrazin-2-yl]-beta-alanine;
N-[6-(3,5-dimethyl-1,2-oxazol-4-yl)-3-oxo-4-(tetrahydro-2H-pyran-4-yl)-3,4-dihydropyrido[2,3-b]pyrazin-2-yl]-beta-alanine;
N³-{6-(3,5-dimethyl-1,2-oxazol-4-yl)-3-oxo-4-[(1S)-1-phenylethyl]-3,4-dihydropyrido[2,3-b]pyrazin-2-yl}-N-(methylsulfonyl)-beta-alaninamide;
6-(3,5-dimethyl-1,2-oxazol-4-yl)-4-[(1S)-1-phenylethyl]-2-{[2-(1H-tetrazol-5-yl)ethyl]amino}pyrido[2,3-b]pyrazin-3(4H)-one;
N-{6-(3,5-dimethyl-1,2-oxazol-4-yl)-3-oxo-4-[(1S)-1-phenylbutyl]-3,4-dihydropyrido[2,3-b]pyrazin-2-yl}-beta-alanine;
N-{6-(3,5-dimethyl-1,2-oxazol-4-yl)-4-[(1S)-2-methyl-1-phenylpropyl]-3-oxo-3,4-dihydropyrido[2,3-b]pyrazin-2-yl}-beta-alanine;
N-{6-(3,5-dimethyl-1,2-oxazol-4-yl)-4-[(1S)-2-methyl-1-(pyridin-2-yl)propyl]-3-oxo-3,4-dihydropyrido[2, 3-b]pyrazin-2-yl}-beta-alanine;
N-{4-[(1S)-1-cyclohexylethyl]-6-(3,5-dimethyl-1,2-oxazol-4-yl)-3-oxo-3,4-dihydropyrido[2,3-b]pyrazin-2-yl}-beta-alanine;
N-[6-(3,5-dimethyl-1,2-oxazol-4-yl)-3-oxo-4-(pentan-3-yl)-3,4-dihydropyrido[2,3-b]pyrazin-2-yl]-beta-alanine; and
N²-{6-(3, 5-dimethyl-1,2-oxazol-4-yl)-4-[(1S)-1-(2-methoxyphenyl)ethyl]-3-oxo-3,4-dihydropyrido[2,3-b]pyrazin-2-yl}-N-methylglycinamide
   or a pharmaceutically acceptable salt thereof.

A preferred compound of Formula (II) is:
N-{6-(3,5-dimethyl-1,2-oxazol-4-yl)-4-[(2R)-1-methoxybutan-2-yl]-3-oxo-3,4-dihydropyrido[2,3-b]pyrazin-2-yl}-beta-alanine
   or a pharmaceutically acceptable salt thereof.

The compounds of the invention not only include compounds as hereinbefore defined, but also all forms of the compounds of the invention, including isomers (including optical, geometric and tautomeric isomers), hydrates, solvates, complexes, salts (including solvates and complexes thereof) crystalline and non-crystalline forms, isomorphs, polymorphs, isotopically-labeled derivatives, metabolites and prodrugs (including tautomeric forms of such prodrugs) thereof.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to for example, chiral chromatography, diastereomeric salt formation, kinetic resolution, and asymmetric synthesis. The present invention also includes cis and trans or E/Z isomers of compounds of the invention containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The compounds of the invention may exist in both unsolvated and solvated forms. The term “solvate” as used herein means a physical association of a compound with one or more solvent molecules, whether organic or inorganic, including water (‘hydrate’). As noted above, the compounds of the invention, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of the invention (e.g. a compound of Formula (I)) with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include but are not limited to aliphatic, cycloaliphatic, aromatic, araliphatic, heterocyclic, carboxylic, and sulfonic classes of organic acids.

Specific examples of suitable organic acids include but are not limited to acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, beta-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-benzylethylenediamine, chloroprocaine, choline, diethanolamine, ethylenediamine, meglumine (N-methylglucamine), and procaine. Basic nitrogen-containing groups may be quaternized with agents such as lower alkyl (C.sub.1-C.sub.6) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The present invention includes all pharmaceutically acceptable isotopically-labelled compounds of the invention wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, and ¹²⁵I are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996), incorporated herein by reference. Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

In some embodiments, compounds described herein could be prepared as prodrugs. A “prodrug” refers to an agent that is converted (e.g., either spontaneous or enzymatic) within the target physiological system into the parent drug in vivo. Prodrugs are designed to overcome problems associated with stability, toxicity, lack of specificity, or limited bioavailability. In some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985). Prodrugs may be designed as reversible drug derivatives, for use as modifiers to enhance drug transport to site-specific tissues. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Some preferred prodrugs are variations or derivatives of compounds that have groups cleavable under metabolic conditions. Common prodrugs include acid derivatives such as esters, such as carboxylic esters (eg ethyl esters) and phosphate esters prepared by reaction of parent acids with a suitable alcohol (e.g., a lower alkanol), or of parent alcohols with a suitable acid (e.g. phosphate esters of hydroxyl groups); amides prepared by reaction of the parent acid compound with an amine, or basic groups reacted to form an acylated base derivative (e.g., a lower alkylamide).

In one Embodiment, the invention relates to prodrugs of compounds of Formula (I), or a pharmaceutically acceptable salt thereof. In another Embodiment, the prodrug is an ester of a compound of Formula (I), or a pharmaceutically acceptable salt thereof. In another Embodiment, the prodrug is a phosphate ester of a compound of Formula (I), or a pharmaceutically acceptable salt thereof. In another Embodiment, the prodrug is a carboxylic ester of a compound of Formula (I), or a pharmaceutically acceptable salt thereof.

In one Embodiment, the invention relates to prodrugs of compounds of Formula (II), or a pharmaceutically acceptable salt thereof. In another Embodiment, the prodrug is an ester of a compound of Formula (II), or a pharmaceutically acceptable salt thereof. In another Embodiment, the prodrug is a phosphate ester of a compound of Formula (II), or a pharmaceutically acceptable salt thereof. In another Embodiment, the prodrug is a carboxylic ester of a compound of Formula (II), or a pharmaceutically acceptable salt thereof.

The present invention also relates to a pharmaceutical composition comprising a compound of Formula (I), or a pharmaceutically acceptable salt thereof, and a pharmaceutically acceptable excipient.

Methods of formulation are well known in the art and are disclosed, for example, in Remington: The Science and Practice of Pharmacy, Mack Publishing Company, Easton, Pa., 21st Edition (2005), incorporated herein by reference.

Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent.

In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Formulations comprising crystalline forms of the compositions described herein for slow absorption from subcutaneous or intramuscular injection are provided herein. Additionally, delayed absorption of a parenterally administered drug form may be accomplished by dissolving or suspending the compounds in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The compounds described herein can also be in micro-encapsulated form with one or more excipients as noted above. The solid dosage forms of tablets, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

Compositions of the invention may also be formulated for delivery as a liquid aerosol or inhalable dry powder. Liquid aerosol formulations may be nebulized predominantly into particle sizes that can be delivered to the terminal and respiratory bronchioles.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in p harmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

The heterocyclic compounds of the present invention, or pharmaceutically acceptable salts thereof, may inhibit the BET family of bromodomains. Such compounds may therefore be useful for treating diseases or disorders that are BET family bromodomain-dependent.

The heterocyclic compounds of the present invention, or pharmaceutically acceptable salts thereof, may also demonstrate selective inhibition activity, for example selective inhibition activity among the members of the BET family bromodomains meaning that they are for example selective inhibitors of BRD-4 BET family bromodomain when to compared to their inhibition against other BET family bromodomains such as BRDT, or for example the compounds may demonstrate selectivity inhibition activity for BET family bromodomains when compared to other targets, such as, for example, phosphodiesterase enzymes.

The heterocyclic compounds of the present invention, or pharmaceutically acceptable salts thereof, may also demonstrate other properties which make them suitable for development as pharmaceutical products, for example they demonstrate one or more of the following properties including suitable bioavailability, stability, toxicity and/or pharmacokinetic profile.

The present invention also relates to a method of treating a disease or a disorder in a patient, comprising administering to a patient in need thereof a therapeutically effective amount of a compound of Formula (I), or a pharmaceutically acceptable salt thereof, or a pharmaceutical composition comprising a therapeutically effective amount of a compound of Formula (I), or a pharmaceutically acceptable salt thereof.

In another embodiment, the present invention further provides a method of inhibiting a BET family bromodomain in a cell, comprising contacting the cell with a therapeutically effective amount of a compound of Formula (I), or a pharmaceutically acceptable salt thereof, or a pharmaceutical composition comprising a therapeutically effective amount of a compound of Formula (I), or a pharmaceutically acceptable salt thereof.

In another embodiment, the present invention further provides a method of inhibiting the BRD-4 BET family bromodomain in a cell, comprising contacting the cell with a therapeutically effective amount of a compound of Formula (I), or a pharmaceutically acceptable salt thereof, or a pharmaceutical composition comprising a therapeutically effective amount of a compound of Formula (I), or a pharmaceutically acceptable salt thereof.

In another embodiment, the present invention relates to a method of treating a BET family bromodomain-dependent disease or disorder in a patient, comprising administering to a patient in need thereof a therapeutically effective amount of a compound of Formula (I), or a pharmaceutically acceptable salt thereof, or a pharmaceutical composition comprising a therapeutically effective amount of a compound of Formula (I), or a pharmaceutically acceptable salt thereof.

In another embodiment, the present invention relates to a method of treating a BRD-4 BET family bromodomain-dependent disease or disorder in a patient, comprising administering to a patient in need thereof a therapeutically effective amount of a compound of Formula (I), or a pharmaceutically acceptable salt thereof, or a pharmaceutical composition comprising a therapeutically effective amount of a compound of Formula (I), or a pharmaceutically acceptable salt thereof.

In another embodiment, the present invention relates to a compound of Formula (I), or a pharmaceutically acceptable salt thereof, for use as a medicament.

In another embodiment, the present invention relates to a compound of Formula (I), or a pharmaceutically acceptable salt thereof, for use in the treatment of a BET family bromodomain-dependent disease or disorder.

In another embodiment, the present invention relates to a compound of Formula (I), or a pharmaceutically acceptable salt thereof, for use in the treatment of a BRD-4 BET family bromodomain-dependent disease or disorder.

In another embodiment, the present invention relates to the use of a compound of Formula (I), or a pharmaceutically acceptable salt thereof, for the manufacture of a medicament to treat a BET-family bromodomain-dependent disease or disorder.

In yet another embodiment, the present invention relates to a pharmaceutical composition for use in the treatment of a BET family bromodomain-dependent disease or disorder, which composition comprises a compound of Formula (I), or a pharmaceutically acceptable salt thereof, and a pharmaceutically acceptable excipient.

The present invention also relates to a method of treating a disease or a disorder in a patient, comprising administering to a patient in need thereof a therapeutically effective amount of a compound of Formula (II), or a pharmaceutically acceptable salt thereof, or a pharmaceutical composition comprising a therapeutically effective amount of a compound of Formula (II), or a pharmaceutically acceptable salt thereof.

In another embodiment, the present invention further provides a method of inhibiting a BET family bromodomain in a cell, comprising contacting the cell with a therapeutically effective amount of a compound of Formula (II), or a pharmaceutically acceptable salt thereof, or a pharmaceutical composition comprising a therapeutically effective amount of a compound of Formula (II), or a pharmaceutically acceptable salt thereof.

In another embodiment, the present invention further provides a method of inhibiting the BRD-4 BET family bromodomain in a cell, comprising contacting the cell with a therapeutically effective amount of a compound of Formula (II), or a pharmaceutically acceptable salt thereof, or a pharmaceutical composition comprising a therapeutically effective amount of a compound of Formula (II), or a pharmaceutically acceptable salt thereof.

In another embodiment, the present invention relates to a method of treating a BET family bromodomain-dependent disease or disorder in a patient, comprising administering to a patient in need thereof a therapeutically effective amount of a compound of Formula (II), or a pharmaceutically acceptable salt thereof, or a pharmaceutical composition comprising a therapeutically effective amount of a compound of Formula (II), or a pharmaceutically acceptable salt thereof.

In another embodiment, the present invention relates to a method of treating a BRD-4 BET family bromodomain-dependent disease or disorder in a patient, comprising administering to a patient in need thereof a therapeutically effective amount of a compound of Formula (II), or a pharmaceutically acceptable salt thereof, or a pharmaceutical composition comprising a therapeutically effective amount of a compound of Formula (II), or a pharmaceutically acceptable salt thereof.

In another embodiment, the present invention relates to a compound of Formula (II), or a pharmaceutically acceptable salt thereof, for use as a medicament.

In another embodiment, the present invention relates to a compound of Formula (II), or a pharmaceutically acceptable salt thereof, for use in the treatment of a BET family bromodomain-dependent disease or disorder.

In another embodiment, the present invention relates to a compound of Formula (II), or a pharmaceutically acceptable salt thereof, for use in the treatment of a BRD-4 BET family bromodomain-dependent disease or disorder.

In another embodiment, the present invention relates to the use of a compound of Formula (II), or a pharmaceutically acceptable salt thereof, for the manufacture of a medicament to treat a BET-family bromodomain-dependent disease or disorder.

In yet another embodiment, the present invention relates to a pharmaceutical composition for use in the treatment of a BET family bromodomain-dependent disease or disorder, which composition comprises a compound of Formula (II), or a pharmaceutically acceptable salt thereof, and a pharmaceutically acceptable excipient.

As used herein, the terms “treat” and “treating,” as used herein, refer to partially or completely alleviating, inhibiting, ameliorating and/or relieving a condition from which a patient is suspected to suffer.

As used herein, the term “therapeutically effective” refers to a substance or an amount that elicits a desirable biological activity or effect.

The term “therapeutically effective amount” as used herein, refers to that amount of the therapeutic agent sufficient to result in amelioration of one or more symptoms of a disorder, or prevent advancement of a disorder, or cause regression of the disorder. For example, with respect to the treatment of asthma, a therapeutically effective amount preferably refers to the amount of a therapeutic agent that increases peak air flow by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.%. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

The term “abnormal cell growth” as used herein, unless otherwise indicated, refers to cell growth that is independent of normal regulatory mechanisms (e.g., loss of contact inhibition). Abnormal cell growth may be benign (not cancerous), or malignant (cancerous).

As used herein “cancer” refers to any malignant and/or invasive growth or tumor caused by abnormal cell growth. As used herein “cancer” refers to solid tumors named for the type of cells that form them, or cancers of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein, except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, but are not limited to for example, conventional work-ups to determine risk factors that may be associated with the targeted or suspected disease or condition. These and other routine methods allow the clinician to select patients in need of therapy using the methods and compounds of the present invention.

As used herein, the term “BET family bromodomain” refers to members of the bromodomain family which contain to N-terminal bromodomains.

As used herein, the term “BRD-4” refers to a BET family bromodomain-containing protein 4, which is a member of the BET bromodomain family.

As used herein, the term the term “BET family bromodomain inhibitor” refers to a compound that binds to the BET family bromodomain and decreases the resulting activity.

As used herein, the term “mammal” as used herein, refers to a human, a non-human primate, canine, feline, bovine, ovine, porcine, murine, or other veterinary or laboratory mammal. Those skilled in the art recognize that a therapy which reduces the severity of pathology in one species of mammal can be predictive of the effect of the therapy on another species of mammal.

As used herein, the term “modulate” as used herein, refers to encompasses either a decrease or an increase in activity or expression depending on the target molecule.

As used herein, the term “other therapeutic agents” as used herein, refers to any therapeutic agent that has been used, is currently used or is known to be useful for treating a disease or a disorder encompassed by the present invention.

A “pharmaceutically/therapeutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate, half-life and maximum tolerated dose (MTD) have yet to be determined but one of ordinary skill in the art can determine these using standard procedures.

As used herein, the term “IC₅₀” refers to an amount, concentration or dosage of a particular test compound that achieves a 50% inhibition of a maximal response in an assay that measures such response. The value depends on the assay used.

BET family bromodomain inhibitors may be used in the treatment of a variety of diseases or disorders related to systemic or tissue inflammation, inflammatory responses to infection or hypoxia, cellular activation and proliferation, lipid metabolism, fibrosis and in the treatment of viral infections.

The disease may be, but not limited to, one of the following classes: auto-immune diseases, inflammatory diseases, allergic diseases, metabolic diseases, infection-based diseases, trauma or tissue-injury based diseases, fibrotic diseases, genetic diseases, cardiovascular diseases, vascular diseases, heart diseases, neurological diseases, neurodegenerative diseases, pulmonary diseases, renal diseases, skin and/or dermatological diseases, liver diseases, gastrointestinal diseases, oral diseases, pain and sensory diseases, hematopoietic diseases, joint diseases, muscle diseases, bone diseases, and ophthalmic and/or ocular diseases.

Specific autoimmune diseases include, but are not limited to: rheumatoid arthritis, osteoarthritis, psoriasis, allergic dermatitis, systemic lupus erythematosus (and resulting complications), Sjögren's syndrome, multiple sclerosis, ankylosing spondylitis, Behçet's disease, lupus nephritis, scleroderma, systemic scleroderma, type 1 or juvenile on-set diabetes, alopecia universalis, Addison's disease, antiphospholipid antibody syndrome, autoimmune alopecia, autoimmune hemolytic anemia, autoimmune hepatitis, autoimmune encephalomyelitis, autoimmune thrombocytopenia, Bullous pemphigoid, Chagas disease, Celiac disease, Goodpasture's syndrome, Graves' disease, Guillain-Barré syndrome, Hashimoto's disease (or Hashimoto's thyroiditis), idiopathic thrombocytopenia purpura, mystenia gravis, pemphigus, primary biliary cirrhosis, Reiter's syndrome, systemic sclerosis, systemic-onset Juvenile Idiopathic Arthritis (SJIA), or an indication listed in a separate category herein.

Specific inflammatory diseases include, but are not limited to: interstitial cystitis, endometriosis, inflammatory bowel disease including: Crohn's disease and ulcerative colitis, vasculitis, vitiglio, vulvodynia, Wegner's granulomatosis, polyarteritis nodosa, thyroiditis, sinusitis, gingivitis, atherosclerosis, chronic prostatitis, glomerular nephritis, gout, acute kidney injury, uveitis, periodontal disease, or an indication listed in a separate category herein.

Specific pain conditions include, but are not limited to: inflammatory pain, surgical pain, visceral pain, dental pain, premenstrual pain, central pain, pain due to burns, migraine or cluster headaches, nerve injury, interstitial cystitis pain, cancer pain, viral, post-traumatic injury, pain associated with irritable bowel syndrome, gout pain, pain associated with gastrointestinal distension, pain associated with any of the other indications listed within this specification, or an indication listed in a separate category herein.

Specific respiratory, airway and pulmonary conditions include, but are not limited to: asthma (which may encompass chronic, late, bronchial, allergic, intrinsic, extrinsic or dust), chronic obstructive pulmonary disease, idiopathic pulmonary fibrosis, pulmonary arterial hypertension, cystic fibrosis, interstitial lung disease, acute lung injury, sarcoidosis, allergic rhinitis, chronic cough, airways hyperrresponsibeness, bronchitis, recurrent airway obstruction, emphysema, or bronchospasm, allergic rhinitis with nasal polyps, or an indication listed in a separate disease category herein.

Specific gastrointestinal (GI) disorders include, but are not limited to: Irritable Bowel Syndrome (IBS), Inflammatory Bowel Disease (IBD), biliary colic and other biliary disorders, renal colic, diarrhea-dominant IBS, ulcerative colitis, Crohn's Disease, Celiac disease, proctitis, eosinophilic gastroenteritis, mastocytosis, eosinophilic eosophagitis, or an indication listed in a separate disease category herein.

Specific allergic diseases include, but are not limited to: anaphylaxis, allergic rhinitis, allergic dermatitis, allergic urticaria, angioedema, allergic asthma, allergic reactions to: food, drugs, insect bites, pollen allergic conjunctivitis, or an indication listed in a separate disease category herein.

BET family bromodomain inhibiiros may be useful in the treatment of infectious diseases or conditions including those that involve inflammatory responseos to infections with bacteria, viruses, fungi, parasites, or their toxins and/or the ensuring relates of host resonse mediations that are collectively known as Damage Associations Moelcular Patterns (DAMPSs).

Specific infection-based diseases include, but are not limited to: sepsis, acute sepsis, sepsis syndrome, septic shock, endotoxaemia, systemic inflammatory response syndrome (SIRS), multi-organ dysfunction syndrome, toxic shock syndrome, acute lung injury, ARDS (acute respiratory distress syndrome), acute renal failure, fulminant hepatitis, burns, acute pancreatitis, post-surgical syndromes, sarcoidosis, Herxheimer reactions, encephalitis, myelitis, meningitis, malaria, Lyme disease, ocular infections, conjunctivitis, Whipple Disease, and SIRS associated with viral infections, influenza, herpes zoster, coronavirus, herpes simplex infections and reactivations, cold sores, herpes zoster infections and reactivations, chickenpox, shingles, human papilloma virus (HPV), human immunodeficiency virus (HIV), cervical neoplasia, adenovirus infections, including acute respiratory disease, poxvirus infections such as cowpox and smallpox, Ebola virus and other viruses of the Filoviridae family, African swine fever virus and other DNA viruses, or an indication listed in a separate disease category herein.

Specific trauma and tissue injury-based conditions include, but are not limited to: Renal glomerular damage, reperfusion injury (for example to heart, kidney, lung), spinal cord injury, tissue scarring, tissue adhesion, tissue repair, transplant rejection (for examples to heart, lung, bone marrow, cartilage, cornea, kidney, limb, liver, muscle, myoblast, pancreas, pancreatic islet, skin, nerve, small intestine, trachea), hypersensitivities, or an indication listed in a separate disease category herein.

Specific fibrotic diseases include, but are not limited to: Idiopathic pulmonary fibrosis, liver fibrosis, renal fibrosis, scleroderma, morphea, or an indication listed in a separate disease category herein.

Specific ophthalmic/ocular diseases include, but are not limited to: uveitis, age-related macular degeneration, diabetic macular edema, keratoconjuctivitis, uveitis associated with Behçet's disease, vernal conjunctivitis, ketatitis, lens-induced uveitis, herpetic keratitis, conical keratitis, corneal epithelial dystrophy, ocular pemphigus, Mooren's ulcer, Scleritis, Graves' ophthalmopathy, Cogan's syndrome, keratoconjunctivitis sicca, phlyctenule, iridocyclitis, sympathetic ophthalmia, ocular neovascularization, dry eye syndrome, or an indication listed in a separate disease category herein.

Specific joint, muscle and bone disorders include, but are not limited to: osteoarthritis, osteoporosis, rheumatoid arthritis, juvenile arthritis, psoriatic arthritis, erosive osteoarthritis of the hand, arthrofibrosis/traumatic knee injury, anterior cruciate knee ligament tear, relapsing polychondritis, recurrent multifocal osteomyelitis, ankylosing spondylitis, gout of the lumbar spine, antisynthetase syndrome, idiopathic inflammatory myopathies, articular chondrocalcinosis, systemic-onset Juvenile Idiopathic Arthritis (SJIA), gout and pyrophosphate crystal arthritis, or an indication listed in a separate disease category herein.

Specific skin/dermatological diseases include, but are not limited to: psoriasis, atopic dermatitis, cutaneous lupus, acne, dermatomyositis, eczema, pruritus, scleroderma, Sweet Syndrome/neutrophilic dermatosis, neutrophilic panniculitis, acrodermatitis (form of pustular psoriasis), hidradentitis suppurative, Majeed Syndrome, or an indication listed in a separate disease category herein.

Specific renal diseases include, but are not limited to: acute kidney injury (AKI) (sepsis-AKI, coronary artery bypass graft-AKI, cardiac surgery-AKI, non-cardiac surgery-AKI, transplant surgery-AKI cisplatin-AKI, contrast/imaging agent induced-AKI), glomerulonephritis, IgA nephropathy, crescentic GN, lupus nephritis, HIV associated nephropathy, membraneous nephropathy, C3 glomerulopathy, Dense deposit disease, ANCA vasculitis, diabetic nephropathy, hemolytic-uremic syndrome, atypical Hemolytic-uremic syndrome, nephrotic syndrome, nephritic syndrome, hypertensive nephrosclerosis, ApoL1 nephropathy, focal segmental glomerulosclerosis, Alport syndrome, Fanconi, syndrome, crystal nephropathy, nephrolithiasis, nephrotic syndrome, renal transplant rejection, amyloidosis, glomerulonephritis in SJIA, or an indication listed in a separate disease category herein.

Specific hematopoietic diseases include, but are not limited to: hemolytic anemia, or an indication listed in a separate disease category herein.

Specific liver diseases include, but are not limited to: liver fibrosis, liver cirrhosis, nonalcoholic steatohepatitis (NASH), or an indication listed in a separate disease category herein.

Specific oral diseases include, but are not limited to: gingivitis, periodontal disease or an indication listed in a separate disease category herein.

Specific metabolic diseases include, but are not limited to: Type 2 diabetes (and resulting complications), gout and hyperuricemia, metabolic syndrome, insulin resistance, obesity, or an indication listed in a separate disease category herein.

Cardiovascular conditions include, but are not limited to coronary heart disease, acute coronary syndrome, ischaemic heart disease, first or recurrent myocardial infarction, secondary myocardial infarction, non-ST segment elevation myocardial infarction, or ST segment elevation myocardial infarction, ischemic sudden death, transient ischemic attack, peripheral occlusive arterial disease, angina, atherosclerosis, hypertension, heart failure (such as congestive heart failure), diastolic dysfunction (such as left ventricular diastolic dysfunction, diastolic heart failure, and impaired diastolic filling), systolic dysfunction (such as systolic heart failure with reduced ejection fraction), vasculitis, ANCA vasculitis, post-myocardial infarction cardiac remodeling atrial fibrillation, arrhythmia (ventricular), ischemia, hypertrophic cardiomyopathy, sudden cardiac death, myocardial and vascular fibrosis, impaired arterial compliance, myocardial necrotic lesions, vascular damage, left ventricular hypertrophy, decreased ejection fraction, cardiac lesions, vascular wall hypertrophy, endothelial thickening, fibrinoid necrosis of coronary arteries, adverse remodeling, stroke, and the like, or an indication listed in a separate disease category herein. Also, included are venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g., after a bypass) and reocclusion (e.g., during or after percutaneous transluminal coronary angioplasty). Conditions assoeciate with ischaemia-reperfusion injury includes, but are not limited to, myocardial infarction, cerebro-vascular Ichaemia (stroke), acute choronary syndromes, renal reperfusion injury, organ transplantation, coronary artery bypass grafting, cardio-pulmonary bypass procedures, pulmonary, renal, hepatic, gastro-intestinal or peripheral limb embolism.

Cardiovascular complications of type 2 diabetes are associated with inflammation, accordingly, the compounds of the present invention may be used to treat diabetes and diabetic complications such as macrovascular disease, hyperglycemia, metabolic syndrome, impaired glucose tolerance, hyperuricemia, glucosuria, cataracts, diabetic neuropathy, diabetic nephropathy, diabetic retinopathy, obesity, dyslipidemia, hypertension, hyperinsulinemia, and insulin resistance syndrome, or an indication listed in a separate disease category herein.

The compounds of the present invention are also indicated for use in the treatment of neuroinflammatory and neurodegenerative conditions (i.e., disorders or diseases) in humans such as multiple sclerosis, migraine; epilepsy; Alzheimer's disease; Parkinson's disease; brain injury; stroke; cerebrovascular diseases (including cerebral arteriosclerosis, cerebral amyloid angiopathy, hereditary cerebral hemorrhage, and brain hypoxia-ischemia); cognitive disorders (including amnesia, senile dementia, HIV associated dementia, Alzheimer's associated dementia, Huntington's associated dementia, Lewy body dementia, vascular dementia, drug related dementia, delirium, and mild cognitive impairment); mental deficiency (including Down syndrome and fragile X syndrome); sleep disorders (including hypersomnia, circadian rhythm sleep disorder, insomnia, parasomnia, and sleep deprivation) and psychiatric disorders (such as anxiety (including acute stress disorder, generalized anxiety disorder, social anxiety disorder, panic disorder, post-traumatic stress disorder and obsessive-compulsive disorder); factitious disorder (including acute hallucinatory mania); impulse control disorders (including compulsive gambling and intermittent explosive disorder); mood disorders (including bipolar I disorder, bipolar II disorder, mania, mixed affective state, major depression, chronic depression, seasonal depression, psychotic depression, and postpartum depression); psychomotor disorder; psychotic disorders (including schizophrenia, schizoaffective disorder, schizophreniform, and delusional disorder); drug dependence (including narcotic dependence, alcoholism, amphetamine dependence, cocaine addiction, nicotine dependence, and drug withdrawal syndrome); eating disorders (including anorexia, bulimia, binge eating disorder, hyperphagia, and pagophagia); and pediatric psychiatric disorders (including attention deficit disorder, attention deficit/hyperactive disorder, conduct disorder, and autism), myotrophic lateral sclerosis, chronic fatigue syndrome, or an indication listed in a separate disease category herein.

In one embodiment the acute or chronic autoimmune and/or inflammatory condition is a disorder of lipid metabolism via the regulation of APO-A1 such as hypercholesterolemia, atherosclerosis and Alzheimer's disease.

In another embodiment the acute or chronic autoimmune and/or inflammatory condition are respiratory disorders such as asthma, chronic obstructive pulmonary disease, pulmonary arterial hypertension or idiopathic pulmonary fibrosis.

In another embodiment the acute or chronic autoimmune and/or inflammatory condition is a systemic inflammatory disorder such as rheumatoid arthritis, osteoarthritis, acute gout, psoriasis, systemic lupus erythematosus, multiple sclerosis, scleroderma or inflammatory bowel disease (Crohn's disease and Ulcerative colitis).

In another embodiment the acute or chronic autoimmune and/or inflammatory condition is multiple sclerosis.

In a further embodiment the acute or chronic autoimmune and/or inflammatory condition is Type I diabetes.

In one embodiment the disease or condition which involves an inflammatory response to an infection with bacteria, a virus, fungi, a parasite or their toxins is acute sepsis.

In one embodiment a BET family bromodomain inhibitor is indicated for the treatment of Human papilloma virus infections of skin or cervical epithelia. In another embodiment the viral infection is a latent HIV infection.

In one embodiment the disease or disorder for which a BET family bromodomain inhibitor is indicated is selected from diseases associated with systemic inflammatory response syndrome, such as sepsis, burns, pancreatitis, major trauma, haemorrhage and ischaemia. In this embodiment the BET family bromodomain inhibitor would be administered at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality. In another embodiment the BET family bromodomain inhibitor would be administered prior to surgical or other procedures associated with a high risk of sepsis, haemorrhage, extensive tissue damage, SIRS or MODS (multiple organ dysfunction syndrome). In a particular embodiment the disease or condition for which a BET family bromodomain inhibitor is indicated is sepsis, sepsis syndrome, septic shock or endotoxaemia. In another embodiment, the BET family bromodomain inhibitor is indicated for the treatment of acute or chronic pancreatitis. In another embodiment the BET family bromodomain is indicated for the treatment of burns.

Compounds of the current invention are also useful in the treatment of a proliferative disease selected from a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, a mammary carcinoma, follicular carcinoma, undifferentiated carcinoma, papillary carcinoma, seminoma, melanoma, smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma), or an indication listed in a separate disease category herein.

BET family bromodomain inhibitors may be useful in the treatment of cancer, including hematological (such as leukaemia, lymphoma and multiple myeloma), epithelial including lung, breast, prostate and colon carcinomas, midline carcinomas (such as NMT), mesenchymal, hepatic, renal and neurological tumours.

BET family bromodomain inhibitors may be useful in the treatment of one or more cancers selected from brain cancer (gliomas), glioblastomas, Bannayan-Zonana syndrome, Cowden disease, Lhermitte-Duclos disease, breast cancer, inflammatory breast cancer, colorectal cancer, Wilm's tumor, Ewing's sarcoma, rhabdomyosarcoma, ependymoma, medulloblastoma, colon cancer, head and neck cancer, kidney cancer, lung cancer, liver cancer, melanoma, squamous cell carcinoma, ovarian cancer, pancreatic cancer, prostate cancer, sarcoma cancer, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukaemia, erythroleukemia, malignant lymphoma, Hodgkins lymphoma, non-Hodgkins lymphoma, lymphoblastic T-cell lymphoma, Burkitt's lymphoma, diffuse large B-cell lymphoma (DLBCL), follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), NUT-midline carcinoma (NMT) and testicular cancer.

In one embodiment the cancer is a leukaemia, for example a leukaemia selected from acute monocytic leukemia, acute myelogenous leukemia, chronic myelogenous leukemia, chronic lymphocytic leukemia and mixed lineage leukaemia (MLL). In another embodiment the cancer is NUT-midline carcinoma (NMT). In another embodiment the cancer is multiple myeloma. In another embodiment the cancer is a lung cancer such as small cell lung cancer (SCLC) or non-small cell lung cancer (NSCLC). In another embodiment the cancer is a prostate cancer, such as castrate-resistant prostate cancer (CRPC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is breast cancer. In another embodiment the cancer is colorectal cancer.

In certain embodiments, the present invention relates to any of the aforementioned embodiments, wherein the BET family bromodomain-dependent disease or disorder is selected from the group consisting of chronic autoimmune disease, inflammatory disease and cancer. In particular embodiments, the BET family bromodomain-dependent disease or disorder is cancer. In some such embodiments, the cancer is a hematological cancer, such as acute myeloid leukemia, multiple myeloma, or lymphoma. In other such embodiments, the cancer is lung, breast, prostate or colon cancer.

The compounds described herein may be administered to humans and other animals orally, parenterally, sublingually, by aerosolization or inhalation spray, intranasal spray or via dry powder inhalation, rectally, intracisternally, intravaginally, intraperitoneally, bucally, intrathecally or topically in dosage unit formulations containing conventional nontoxic pharmaceutically acceptable carriers, adjuvants, and vehicles as desired. The term parenteral as used herein includes subcutaneous injection, intravenous injection, intramuscular injection, intrasternal injection, or infusion techniques. Topical administration may also involve the use of transdermal administration such as transdermal patches or ionophoresis devices.

Effective amounts of the compounds of the invention generally include any amount sufficient to detectably modulate BET family bromodomain activity, or to alleviate symptoms of diseases associated with BET family bromodomain activity or susceptible to BET family bromodomain activity modulation.

The amount of active ingredient that may be combined with the carrier materials to produce a single dosage form will vary depending upon the host treated and the particular mode of administration. It will be understood, however, that the specific dose level for any particular subject will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

In certain embodiments, the present invention relates to any of the aforementioned embodiments, wherein the treatment of a BET family bromodomain-dependent disease or a disorder further comprises administering an additional therapeutic agent.

In one embodiment, the invention relates to a combination of a compound of Formula (I), or a pharmaceutically acceptable salt thereof, and a second pharmaceutically active ingredient, or pharmaceutically acceptable salt thereof.

In one embodiment, the invention relates to a combination of a compound of Formula (II), or a pharmaceutically acceptable salt thereof, and a second pharmaceutically active ingredient, or pharmaceutically acceptable salt thereof.

As used herein, the terms “co-administration”, “co-administered”, “a combination of” or “in combination with”, refers to a combination of a compound of the invention and one or more other pharmaceutically active ingredient, or a pharmaceutically acceptable salt thereof, includes the following:
a. simultaneous administration of such a combination of a compound of Formula (I) and a further pharmaceutically active agent to a patient in need of treatment, when such components are formulated together into a single dosage form which releases said components at substantially the same time to said patient,
b. substantially simultaneous administration of such a combination of a compound of Formula (I) and a further pharmaceutically active agent to a patient in need of treatment, when such components are formulated apart from each other into separate dosage forms which are taken at substantially the same time by said patient, whereupon said components are released at substantially the same time to said patient,
c. sequential administration of such a combination of a compound of Formula (I) and a further pharmaceutically active agent to a patient in need of treatment, when such components are formulated apart from each other into separate dosage forms which are taken at consecutive times by said patient with a significant time interval between each administration, whereupon said components are released at substantially different times to said patient; and,
d. sequential administration of such a combination of a compound of Formula (I) and a further pharmaceutically active agent to a patient in need of treatment, when such components are formulated together into a single dosage form which releases said components in a controlled manner.
   In particular, it is contemplated that the compounds of the invention may be administered with the following therapeutic agents:

Non-steroidal anti-inflammatory drugs (NSAIDs), including but not limited to, non-selective COX1/2 inhibitors such as piroxicam, naproxen, flubiprofen, fenoprofen, ketoprofen, ibuprofen, etodolac (Lodine), mefanamic acid, sulindac, apazone, pyrazolones (such as phenylbutazone), salicylates (such as aspirin); selective COX2 inhibitors such as: celecoxib, rofecoxib, etoricoxib, valdecoxib, meloxicam;

Immunomodulatory and/or anti-inflammatory agents, including but not limited to, methotrexate, leflunomide, ciclesonide chloroquine, hydroxychloroquine, d-penicillamine, auranofin, sulfasalazine, sodium aurothiomalate, cyclosporine, azathioprine, cromolyn, hydroxycarbamide, retinoids, fumarates (such as monomethyl and dimethyl fumarate), glatiramer acetate, mitoxantrone, teriflunomide, suplatast tosilate, mycophenolate mofetil and cyclophosphamide, laquinimod, voclosporin, PUR-118, AMG 357, AMG 811, BCT197;

Antimalarials, including but not limited to, hydroxychloroquine (Plaquenil) and chloroquine (Aralen),cyclophosphamide (Cytoxan), methotrexate (Rheumatrex), azathioprine (Imuran), mesalamine (Asacol) and sulfasalazine (Azulfidine):

Antibiotics, including but not limited to, Flagyl or ciprofloxacin;

Anti-TNFα agents, including but not limited to, infliximab, adalimumab, certolizumab pegol, golimumab and etanercept;

Anti-CD20 agents, including but not limited to, rituximab, ocrelizumab, ofatumumab and PF-05280586;

Antidiarrheals, such as diphenoxylate (Lomotil) and loperamide (Imodium);

Bile acid binding agents, such as cholestyramine, alosetron (Lotronex) and ubiprostone (Amitiza);

Laxatives, such as Milk of Magnesia, polyethylene glycol (MiraLax), Dulcolax, Correctol and Senokot, and anticholinergics or antispasmodics such as dicyclomine (Bentyl);

T lymphocyte activation inhibitors, including but not limited to, abatacept:

Anti-IL1 treatments, including but not limited to, anakinra, rilonacept, canakinumab, gevokizumab, MABpl and MEDI-8968;

Glucocorticoid receptor modulators that may be dosed orally, by inhalation, by injection, topically, rectally, by ocular delivery, including but not limited to, betamethasone, prednisone, hydrocortisone, prednisolone, flunisolide, triamcinoline acetonide, beclomethasone, dipropionate, budesonide, fluticasone propionate, ciclesonide, mometasone furoate, fluocinonide, desoximetasone, methylprednisolone or PF-04171327;

Aminosalicyic acid derivatives, including but not limited to, sulfasalazine and mesalazine;

Anti-α4 integrin agents, including but not limited to, natalizumab;

α1- or α2-adrenergic agonist agents including but not limited to: propylhexidrine, phenylephrine, phenylpropanolamine, pseudoephedrine or naphazoline hydrochloride, oxymethazoline hydrochloride, tetrahydrozoline hydrochloride, xylometazoline hydrochloride or ethylnorepinephrine hydrochloride;

β-adrenergic agonists, including but not limited to, metaproterenol, isoprotenerol, isoprenaline, albuterol, salbutamol, formoterol, salmeterol, terbutaline, orciprenaline, botolterol mesylate, pirbuterol;

Anticholinergic agents, including but not limited to, ipratropium bromide, tiotropium bromide, oxitropium bromide, aclindinium bromide, glycopyrrolate, pirenzipine or telenzepine;

Inhaled long acting beta-agonists, long acting muscarinic antagonists and long acting corticosteroids, including but not limited, to those included in the following reference: Y. Mushtaq, The COPD pipeline, Nat Rev Drug Discov, 2014, 13(4), 253-254. http://dx.doi.orq/10.1038/nrd425;

Leukotriene pathway modulators, including but not limited to, 5-LO Inhibitors (such as zileuton), FLAP antagonists (such as veliflapon, fiboflapon), LTD4 antagonists (such as montelukast, zafirlukast or pranlukast;

H1 receptor antagonists, including but not limited to, cetirizine, loratidine, desloratidine, fexofenadine, astemizole, azelastine or chlorpheniramine;

PDE4 inhibitors, including but not limited to, apremilast, roflumilast or AN2728;

Vitamin D receptor modulators, including but not limited to, paricalcitol;

Nrf2 pathway activators, including but not limited to, fumarates, sulfurophane and bardoxolone methyl;

Modulators of the RAR-related orphan receptor (ROR) family, in particular RORg;

Modulator and/or antagonists of the chemokine receptors, including but not limited to, CCR2 antagonists (such as CCX140, BMS-741672, PF-4634817, CCX-872, NOX-E36), CCR2/5 antagonists (such as PF-4634817), CCR9 (such as vercirnon, CCX507), CCR1 modulators, CCR4 modulators, CCR5 modulators, CCR6 modulators, CXCR6 modulators, CXCR7 modulators) and CXCR2 modulators (such as danirixin, AZD5069);

Prostaglandins, including but not limited to, prostacyclin;

PDE5 inhibitors, including but not limited to, sildenafil, PF-489791, vardenafil and tadalafil;

Endothelin receptor antagonists, including but not limited to, bosentan, ambrisentan, sparsentan, atrasentan, zibotentan and macitentan;

Soluble guanylate cyclase activators, including but not limited to, riociguat;

Interferons, including but not limited to, interferon beta-la interferon beta-1b;

Sphingosine 1-phosphate receptor modulators, including but not limited to, fingolimod, ponesimod;

Inhibitors of the complement pathway, including but not limited to, C5aR antagonists (such as CCX168, PMX-53, NN8210), C₅ inhibitors (such as eculizumab), inhibitors of complement factors B and D, inhibitors of MASP2 (such as OMS-721) and ARC-1905;

Inhibitors of Janus kinases (one of more of JAK1, JAK2, JAK3, TYK2), including but not limited to, decernotinib, cerdulatinib, JTE-052, ruxolitinib, tofacitnib, Baricitinib, Peficitinib, GLPG-0634, INCB-47986, INCB-039110, PF-04965842, XL-019, ABT-494, R-348, GSK-2586184, AC-410, BMS-911543 and PF-06263276;

Inhibitors of other anti-inflammatory or immunomodulatory kinases, including but not limited to, spleen tyrosine kinase (SYK) inhibitors, p38 MAP kinase inhibitors (such as PF-3715455, PH-797804, AZD-7624, AKP-001, UR-13870, FX-005, semapimod, pexmetinib, ARRY-797, RV-568, dilmapimod, ralimetinib), PI3K inhibitors (such as GSK-2126458, pilaralisib, GSK-2269557), PI3Kg and/or PI3Kd inhibitors (such as CAL-101/GS-1101, duvelisib), JNK inhibitors, ERK1 and/or 2 inhibitors, IKKb inhibitors, BTK inhibitors, ITK inhibitors, ASK1 inhibitors (such as GS-4997), PKC inhibitors (such as sotrastaurin), TrkA antagonists (such as CT-327), MEK1 inhibitors (such as E6201);

Antioxidants, including but not limited to, myeloperoxidase inhibitors (such as AZD-3241), NOX4 and other NOX enzymes (such as GKT-137831) and N-acetyl cysteine;

Inhibitors of IL5, including but not limited to, mepolizumab, reslizumab and benralizumab;

Inhibitors of IL4, including but not limited to, pascolizumab, altrakincept and pitrakinra;

Inhibitors of IL13, including but not limited to, tralokinumab, anrukinzumab and lebrikizumab;

Anti-IL6 agents, including but not limited to, tocilizumab, olokizumab, siltuximab, PF-4236921 and sirukumab;

Inhibitors/Antagonists of IL17/IL17R, including but not limited to, secukinumab, RG-7624, brodalumab and ixekizumab;

Antagonists of IL12 and/or IL23, including but not limited to, tildrakizumab, guselkumab, MEDI2070 and AMG 139;

Inhibitors of IL33, including but not limited to, AMG 282;

Inhibitors of IL9, including but not limited to, MEDI-528;

Inhibitors of GM-CSF, including but not limited to, MT203;

Anti CD4 agents, including but not limited to, tregalizumab and rigerimod;

CRTH2 antagonists, including but not limited to, AZD-1981;

Inhibitors of B lymphocyte stimulator (BLYS; also known as BAFF), a protein that is often increased in patients with SLE, including but not limited to, belimumab, tabalumab, blisibimod, and atacicept;

CD22-specific monoclonal antibodies, including but not limited to, epratuzumab;

Inhibitors of interferon-α, including but not limited to, sifalimumab and rontalizumab;

Inhibitor of type I interferon receptors, including but not limited to, MEDI-546;

FcγRIIB agonists, including but not limited to, SM-101;

Modified and/or recombinant versions of Heat Shock Protein 10 (Hsp10, also known as Chaperonin 10 or EPF), including but not limited to, INV-103;

Inhibitors of the TNF superfamily receptor 12A (TWEAK receptor), including but not limited to, BIIB-023, enavatuzumab, and RG-7212;

Inhibitors of xanthine oxidase, including but not limited to, allopurinol, benzbromarone, febuxostat, topiroxostat, tisopurine and inositols;

Inhibitors of URAT1 (also known as SLC22A12), including but not limited to, lesinurad, RDEA 3170, UR1102 and levotofispam;

Additional treatments for gout and/or lowering of uric acid levels, including but not limited to, colchicines, pegloticase, benziodarone, isobrominidione, BCX4208 and arhalofenate;

Inhibitors of toll-like receptors (TLRs), including but not limited to, one or more of TLR7, TLR8, TLR9 (such as IMO-8400, IMO-3100, DV-1179), TLR2 and/or TLR 4 (such as VB-201, OPN-305);

Agonists of TLRs, including but not limited to, TLR7 (such as GSK2245035, AZD8848), TLR9 (such as AZD1419);

Activators SIRT1, including but not limited to, SRT2104;

A3 receptor agonists, including but not limited to, CF101;

Other agents of use of the treatment of psoriasis, including but not limited to, IDP-118, LAS41004, LEO 80185, LEO 90100, PH-10, WBI-1001, CNT01959, BT-061, cimzia, ustekinumab, MK-3222/SCH 900222, ACT-128800, AEB071, alitretinoin, ASP015K, Apo805K1, BMS-582949, FP187, hectoral (doxercalciferol), LEO 22811, Ly3009104 (INCB28050), calcipotriene foam (STF 115469), tofacitinib (CP-690,550), M518101 and CycloPsorb™;

Antifibrotic agents, including but not limited to: pirfenidone, inhibitors of LOXL2 (such as Simtuzumab), FT-011, modulators of epiregulin and/or TGFβ (such as LY-3016859), modulators of TGFβ (such as LY-2382770, fresolimumab);

Prolyl hydroxylase inhibitors, including but not limited to, GSK1278863, FG-2216, ASP-1517/FG-4592, AKB-6548, JTZ-951, BAY-85-3934 and DS-1093;

Inhibitors of granulocyte macrophage colony-stimulating factor, including but not limited to, GSK3196165 (MOR103), PD-0360324 and mavrilimumab;

Inhibitors of MAdCAM and/or α4β7 integrin, including but not limited to, PF-00547659 and MEDI7183 (abrilumab);

Inhibitors of connective tissue growth factor (CTGF), including but not limited to, PF-06473871; Inhibitors of cathepsin C, including but not limited to, GSK2793660;

Inhibitors of soluble epoxide hydrolase, including but not limited to, GSK2269557;

Inhibitors of the TNFR1 associated death domain protein, including but not limited to, GSK2862277;

Anti-CD19 agents, including but not limited to, MEDI-551 and AMG 729;

Anti-B7RP1 agents/inhibitors of ICOS ligand, including but not limited to, MEDI5872 and AMG-557;

Inhibitors of thymic stromal lymphoprotein, including but not limited to, AMG157;

Inhibitors of IL2, including but not limited to, daclizumab;

Inhibitors of Leucine rich repeat neuronal protein 6A, including but not limited to, Anti-Lingo (Biogen);

Inhibitors of integrins, including but not limited to, alpha-V/beta-6 (STX-100) and alpha-V/beta-3 (VPI-2690B);

Anti-CD40L agents, including but not limited to, CDP-7657;

Modulators of the dopamine D3 receptor, including but not limited to, ABT-614;

Inhibitors and/or modulators of galectin-3, including but not limited to, GCS-100 and GR-MD-02;

Agents for treating diabetic nephropathy, including but not limited to, DA-9801 and ASP-8232;

Agents for treating acute kidney injury, including but not limited to, THR-184, TRC-160334, NX-001, EA-230, ABT-719, CMX-2043, BB-3 and MTP-131;

Modulators of inflammasomes, including but not limited to, inhibitors of NLRP3;

Modulators of bromodomains, including but not limited to, BRD4;

Modulators of GPR43; and

Inhibitors of TRP channels, including but not limited to, TRPA1, TRPC₃, TRPC₅, TRPC₆ and TRPC7.

Additional therapeutic agents include anti-coagulant or coagulation inhibitory agents, anti-platelet or platelet inhibitory agents, thrombin inhibitors, thrombolytic or fibrinolytic agents, anti-arrhythmic agents, anti-hypertensive agents, calcium channel blockers (L-type and T-type), cardiac glycosides, diuretics, mineralocorticoid receptor antagonists, NO donating agents such as organonitrates, NO promoting agents such as phosphodiesterase inhibitors, cholesterol/lipid lowering agents and lipid profile therapies, anti-diabetic agents, anti-depressants, anti-inflammatory agents (steroidal and non-steroidal), anti-osteoporosis agents, hormone replacement therapies, oral contraceptives, anti-obesity agents, anti-anxiety agents, anti-proliferative agents, anti-tumor agents, anti-ulcer and gastroesophageal reflux disease agents, growth hormone and/or growth hormone secretagogues, thyroid mimetics (including thyroid hormone receptor antagonist), anti-infective agents, anti-viral agents, anti-bacterial agents, anti-fungal agents.

Agents used in an ICU setting are included, for example, dobutamine, dopamine, epinephrine, nitroglycerin, nitroprusside, etc.

Combination agents useful for treating vasculitis are included, for example, azathioprine, cyclophosphamide, mycophenolate, mofetil, rituximab, etc.

In another embodiment, the present invention provides a combination wherein the second agent is at least one agent selected from a factor Xa inhibitor, an anti-coagulant agent, an anti-platelet agent, a thrombin inhibiting agent, a thrombolytic agent, and a fibrinolytic agent. Exemplary factor Xa inhibitors include apixaban and rivaroxaban. Examples of suitable anti-coagulants for use in combination with the compounds of the present invention include heparins (e.g., unfractioned and low molecular weight heparins such as enoxaparin and dalteparin).

In another embodiment the second agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

In another embodiment, the agent is at least one anti-platelet agent. Especially preferred anti-platelet agents are aspirin and clopidogrel. The term anti-platelet agents (or platelet inhibitory agents), as used herein, denotes agents that inhibit platelet function, for example by inhibiting the aggregation, adhesion or granular secretion of platelets. Agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. Of the NSAIDS, aspirin (acetylsalicyclic acid or ASA) and COX-2 inhibitors such as celecoxib or piroxicam are preferred. Other suitable platelet inhibitory agents include IIb/IIIa antagonists (e.g., tirofiban, eptifibatide, and abciximab), throm boxane-A2-receptor antagonists (e.g., ifetroban), thromboxane-A2-synthetase inhibitors, PDE3 inhibitors (e.g., Pletal, dipyridamole), and pharmaceutically acceptable salts or prodrugs thereof.

The term anti-platelet agents (or platelet inhibitory agents), as used herein, is also intended to include ADP (adenosine diphosphate) receptor antagonists, preferably antagonists of the purinergic receptors P₂Y₁ and P₂Y₁₂, with P₂Y₁₂ being even more preferred. Preferred P₂Y₁₂ receptor antagonists include ticagrelor, prasugrel, ticlopidine and clopidogrel, including pharmaceutically acceptable salts or prodrugs thereof. Clopidogrel is an even more preferred agent. Ticlopidine and clopidogrel are also preferred compounds since they are known to be gentle on the gastro-intestinal tract in use.

The term thrombin inhibitors (or anti-thrombin agents), as used herein, denotes inhibitors of the serine protease thrombin. By inhibiting thrombin, various thrombin-mediated processes, such as thrombin-mediated platelet activation (that is, for example, the aggregation of platelets, and/or the granular secretion of plasminogen activator inhibitor-1 and/or serotonin) and/or fibrin formation are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, argatroban, and melagatran, including pharmaceutically acceptable salts and prodrugs thereof. Boroarginine derivatives and boropeptides include N-acetyl and peptide derivatives of boronic acid, such as C-terminal alpha-aminoboronic acid derivatives of lysine, ornithine, arginine, homoarginine and corresponding isothiouronium analogs thereof. The term hirudin, as used herein, includes suitable derivatives or analogs of hirudin, referred to herein as hirulogs, such as disulfatohirudin. The term thrombolytics or fibrinolytic agents (or thrombolytics or fibrinolytics), as used herein, denote agents that lyse blood clots (thrombi). Such agents include tissue plasminogen activator (natural or recombinant) and modified forms thereof, anistreplase, urokinase, streptokinase, tenecteplase (TNK), lanoteplase (nPA), factor Vlla inhibitors, PAI-1 inhibitors (i.e., inactivators of tissue plasminogen activator inhibitors), alpha2-antiplasmin inhibitors, and anisoylated plasminogen streptokinase activator complex, including pharmaceutically acceptable salts or prodrugs thereof. The term anistreplase, as used herein, refers to anisoylated plasminogen streptokinase activator complex, as described, for example, in EP 028,489, the disclosure of which is hereby incorporated herein by reference herein. The term urokinase, as used herein, is intended to denote both dual and single chain urokinase, the latter also being referred to herein as prourokinase. Examples of suitable anti-arrythmic agents include: Class I agents (such as propafenone); Class II agents (such as metoprolol, atenolol, carvadiol and propranolol); Class III agents (such as sotalol, dofetilide, amiodarone, azimilide and ibutilide); Class IV agents (such as ditiazem and verapamil); K⁺ channel openers such as I_{Ach} inhibitors, and I_{Kur} inhibitors (e.g., compounds such as those disclosed in WO01/40231).

The compounds of the present invention may be used in combination with antihypertensive agents and such antihypertensive activity is readily determined by those skilled in the art according to standard assays (e.g., blood pressure measurements). Examples of suitable anti-hypertensive agents include: alpha adrenergic blockers; beta adrenergic blockers; calcium channel blockers (e.g., diltiazem, verapamil, nifedipine and amlodipine); vasodilators (e.g., hydralazine), diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors; vasopepsidase inhibitors (dual NEP-ACE inhibitors) (e.g., gemopatrilat and nitrates). An exemplary antianginal agent is ivabradine. Examples of suitable calcium channel blockers (L-type or T-type) include diltiazem, verapamil, nifedipine and amlodipine and mybefradil. Examples of suitable cardiac glycosides include digitalis and ouabain.

In one embodiment, a compound of the invention may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™, ESIDRIX™ or HYDRODIURIL™), hydrochlorothiazide (such as MICROZIDE™ or ORETIC™), benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™). In another embodiment, a compound of the invention may be co-administered with a loop diuretic. In still another embodiment, the loop diuretic is selected from furosemide and torsemide. In still another embodiment, one or more compounds of the invention may be co-administered with furosemide. In still another embodiment, one or more compounds of the invention may be co-administered with torsemide which may optionally be a controlled or modified release form of torsemide.

In another embodiment, a compound of the invention may be co-administered with a thiazide-type diuretic. In still another embodiment, the thiazide-type diuretic is selected from the group consisting of chlorothiazide and hydrochlorothiazide. In still another embodiment, one or more compounds of the invention may be co-administered with chlorothiazide. In still another embodiment, one or more compounds of the invention may be co-administered with hydrochlorothiazide. In another embodiment, one or more compounds of the invention may be co-administered with a phthalimidine-type diuretic. In still another embodiment, the phthalimidine-type diuretic is chlorthalidone.

Examples of suitable combination mineralocorticoid receptor antagonists include sprionolactone and eplerenone. Examples of suitable combination phosphodiesterase inhibitors include: PDE3 inhibitors (such as cilostazol); and PDE5 inhibitors (such as sildenafil). The compounds of the present invention may be used in combination with cholesterol modulating agents (including cholesterol lowering agents) such as a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, an ion-exchange resin, an antioxidant, an ACAT inhibitor or a bile acid sequestrant or an agent such as mipomersen.

Examples of suitable cholesterol/lipid lowering agents and lipid profile therapies include: HMG-CoA reductase inhibitors (e.g., pravastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin)); squalene synthetase inhibitors; fibrates; bile acid sequestrants (such as questran); ACAT inhibitors; MTP inhibitors; lipooxygenase inhibitors; cholesterol absorption inhibitors; and cholesteryl ester transfer protein inhibitors. Anti-inflammatory agents also include sPLA2 and IpPLA2 inhibitors (such as darapladib), 5 LO inhibitors (such as atrelueton) and IL-1 and IL-1r antagonists (such as canakinumab).

Other atherosclerotic agents include agents that modulate the action of PCSK9, for example, called bococizumab.

The compounds of the present invention may be used in combination with anti-diabetic agents, particularly type 2 anti-diabetic agents. Examples of suitable anti-diabetic agents include (e.g. insulins, metformin, DPPIV inhibitors, GLP-1 agonists, analogues and mimetics, SGLT1 and SGLT2 inhibitors) Suitable anti-diabetic agents include an acetyl-CoA carboxylase-(ACC) inhibitor such as those described in WO2009144554, WO2003072197, WO2009144555 and WO2008065508, a diacylglycerol O-acyltransferase 1 (DGAT-1) inhibitor, such as those described in WO09016462 or WO2010086820, AZD7687 or LCQ908, diacylglycerol O-acyltransferase 2 (DGAT-2) inhibitor, monoacylglycerol O-acyltransferase inhibitors, a PDE10 inhibitor, an AMPK activator, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARy agonist (e.g., balaglitazone, ciglitazone, darglitazone, englitazone, isaglitazone, pioglitazone and rosiglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) modulator such as an agonist (e.g., exendin-3 and exendin-4), liraglutide, albiglutide, exenatide (Byetta®), albiglutide, lixisenatide, dulaglutide, semaglutide, NN-9924, TTP-054, a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., resveratrol, GSK2245840 or GSK184072), a dipeptidyl peptidease IV (DPP-IV) inhibitor (e.g., those in WO2005116014, sitagliptin, vildagliptin, alogliptin, dutogliptin, linagliptin and saxagliptin), an insulin secreatagogue, a fatty acid oxidation inhibitor, an A2 antagonist, a c-jun amino-terminal kinase (JNK) inhibitor, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, an insulin mimetic, a glycogen phosphorylase inhibitor (e.g. GSK1362885), a VPAC2 receptor agonist, SGLT2 inhibitors, such as those described in E. C. Chao et al. Nature Reviews Drug Discovery 9, 551-559 (July 2010) including dapagliflozin, canagliflozin, empagliflozin, tofogliflozin (CSG452), ASP-1941, THR1474, TS-071, ISIS388626 and LX4211 as well as those in WO2010023594, a glucagon receptor modulator such as those described in Demong, D. E. et al. Annual Reports in Medicinal Chemistry 2008, 43, 119-137, GPR119 modulators, particularly agonists, such as those described in WO2010140092, WO2010128425, WO2010128414, WO2010106457, Jones, R. M. et al. in Medicinal Chemistry 2009, 44, 149-170 (e.g. MBX-2982, GSK1292263, APD597 and PSN821), FGF21 derivatives or analogs such as those described in Kharitonenkov, A. et al. et al., Current Opinion in Investigational Drugs 2009, 10(4)359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high affinity nicotinic acid receptor (HM74A) activators, and SGLT1 inhibitors, such as GSK1614235. A further representative listing of anti-diabetic agents that can be combined with the compounds of the present invention can be found, for example, at page 28, line 35 through page 30, line 19 of WO2011005611. Preferred anti-diabetic agents are metformin and DPP-IV inhibitors (e.g., sitagliptin, vildagliptin, alogliptin, dutogliptin, linagliptin and saxagliptin). Other antidiabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC₂, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors (e.g. SSTR1, SSTR2, SSTR3 and SSTR5), inhibitors or modulators of PDHK2 or PDHK4, inhibitors of MAP4K4, modulators of IL1 family including IL1beta, modulators of RXRalpha. In addition suitable anti-diabetic agents include mechanisms listed by Carpino, P. A., Goodwin, B. Expert Opin. Ther. Pat, 2010, 20(12), 1627-51.

Those skilled in the art will recognize that the compounds of this invention may also be used in conjunction with other cardiovascular or cerebrovascular treatments including PCI, stenting, drug eluting stents, stem cell therapy and medical devices such as implanted pacemakers, defibrillators, or cardiac resynchronization therapy.

The compounds of the present invention may be used in combination with neuroinflammatory and neurodegenerative agents in mammals. Examples of additional neuroinflammatory and neurodegenerative agents include antidepressants, antipsychotics, anti-pain agents, anti-Alzheimer's agents, and anti-anxiety agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, and atypical antidepressants. Suitable norepinephrine reuptake inhibitors include tertiary amine tricyclics and secondary amine tricyclics. Examples of suitable tertiary amine tricyclics and secondary amine tricyclics include amitriptyline, clomipramine, doxepin, imipramine, trimipramine, dothiepin, butriptyline, nortriptyline, protriptyline, amoxapine, desipramine and maprotiline. Examples of suitable SSRIs include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Examples of suitable SNRIs of use in the present invention include venlafaxine. Examples of suitable atypical anti-depressants include bupropion, lithium, trazodone and viloxazine. Examples of anti-Alzheimer's agents include NMDA receptor antagonists such as memantine; and cholinesterase inhibitors such as donepezil and galantamine. Examples of suitable classes of anti-anxiety agents that can be used in combination with the compounds of the invention include benzodiazepines and serotonin 1A receptor (5-HT1A) agonists, and CRF antagonists. Suitable benzodiazepines include alprazolam, chlordiazepoxide, clonazepam, chlorazepate, diazepam, lorazepam, oxazepam, and prazepam. Suitable 5-HT1A receptor agonists include buspirone and ipsapirone. Suitable CRF antagonists include verucerfont. Suitable atypical antipsychotics include paliperidone, ziprasidone, risperidone, aripiprazole, olanzapine, and quetiapine. Suitable nicotine acetylcholine agonists include CP-601927 and varenicline. Anti-pain agents include pregabalin, gabapentin, clonidine, neostigmine, baclofen, midazolam, ketamine and ziconotide.

In another embodiment, compounds of the invention may be used in combination with one or more of the following anti-angiogenesis agents, signal transduction inhibitors, chemotherapeutic agents (such as alkylating agents, antimetabolites, cytotoxic antibiotics, mitotic inhibitors and topoisomerase inhibitors), radiation, cell cycle inhibitors, enzyme inhibitors, biological response modifiers (such as glycoproteins, growth factor inhibitors and cytokines), and anticancer antibodies.

Inasmuch as it may be desirable to administer a combination of active compounds, for example, for the purpose of treating a particular disease or condition, it is within the scope of the present invention that two or more pharmaceutical compositions, at least one of which comprises a compound of the invention, may conveniently be combined in the form of a kit suitable for co-administration of the compositions. Representative kits include a compound described herein (e.g., a compound of Formula I) and a package insert or other labeling including directions for treating a BET family bromodomain dependent disease or condition, including, but not limited to, chronic autoimmune and/or inflammatory condition and cancer, by administering an effective amount of a compound of the present invention.

Compounds of the present invention can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given; other process conditions can also be used unless otherwise stated. Optimum reaction conditions can vary with the particular reactants or solvent used. Those skilled in the art will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

Preparation of the compounds can involve protection and deprotection of various chemical groups. The chemistry of protecting groups can be found, for example, in Greene et al., Protective Groups in Organic Synthesis, 4th. Ed. (John Wiley & Sons, 2007), the entire disclosure of which is incorporated by reference herein for all purposes.

The reactions or the processes described herein can be carried out in suitable solvents, which can be readily selected by one skilled in the art. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art. The reagents used in the preparation of the compounds of these teachings can be either commercially obtained or can be prepared by standard procedures described in the literature. For example, compounds of the present invention can be prepared according to the methods illustrated in the following Synthetic Schemes.

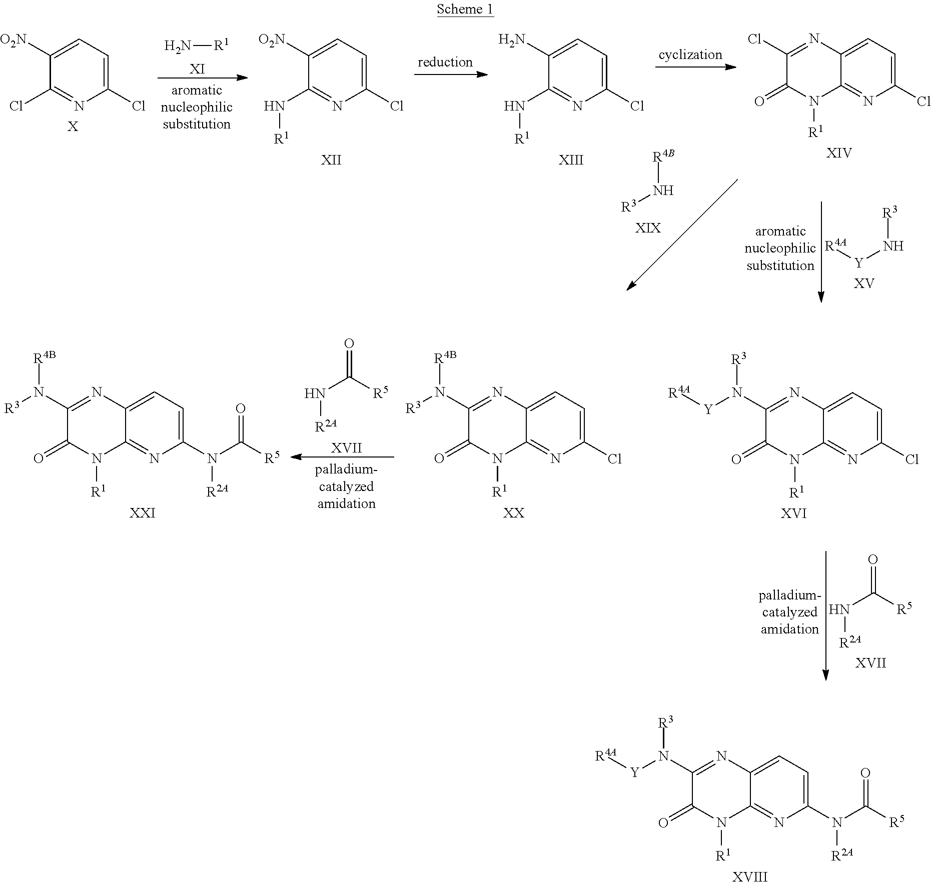

According to Scheme 1, the Formula XVIII and XXI compounds wherein R¹, R^{2A}, R³, R^{4A}, R^{4B} and Y are defined as above and R⁵ is alkyl, cycloalkyl or heterocyclyl, may be prepared from the Formula X compound by aromatic nucleophilic substitution reaction with an appropriate Formula XI compound wherein R¹ is defined as above, followed by reduction, cyclization, aromatic nucleophilic substitution reaction with an appropriate Formula XV or Formula XIX compounds wherein R³, R^{4A} and R^{4B} are defined as above, and a palladium-catalyzed amidation with an appropriate Formula XVII compound wherein R^{2A} is defined as above and R⁵ is alkyl, cycloalkyl or heterocyclyl.

The Formula XII compound wherein R¹ is defined as above may be prepared from the Formula X compound by aromatic nucleophilic substitution reaction. For example, the Formula X compound can be combined with the Formula XI compound in the presence of a base such as sodium carbonate or diisopropylethyl amine in a solvent such as isopropanol at a temperature of 0° C. for about 4 h and then stirring the mixture at room temperature for about 18 h.

The Formula XIII compounds wherein R¹ is defined as above may be prepared from the Formula XII compounds by reduction methods such as hydrogenation. For example, the Formula XII compound is hydrogenated in the presence of a catalyst such as Raney nickel in a solvent such as ethyl acetate in a Parr shaker apparatus at a hydrogen pressure of about 50 psi for about 10 h to about 8 h, typically 8 h.

The Formula XIV compounds wherein R¹ is defined as above may be prepared by cyclization of the Formula XIII compounds. For example, a solution of the Formula XIII compound in a solvent such as 1,2-dichlorobenzene is added to a solution of oxalyl chloride in a solvent such as 1,2-dichlorobenzene at a temperature of 65° C. to about 50° C., typically 65° C. over a period of about 20 minutes to about 10 minutes. The resulting mixture is then heated at a high temperature of about 140° C. to about 120° C., typically 130° C. for about 5 h to about 4 h.

The Formula XVI compounds wherein R¹, R³, R^{4A}, and Y are defined as above may be prepared from the Formula XIV compounds by aromatic nucleophilic substitution reaction with an appropriate Formula XV compound. For example, the Formula XIV compound is combined with the Formula XV compound in a solvent such as dichloromethane in the presence of base such as diisopropylethylamine and the mixture is stirred at a temperature of about 25° C. for about 24 h to about 12 h.

The Formula XVIII compounds may be prepared from the Formula XVI compounds by palladium-catalyzed amidation reaction with an appropriate Formula XVII compound. Recent reviews have discussed the application of biarylphosphine ligands in palladium-catalyzed amination, for example Surry and Buchwald Chem. Sci. 2011, 2, 27-50, and Angew. Chem. Int. Ed. 2008, 47, 6338-6361. An example of the preparation of Formula XVIII compounds is described next. The Formula XVI compound is combined with the Formula XVII compound in a solvent such as dioxane in the presence of base such as potassium triphosphate in a pressure reactor, such as a sealed tube. The mixture is degassed with an inert gas such as argon for about 20 minutes to about 10 minutes. This process is repeated several times, typically three times. A palladium catalyst such as palladium acetate and a phosphine ligand such as S-Phos is added and the resulting mixture is degassed with an inert gas such as argon for about 10 minutes to about 5 minutes. The resulting mixture is heated at a temperature of 140° C. to about 100° C., typically 130° C. for about 20 h to about 12 h, typically 16 h.

The Formula XX compounds wherein R¹, R³ and R^{4B} are defined as above may be prepared from the Formula XIV compounds by aromatic nucleophilic substitution reaction with an appropriate Formula XIX compound following the procedure described for the Formula XVI compounds.

The Formula XXI compounds may be prepared from the Formula XX compounds by palladium-catalyzed amidation reaction with an appropriate Formula XVII compound following the procedure described for the Formula XVIII compounds.

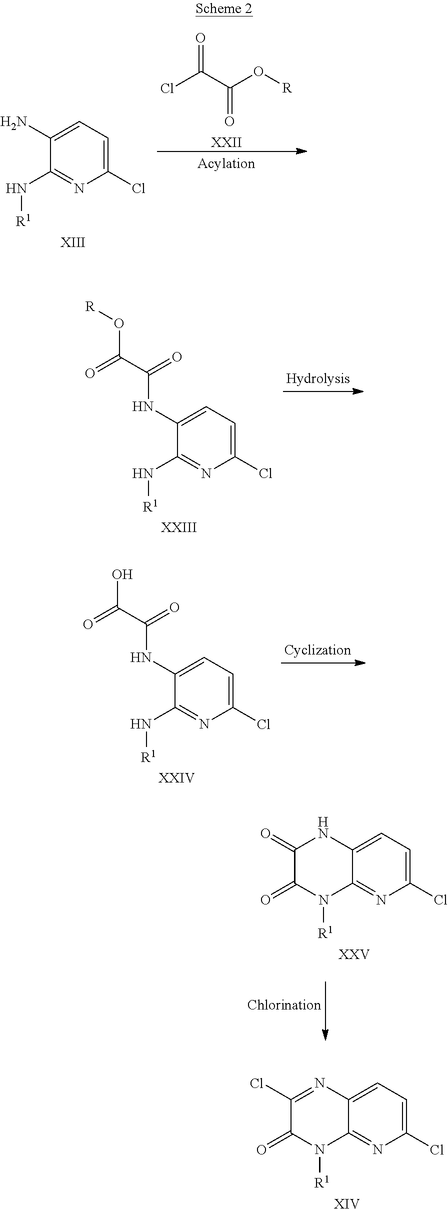

Alternatively, and according to Scheme 2, the Formula XIV compounds may be prepared from Formula XIII compounds by acylation with a Formula XXII compound wherein R is an alkyl, followed by hydrolysis, cyclization, and chlorination.

The Formula XXIII compounds wherein R¹ is defined as above may be prepared from the Formula XIII compounds by acylation with a Formula XXII compound. For example, the Formula XIII compound is combined with a Formula XXII compound such as ethyl chloro(oxo)acetate in the presence of base such as sodium carbonate in an anhydrous aprotic solvent such as tetrahydrofuran at a temperature of about 25° C. The resulting mixture is stirred at the above temperature for about 24 h to about 12 h, typically 18 h to provide the Formula XXIII compound.

The Formula XXIV compounds wherein R¹ is defined as above may be prepared from the Formula XXIII compounds by hydrolysis. For example, the Formula XXIII compound is combined with of a solution of a strong base such as 2M sodium hydroxide in water in a solvent such as tetrahydrofuran at a temperature of 10° C. to about 0° C., typically 0° C. for about 6 h to about 1 h, typically 1 h, to provide the Formula XXIV compound.

The Formula XXV compounds wherein R¹ is defined as above may be prepared from the Formula XXIV compounds by cyclization. For example, the Formula XXIV compound in an anhydrous aprotic solvent such as tetrahydrofuran is heated at a temperature of 40° C. to about 30° C., typically 30° C. To this mixture, oxalyl chloride is added dropwise followed by a catalytic amount of dimethylformamide. The resulting mixture is heated at a temperature of 60° C. to about 40° C., typically 50° C. for about 6 h to about 4 h to produce the Formula XXV compounds.

The Formula XIV compounds may be prepared from the Formula XXV compounds by chlorination. In a typical example, the Formula XXV compound in an anhydrous aprotic solvent such as tetrahydrofuran is heated at a temperature of 40° C. to about 30° C., typically 30° C. To this mixture, oxalyl chloride is added dropwise followed by a catalytic amount of dimethylformamide. The resulting mixture is heated at a temperature of 80° C. to about 60° C., typically 80° C. for about 24 h to about 16 h to produce the Formula XIV compounds.

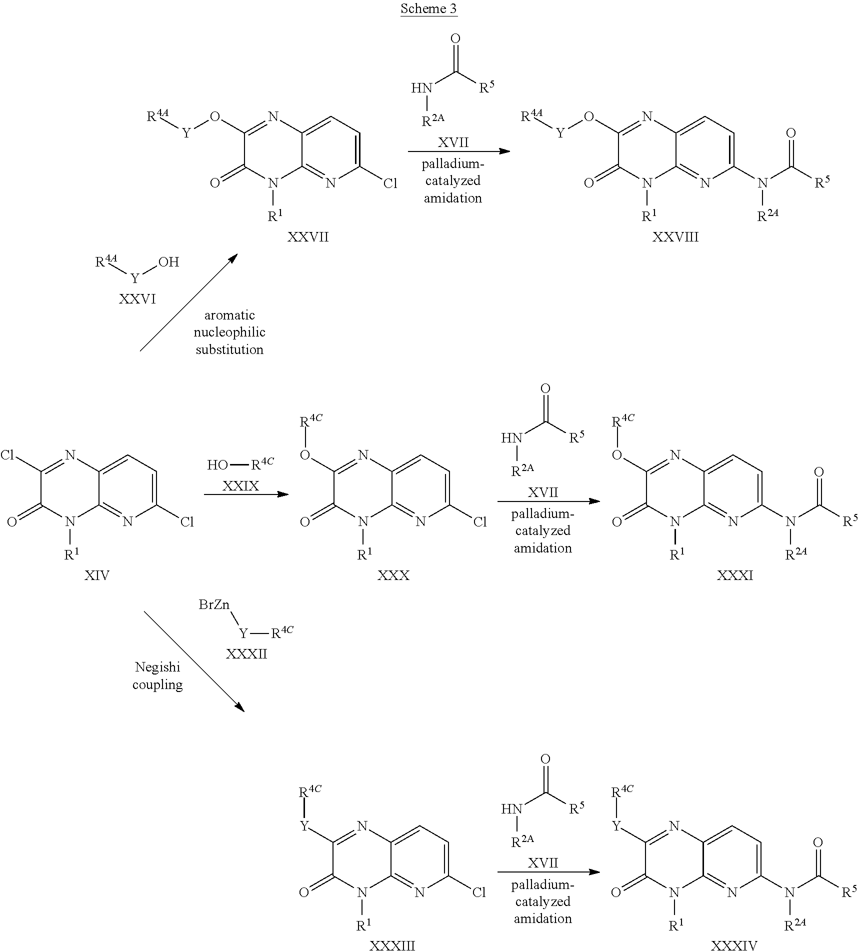

According to Scheme 3, the Formula XXVIII and Formula XXXI compounds wherein R¹, R^{2A}, R^{4A}, R^{4C} and Y are defined as above and R⁵ is alkyl, cycloalkyl or heterocyclyl, may be prepared from the Formula XIV compounds by aromatic nucleophilic substitution reaction with an appropriate Formula XXVI or Formula XXIX compounds wherein Y, R^{4A} and R^{4C} are defined as above, followed by palladium-catalyzed amidation with an appropriate Formula XVII compound.

The Formula XXVII compounds wherein R¹, R^{4A} and Y are defined as above may be prepared from the Formula XIV compounds by aromatic nucleophilic substitution reaction with an appropriate Formula XXVI compound. For example, the Formula XXVI compound is stirred in an aprotic solvent such as dimethylformamide in the presence of a strong base such as sodium hydride at a temperature of about 25° C. for 20 minutes to about 5 minutes. A solution of the Formula XIV compound in an aprotic solvent such as dimethylformamide is added to the above mixture and the resulting mixture is stirred at a temperature of 25° C. of about 2 h to about 30 minutes.

The Formula XXVIII compounds may be prepared from the Formula XXVII compounds by palladium-catalyzed amidation reaction with an appropriate Formula XVII compound following the procedure described for the Formula XVIII compounds.

The Formula XXX compounds wherein R¹ and R^{4C} are defined as above may be prepared from the Formula XIV compounds by aromatic nucleophilic substitution reaction with an appropriate Formula XXIX compound following the procedure described for the Formula XXVII compounds.

The Formula XXXI compounds may be prepared from the Formula XXX compounds by palladium-catalyzed amidation reaction with an appropriate Formula XVII compound following the procedure described for the Formula XVIII compounds.

According to Scheme 3, the Formula XXXIV compounds wherein R¹, R^{2A}, R^{4C}, and Y are defined as above and R⁵ is alkyl, cycloalkyl or heterocyclyl, may be prepared from the Formula XIV compounds by Negishi coupling of an appropriate Formula XXXII compound wherein Y and R^{4C} are defined as above, followed by palladium-catalyzed amidation with an appropriate Formula XVII compound.

The Formula XXXIII compounds wherein R¹, R^{4C}, and Y are defined as above may be prepared from the Formula XIV compounds by Negishi coupling of an appropriate Formula XXXII compound. There are numerous book chapters and review articles discussing the Negishi coupling, for example: Xu et al. in “Metal-Catalyzed Cross Coupling Reactions and More”, 3rd Eds. Wiley-VCH, Weinheim. 2014, 133-278. Furthermore, the application of the palladium-PEPPSI complexes and its application in the Negishi coupling have been recently reviewed: Valente et al. Eur. J. Org. Chem. 2010, 4343-4354. An example of the preparation of Formula XXXIII compounds using this method is described next. The Formula XIV compound is combined with a palladium catalyst such as PEPPSI-IPr. The mixture is degassed with an inert gas such as argon for about 20 minutes to about 10 minutes. A solution of the Formula XXXII compound in an aprotic solvent such as tetrahydrofuran is added to the above mixture and the combined mixture is stirred at a temperature of about 25° C. of about 24 h to about 12 h to provide the Formula XXXIII compound.

The Formula XXXIV compounds may be prepared from the Formula XXXIII compounds by palladium-catalyzed amidation reaction with an appropriate Formula XVII compound following the procedure described for the Formula XVIII compounds.

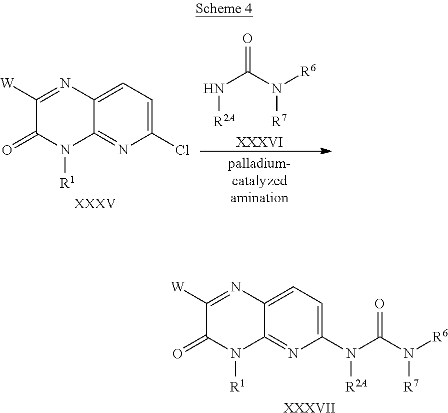

According to Scheme 4, the Formula XXXVII compounds wherein R¹, R^{2A} and W are defined as above and R⁶ and R⁷ are H or alkyl may be prepared from the Formula XXXV compounds by palladium-catalyzed amination with an appropriate Formula XXXVI compound wherein R^{2A} is defined as above and R⁶ and R⁷ are H or alkyl.

The Formula XXXV compounds may be prepared from the Formula XIV compounds by the methods described above for the Formula XVI, Formula XX, Formula XXVII, Formula XXX, and Formula XXXIII compounds according to Schemes 1 and 3.

The Formula XXXVII compounds may be prepared from the Formula XXXV compounds by palladium-catalyzed amination with an appropriate Formula XXXVI compound. For example, the Formula XXXV compound is combined with the Formula XXXVI compound in a polar solvent such as dioxane/water in a ratio of 4:1, in the presence of a strong base such as sodium tert-butoxide. The mixture is degassed with an inert gas such as argon for about 20 minutes to about 5 minutes. This process is repeated several times, typically three times. A palladium catalyst such as Brettphos palladacycle is added and the resulting mixture is heated at a temperature of 120° C. to about 100° C., typically 100° C. under microwave irradiation for about 5 h to about 2 h, typically 2 h, to provide the Formula XXXVII compound.

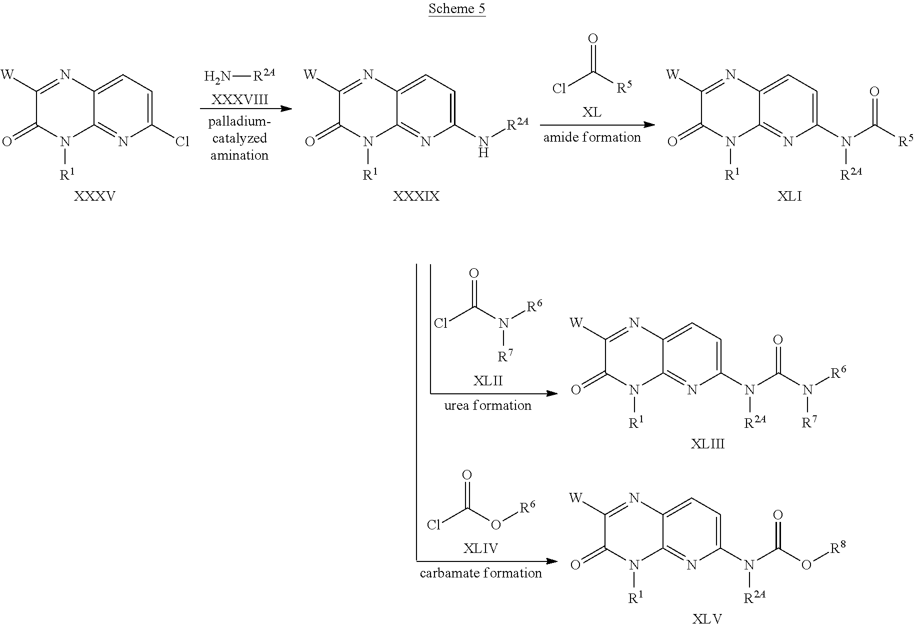

According to Scheme 5, the Formula XLI compounds wherein R¹, R^{2A} and W are defined as above and R⁵ is alkyl, cycloalkyl or heterocyclyl, may be prepared from the Formula XXXV compounds by palladium-catalyzed amination with an appropriate Formula XXXVIII compound wherein R^{2A} is defined as above, followed by amide formation with Formula XL compounds wherein R⁵ is alkyl, cycloalkyl or heterocyclyl.

The Formula XXXIX compounds may be prepared from the Formula XXXV compounds by palladium-catalyzed amination with an appropriate Formula XXXVIII compounds by the method described for Formula XVIII compounds according to Scheme 1.

The Formula XLI compounds may be prepared from the Formula XXXIX compounds by amide formation with a Formula XL compounds. For example, a Formula XXXIX compound is combined with Formula XL compound in an aprotic anhydrous solvent such as tetrahydrofuran at a temperature of 25° C. to about 0° C., typically 0° C. To this solution, a strong base such as a solution of lithium bis(trimethylsilyl)amide in tetrahydrofuran is added and the resulting mixture is stirred at low temperature, typically 0° C. for about 2 h. Then, the temperature is gradually increased to about 25° C. and the mixture stirred for about 24 h to about 12 h, typically 16 h, to prepare the Formula XLI compounds.

According to Scheme 5, the Formula XLIII compounds wherein R¹, R^{2A} and W are defined as above and R⁶ and R⁷ are alkyl, may be prepared from the Formula XXXIX compounds by urea formation with Formula XLII compounds wherein R⁶ and R⁷ are alkyl. For example, the Formula XXXIX compound is combined with Formula XLII compound in a nonpolar solvent such toluene in the presence of a base such triethylamine and a catalytic amount of 4-dimethylaminopyridine. The combined mixture is heated at a temperature of about 110° C. to about 80° C., typically 110° C. for about 24 h to about 12 h to prepare the Formula XLIII compounds.

According to Scheme 5, the Formula XLV compounds wherein R¹, R^{2A} and W are defined as above and R⁸ is alkyl may be prepared from the Formula XXXIX compounds by carbamate formation with an appropriate Formula XLIV compound wherein R⁸ is alkyl. For example, a Formula XXXIX compound is combined with Formula XLIV compound in an anhydrous aprotic solvent such as tetrahydrofuran at a temperature of 25° C. to about 0° C., typically 25° C. To this solution, a strong base such as a solution of sodium bis(trimethylsilyl)amide in tetrahydrofuran, is added and the resulting mixture is stirred at a temperature of about 25° C. for about 6 h to about 0.5 h.

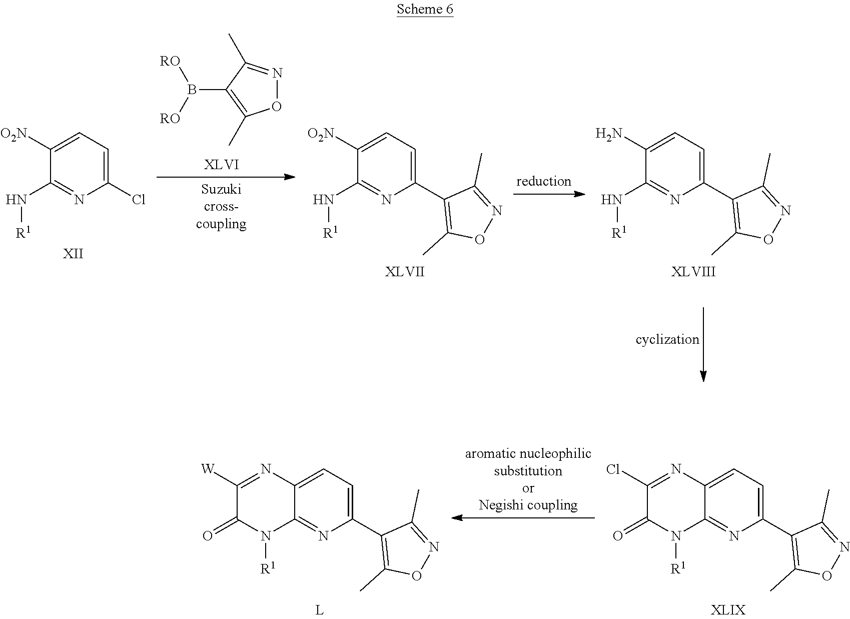

According to Scheme 6, the Formula L compounds wherein R¹ and W are defined as above may be prepared from the Formula XII compounds by Suzuki coupling with an appropriate Formula XLVI compound wherein R is H or alkyl, followed by reduction, cyclization, and aromatic nucleophilic substitution reaction or Negishi coupling.

The Formula XLVII compounds wherein R¹ is defined as above may be prepared from the Formula XII compounds by Suzuki cross-coupling with Formula XLVI compounds. The preparation of the Formula XII compounds may be carried out by the methods described above in Scheme 1. An example of the preparation of Formula XLVII compounds is described next. The Formula XII is combined with a Formula XLVI compound such as 3,5-dimethylisoxazole-4-boronic acid and a base such as sodium carbonate in a polar solvent such as dioxane and water combined in about a 12:1 ratio, in a pressure reactor, such as a sealed tube. The mixture is degassed with an inert gas such as argon for about 15 minutes to about 5 min. This process is repeated several times, typically three times. A palladium catalyst such as palladium acetate and a phosphine ligand such as RuPhos are added to the mixture. The mixture is degassed again with an inert gas such as argon for about 10 min. The mixture is heated at a temperature of 100° C. to about 60° C., typically 80° C., for about 6 h to about 2 h to provide the Formula XLVII compound.

The Formula XLVIII compounds wherein R¹ is defined as above may be prepared from the Formula XLVII compounds by reduction methods such as hydrogenation following the procedure described for the Formula XIII compounds according to Scheme 1.

The Formula XLIX compounds wherein R¹ is defined as above may be prepared from the Formula XLVIII compounds by cyclization following the procedure described for the Formula XIV compounds according to Scheme 1.

The Formula L compounds wherein W is —N(R³)—Y—R^{4A}, —N(R³)—R^{4B}, —O—Y—R^{4A}, or —O—R^{4C}, and wherein R³, R^{4A}, R^{4B} and Y are defined as above, may be prepared from the Formula XLIX compounds by aromatic nucleophilic substitution following the procedure described for the Formula XVI, Formula XX, Formula XXVII, and Formula XXX compounds according to Scheme 1 and 3.

The Formula L compounds wherein W is —Y—R^{4A}, and wherein R^{4A} and Y are defined as above, may be prepared from the Formula XLIX compounds by Negishi coupling following the procedure described for the Formula XXXIII compounds according to Scheme 3.

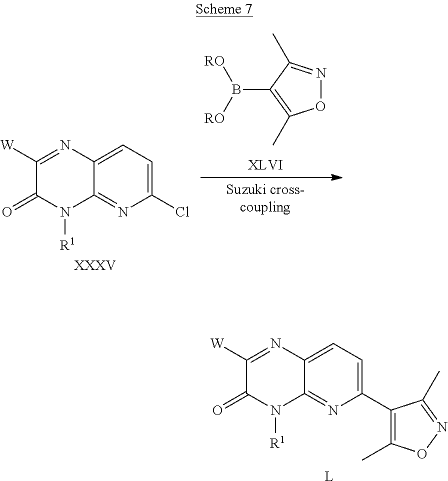

Alternatively, the Formula L compounds may be prepared from the Formula XXXV compounds by Suzuki cross-coupling with an appropriate Formula XLVI compound. The Formula XXXV compounds may be prepared as previously described in Scheme 4. The Suzuki cross-coupling of Formula XXXV compound with Formula XLVI compound may be carried out as described for Formula XLIX compounds according to Scheme 6.

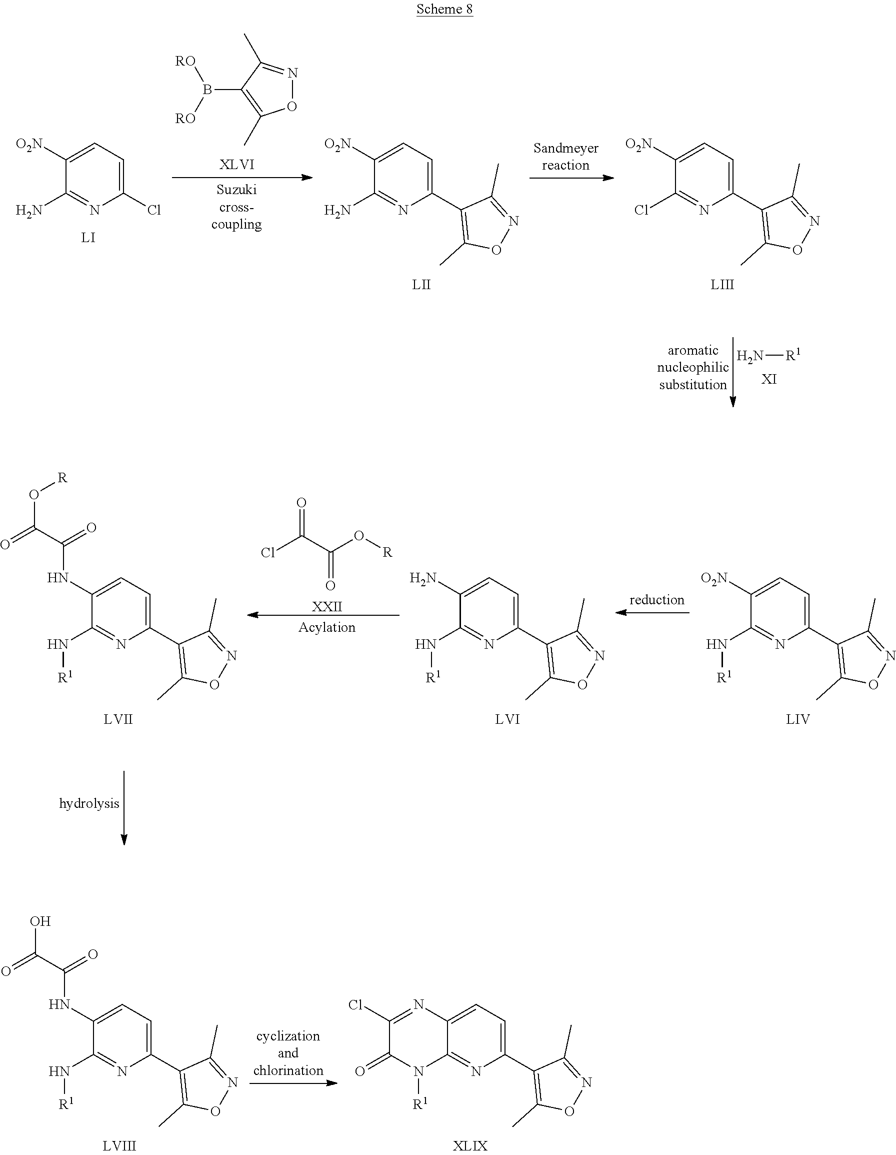

In a yet another method, and according to Scheme 8, the Formula XLIX compounds may be prepared from the Formula LI compound by Suzuki cross-coupling with an appropriate Formula XLVI compound, followed by a Sandmeyer reaction, aromatic nucleophilic substitution with an appropriate Formula XI compound, reduction of the nitro group, acylation, hydrolysis, cyclization and chlorination.

The Formula LII compounds may be prepared from the Formula LI compounds by Suzuki cross-coupling with an appropriate Formula XLVI compound such as such as 3,5-dimethylisoxazole-4-boronic acid following the method described for the Formula XLVII compounds in Scheme 6.

The Formula LIII compound may be prepared from the Formula LII compounds by Sandmeyer reaction. For example, copper(II) chloride, lithium chloride and tert-butyl nitrite in a polar solvent such as acetonitrile is heated at a temperature of about of 65° C. To this mixture, the Formula LII compound is added portion wise. The combined mixture is heated at a temperature of about 65° C. for about 6 h to about 2 h, typically 4 h. Additional copper(II) chloride, lithium chloride, and tert-butyl nitrite may be added to complete the conversion of the Formula LII compound into the desired Formula LIII compound.

The Formula LIV compounds may be prepared from the Formula LIII and Formula XI compounds by aromatic nucleophilic substitution following the method described for the preparation of Formula XII compound according to Scheme 1.

The Formula LVI compounds may be prepared from the Formula LIV compounds by reduction methods such as hydrogenation following the method described for the preparation of Formula XIII compound according to Scheme 1.

The Formula LVII compounds may be prepared from the Formula LVI compounds by acylation with an appropriate Formula XXII compounds following the method described for the preparation of Formula XXIII compounds according to Scheme 2.

The Formula LVIII compounds may be prepared from the Formula LVII compounds by hydrolysis following the method described for the preparation of Formula XXIV compounds according to Scheme 2.

The Formula XLIX compounds may be prepared from the Formula LVIII compounds by cyclization. For example, oxalyl chloride is added to a solution of the Formula LVIII compound in an anhydrous aprotic solvent such as tetrahydrofuran, followed by a catalytic amount of dimethylformamide. The resulting mixture is heated at a temperature of 60° C. to about 40° C., typically 50° C. for about 6 h to about 4 h to produce the Formula XLIX compounds.

### EXAMPLES AND PREPARATIONS

In the non-limiting Examples and Preparations that are set out later in the description, and in the aforementioned Schemes, the following the abbreviations, definitions and analytical procedures may be referred to:
Brettphos palladacycle is Chloro[2-(dicyclohexylphosphino)-3,6-dimethoxy-2′-4′-6′-triisopropyl-1,1′-biphenyl][2-(2-aminoethyl)phenyl]palladium(I)
Cs₂CO₃ is cesium carbonate
CuCl₂ is copper (11) chloride
DCM is dichloromethane
DIPEA is diisopropylethylamine
DMAP is 4-dimethylaminopyridine
DMF is N,N-dimethylformamide
EA is ethyl acetate
EDCl is 1-ethyl-3-(3-dimethylaminopropyl) carbodiimide
EtOAc is ethyl acetate
Et₃N is triethylamine
H₂O is water
HCl is hydrochloric acid
HCOOH is formic acid
IPA is isopropyl alcohol
Jackiephos is 2-{Bis[3,5-bis(trifluoromethyl)phenyl]phosphino}-3,6-dimethoxy-2′,4′,6′-triisopropyl-1,1′-biphenyl
K₂CO₃ is potassium carbonate
K₃PO₄ is potassium phosphate tribasic
KF is potassium fluoride
KOH is potassium hydroxide
LiCl is lithium chloride
LiHMDS is lithium bis(trimethylsilyl)amide
MeCN is acetonitrile
MeOH is methyl alcohol
MeNH₂.HCl is methylamine hydrochloride
NaOH is sodium hydroxide
NaHCO₃ is sodium bicarbonate
Na₂CO₃ is sodium carbonate
Na₂SO₄ is sodium sulfate
NaOt-Bu is sodium tert-butoxide
Pd(OAc)₂ is palladium acetate
Pd(PPh₃)₄ is tetrakis(triphenylphosphine)palladium (0)
Pd₂(dba)₃ is tris(dibenzylideneacetone)dipalladium
PdCl₂(dppf).is [1,1′-Bis(diphenylphosphino)ferrocene]dichloropalladium(II), complex with dichloromethane
PEPPSI-IPr is [1,3-Bis(2,6-Diisopropylphenyl)imidazol-2-ylidene](3-chloropyridyl)palladium(II) dichloride
RuPhos is 2-dicyclohexylphosphino-2,6-diisopropoxybiphenyl
Sn is tin
SnCl₂.H₂O is tin (II) chloride hydrate
SPhos is 2-Dicyclohexylphosphino-2′,6′-dimethoxybiphenyl
TFA is trifluoroacetic acid
THF is tetrahydrofuran
TLC is thin layer chromatography
XantPhos is 4,5-Bis(diphenylphosphino)-9,9-dimethylxanthene

¹H Nuclear magnetic resonance (NMR) spectra were in all cases consistent with the proposed structures. Characteristic chemical shifts (6) are given in parts-per-million downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, deuterochloroform; DMSO-d₆, deuterodimethylsulfoxide; and MeOH-d₄, deuteromethanol. Where appropriate, tautomers may be recorded within the NMR data; and some exchangeable protons may not be visible. Mass spectra, MS (m/z), were recorded using either electrospray ionisation (ESI) or atmospheric pressure chemical ionisation (APCI). Where relevant and unless otherwise stated the m/z data provided are for isotopes ¹⁹F, ³⁵Cl, ⁷⁹Br and ¹²⁷I. Wherein preparative TLC or silica gel chromatography has been used, one skilled in the art may choose any combination of solvents to purify the desired compound.

Analytical liquid chromatography-mass spectrometry (LCMS) QC: Column: Zorbax Extend C18 50×4.6 mm, 5 micron; 5 minutes run. Gradient initial—95% A, 5% B; 3 mins—95% B; hold to 4 mins then back to 5% B at 4.1-5 mins. Flow rate 1.5 mL/min. Conditions: Mobile Phase A: 0.1% ammonium acetate in water; Mobile Phase B: acetonitrile.

Either IUPAC or ACD Labs have been used as naming packages, and are interchangeable throughout the Examples and Preparations.

The following compounds of the invention and related intermediates were prepared using general synthetic processes and schemes described herein.

### Preparation 1

### 6-chloro-N-[(1S)-1-(2-methoxyphenyl)ethyl]-3-nitropyridin-2-amine

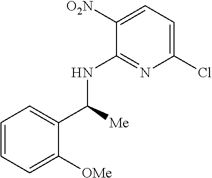 [SMILES: COc1ccccc1[C@H](C)Nc1nc(Cl)ccc1[N+](=O)[O-]]

To a stirred solution of 2,6-dichloro-3-nitropyridine (6.5 g, 33.68 mmol) in IPA (40 mL) was added Na₂CO₃ (10.71 g, 101.04 mmol) and the resultant mixture was allowed to stir at room temperature for 1 hour. A solution of (S)-1-(2-methoxyphenyl)ethan-1-amine (5.09 g, 33.68 mmol) in IPA (20 mL) was slowly added at 0° C. and continued stirring at 0° C. for 4 hours followed by warming to room temperature for 16 hours. The reaction was concentrated in vacuo, diluted with EtOAc and washed with water. The organic layer was collected, dried over sodium sulfate and concentrated in vacuo. The residue was purified using silica gel column chromatography eluting with 10% EtOAc in hexanes to afford the title compound as a yellow solid (8.5 g, 82%). ¹H NMR (400 MHz, DMSO-d₆): δ ppm 1.50 (d, 3H), 3.90 (s, 3H), 5.53-5.61 (m, 1H), 6.77-6.79 (d, 1H), 6.90-6.94 (m, 1H), 7.05-7.07 (m, 1H), 7.25-7.34 (m, 2H), 8.42-8.44 (m, 1H), 9.13-9.15 (m, 1H). MS m/z 308 [M+H]⁺

### Preparation 1a

### 6-chloro-3-nitro-N-[(1S)-1-phenylpropyl]pyridin-2-amine

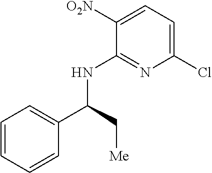 [SMILES: CC[C@H](Nc1nc(Cl)ccc1[N+](=O)[O-])c1ccccc1]

To a solution of 2,6-dichloro-3-nitropyridine (133 g, 0.689 mol) in DCM (2.5 L) was added (1S)-1-phenylpropan-1-amine (105 g, 0.724 mol) drop-wise at −70° C. The mixture was stirred at −70° C. for 5 minutes. DIPEA (260 mL, 1.46 mol) was added drop-wise slowly over 30 minutes at −70° C. The mixture was warmed to 20° C. slowly and stirred for 18 hours. TLC (petroleum ether/ethyl acetate (2:1)) showed most of the starting material was consumed. Water (500 mL) was added and the layers separated. The organic layer was concentrated in vacuo and purified by column chromatography on silica (petroleum ether/ethyl acetate (20:1)) to give the title compound as a yellow solid (200 g, 99.7%). ¹H NMR (DMSO-d₆): □ 8.69 (d, 1E 7.43 (d, 2H), 7.33 (t, 2H), 7.19-7.28 (m, 1H), 6.78 (d, 1H), 5.14 (q, 1H), 1.96-2.08 (m, 1H), 1.90 (dt, 1H), 0.88 (t, 3H). SFC: Chiralcel OJ-3 100×4.6 mm I.D., 3 um. Mobile phase: A: CO₂ B: ethanol (0.05% DEA) Gradient: from 5% to 40% of B in 4.5 minutes and hold 40% for 2.5 minutes, then 5% of B for 1 minute, Flow rate: 2.8 mL/min,

Column temperature: 40° C., Retention time: 2.245 min.

### Preparation 2

### 6-chloro-N²-[(1S)-1-(2-methoxyphenyl)ethyl]pyridine-2,3-diamine

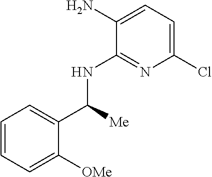 [SMILES: COc1ccccc1[C@H](C)Nc1nc(Cl)ccc1N]

To a degassed solution of 6-chloro-N-[(1S)-1-(2-methoxyphenyl)ethyl]-3-nitropyridin-2-amine (Preparation 1, 8.5 g, 27.62 mmol) in ethyl acetate (100 mL) was added Raney nickel (4 g) under an inert atmosphere. The reaction mixture was hydrogenated in Parr shaker apparatus for 8 hours at 50 psi. The reaction was filtered through Celite and the filtrate was concentrated in vacuo. The residue was purified by silica gel column chromatography to afford the title compound as a brown gum (5 g, 65%). ¹H NMR (400 MHz, DMSO-d₆): δ ppm 1.36-1.38 (d, 3H), 3.84 (s, 3H), 4.98 (br s, 2H), 5.40-5.47 (m, 1H), 6.06-6.08 (m, 1H), 6.28-6.30 (m, 1H), 6.66-6.68 (m, 1H), 6.87-6.89 (m, 1H), 6.95-6.97 (m, 1H), 7.15-7.19 (m, 1H), 7.27-7.29 (m, 1H).MS m/z 278 [M+H]⁺

### Preparation 3

### 2,6-dichloro-4-[(1S)-1-(2-methoxyphenyl)ethyl]pyrido[2,3-b]pyrazin-3(4H)-one

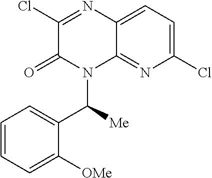 [SMILES: COc1ccccc1[C@H](C)n1c(=O)c(Cl)nc2ccc(Cl)nc21]

A stirred solution of oxalyl chloride (0.31 mL, 3.6 mmol) in 1,2-dichlorobenzene (2 mL) was heated to 65° C. A solution of 6-chloro-N²-[(1S)-1-(2-methoxyphenyl)ethyl]pyridine-2,3-diamine (Preparation 2, 1 g, 3.6 mmol) in 1,2-dichlorobenzene (4 mL) was added over a period of 10 minutes and the reaction heated to 130° C. for 2 hours. The reaction mixture was cooled and further oxalyl chloride (0.62 mL, 7.2 mmol) was added with further heating to 130° C. for 2 hours. The reaction was cooled, quenched with aqueous NaHCO₃ solution and extracted with EtOAc. The organic layer was dried over sodium sulfate and concentrated in vacuo. The residue was purified by silica gel column chromatography eluting with 8% EtOAc-hexane to afford the title compound as a yellow solid (400 mg, 32%).

¹H NMR (400 MHz, DMSO-d₆): δ ppm 1.86-1.88 (m, 3H), 3.46 (s, 3H), 6.69-6.70 (m, 1H), 6.85-6.87 (m, 1H), 6.97-6.99 (m, 1H), 7.22-7.26 (m, 1H), 7.50-7.52 (m, 1H), 7.59-7.60 (m, 1H), 8.23-8.25 (m, 1H). MS m/z 350 [M+H]⁺

### Preparation 4

### tert-butyl N-{6-chloro-4-[(1S)-1-(2-methoxyphenyl)ethyl]-3-oxo-3,4-dihydropyrido[2,3-b]pyrazin-2-yl}-beta-alaninate

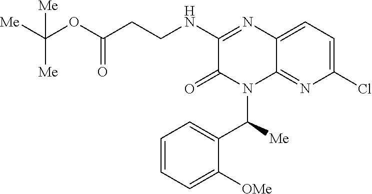 [SMILES: COc1ccccc1[C@H](C)n1c(=O)c(NCCC(=O)OC(C)(C)C)nc2ccc(Cl)nc21]

To a stirred solution of 2,6-dichloro-4-[(1S)-1-(2-methoxyphenyl)ethyl]pyrido[2,3-b]pyrazin-3(4H)-one (Preparation 3, 2.8 g, 7.99 mmol) in DCM (30 mL) was added tert-butyl 3-aminopropanoate (2.54 g, 13.99 mmol) and DIPEA (5.53 mL, 31.98 mmol). The resulting mixture was stirred at room temperature for 16 hours. The reaction was diluted with DCM and washed with water. The organic layer was washed with brine, dried over sodium sulfate and concentrated in vacuo. The residue was purified by silica gel column chromatography eluting with 20% EtOAc in hexanes to afford the title compound as a yellow solid (2.5 g, 68%). ¹H NMR (400 MHz, DMSO-d₆): δ ppm 1.35 (s, 9H), 1.85-1.87 (d, 3H), 2.50-2.56 (m, 2H), 3.45 (s, 3H), 3.54-3.59 (m, 2H), 6.70-6.71 (m, 1H), 6.84-6.86 (m, 1H), 6.93-6.95 (m, 1H), 7.20-7.24 (m, 2H), 7.58-7.60 (m, 1H), 7.74-7.76 (m, 1H), 7.84-7.86 (m, 1H). MS m/z 459 [M+H]⁺

### Preparation 5

### 6-chloro-N²-[(1S)-1-phenylpropyl]pyridine-2,3-diamine

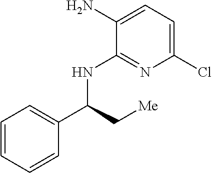 [SMILES: CC[C@H](Nc1nc(Cl)ccc1N)c1ccccc1]

To a solution of SnCl₂.2H₂O (620 g, 2.75 mol) in THF (1 L) was added concentrated HCl (12 M, 340 mL, 4.08 mol) at 25° C. Then a solution of 6-chloro-3-nitro-N-[(1S)-1-phenylpropyl]pyridin-2-amine (Preparation 1a, 200 g, 0.687 mol) in THF (500 mL) was added. The mixture was stirred at 25° C. for 60 hours. TLC (petroleum ether/ethyl acetate (2:1)) showed most of the starting material was consumed. The mixture was adjusted to pH 11 by adding aq. KOH (5M). The mixture was extracted with ethyl acetate (3 L×2). The combined organic layers were concentrated in vacuo to give crude product, which was purified by column chromatography on silica gel (petroleum ether/ethyl acetate (10:1)) to give the title compound as a grey oil (187 g, 100%, contained THF). ¹H NMR (400 MHz, DMSO-d₆): δ ppm 0.86-0.90 (t, 3H), 1.71-1.86 (m, 2H), 4.86-4.92 (m, 1H), 4.96 (s, 2H), 6.13-6.15 (m, 1H), 6.27-6.29 (m, 1H), 6.64 (m, 1H), 7.15-7.36 (m, 5H).

MS m/z 262 [M+H]⁺

### Preparation 6

### 2,6-dichloro-4-[(1S)-1-phenylpropyl]pyrido[2,3-b]pyrazin-3(4H)-one

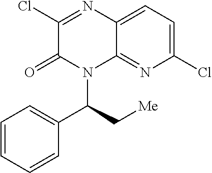 [SMILES: CC[C@@H](c1ccccc1)n1c(=O)c(Cl)nc2ccc(Cl)nc21]

To a stirred solution of 6-chloro-N²-[(1S)-1-phenylpropyl]pyridine-2,3-diamine (Preparation 5, 500 mg, 1.91 mmol) in dioxane (10 mL) was added methyl 2-chloro-2-oxoacetate (0.21 mL, 2.29 mmol) and Cs₂CO₃ (1.86 g, 5.73 mmol). The reaction was allowed to stir at room temperature for 2 hours before heating to 120° C. for 16 hours. The reaction was filtered and the filtrate was concentrated in vacuo. The residue was dissolved in THF (7 mL) and oxalyl chloride (0.27 mL, 3.17 mmol) and DMF (catalytic amount) were added and the reaction was heated at 50° C. for 4 hours. The reaction was concentrated in vacuo and the residue was used directly in the next step (500 mg, 80% over two steps).

### Preparation 7

### Methyl N-{6-chloro-3-oxo-4-[(1S)-1-phenylpropyl]-3,4-dihydropyrido[2,3-b]pyrazin-2-yl}-beta-alaninate

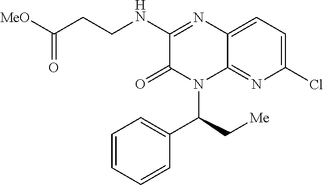 [SMILES: CC[C@@H](c1ccccc1)n1c(=O)c(NCCC(=O)OC)nc2ccc(Cl)nc21]

The title compound was prepared according to the method described for Preparation 4 using 2,6-dichloro-4-[(1S)-1-phenylpropyl]pyrido[2,3-b]pyrazin-3(4H)-one (Preparation 6) and methyl-3-aminopropanoate.

¹H NMR (400 MHz, CDCl₃): δ ppm 0.80 (t, 3H), 2.55-2.60 (m, 1H), 2.70-2.85 (m, 4H), 3.70 (s, 3H), 3.80-3.85 (m, 2H), 7.20-7.40 (m, 5H), 7.60 (m, 1H), 7.80 (m, 1H). MS m/z 401 [M+H]⁺

### Preparation 7A

### Tert-butyl N-{6-chloro-3-oxo-4-[(1S)-1-phenylpropyl]-3,4-dihydropyrido[2,3-b]pyrazin-2-yl}-beta-alaninate

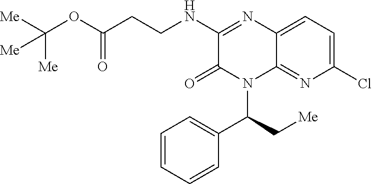 [SMILES: CC[C@@H](c1ccccc1)n1c(=O)c(NCCC(=O)OC(C)(C)C)nc2ccc(Cl)nc21]

The title compound was prepared according to the method described for Preparation 4 using 2,6-dichloro-4-[(1S)-1-phenylpropyl]pyrido[2,3-b]pyrazin-3(4H)-one (Preparation 6) and tert-butyl-3-aminopropanoate. MS m/z 443 [M+H]⁺

### Preparation 8

### 6-chloro-4-[(1S)-1-(2-methoxyphenyl)ethyl]-2-(methylamino)pyrido[2,3-b]pyrazin-3(4H)-one

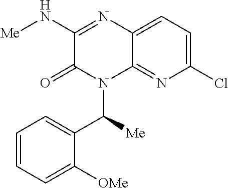 [SMILES: CNc1nc2ccc(Cl)nc2n([C@@H](C)c2ccccc2OC)c1=O]

The title compound was prepared according to the method described for Preparation 4 using methylamine hydrochloride.

¹H NMR (400 MHz, DMSO-d₆): δ ppm 1.87 (d, 3H), 2.86 (d, 3H), 3.46 (s, 3H), 6.67-6.72 (m, 1H), 6.84-6.86 (m, 1H), 6.93-6.97 (m, 1H), 7.20-7.24 (m, 2H), 7.58-7.60 (m, 1H), 7.73-7.75 (m, 1H), 7.92-7.93 (m, 1H). MS m/z 345 [M+H]⁺

### Preparation 9

### 6-chloro-N-[(1R)-2-methoxy-1-phenylethyl]-3-nitropyridin-2-amine

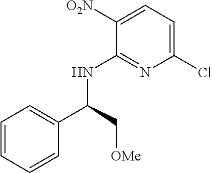 [SMILES: COC[C@H](Nc1nc(Cl)ccc1[N+](=O)[O-])c1ccccc1]

To a mixture of 2,6-dichloro-3-nitropyridine 1 (3.5 g, 18.135 mmol) and (R)-2-methoxy-1-phenylethan-1-amine (2.879 g, 19.041 mmol) in DCM (50 mL) was added DIPEA (4.922 g, 38.083 mmol) at 0° C. followed by stirring at room temperature overnight. The reaction was quenched with ice water and the layers separated. The organic layer was washed with water, brine, dried over sodium sulfate and concentrated in vacuo. The residue was purified by silica gel column chromatography eluting with 5.2% EtOAc in hexanes to afford the title compound as a yellow solid (4.2 g, 75.27%).

¹H NMR (400 MHz, DMSO-d₆): δ ppm 3.30-3.34 (m, 3H), 3.69-3.73 (m, 1H), 3.81-3.85 (m, 1H), 5.43-5.48 (m, 1H), 6.80-6.82 (m, 1H), 7.23-7.44 (m, 5H), 8.44-8.46 (m, 1H), 8.91-8.93 (m, 1H).

MS m/z 308 [M+H]⁺

### Preparation 9A

### 6-chloro-3-nitro-N-[(1S)-1-(pyridin-2-yl)propyl]pyridin-2-amine

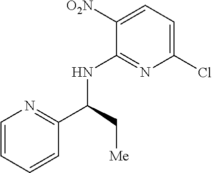 [SMILES: CC[C@H](Nc1nc(Cl)ccc1[N+](=O)[O-])c1ccccn1]

The title compound was prepared according to the method described for Preparation 9 using (S)-1-(pyridin-2-yl)propan-1-amine at −78° C.

¹H NMR (400 MHz, DMSO-de): 8 ppm 0.84-0.87 (t, 3H), 1.89-2.02 (m, 2H), 5.33-5.38 (m, 1H), 6.81-6.83 (m, 1H), 7.32-7.35 (m, 1H), 7.51-7.53 (m, 1H), 7.80-7.90 (m, 1H), 8.45-8.48 (m, 1H), 8.61-8.65 (m, 1H), 9.39-9.41 (m, 1H).

MS m/z 293 [M+H]⁺

### Preparation 9B

### 6-chloro-N-[(1S)-2-methyl-1-(pyridin-2-yl)propyl]-3-nitropyridin-2-amine

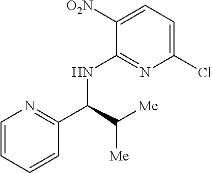 [SMILES: CC(C)[C@H](Nc1nc(Cl)ccc1[N+](=O)[O-])c1ccccn1]

The title compound was prepared according to the method described for Preparation 9 using (S)-2-methyl-1-(pyridin-2-yl)propan-1-amine at −70° C.

¹H NMR (400 MHz, DMSO-d₆): δ ppm 0.78 (d, 3H), 0.89 (d, 3H), 2.24-2.29 (m, 1H), 5.24-5.25 (m, 1H), 6.77 (d, 1H), 7.29-7.32 (m, 1H), 7.42-7.44 (m, 1H), 7.75-7.79 (m, 1H), 8.42 (d, 1H), 8.56-8.57 (m, 1H). MS m/z 307 [M+H]⁺

### Preparation 9C

### 6-chloro-3-nitro-N-[(3S,4S)-4-phenyltetrahydrofuran-3-yl]pyridin-2-amine

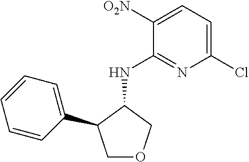 [SMILES: O=[N+]([O-])c1ccc(Cl)nc1N[C@@H]1COC[C@H]1c1ccccc1]

The title compound was prepared according to the method described for Preparation 9 using (3S,4S)-4-phenyltetrahydrofuran-3-amine at −70° C.

¹H NMR (400 MHz, DMSO-d₆): δ ppm 3.79-3.85 (m, 2H), 4.08-4.23 (m, 3H), 4.99-5.06 (m, 1H), 6.74-6.76 (d, 1H), 7.17-7.28 (m, 5H), 7.99-8.01 (m, 1H), 8.28-8.30 (m, 1H). MS m/z 319.8 [M+H]⁺

### Preparation 9D

### 6-chloro-N-(1-cyclopentylcyclopropyl)-3-nitropyridin-2-amine

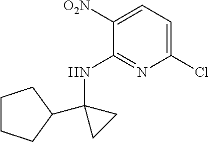 [SMILES: O=[N+]([O-])c1ccc(Cl)nc1NC1(C2CCCC2)CC1]

The title compound was prepared according to the method described for Preparation 9 using 1-cyclopentylcyclopropan-1-amine at −70° C.

¹H NMR (400 MHz, DMSO-d₆): δ ppm 0.77-0.84 (m, 4H), 1.18-1.22 (m, 2H), 1.44-1.63 (m, 6H), 2.26-2.34 (m, 1H), 6.82 (d, 1H), 8.40 (d, 1H), 8.61 (br s, 1H). MS m/z 282 [M+H]⁺

### Preparation 9E

### 6-chloro-3-nitro-N-(1-phenylcyclobutyl)pyridin-2-amine

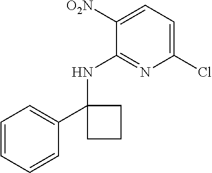 [SMILES: O=[N+]([O-])c1ccc(Cl)nc1NC1(c2ccccc2)CCC1]

The title compound was prepared according to the method described for Preparation 9 using 1-phenylcyclobutan-1-amine.

¹H NMR (400 MHz, DMSO-d₆): δ ppm 1.83-1.88 (m, 1H), 1.99-2.04 (m, 1H), 2.57-2.71 (m, 4H), 6.72 (d, 1H), 7.17-7.20 (m, 1H), 7.29-7.33 (m, 2H), 7.56-7.58 (m, 2H), 8.36 (d, 1H), 8.98 (s, 1H).

MS m/z 304 [M+H]⁺

### Preparation 9F

### 6-chloro-N-(2,5-diethylcyclopentyl)-3-nitropyridin-2-amine

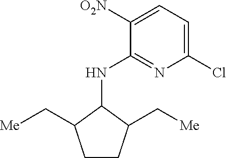 [SMILES: CCC1CCC(CC)C1Nc1nc(Cl)ccc1[N+](=O)[O-]]

The title compound was prepared according to the method described for Preparation 9 using 2,5-diethylcyclopentan-1-amine.

¹H NMR (400 MHz, DMSO-d₆): δ ppm 0.80-0.95 (m, 6H), 1.21-1.58 (m, 7H), 1.85-2.08 (m, 4H), 6.78-6.80 (m, 1H), 8.28-8.44 (m, 2H). MS m/z 298 [M+H]⁺

### Preparation 9G

### 6-chloro-N-[(2R)-1-methoxybutan-2-yl]-3-nitropyridin-2-amine

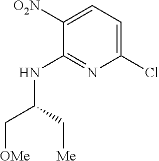 [SMILES: CC[C@H](COC)Nc1nc(Cl)ccc1[N+](=O)[O-]]

The title compound was prepared according to the method described for Preparation 9 using (R)-1-methoxybutan-2-amine at −70° C.

¹H NMR (400 MHz, DMSO-d₆): δ ppm 0.89 (t, 3H), 1.57-1.69 (m, 2H), 3.29 (s, 3H), 3.42-3.45 (m, 1H), 3.53-3.56 (m, 1H), 4.34-4.36 (m, 1H), 6.79 (d, 1H), 8.37-8.44 (m, 2H). MS m/z 260 [M+H]⁺

### Preparation 9H

### 6-chloro-N-[(2R)-1-methoxypentan-2-yl]-3-nitropyridin-2-amine

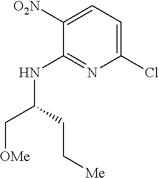 [SMILES: CCC[C@H](COC)Nc1nc(Cl)ccc1[N+](=O)[O-]]

The title compound was prepared according to the method described for Preparation 9 using (R)-1-methoxypentan-2-amine at −78° C.

¹H NMR (400 MHz, DMSO-de): δ ppm 0.87 (t, 3H), 1.28-1.36 (m, 2H), 1.57-1.62 (m, 2H), 3.28 (s, 3H), 3.42-3.45 (m, 1H), 3.51-3.55 (m, 1H), 4.45-4.47 (m, 1H), 6.79 (d, 1H), 8.35-8.44 (m, 2H).

MS m/z 274 [M+H]⁺

### Preparation 9I

### 6-chloro-N-(1,3-dimethoxypropan-2-yl)-3-nitropyridin-2-amine

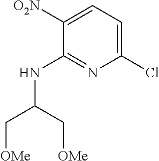 [SMILES: COCC(COC)Nc1nc(Cl)ccc1[N+](=O)[O-]]

The title compound was prepared according to the method described for Preparation 9 using 1,3-dimethoxypropan-2-amine.

¹H NMR (400 MHz, DMSO-d₆): δ ppm 3.30-3.31 (m, 6H), 3.46-3.57 (m, 4H), 4.55-4.60 (m, 1H), 6.83-6.85 (d, 1H), 8.44-8.46 (m, 2H). MS m/z 275 [M+H]⁺

### Preparation 9J

### 6-chloro-N-(2-ethoxybenzyl)-3-nitropyridin-2-amine

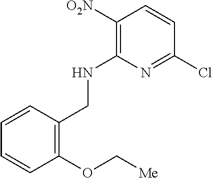 [SMILES: CCOc1ccccc1CNc1nc(Cl)ccc1[N+](=O)[O-]]

The title compound was prepared according to the method described for Preparation 9 using (2-ethoxyphenyl)methanamine.

¹H NMR (400 MHz, DMSO-d₆): δ ppm 8.96 (b s, 1H), 8.30 (d, 1H), 7.36 (d, 1H), 7.25 (t, 1H), 6.90 (dd, 2H), 6.55 (d, 1H), 4.82 (d, 2H), 4.11 (dd, 2H), 1.49 (t, 3H). MS m/z 308 [M+H]⁺

### Preparation 9K

### N-benzyl-6-chloro-3-nitropyridin-2-amine

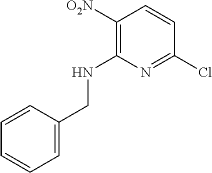 [SMILES: O=[N+]([O-])c1ccc(Cl)nc1NCc1ccccc1]

The title compound was prepared according to the method described for Preparation 9 using benzylamine.

¹H NMR (400 MHz, DMSO-d₆): δ ppm 9.19 (m, 1H), 8.44 (d, 1H), 7.34 (m, 4H), 7.24 (t, 1H), 6.79 (d, 1H), 4.72 (d, 2H). MS m/z 264 [M+H]⁺

### Preparation 9L

### 6-chloro-3-nitro-N-[(1R)-1-phenylpropyl]pyridin-2-amine

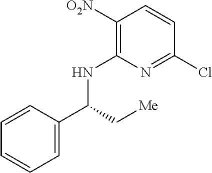 [SMILES: CC[C@@H](Nc1nc(Cl)ccc1[N+](=O)[O-])c1ccccc1]

The title compound was prepared according to the method described for Preparation 9 using (R)-1-phenylpropan-1-amine. MS m/z 292 [M+H]⁺

### Preparation 9M

### 6-chloro-3-nitro-N-[(1S)-1-phenylethyl]pyridin-2-amine

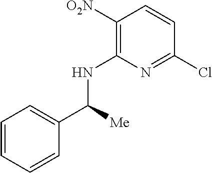 [SMILES: C[C@H](Nc1nc(Cl)ccc1[N+](=O)[O-])c1ccccc1]

The title compound was prepared according to the method described for Preparation 9 using (S)-1-phenylethan-1-amine.

¹H NMR (400 MHz, DMSO-d₆): δ ppm 8.65 (d, 1H), 8.43 (d, 1H), 7.45 (d, 1H), 7.34 (t, 2H), 7.25 (t, 1H), 6.80 (d, 1H), 5.37 (m, 1H), 1.59 (d, 3H). MS m/z 278 [M+H]⁺

### Preparation 9N

### 6-chloro-N-[(1S)-1-(2-methoxyphenyl)ethyl]-3-nitropyridin-2-amine

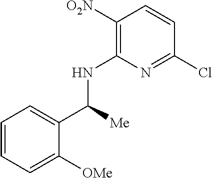 [SMILES: COc1ccccc1[C@H](C)Nc1nc(Cl)ccc1[N+](=O)[O-]]

The title compound was prepared according to the method described for Preparation 9 using (S)-1-(2-methoxyphenyl)ethan-1-amine.

¹H NMR (400 MHz, DMSO-d₆): δ ppm 9.14 (d, 1H), 8.43 (d, 1H), 7.33 (d, 1H), 7.27 (t, 1H), 7.06 (d, 1H), 6.92 (t, 1H), 6.78 (d, 1H), 5.56 (m, 1H), 3.90 (s, 3H), 1.51 (d, 3H). MS m/z 308 [M+H]⁺

### Preparation 9O

### 6-chloro-3-nitro-N-[(1S)-1-phenylpropyl]pyridin-2-amine

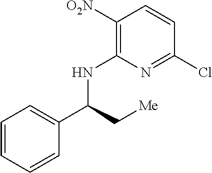 [SMILES: CC[C@H](Nc1nc(Cl)ccc1[N+](=O)[O-])c1ccccc1]

The title compound was prepared according to the method described for Preparation 9 using (S)-1-phenylpropan-1-amine.

¹H NMR (400 MHz, DMSO-d₆): δ ppm 8.69 (d, 1H), 8.42 (d, 1H), 7.43 (d, 2H), 7.33 (t, 2H), 7.24 (t, 1H), 6.79 (d, 1H), 5.14 (dt, 1H), 2.06-1.87 (m. 2H), 0.88 (t, 3H). MS m/z 292 [M+H]⁺

### Preparation 9P

### 6-chloro-N-[(2S)-1-methoxybutan-2-yl]-3-nitropyridin-2-amine

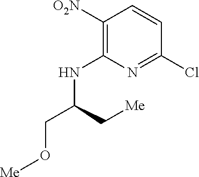 [SMILES: CC[C@@H](COC)Nc1nc(Cl)ccc1[N+](=O)[O-]]

The title compound was prepared according to the method described for Preparation 9 using (S)-1-methoxybutan-2-amine.

¹H NMR (400 MHz, CDCl₃): δ ppm 8.42 (d, 1H), 8.33 (d, 1H), 6.57 (d, 1H), 4.44 (m, 1H), 3.55-3.45 (m, 2H), 3.37 (s, 3H), 1.78-1.65 (m, 2H), 0.97 (t, 3H). MS m/z 260 [M+H]⁺

### Preparation 9Q

### 6-chloro-N-[(2R)-1-methoxybutan-2-yl]-3-nitropyridin-2-amine

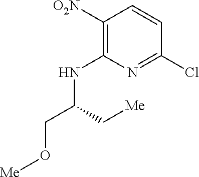 [SMILES: CC[C@H](COC)Nc1nc(Cl)ccc1[N+](=O)[O-]]

The title compound was prepared according to the method described for Preparation 9 using (R)-1-methoxybutan-2-amine.

¹H NMR (400 MHz, DMSO-de): δ ppm 8.44 (d, 1H), 8.38 (d, 1H), 6.80 (d, 1H), 4.34 (m, 1H), 3.56-3.42 (m, 2H), 3.29 (s, 3H), 1.69-1.59 (m, 2H), 0.89 (t, 3H). MS m/z 260 [M+H]⁺

### Preparation 9R

### 6-chloro-N-(1,3-dimethoxypropan-2-yl)-3-nitropyridin-2-amine

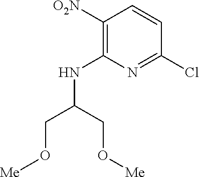 [SMILES: COCC(COC)Nc1nc(Cl)ccc1[N+](=O)[O-]]

The title compound was prepared according to the method described for Preparation 9 using 1,3-dimethoxypropan-2-amine.

¹H NMR (400 MHz, DMSO-de): δ ppm 8.45 (d, 2H), 6.84 (d, 1H), 4.58 (m, 1H), 3.57-3.46 (m, 4H), 3.30 (s, 6H). MS m/z 276 [M+H]⁺

### Preparation 9S

### 6-Chloro-3-nitro-N-(tetrahydro-2H-pyran-4-yl)pyridin-2-amine

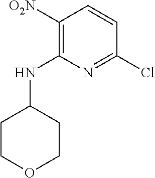 [SMILES: O=[N+]([O-])c1ccc(Cl)nc1NC1CCOCC1]

The title compound was prepared according to the method described for Preparation 9 using 4-aminotetrahydropyran.

¹H NMR (400 MHz, DMSO-de): δ ppm 8.42 (d, 1H), 8.30 (d, 1H), 6.81 (d, 1H), 4.30-4.23 (m, 1H), 3.88 (d, 2H), 3.44 (t, 2H), 1.86 (d, 2H), 1.74-1.64 (m, 2H). MS m/z 256 [M−H]⁺

### Preparation 9T

### 6-chloro-3-nitro-N-[(1S)-1-(pyrimidin-2-yl)propyl]pyridin-2-amine

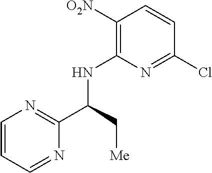 [SMILES: CC[C@H](Nc1nc(Cl)ccc1[N+](=O)[O-])c1ncccn1]

The title compound was prepared according to the method described for Preparation 9 using (S)-1-(pyrimidin-2-yl)propan-1-amine.

¹H NMR (400 MHz, CDCl₃): δ ppm 9.43 (d, 1H), 8.75 (d, 2H), 8.35 (d, 1H), 7.20 (t, 1H), 6.58 (d, 1H), 5.60 (m, 1H), 2.22-2.15 (m, 1H), 2.10-2.03 (m, 1H), 0.87 (t, 3H). MS m/z 294 [M+H]⁺

### Preparation 10

### 6-chloro-N²-[(1R)-2-methoxy-1-phenylethyl]pyridine-2,3-diamine

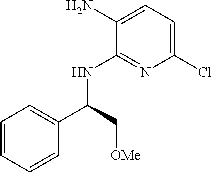 [SMILES: COC[C@H](Nc1nc(Cl)ccc1N)c1ccccc1]

To a mixture of conc. HCl (6.974 g, 191.07 mmol) and SnCl₂ (10.352 g, 54.592 mmol) in THF (50 mL) was added 6-chloro-N-[(1R)-2-methoxy-1-phenylethyl]-3-nitropyridin-2-amine (Preparation 9, 4.2 g, 13.648 mmol) at 0° C. and the reaction was stirred at room temperature for 3 hours. The reaction was quenched with 2M KOH solution, filtered and the filtrate was partitioned between ethyl acetate and water. The organic layer was dried over sodium sulfate and concentrated in vacuo. The residue was purified by silica gel column chromatography eluting with 70% EtOAc in hexanes to afford the title compound: (3.7 g, 97%). ¹H NMR (400 MHz, DMSO-d₆): δ ppm 3.25 (s, 3H), 3.54-3.66 (m, 2H), 4.99 (br s, 2H), 5.25-5.30 (m, 1H), 6.19-6.21 (m, 1H), 6.32-6.34 (m, 1H), 6.68-6.70 (m, 1H), 7.19-7.40 (m, 5H). MS m/z 278 [M+H]⁺

### Preparation 10A

### 6-chloro-N²-[(1S)-1-(pyridin-2-yl)propyl]pyridine-2,3-diamine

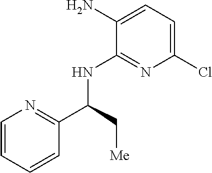 [SMILES: CC[C@H](Nc1nc(Cl)ccc1N)c1ccccn1]

The title compound was prepared according to the method described for Preparation 10 using 6-chloro-3-nitro-N-[(1S)-1-(pyridin-2-yl)propyl]pyridin-2-amine (Preparation 9A).

MS m/z 263 [M+H]⁺

### Preparation 10B

### 6-chloro-N²-[(1S)-2-methyl-1-(pyridin-2-yl)propyl]pyridine-2,3-diamine

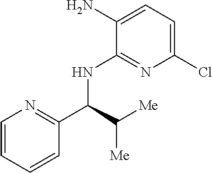 [SMILES: CC(C)[C@H](Nc1nc(Cl)ccc1N)c1ccccn1]

The title compound was prepared according to the method described for Preparation 10 using 6-chloro-N-[(1S)-2-methyl-1-(pyridin-2-yl)propyl]-3-nitropyridin-2-amine (Preparation 9B). ¹H NMR (400 MHz, DMSO-de): δ ppm 0.75 (d, 3H), 0.97 (d, 3H), 2.23-2.32 (m, 1H), 4.87-4.91 (m, 1H), 5.02 (br s, 1H), 6.07 (d, 1H), 6.30 (d, 1H), 6.65 (d, 1H), 7.19-7.22 (m, 1H), 7.36 (d, 1H), 7.68-7.72 (m, 1H), 8.50 (d, 1H). MS m/z 277 [M+H]⁺

### Preparation 10C

### 6-chloro-N²-[(3S,4S)-4-phenyltetrahydrofuran-3-yl]pyridine-2,3-diamine

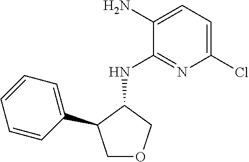 [SMILES: Nc1ccc(Cl)nc1N[C@@H]1COC[C@H]1c1ccccc1]

The title compound was prepared according to the method described for Preparation 10 using 6-chloro-3-nitro-N-[(3S,4S)-4-phenyltetrahydrofuran-3-yl]pyridin-2-amine (Preparation 9C). ¹H NMR (400 MHz, DMSO-d₆): δ ppm 3.70-3.74 (m, 2H), 4.01-4.19 (m, 3H), 4.70 (br s, 2H), 4.81-4.87 (m, 1H), 5.47-5.49 (m, 1H), 6.28 (d, 1H), 6.54 (d, 1H), 7.08-7.19 (m, 5H). MS m/z 290 [M+H]⁺

### Preparation 10D

### 6-chloro-N²-(1-cyclopentylcyclopropyl)pyridine-2,3-diamine

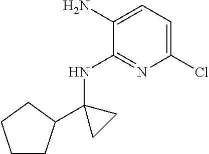 [SMILES: Nc1ccc(Cl)nc1NC1(C2CCCC2)CC1]

The title compound was prepared according to the method described for Preparation 10 using 6-chloro-N-(1-cyclopentylcyclopropyl)-3-nitropyridin-2-amine (Preparation 9D).

¹H NMR (400 MHz, DMSO-d₆): δ ppm 0.55-0.58 (m, 2H), 0.66-0.69 (m, 2H), 1.15-1.20 (m, 2H), 1.42-1.58 (m, 6H), 2.35-2.39 (m, 1H), 4.77 (br s, 2H), 6.13 (br s, 1H), 6.30 (d, 1H), 6.63 (d, 1H).

MS m/z 252 [M+H]⁺

### Preparation 10E

### 6-chloro-N²-(1-phenylcyclobutyl)pyridine-2,3-diamine

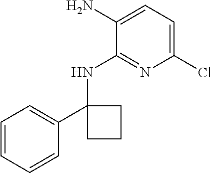 [SMILES: Nc1ccc(Cl)nc1NC1(c2ccccc2)CCC1]

The title compound was prepared according to the method described for Preparation 10 using 6-chloro-3-nitro-N-(1-phenylcyclobutyl)pyridin-2-amine (Preparation 9E).

MS m/z 274 [M+H]⁺

### Preparation 10F

### 6-chloro-N²-(2,5-diethylcyclopentyl)pyridine-2,3-diamine

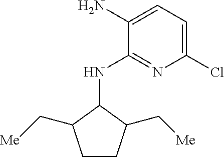 [SMILES: CCC1CCC(CC)C1Nc1nc(Cl)ccc1N]

The title compound was prepared according to the method described for Preparation 10 using 6-chloro-N-(2,5-diethylcyclopentyl)-3-nitropyridin-2-amine (Preparation 9F) and taken on directly to the next step.

### Preparation 10G

### 6-chloro-N²-[(2R)-1-methoxybutan-2-yl]pyridine-2,3-diamine

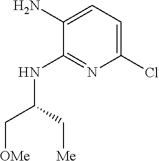 [SMILES: CC[C@H](COC)Nc1nc(Cl)ccc1N]

The title compound was prepared according to the method described for Preparation 2 using 6-chloro-N-[(2R)-1-methoxybutan-2-yl]-3-nitropyridin-2-amine (Preparation 9G).

¹H NMR (400 MHz, DMSO-de): δ ppm 0.86 (t, 3H), 1.40-1.47 (m, 1H), 1.58-1.65 (m, 1H), 3.22 (s, 3H), 3.24-3.37 (m, 2H), 3.97-4.02 (m, 1H), 4.84 (br s, 2H), 6.28 (d, 1H), 6.62 (d, 1H). MS m/z 230 [M+H]⁺

### Preparation 10H

### 6-chloro-N²-[(2R)-1-methoxypentan-2-yl]pyridine-2,3-diamine

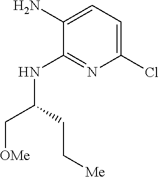 [SMILES: CCC[C@H](COC)Nc1nc(Cl)ccc1N]

The title compound was prepared according to the method described for Preparation 10 using 6-chloro-N-[(2R)-1-methoxypentan-2-yl]-3-nitropyridin-2-amine (Preparation 9H).

¹H NMR (400 MHz, DMSO-de): δ ppm 0.85-0.89 (t, 3H), 1.23-1.60 (m, 4H), 3.25 (s, 3H), 3.27-3.39 (m, 2H), 4.12-4.13 (m, 1H), 4.86 (br s, 2H), 5.54-5.55 (m, 1H), 6.29 (d, 1H), 6.65 (d, 1H).

MS m/z 244 [M+H]⁺

### Preparation 10I

### 6-chloro-N²-(1,3-dimethoxypropan-2-yl)pyridine-2,3-diamine

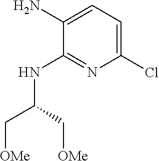

The title compound was prepared according to the method described for Preparation 10 using 6-chloro-N-(1,3-dimethoxypropan-2-yl)-3-nitropyridin-2-amine (Preparation 9I).

¹H NMR (400 MHz, DMSO-d₆): δ ppm 3.23 (s, 3H), 3.31 (s, 3H), 3.40-3.46 (m, 4H), 4.27-4.32 (m, 1H), 4.90 (s, 2H), 5.67-5.69 (m, 1H), 6.35 (d, 1H), 6.68 (d, 1H). MS m/z 246 [M+H]⁺

### Preparation 10J

### 6-chloro-N²-(2-ethoxybenzyl)pyridine-2,3-diamine

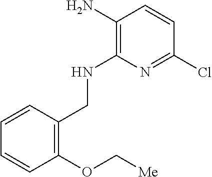 [SMILES: CCOc1ccccc1CNc1nc(Cl)ccc1N]

The title compound was prepared according to the method described for Preparation 10 using 6-chloro-N-(2-ethoxybenzyl)-3-nitropyridin-2-amine (Preparation 9J).

¹H NMR (400 MHz, DMSO-d₆): δ ppm 7.20 (m, 2H), 6.96 (d, 1H), 6.86 (t, 1H), 6.70 (d, 1H), 6.35 (d, 1H), 6.15 (t, 1H), 4.90 (s, 2H), 4.48 (d, 2H), 4.10-4.00 (m, 2H), 1.35 (t, 3H).

MS m/z 278 [M+H]⁺

### Preparation 10K

### N²-benzyl-6-chloropyridine-2,3-diamine

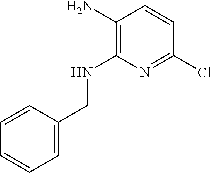 [SMILES: Nc1ccc(Cl)nc1NCc1ccccc1]

The title compound was prepared according to the method described for Preparation 10 using 6-chloro-N2-(2-ethoxybenzyl)pyridine-2,3-diamine (Preparation 9K).

¹H NMR (400 MHz, DMSO-d₆): δ ppm 7.33 (m, 4H), 7.22 (m, 1H), 6.70 (d, 1H), 6.39 (t, 1H), 6.36 (d, 1H), 4.88 (s, 2H), 4.51 (d, 2H). MS m/z 234 [M+H]⁺

### Preparation 10L

### 6-chloro-N²-[(1R)-1-phenylpropyl]pyridine-2,3-diamine

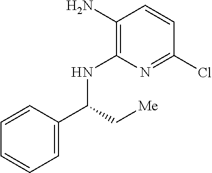 [SMILES: CC[C@@H](Nc1nc(Cl)ccc1N)c1ccccc1]

The title compound was prepared according to the method described for Preparation 10 using 6-chloro-3-nitro-N-[(1R)-1-phenylpropyl]pyridin-2-amine (Preparation 9L).

¹H NMR (400 MHz, DMSO-d₆): δ ppm 7.35 (d, 2H), 7.28 (t, 2H), 7.17 (t, 1H), 6.65 (d, 1H), 6.28 (d, 1H), 6.14 (d, 1H), 4.97 (s, 2H), 4.89 (dd, 1H), 1.86-1.71 (m, 2H), 0.88 (t, 3H).

MS m/z 262 [M+H]⁺

### Preparation 11

### 2,6-dichloro-4-[(1R)-2-methoxy-1-phenylethyl]pyrido[2,3-b]pyrazin-3(4H)-one

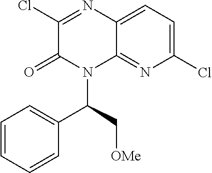 [SMILES: COC[C@@H](c1ccccc1)n1c(=O)c(Cl)nc2ccc(Cl)nc21]

To a stirred solution of 6-chloro-N²-[(1R)-2-methoxy-1-phenylethyl]pyridine-2,3-diamine (3.7 g, 13.321 mmol) in tetrahydrofuran (100 mL) was added methyl chloro oxoacetate (1.35 mL, 14.653 mmol) and sodium carbonate (3.247 g, 30.638 mmol) and the reaction was allowed to stir at room temperature for 2 hours. The reaction was quenched with brine and extracted with ethyl acetate. The organic layer was dried over sodium sulfate and concentrated in vacuo. The residue (4.5 g, 12.369 mmol) was dissolved in THF (50 mL) and 1N NaOH (1.484 g, 37.108 mmol) was added at room temperature. The reaction was quenched with 2N HCl and extracted with ethyl acetate. The organic layer was dried over sodium sulfate and concentrated in vacuo) to afford the acid intermediate.

To a stirred solution of the acid intermediate (2.3 g, 6.576 mmol) in THF (100 mL) at room temperature was added oxalyl chloride (0.565 mL, 6.576 mmol) drop wise in the presence of a catalytic amount of DMF. The reaction mass was allowed to stir at 50° C. overnight. The reaction was concentrated in vacuo and purified by silica gel column chromatography eluting with 70% EtOAc in hexanes to afford the title compound (1.5 g, 33%). MS m/z 350 [M+H]⁺

### Preparation 11A

### 2,6-dichloro-4-[(1S)-1-(pyridin-2-yl)propyl]pyrido[2,3-b]pyrazin-3(4H)-one

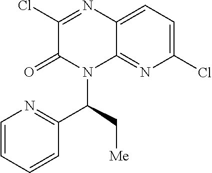 [SMILES: CC[C@@H](c1ccccn1)n1c(=O)c(Cl)nc2ccc(Cl)nc21]

The title compound was prepared according to the method described for Preparation 11 using (6-chloro-N²-[(1S)-1-(pyridin-2-yl)propyl]pyridine-2,3-diamine (Preparation 10A).

MS m/z 335 [M+H]⁺

### Preparation 11B

### 2,6-dichloro-4-[(1S)-2-methyl-1-(pyridin-2-yl)propyl]pyrido[2,3-b]pyrazin-3(4H)-one

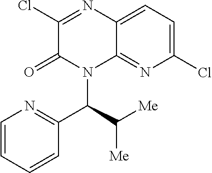 [SMILES: CC(C)[C@@H](c1ccccn1)n1c(=O)c(Cl)nc2ccc(Cl)nc21]

The title compound was prepared according to the method described for Preparation 11 using 6-chloro-N²-[(1S)-2-methyl-1-(pyridin-2-yl)propyl]pyridine-2,3-diamine (Preparation 10B). MS m/z 348 [M+H]⁺

### Preparation 11C

### 2,6-dichloro-4-[(3S,4S)-4-phenyltetrahydrofuran-3-yl]pyrido[2, 3-b]pyrazin-3(4H)-one

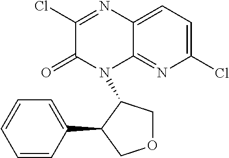 [SMILES: O=c1c(Cl)nc2ccc(Cl)nc2n1[C@@H]1COC[C@H]1c1ccccc1]

The title compound was prepared according to the method described for Preparation 11 using 6-chloro-N²-[(3S,4S)-4-phenyltetrahydrofuran-3-yl]pyridine-2,3-diamine (Preparation 10C). MS m/z 362 [M+H]⁺

### Preparation 11D

### 2,6-dichloro-4-(1-cyclopentylcyclopropyl)pyrido[2,3-b]pyrazin-3(4H)-one

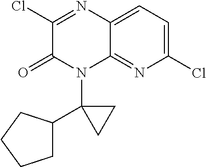 [SMILES: O=c1c(Cl)nc2ccc(Cl)nc2n1C1(C2CCCC2)CC1]

The title compound was prepared according to the method described for Preparation 11 using 6-chloro-N²-(1-cyclopentylcyclopropyl)pyridine-2,3-diamine (Preparation 10D), MS m/z 324 [M+H]⁺

### Preparation 11E

### 2,6-dichloro-4-(1-phenylcyclobutyl)pyrido[2,3-b]pyrazin-3(4H)-one

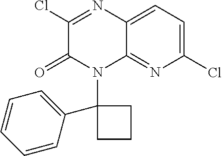 [SMILES: O=c1c(Cl)nc2ccc(Cl)nc2n1C1(c2ccccc2)CCC1]

The title compound was prepared according to the method described for Preparation 11 using 6-chloro-N²-(1-phenylcyclobutyl)pyridine-2,3-diamine (Preparation 10E) and taken on directly to the next step.

### Preparation 11F

### 2,6-dichloro-4-(2,5-diethylcyclopentyl)pyrido[2,3-b]pyrazin-3(4H)-one

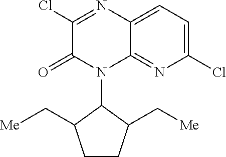 [SMILES: CCC1CCC(CC)C1n1c(=O)c(Cl)nc2ccc(Cl)nc21]

The title compound was prepared according to the method described for Preparation 11 using 6-chloro-N²-(2,5-diethylcyclopentyl)pyridine-2,3-diamine (Preparation 10F) and taken on directly to the next step.

### Preparation 11G

### 2,6-dichloro-4-[(2R)-1-methoxybutan-2-yl]pyrido[2,3-b]pyrazin-3(4H)-one

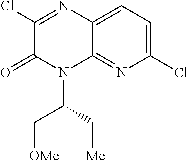 [SMILES: CC[C@H](COC)n1c(=O)c(Cl)nc2ccc(Cl)nc21]

The title compound was prepared according to the method described for Preparation 11 using 6-chloro-N²-[(2R)-1-methoxybutan-2-yl]pyridine-2,3-diamine (Preparation 10G) and taken on directly to the next step.

### Preparation 11H

### 2,6-dichloro-4-[(2R)-1-methoxypentan-2-yl]pyrido[2,3-b]pyrazin-3(4H)-one

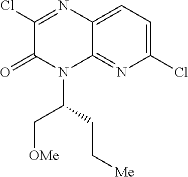 [SMILES: CCC[C@H](COC)n1c(=O)c(Cl)nc2ccc(Cl)nc21]

The title compound was prepared according to the method described for Preparation 11 using 6-chloro-N²-[(2R)-1-methoxypentan-2-yl]pyridine-2,3-diamine (Preparation 10H).

MS m/z 316 [M+H]⁺

### Preparation 11I

### 2,6-dichloro-4-(1,3-dimethoxypropan-2-yl)pyrido[2,3-b]pyrazin-3(4H)-one

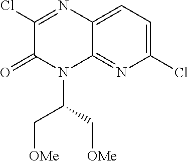

The title compound was prepared according to the method described for Preparation 11 using 6-chloro-N²-(1,3-dimethoxypropan-2-yl)pyridine-2,3-diamine (Preparation 101).

MS m/z 350 [M+H]⁺

### Preparation 11J

### 2,6-dichloro-4-(2-ethoxybenzyl)pyrido[2,3-b]pyrazin-3(4H)-one

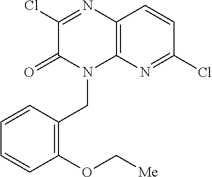 [SMILES: CCOc1ccccc1Cn1c(=O)c(Cl)nc2ccc(Cl)nc21]

The title compound was prepared according to the method described for Preparation 11 using 6-chloro-N²-(2-ethoxybenzyl)pyridine-2,3-diamine (Preparation 10J).

¹H NMR (400 MHz, DMSO-d₆): δ ppm 8.02 (d, 1H), 7.27 (d, 1H), 7.20 (t, 1H), 7.11 (d, 1H), 6.83 (dd, 2H), 5.68 (s, 2H), 4.00 (dd, 2H), 1.28 (t, 3H). MS m/z 350 [M+H]⁺

### Preparation 11K

### 4-benzyl-2,6-dichloropyrido[2,3-b]pyrazin-3(4H)-one

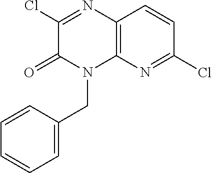 [SMILES: O=c1c(Cl)nc2ccc(Cl)nc2n1Cc1ccccc1]

The title compound was prepared according to the method described for Preparation 11 using N²-benzyl-6-chloropyridine-2,3-diamine (Preparation 10K).

¹H NMR (400 MHz, DMSO-d₆): δ ppm 8.30 (d, 1H), 7.57 (d, 1H), 7.37 (d, 2H), 7.27 (m, 3H), 5.47 (s, 2H). MS m/z 306 [M+H]⁺

### Preparation 11L

### 2,6-dichloro-4-[(1R)-1-phenylpropyl]pyrido[2,3-b]pyrazin-3(4H)-one

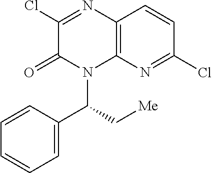 [SMILES: CC[C@H](c1ccccc1)n1c(=O)c(Cl)nc2ccc(Cl)nc21]

The title compound was prepared according to the method described for Preparation 11 using 6-chloro-N²-[(1R)-1-phenylpropyl]pyridine-2,3-diamine (Preparation 10L).

¹H NMR (400 MHz, DMSO-d₆): δ ppm 8.28 (d, 1H), 7.55 (d, 1H), 7.44 (d, 2H), 7.31 (t, 2H), 7.24 (m, 1H), 6.46 (bs, 1H), 2.55 (m, 2H), 0.86 (t, 3H). MS m/z 334 [M+H]⁺

### Preparation 12

### tert-butyl N-{6-chloro-3-oxo-4-[(1S)-1-(pyridin-2-yl)propyl]-3,4-dihydropyrido[2,3-b]pyrazin-2-yl}-beta-alaninate

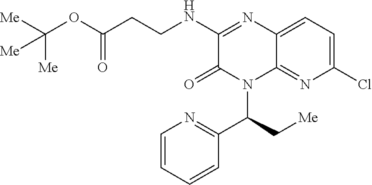 [SMILES: CC[C@@H](c1ccccn1)n1c(=O)c(NCCC(=O)OC(C)(C)C)nc2ccc(Cl)nc21]

The title compound was prepared according to the method described for Preparation 4 using 2,6-dichloro-4-[(1S)-1-(pyridin-2-yl)propyl]pyrido[2,3-b]pyrazin-3(4H)-one (Preparation 11A) and tert-butyl-3-aminopropanoate. MS m/z 444 [M+H]⁺

### Preparation 12A

### tert-butyl N-{6-chloro-4-[(1S)-2-methyl-1-(pyridin-2-yl)propyl]-3-oxo-3,4-dihydropyrido[2,3-b]pyrazin-2-yl}-beta-alaninate

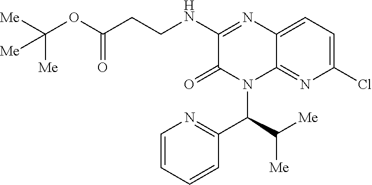 [SMILES: CC(C)[C@@H](c1ccccn1)n1c(=O)c(NCCC(=O)OC(C)(C)C)nc2ccc(Cl)nc21]

The title compound was prepared according to the method described for Preparation 4 using 2,6-dichloro-4-[(1S)-2-methyl-1-(pyridin-2-yl)propyl]pyrido[2,3-b]pyrazin-3(4H)-one (Preparation 11B) and tert-butyl-3-aminopropanoate.

¹H NMR (400 MHz, DMSO-d₆): δ ppm 0.77 (d, 3H), 1.24 (d, 3H), 1.39 (s, 9H), 1.54-1.57 (m, 1H), 1.75-1.80 (m, 2H), 2.54-2.57 (m, 2H), 6.22-6.24 (m, 1H), 7.15-7.21 (m, 2H), 7.55-7.57 (m, 2H), 7.67-7.73 (m, 2H), 8.39-8.40 (m, 1H). MS m/z 458 [M+H]⁺

### Preparation 12B

### N²-{6-chloro-3-oxo-4-[(1S)-1-phenylpropyl]-3,4-dihydropyrido[2,3-b]pyrazin-2-yl}-N-methylglycinamide

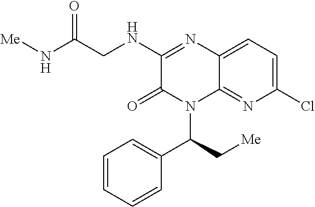 [SMILES: CC[C@@H](c1ccccc1)n1c(=O)c(NCC(=O)NC)nc2ccc(Cl)nc21]

The title compound was prepared according to the method described for Preparation 4 using 2,6-dichloro-4-[(1S)-1-phenylpropyl]pyrido[2,3-b]pyrazin-3(4H)-one (Preparation 6) and 3-amino-N-methylacetamide.

¹H NMR (400 MHz, DMSO-d₆): δ ppm 0.88-0.93 (t, 3H), 2.56-2.75 (m, 5H), 4.03-4.09 (m, 2H), 6.49-6.53 (m, 1H), 7.21-7.32 (m, 4H), 7.46-7.48 (m, 2H), 7.55-7.64 (m, 2H), 7.75-7.77 (m, 1H).

MS m/z 384 [M−H]⁻.

### Preparation 12C

### N³-{6-chloro-3-oxo-4-[(1S)-1-phenylpropyl]-3,4-dihydropyrido[2,3-b]pyrazin-2-yl}-N-methyl-beta-alaninamide

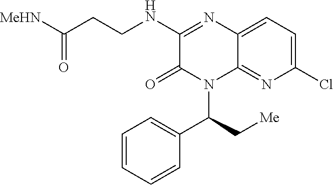 [SMILES: CC[C@@H](c1ccccc1)n1c(=O)c(NCCC(=O)NC)nc2ccc(Cl)nc21]

The title compound may be prepared according to the method described for Preparation 4 using 2,6-dichloro-4-[(1S)-1-phenylpropyl]pyrido[2,3-b]pyrazin-3(4H)-one (Preparation 6) and 3-amino-N-ethylacetamide and taken directly on to the next step.

### Preparation 12D

### N²-{6-chloro-4-[(1S)-2-methyl-1-(pyridin-2-yl)propyl]-3-oxo-3,4-dihydropyrido[2,3-b]pyrazin-2-yl}-N-methylglycinamide

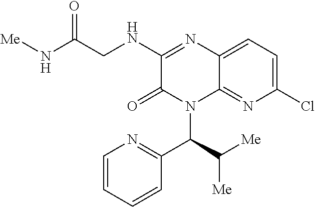 [SMILES: CNC(=O)CNc1nc2ccc(Cl)nc2n([C@H](c2ccccn2)C(C)C)c1=O]

The title compound was prepared according to the method described for Preparation 4 using (S)-2,6-dichloro-4-(2-methyl-1-(pyridin-2-yl)propyl)pyrido[2,3-b]pyrazin-3(4H)-one (Preparation 11B) and 3-amino-N-methylacetamide. MS m/z 401 [M+H]⁺

### Preparation 12E

### tert-butyl N-{6-chloro-3-oxo-4-[(3S,4S)-4-phenyltetrahydrofuran-3-yl]-3,4-dihydropyrido[2,3-b]pyrazin-2-yl}-beta-alaninate

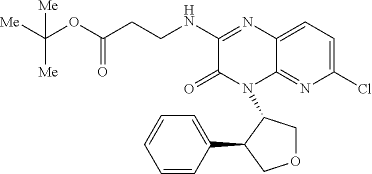 [SMILES: CC(C)(C)OC(=O)CCNc1nc2ccc(Cl)nc2n([C@@H]2COC[C@H]2c2ccccc2)c1=O]

The title compound was prepared according to the method described for Preparation 4 using 2,6-dichloro-4-[(3S,4S)-4-phenyltetrahydrofuran-3-yl]pyrido[2,3-b]pyrazin-3(4H)-one (Preparation 11C) and tert-butyl-3-aminopropanoate. MS m/z 471 [M+H]⁺

### Preparation 12F

### tert-butyl N-[6-chloro-4-(1-cyclopentylcyclopropyl)-3-oxo-3,4-dihydropyrido[2,3-b]pyrazin-2-yl]-beta-alaninate

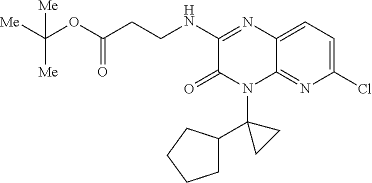 [SMILES: CC(C)(C)OC(=O)CCNc1nc2ccc(Cl)nc2n(C2(C3CCCC3)CC2)c1=O]

The title compound was prepared according to the method described for Preparation 4 using 2,6-dichloro-4-(1-cyclopentylcyclopropyl)pyrido[2,3-b]pyrazin-3(4H)-one (Preparation 11D) and tert-butyl-3-aminopropanoate. ¹H NMR (400 MHz, DMSO-d₆): δ ppm 0.96-1.00 (m, 2H), 1.09-1.43 (m, 6H), 1.50-1.54 (m, 4H), 2.41-2.67 (m, 3H), 3.53-3.63 (m, 2H), 7.62 (d, 1H), 7.82 (d, 1H), 7.84 (t, 1H). MS m/z 433 [M+H]⁺

### Preparation 12G

### tert-butyl N-[6-chloro-3-oxo-4-(1-phenylcyclobutyl)-3,4-dihydropyrido[2,3-b]pyrazin-2-yl]-beta-alaninate

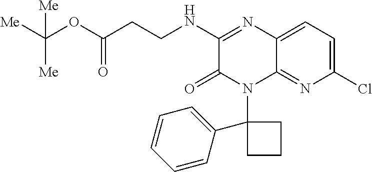 [SMILES: CC(C)(C)OC(=O)CCNc1nc2ccc(Cl)nc2n(C2(c3ccccc3)CCC2)c1=O]

The title compound was prepared according to the method described for Preparation 4 using 2,6-dichloro-4-(1-phenylcyclobutyl)pyrido[2,3-b]pyrazin-3(4H)-one (Preparation 11E) and tert-butyl-3-aminopropanoate. ¹H NMR (400 MHz, DMSO-d₆): δ ppm 1.36 (s, 9H), 1.63-1.77 (m, 2H), 2.55-2.59 (m, 2H), 2.66-3.20 (br m, 4H), 3.56-3.60 (m, 2H), 7.22-7.36 (m, 4H), 7.73 (d, 1H), 7.81-7.83 (m, 2H), 7.94 (t, 1H). MS m/z 455 [M+H]⁺

### Preparation 12H

### tert-butyl N-[6-chloro-4-(2,5-diethylcyclopentyl)-3-oxo-3,4-dihydropyrido[2,3-b]pyrazin-2-yl]-beta-alaninate

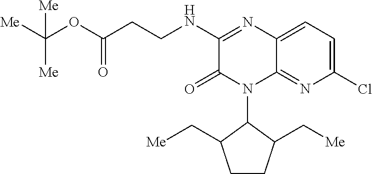 [SMILES: CCC1CCC(CC)C1n1c(=O)c(NCCC(=O)OC(C)(C)C)nc2ccc(Cl)nc21]

The title compound was prepared according to the method described for Preparation 4 using 2,6-dichloro-4-(2,5-diethylcyclopentyl)pyrido[2,3-b]pyrazin-3(4H)-one (Preparation 11F) and tert-butyl-3-aminopropanoate. MS m/z 449 [M+H]⁺

### Preparation 12I

### tert-butyl N-{6-chloro-4-[(2R)-1-methoxybutan-2-yl]-3-oxo-3,4-dihydropyrido[2,3-b]pyrazin-2-yl}-beta-alaninate

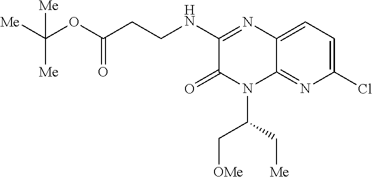 [SMILES: CC[C@H](COC)n1c(=O)c(NCCC(=O)OC(C)(C)C)nc2ccc(Cl)nc21]

The title compound was prepared according to the method described for Preparation 4 using 2,6-dichloro-4-[(1R)-2-methoxy-1-phenylethyl]pyrido[2,3-b]pyrazin-3(4H)-one (Preparation 11G) and tert-butyl-3-aminopropanoate. MS m/z 411 [M+H]⁺

### Preparation 12J

### tert-butyl N-{6-chloro-4-[(2R)-1-methoxypentan-2-yl]-3-oxo-3,4-dihydropyrido[2,3-b]pyrazin-2-yl}-beta-alaninate

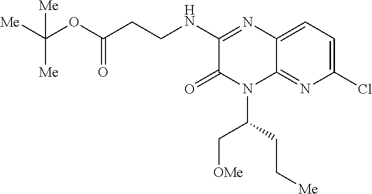 [SMILES: CCC[C@H](COC)n1c(=O)c(NCCC(=O)OC(C)(C)C)nc2ccc(Cl)nc21]

The title compound was prepared according to the method described for Preparation 4 using 2,6-dichloro-4-[(1R)-2-methoxy-1-phenylethyl]pyrido[2,3-b]pyrazin-3(4H)-one (Preparation 11H) and tert-butyl-3-aminopropanoate. MS m/z 425 [M+H]⁺

### Preparation 12K

### N²-{6-chloro-4-[(2R)-1-methoxybutan-2-yl]-3-oxo-3,4-dihydropyrido[2,3-b]pyrazin-2-yl}-N-methylglycinamide

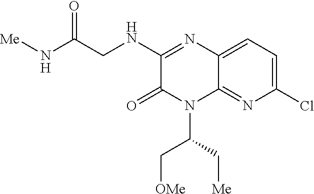 [SMILES: CC[C@H](COC)n1c(=O)c(NCC(=O)NC)nc2ccc(Cl)nc21]

The title compound was prepared according to the method described for Preparation 4 using 2,6-dichloro-4-[(1R)-2-methoxy-1-phenylethyl]pyrido[2,3-b]pyrazin-3(4H)-one (Preparation 11G) and 3-amino-N-methylacetamide. MS m/z 354 [M+H]⁺

### Preparation 12L

### N²-[6-chloro-4-(1,3-dimethoxypropan-2-yl)-3-oxo-3,4-dihydropyrido[2,3-b]pyrazin-2-yl]-N-methylglycinamide

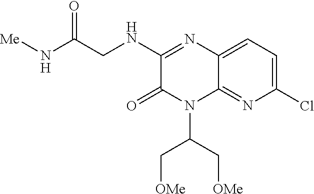 [SMILES: CNC(=O)CNc1nc2ccc(Cl)nc2n(C(COC)COC)c1=O]

The title compound was prepared according to the method described for Preparation 4 using 2,6-dichloro-4-[(1R)-2-methoxy-1-phenylethyl]pyrido[2,3-b]pyrazin-3(4H)-one (Preparation 11I) and 3-amino-N-methylacetamide. MS m/z 370 [M+H]⁺

### Preparation 12M

### 6-chloro-4-(1,3-dimethoxypropan-2-yl)-2-(methylamino)pyrido[2,3-b]pyrazin-3(4H)-one

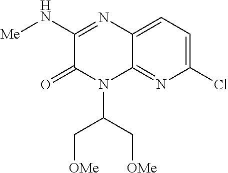 [SMILES: CNc1nc2ccc(Cl)nc2n(C(COC)COC)c1=O]

The title compound was prepared according to the method described for Preparation 4 using 2,6-dichloro-4-[(1R)-2-methoxy-1-phenylethyl]pyrido[2,3-b]pyrazin-3(4H)-one (Preparation 11I) and methylamine. MS m/z 313 [M+H]⁺

### Preparation 12N

### N²-{6-chloro-4-[(1S)-1-(2-methoxyphenyl)ethyl]-3-oxo-3,4-dihydropyrido[2,3-b]pyrazin-2-yl}-N-methylglycinamide

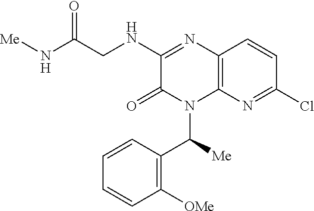 [SMILES: CNC(=O)CNc1nc2ccc(Cl)nc2n([C@@H](C)c2ccccc2OC)c1=O]

The title compound was prepared according to the method described for Preparation 4 using 2,6-dichloro-4-[(1S)-1-(2-methoxyphenyl)ethyl]pyrido[2,3-b]pyrazin-3(4H)-one (Preparation 3) and 3-amino-N-methylacetamide. MS m/z 403 [M−H]⁻

### Preparation 12O

### N-{6-chloro-3-oxo-4-[(1S)-1-phenylpropyl]-3,4-dihydropyrido[2,3-b]pyrazin-2-yl}acetamide

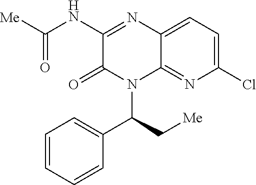 [SMILES: CC[C@@H](c1ccccc1)n1c(=O)c(NC(C)=O)nc2ccc(Cl)nc21]

To a stirring solution of 2,6-dichloro-4-[(1S)-1-phenylpropyl]pyrido[2,3-b]pyrazin-3(4H)-one (Preparation 6, 250 mg, 0.716 mmol), in DCM (10 mL) was added ammonia in THF (10 mL) at 0° C. The reaction was stirred at room temperature for 6 hours. The reaction was concentrated in vacuo and purified by silica gel column chromatography (13% EtOAc in hexane). The residue was dissolved in DCM (15 mL) and treated with DIPEA (0.112 mL, 0.637 mmol) and acetyl chloride (0.023 mL, 0.35 mmol) at 0° C. and stirred at room temperature for 3 hours. The reaction was concentrated in vacuo and purified using silica gel column chromatography eluting with 13% EtOAc in hexanes to afford the title compound as an off-white solid (80 mg, 70%). ¹H NMR (400 MHz, DMSO-d₆): δ ppm 0.80-0.86 (m, 3H), 2.29 (s, 3H), 2.46-2.66 (m, 2H), 6.40-6.55 (br m, 1H), 7.20-7.47 (m, 5H), 7.70-7.72 (m, 1H), 8.06-8.08 (m, 1H), 10.05 (br s, 1H). MS m/z 357 [M+H]⁺

### Example 1

### N-{6-[acetyl(methyl)amino]-4-[(1S)-1-(2-methoxyphenyl)ethyl]-3-oxo-3,4-dihydropyrido[2,3-b]pyrazin-2-yl}-beta-alanine

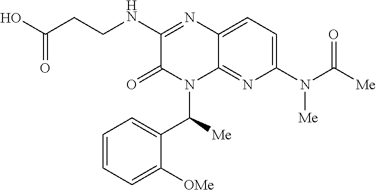 [SMILES: COc1ccccc1[C@H](C)n1c(=O)c(NCCC(=O)O)nc2ccc(N(C)C(C)=O)nc21]

### Step 1

A mixture of tert-butyl N-{6-chloro-4-[(1S)-1-(2-methoxyphenyl)ethyl]-3-oxo-3,4-dihydropyrido[2,3-b]pyrazin-2-yl}-beta-alaninate (Preparation 4, 50 mg, 0.11 mmol), N-methyl acetamide (9.5 mg, 0.13 mmol) and K₃PO₄ (69.4 mg, 0.33 mmol) in dioxane (3 mL) was degassed with argon in a sealed tube for 10 minutes. S-Phos (3.6 mg, 0.009 mmol) and Pd(OAc)₂ (1.2 mg, 0.005 mmol) were added and again degassed for 5 minutes. The reaction was heated at 130° C. for 16 hours before cooling and extracting into EtOAc. The organic layer was washed with water, brine, dried over sodium sulfate and concentrated in vacuo. The residue was purified by silica gel column chromatography eluting with 0-30% EtOAc in 100% hexanes.

### Step 2

The residue was dissolved in DCM (2 mL) and TFA (0.66 mL, 8.48 mmol) was added and resulting mixture was stirred at room temperature for 4 hours. The reaction mixture was concentrated in vacuo and azeotroped with DCM. The residue was purified using preparative TLC eluting with 3% MeOH in DCM to afford the title compound as a yellow solid (10 mg, 19%). ¹H NMR (400 MHz, MeOH-d₄): δ ppm 1.70-1.80 (br m, 2H), 1.90-1.95 (m, 3H), 2.66-2.70 (m, 3H), 3.18 (s, 3H), 3.48 (s, 3H), 3.74-3.77 (m, 2H), 6.74-6.80 (m, 2H), 6.92-6.95 (m, 1H), 7.13-7.19 (m, 2H), 7.60-7.61 (m, 1H), 7.80-7.82 (m, 1H). MS m/z 440 [M+H]⁺

### Example 2

### N-{6-[acetyl(ethyl)amino]-4-[(1S)-1-(2-methoxyphenyl)ethyl]-3-oxo-3,4-dihydropyrido[2,3-b]pyrazin-2-yl}-beta-alanine

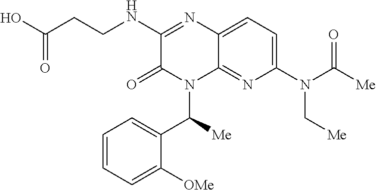 [SMILES: CCN(C(C)=O)c1ccc2nc(NCCC(=O)O)c(=O)n([C@@H](C)c3ccccc3OC)c2n1]

The title compound was prepared according to the method described for Example 1 using tert-butyl N-{6-chloro-4-[(1S)-1-(2-methoxyphenyl)ethyl]-3-oxo-3,4-dihydropyrido[2,3-b]pyrazin-2-yl}-beta-alaninate (Preparation 4) and N-ethylacetamide.

¹H NMR (400 MHz, MeOH-d₄): δ ppm 0.89-0.93 (m, 3H), 1.28-1.30 (br m, 3H), 1.65-1.75 (br m, 2H), 1.95-1.97 (d, 3H), 2.68-2.71 (m, 2H), 3.51 (s, 3H), 3.76-3.83 (m, 2H), 6.75-6.82 (m, 2H), 6.94-6.98 (m, 1H), 7.11-7.12 (m, 1H), 7.19-7.23 (m, 1H), 7.61-7.63 (m, 1H), 7.83-7.85 (m, 1H).

MS m/z 454 [M+H]⁺

### Example 3

### N-{6-[acetyl(methyl)amino]-3-oxo-4-[(1S)-1-phenylpropyl]-3,4-dihydropyrido[2,3-b]pyrazin-2-yl}-beta-alanine

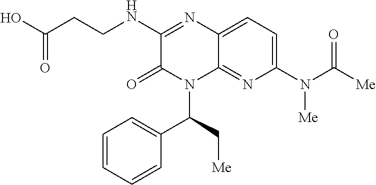 [SMILES: CC[C@@H](c1ccccc1)n1c(=O)c(NCCC(=O)O)nc2ccc(N(C)C(C)=O)nc21]

The title compound was prepared according to the method described for Preparation 4 and Example 1 using 2,6-dichloro-4-[(1S)-1-phenylpropyl]pyrido[2,3-b]pyrazin-3(4H)-one (Preparation 6), tert-butyl 3-aminopropanoate and N-methylacetamide.

¹H NMR (400 MHz, MeOH-d₄): δ ppm 0.88-0.92 (m, 3H), 1.79-1.80 (br m, 3H), 2.51-2.80 (m, 5H), 3.12-3.18 (br m, 3H), 3.76-3.79 (t, 2H), 6.59-6.61 (m, 1H), 7.15-7.37 (m, 6H), 7.84-7.86 (m, 1H). MS m/z 424 [M+H]⁺

### Example 4

### N-{6-[(hydroxyacetyl)(methyl)amino]-3-oxo-4-[(1S)-1-phenylpropyl]-3,4-dihydropyrido[2,3-b]pyrazin-2-yl}-beta-alanine

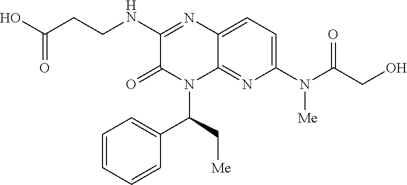 [SMILES: CC[C@@H](c1ccccc1)n1c(=O)c(NCCC(=O)O)nc2ccc(N(C)C(=O)CO)nc21]

To a mixture of tert-butyl N-{6-chloro-3-oxo-4-[(1S)-1-phenylpropyl]-3,4-dihydropyrido[2,3-b]pyrazin-2-yl}-beta-alaninate (Preparation 7A, 0.13 g, 0.32 mmol) in dioxane (1.5 mL) was added cesium carbonate (211 mg, 0.65 mmol), N-methyl-2-((tetrahydro-2H-pyran-2-yl)oxy)acetamide (70 mg, 0.40 mmol) and 4A molecular sieves (200 mg). The mixture was degassed with nitrogen for 1 minute before the addition of allyl palladium (II) chloride dimer (2.7 mg, 0.007 mmol) and Jackiephos (15 mg, 0.019 mmol) and heating in a sealed tube for 130° C. overnight. The reaction was cooled and purified directly using silica gel column chromatography eluting with (0-100% EtOAc in heptane). The residue was dissolved in DCM (2 mL) and treated with TFA (1 mL). The reaction was stirred at room temperature until reaction completion then concentrated in vacuo. The residue was dissolved in MeOH (1 mL) and treated with 1N NaOH (1 mL) and stirred at room temperature for 2 hours. The solution was then treated with 1N HCl (3 mL) and extracted three times with EtOAc (3×15 mL). The organic layer was collected, dried over sodium sulfate, concentrated in vacuo and purified using preparative HPLC (Column: Waters Sunfire C18 19×100, 5u; Mobile phase A: 0.05% TFA in water (v/v); Mobile phase B: 0.05% TFA in acetonitrile (v/v); Gradient: 85.0% H20/15.0% Acetonitrile linear to 45% H2O/55% Acetonitrile in 8.5 min to 0% H2O/100% MeCN to 9.0 min, HOLD at 0% H₂O/100% Acetonitrile from 9.0 to 10.0 min. Flow:25 mL/min) to afford the title compound (28 mg, 20% over three steps). Column: Waters Atlantis dC18 4.6×50, 5u; Mobile phase A: 0.05% TFA in water (v/v); Mobile phase B: 0.05% TFA in acetonitrile (v/v); Gradient: 95.0% H₂O/5.0% Acetonitrile linear to 5% H₂O/95% Acetonitrile in 4.0 min, HOLD at 5% H₂O/95% Acetonitrile to 5.0 min. Flow rate: 2 mL/min. Retention time: 2.39 min. MS m/z 440.2 [M+H]⁺

### Example 5

### N-{4-[(1S)-1-(2-methoxyphenyl)ethyl]-2-(methylamino)-3-oxo-3,4-dihydropyrido[2,3-b]pyrazin-6-yl}-N-methylacetamide

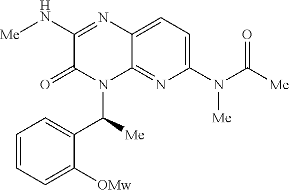

The title compound was prepared according to the method described for Example 1 Step 1 using (S)-6-chloro-4-(1-(2-methoxyphenyl)ethyl)-2-(methylamino)pyrido[2,3-b]pyrazin-3(4H)-one (Preparation 8) and N-methylacetamide with xantphos as ligand.

¹H NMR (400 MHz, DMSO-de): δ ppm 1.85-1.91 (m, 5H), 2.86-2.87 (m, 3H), 3.18 (s, 3H), 3.43 (s, 3H), 6.74-6.95 (m, 3H), 7.18-7.27 (m, 2H), 7.54-7.56 (m, 1H), 7.77-7.82 (m, 2H).

MS m/z 382 [M+H]⁺

### Example 6

### N-{6-[methyl(2-methylpropanoyl)amino]-3-oxo-4-[(1S)-1-phenylpropyl]-3,4-dihydropyrido[2,3-b]pyrazin-2-yl}-beta-alanine

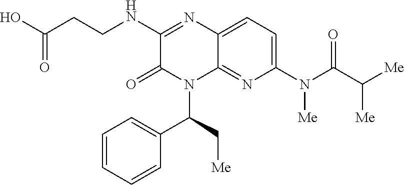 [SMILES: CC[C@@H](c1ccccc1)n1c(=O)c(NCCC(=O)O)nc2ccc(N(C)C(=O)C(C)C)nc21]

The title compound was prepared according to the method described for Preparation 4 and Example 1 using 2,6-dichloro-4-[(1S)-1-phenylpropyl]pyrido[2,3-b]pyrazin-3(4H)-one (Preparation 6), tert-butyl 3-aminopropanoate and N-methylisobutyramide.

¹H NMR (400 MHz, DMSO-d₆): δ ppm 0.79-0.96 (m, 9H), 2.61-2.67 (m, 5H), 3.13-3.19 (s, 3H), 3.56-3.60 (m, 2H), 6.52-6.56 (m, 1H), 7.17-7.34 (m, 5H), 7.87 (m, 2H), 12.28 (br s, 1H).

MS m/z 452 [M+H]⁺

### Example 7

### N-{6-[butanoyl(methyl)amino]-3-oxo-4-[(1S)-1-phenylpropyl]-3,4-dihydropyrido[2,3-b]pyrazin-2-yl}-beta-alanine

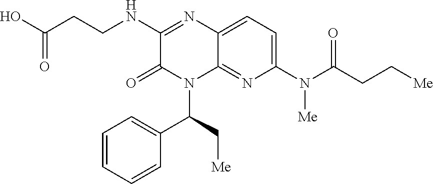 [SMILES: CCCC(=O)N(C)c1ccc2nc(NCCC(=O)O)c(=O)n([C@@H](CC)c3ccccc3)c2n1]

The title compound was prepared according to the method described for Preparation 4 and Example 1 using 2,6-dichloro-4-[(1S)-1-phenylpropyl]pyrido[2,3-b]pyrazin-3(4H)-one (Preparation 6), tert-butyl 3-aminopropanoate and N-methylbutyramide.

¹H NMR (400 MHz, DMSO-d₆): δ ppm 0.72-0.83 (m, 6H), 1.33-1.40 (br m, 2H), 2.07-2.32 (m, 4H), 2.60-2.66 (m, 3H), 3.15-3.17 (m, 2H), 3.59-3.61 (m, 2H), 6.54-6.56 (m, 1H), 7.17-7.34 (m, 5H), 7.83-7.85 (m, 2H), 12.28 (br s, 1H). MS m/z 452 [M+H]⁺

### Example 8

### N-{6-[(cyclobutylcarbonyl)(methyl)amino]-3-oxo-4-[((1S)-1-phenylpropyl]-3,4-dihydropyrido[2,3-b]pyrazin-2-yl}-beta-alanine

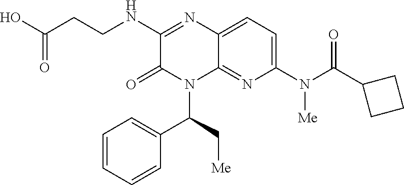 [SMILES: CC[C@@H](c1ccccc1)n1c(=O)c(NCCC(=O)O)nc2ccc(N(C)C(=O)C3CCC3)nc21]

The title compound was prepared according to the method described for Preparation 4 and Example 1 using 2,6-dichloro-4-[(1S)-1-phenylpropyl]pyrido[2,3-b]pyrazin-3(4H)-one (Preparation 6), tert-butyl 3-aminopropanoate and N-methylcyclobutylamine.

¹H NMR (400 MHz, DMSO-d₆): δ ppm 0.80-0.84 (t, 3H), 1.50-1.70 (br m, 4H), 1.90-2.10 (br m, 2H), 2.63-2.67 (m, 4H), 2.80-3.30 (br m, 4H), 3.60-3.62 (m, 2H), 6.55-6.57 (m, 1H), 7.20-7.47 (m, 6H), 7.79-7.81 (m, 1H). MS m/z 464 [M+H]⁺

### Example 9

### N-{6-[methyl(methylcarbamoyl)amino]-3-oxo-4-[(1S)-1-phenylpropyl]-3,4-dihydropyrido[2,3-b]pyrazin-2-yl}-beta-alanine

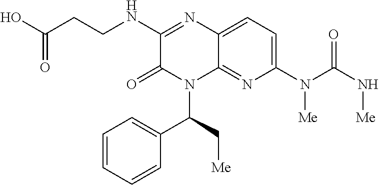 [SMILES: CC[C@@H](c1ccccc1)n1c(=O)c(NCCC(=O)O)nc2ccc(N(C)C(=O)NC)nc21]

To a mixture of tert-butyl (S)-3-((6-chloro-3-oxo-4-(1-phenylpropyl)-3,4-dihydropyrido[2,3-b]pyrazin-2-yl)amino)propanoate (prepared according to Preparation 4 using 2,6-dichloro-4-[(1S)-1-phenylpropyl]pyrido[2,3-b]pyrazin-3(4H)-one (Preparation 6) and tert-butyl 3-aminopropanoate, 200 mg, 0.452 mmol) and NaOtBu (60.747 mg, 0.632 mmol) in dioxane and water (2 mL, 0.5 mL) was added 1,3-dimethyl-urea (39 mg, 0.452 mmol) at room temperature. The reaction was degassed for 5 minutes followed by the addition of Brettphos palladacycle (21 mg, 0.027 mmol) and heating to 100° C. under microwave irradiation for 2 hours. The reaction was concentrated in vacuo and the residue was purified by silica gel column chromatography eluting with 25% EtOAc in hexanes. The residue was treated with TFA (5 mL) for 1 hour before concentrating in vacuo and purified using preparative TLC eluting with 2% MeOH in DCM to afford the title compound as a white solid (65 mg, 65% over two steps). ¹H NMR (400 MHz, DMSO-d₆): δ ppm 0.80-0.82 (m, 3H), 2.49-2.66 (m, 6H), 2.80-3.32 (br m, 3H), 3.56-3.60 (br m, 3H), 6.50-6.60 (br m, 1H), 7.19-7.75 (m, 7H). MS m/z 439 [M+H]⁺

### Example 10

### N-{6-[methyl(propanoyl)amino]-3-oxo-4-[(1S)-1-phenylpropyl]-3,4-dihydropyrido[2,3-b]pyrazin-2-yl}-beta-alanine

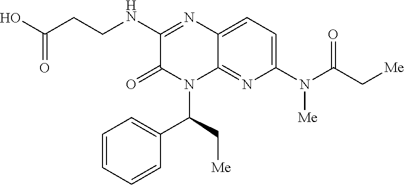 [SMILES: CCC(=O)N(C)c1ccc2nc(NCCC(=O)O)c(=O)n([C@@H](CC)c3ccccc3)c2n1]

To a solution of tert-butyl N-{6-[methyl(propanoyl)amino]-3-oxo-4-[(1S)-1-phenylpropyl]-3,4-dihydropyrido[2,3-b]pyrazin-2-yl}-beta-alaninate (Preparation 29, 53 g, 0.107 mol) in DCM (600 mL) was added TFA (200 mL) at 20° C. The mixture was warmed to 40° C. and stirred for 3 hours. TLC (petroleum ether/ethyl acetate (2:1)) showed most of the starting material was consumed. The mixture was concentrated in vacuo to give crude product, which was purified by column chromatography on silica gel (petroleum ether/ethyl acetate (8:1 to 5:1)) to give 60 g of crude product. The crude product was purified by prep-HPLC (Column: Phenomenex Synergi Max-RP 250×80 10 ul. Mobile phase: from 35% MeCN (0.1% TFA-ACN) in water to 65% MeCN (0.1% TFA-ACN) in water. Gradient Time: 25 min. Flow Rate: 80 mL/min) to give the title compound as a yellow solid (25 g, 53.4%). ¹H NMR (400 MHz, DMSO-d₆): δ ppm 0.84-0.94 (m, 6H), 2.05-2.15 (m, 2H), 2.63-2.66 (m, 4H), 3.10-3.40 (m, 3H), 3.66-3.67 (m, 2H), 6.55-6.60 (m, 1H), 7.26-7.37 (m, 6H), 7.65 (m, 1H), 7.82-7.84 (m, 1H). MS m/z 438 [M+H]⁺

### Example 11

### N-{6-[methyl(propanoyl)amino]-3-oxo-4-[(1S)-1-(pyridin-2-yl)propyl]-3,4-dihydropyrido[2,3-b]pyrazin-2-yl}-beta-alanine

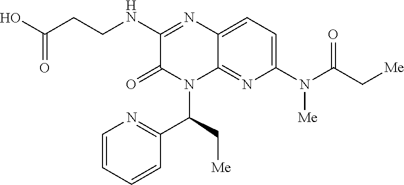 [SMILES: CCC(=O)N(C)c1ccc2nc(NCCC(=O)O)c(=O)n([C@@H](CC)c3ccccn3)c2n1]

The title compound was prepared according to the method described for Example 1 using tert-butyl N-{6-chloro-3-oxo-4-[(1S)-1-(pyridin-2-yl)propyl]-3,4-dihydropyrido[2,3-b]pyrazin-2-yl}-beta-alaninate (Preparation 12) and N-methylpropionamide with xantphos and cesium carbonate. ¹H NMR (400 MHz, DMSO-d₆): δ ppm 0.87-0.93 (m, 6H), 2.04-2.10 (m, 2H), 2.58-2.71 (m, 4H), 2.90-3.21 (m, 3H), 3.68-3.73 (m, 2H), 6.51-6.56 (m, 1H), 7.15-7.82 (m, 6H), 8.39-8.40 (m, 1H), 12.00 (br s, 1H). MS m/z 439 [M+H]⁺

### Example 12

### N-{6-[methyl(propanol)amino]-3-oxo-4-(1-phenylcyclobutyl)-3,4-dihydropyrido[2,3-b]pyrazin-2-yl}-beta-alanine

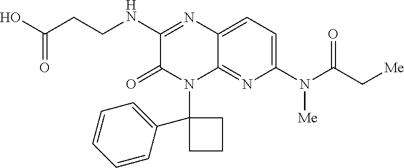 [SMILES: CCC(=O)N(C)c1ccc2nc(NCCC(=O)O)c(=O)n(C3(c4ccccc4)CCC3)c2n1]

The title compound was prepared according to the method described for Example 1 using tert-butyl N-[6-chloro-3-oxo-4-(1-phenylcyclobutyl)-3,4-dihydropyrido[2,3-b]pyrazin-2-yl]-beta-alaninate (Preparation 12G) and N-methylpropionamide with xantphos and cesium carbonate. ¹H NMR (400 MHz, MeOH-d₄): δ ppm 0.87-0.91 (m, 3H), 1.25-1.28 (m, 4H), 1.82-1.89 (m, 4H), 2.68-2.71 (m, 2H), 2.32 (s, 3H), 3.77-3.78 (m, 2H), 7.12-7.30 (m, 4H), 7.75-7.81 (m, 3H). MS m/z 450 [M+H]⁺

### Example 13

### N-{4-(2,5-diethylcyclopentyl)-6-[methyl(propanoyl)amino]-3-oxo-3,4-dihdropyrido[2,3-b]pyrazin-2-yl}-beta-alanine

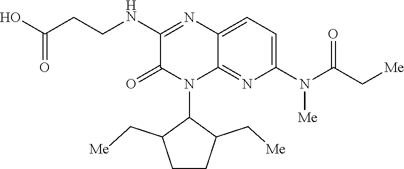 [SMILES: CCC(=O)N(C)c1ccc2nc(NCCC(=O)O)c(=O)n(C3C(CC)CCC3CC)c2n1]

The title compound was prepared according to the method described for Example 1 using tert-butyl N-[6-chloro-4-(2,5-diethylcyclopentyl)-3-oxo-3,4-dihydropyrido[2,3-b]pyrazin-2-yl]-beta-alaninate (Preparation 12H) and N-methylpropionamide with xantphos and cesium carbonate. HPLC Atlantis d-C18 (4.6×50 mm, 3 micron) operating at ambient temperature and flow rate of 1 mL/min. Mobile phase: (0.05% TFA in water) in MeCN. 12 minute run. Mobile phase from 90% [0.05% TFA in water in water] and 10% [MeCN] for 0.01 min, Mobile phase from 90% [0.05% TFA in water] and 10% [MeCN] for 0.5 min, 50% [0.05% TFA in water] and 50% [MeCN] for 5.0 min, then to 10% [0.05% TFA in Water] and 90% [MeCN] for 8.0 min, held in this composition up to 11.0 min, then returned to initial composition in 12.0 min. Rt=6.56 minutes. MS m/z 444 [M+H]⁺

### Example 14

### N-{6-[acetyl(methyl)amino]-4-[(1R)-2-methoxy-1-phenylethyl]-3-oxo-3,4-dihydropyrido[2,3-b]pyrazin-2-yl}-beta-alanine

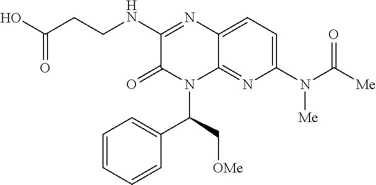 [SMILES: COC[C@@H](c1ccccc1)n1c(=O)c(NCCC(=O)O)nc2ccc(N(C)C(C)=O)nc21]

The title compound was prepared according to the method described for Preparation 4 and Example 1 using 2,6-dichloro-4-[(1R)-2-methoxy-1-phenylethyl]pyrido[2,3-b]pyrazin-3(4H)-one (Preparation 11) and N-methylacetamide with xantphos and cesium carbonate.

¹H NMR (400 MHz, DMSO-d₆): δ ppm: 7.82 (d, 1H), 7.54 (br s, 1H), 7.26-7.39 (m, 5H), 7.23 (d, 1H), 6.78 (t, 1H), 4.33-4.51 (m, 2H), 3.68 (br s, 2H), 3.31 (s, 3H), 3.16 (s, 3H), 2.62 (t, 2H), 1.88 (s, 3H). MS m/z 440 [M+H]⁺

### Example 15

### N-{6-[methyl(propanoyl)amino]-3-oxo-4-[(3S,4S)-4-phenyltetrahydrofuran-3-yl]-3,4-dihydropyrido[2,3-b]pyrazin-2-yl}-beta-alanine

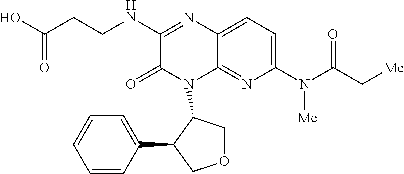 [SMILES: CCC(=O)N(C)c1ccc2nc(NCCC(=O)O)c(=O)n([C@@H]3COC[C@H]3c3ccccc3)c2n1]

The title compound was prepared according to the method described for Example 1 using tert-butyl N-{6-chloro-3-oxo-4-[(3S,4S)-4-phenyltetrahydrofuran-3-yl]-3,4-dihydropyrido[2,3-b]pyrazin-2-yl}-beta-alaninate (Preparation 12E) and N-methylpropionamide with xantphos and cesium carbonate. ¹H NMR (400 MHz, DMSO-d₆): δ ppm: 12.30 (br s, 1H), 7.77 (br s, 1H), 7.64 (d, 1H), 7.21 (d, 1H), 6.85-7.05 (m, 5H), 6.48 (brs, 1H), 4.68 (br s, 1H), 4.47-4.59 (m, 1H), 4.15-4.35 (m, 2H), 3.96 (q, 1H), 3.50 (d, 2H), 3.29 (s, 5H), 2.29 (dt, 1H), 2.17 (br s, 1H), 1.01 (t, 3H). MS m/z 466 [M+H]⁺

### Example 16

### N-{6-[acetyl(methyl)amino]-4-[(2R)-1-methoxybutan-2-yl]-3-oxo-3,4-dihydropyrido[2,3-b]pyrazin-2-yl}-beta-alanine

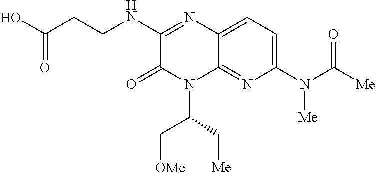 [SMILES: CC[C@H](COC)n1c(=O)c(NCCC(=O)O)nc2ccc(N(C)C(C)=O)nc21]

The title compound was prepared according to the method described for Example 1 using tert-butyl N-{6-chloro-4-[(2R)-1-methoxybutan-2-yl]-3-oxo-3,4-dihydropyrido[2,3-b]pyrazin-2-yl}-beta-alaninate (Preparation 12I) and N-methylacetamide with xantphos and cesium carbonate. ¹H NMR (400 MHz, DMSO-d₆): δ ppm 0.81 (t, 3H), 1.80-1.90 (m, 1H), 2.00 (s, 3H), 2.10-2.20 (m, 1H), 2.61 (d, 2H), 3.21 (s, 3H), 3.30 (s, 3H), 3.65 (br m, 2H), 3.75-3.80 (m, 1H), 4.11-4.16 (m, 1H), 5.60-5.62 (m, 1H), 7.30 (d, 1H), 7.50 (br s, 1H), 7.81 (d, 1H). MS m/z 392 [M+H]⁺

### Example 17

### N-{4-[(2R)-1-methoxypentan-2-yl]-6-[methyl(propanoyl)amino]-3-oxo-3,4-dihydropyrido[2,3-b]pyrazin-2-yl}-beta-alanine

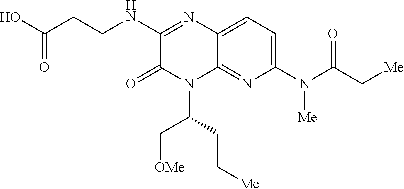 [SMILES: CCC[C@H](COC)n1c(=O)c(NCCC(=O)O)nc2ccc(N(C)C(=O)CC)nc21]

The title compound was prepared according to the method described for Example 1 using tert-butyl N-{6-chloro-4-[(2R)-1-methoxypentan-2-yl]-3-oxo-3,4-dihydropyrido[2,3-b]pyrazin-2-yl}-beta-alaninate (Preparation 12J) and N-methyl propionamide with xantphos and cesium carbonate. ¹H NMR (400 MHz, DMSO-d₆): δ ppm 0.80 (t, 3H), 0.95 (t, 3H), 1.20-1.27 (m, 2H), 1.83-1.84 (m, 1H), 2.17-2.33 (m, 3H), 2.62-2.67 (m, 2H), 3.20 (s, 3H), 3.27 (s, 3H), 3.67-3.77 (m, 3H), 4.10-4.15 (m, 1H), 5.68-5.70 (m, 1H), 7.29 (d, 1H), 7.47 (br s, 1H), 7.80 (d, 1H), 12.30 (br s, 1H). MS m/z 420 [M+H]⁺

### Example 18

### N-(4-[(2R)-1-methoxybutan-2-yl]-2-{[2-(methylamino)-2-oxoethyl]amino}-3-oxo-3,4-dihydropyrido[2,3-b]pyrazin-6-yl)-N-methylpropanamide

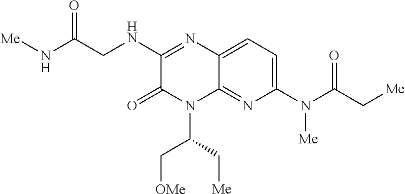 [SMILES: CCC(=O)N(C)c1ccc2nc(NCC(=O)NC)c(=O)n([C@H](CC)COC)c2n1]

The title compound was prepared according to the method described for Example 1 using N²-{6-chloro-4-[(2R)-1-methoxybutan-2-yl]-3-oxo-3,4-dihydropyrido[2,3-b]pyrazin-2-yl}-N-methylglycinamide (Preparation 12K) and N-methyl propionamide.

¹H NMR (400 MHz, DMSO-d₆): δ ppm 0.80 (t, 3H), 1.00 (t, 3H), 1.93-1.96 (m, 1H), 2.15-2.19 (m, 1H), 2.30-2.32 (m, 2H), 2.66 (s, 3H), 3.21 (s, 3H), 3.28 (s, 3H), 3.78-3.81 (m, 1H), 4.04-4.17 (m, 3H), 5.60-5.64 (m, 1H), 7.31 (d, 1H), 7.53-7.60 (br m, 2H), 7.79 (d, 1H).

MS m/z 405 [M+H]⁺

### Example 19

### N-(4-(1,3-dimethoxypropan-2-yl)-2-((2-(methylamino)-2-oxoethyl)amino)-3-oxo-3,4-dihydropyrido[2,3-b]pyrazin-6-yl)-N-methylpropionamide

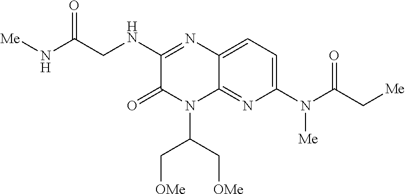 [SMILES: CCC(=O)N(C)c1ccc2nc(NCC(=O)NC)c(=O)n(C(COC)COC)c2n1]

The title compound was prepared according to the method described for Example 1 using 2-((6-chloro-4-(1,3-dimethoxypropan-2-yl)-3-oxo-3,4-dihydropyrido[2,3-b]pyrazin-2-yl)amino)-N-methylacetamide (Preparation 12M) and N-methyl propionamide.

¹H NMR (400 MHz, DMSO-d₆): δ ppm 0.97 (t, 3H), 2.25-2.40 (m, 2H), 2.59-2.60 (m, 3H), 3.20-3.21 (2×s, 6H), 3.26 (s, 3H), 3.70-3.80 (br m, 2H), 3.96-3.98 (m, 2H), 4.00-4.10 (br m, 2H), 6.02 (br s, 1H), 7.35 (d, 1H), 7.80-7.88 (m, 3H). MS m/z 321 [M+H]⁺

### Example 20

### N-(4-(1,3-dimethoxypropan-2-yl)-2-(methylamino)-3-oxo-3,4-dihydropyrido[2,3-b]pyrazin-6-yl)-N-methylpropionamide

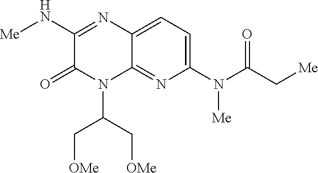 [SMILES: CCC(=O)N(C)c1ccc2nc(NC)c(=O)n(C(COC)COC)c2n1]

The title compound was prepared according to the method described for Example 1 using 6-chloro-4-(1,3-dimethoxypropan-2-yl)-2-(methylamino)pyrido[2,3-b]pyrazin-3(4H)-one (Preparation 12M) and N-methyl propionamide. ¹H NMR (400 MHz, DMSO-d₆): δ ppm 0.98 (t, 3H), 2.29-2.32 (m, 2H), 2.92 (d, 3H), 3.16 (2×s, 6H), 3.31 (s, 3H), 3.72-3.76 (m, 2H), 4.02-4.06 (m, 2H), 6.00-6.05 (br m, 1H), 7.33 (d, 1H), 7.83 (d, 1H), 7.90-7.93 (br m, 1H). MS m/z 364 [M+H]⁺

### Example 21

### N-{2-(acetylamino)-3-oxo-4-[(1S)-1-phenylpropyl]-3,4-dihydropyrido[2,3-b]pyrazin-6-yl}-N-methylacetamide

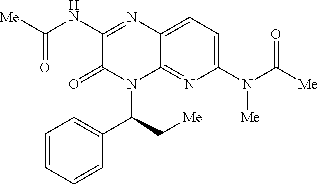 [SMILES: CC[C@@H](c1ccccc1)n1c(=O)c(NC(C)=O)nc2ccc(N(C)C(C)=O)nc21]

The title compound was prepared according to the method described for Preparation 4 and Example 1 using N-{6-chloro-3-oxo-4-[(1S)-1-phenylpropyl]-3,4-dihydropyrido[2,3-b]pyrazin-2-yl}acetamide (Preparation 120) with xantphos and cesium carbonate.

¹H NMR (400 MHz, DMSO-d₆): δ ppm 0.82-0.84 (t, 3H), 2.07 (br s, 3H), 2.31 (br s, 3H), 2.50-2.67 (m, 2H), 3.25 (s, 3H), 6.60-6.70 (m, 1H), 7.22-7.55 (m, 6H), 8.04-8.06 (m, 1H), 9.44 (br s, 1H). MS m/z 394 [M+H]⁺

### Preparation 13

### tert-butyl N-{6-(methylamino)-3-oxo-4-[(1S)-1-phenylpropyl]-3,4-dihydropyrido[2,3-b]pyrazin-2-yl}-beta-alaninate

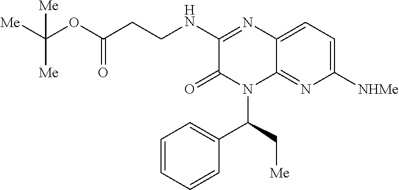 [SMILES: CC[C@@H](c1ccccc1)n1c(=O)c(NCCC(=O)OC(C)(C)C)nc2ccc(NC)nc21]

The title compound was prepared according to the method described for Example 1 Step 1 using tert-butyl N-{6-chloro-3-oxo-4-[(1S)-1-phenylpropyl]-3,4-dihydropyrido[2,3-b]pyrazin-2-yl}-beta-alaninate (Preparation 7A) and methylamine. MS m/z 438 [M+H]⁺

### Preparation 13A

### N-methyl-N²-{6-(methylamino)-3-oxo-4-[(1S)-1-phenylpropyl]-3,4-dihydropyrido[2,3-b]pyrazin-2-yl}glycinamide

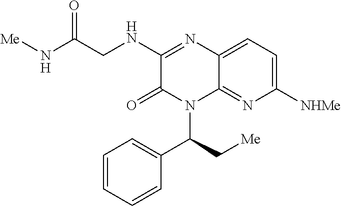 [SMILES: CC[C@@H](c1ccccc1)n1c(=O)c(NCC(=O)NC)nc2ccc(NC)nc21]

The title compound was prepared according to the method described for Example 1 Step 1 using N²-{6-chloro-3-oxo-4-[(1S)-1-phenylpropyl]-3,4-dihydropyrido[2,3-b]pyrazin-2-yl}-N-methylglycinamide (Preparation 12B) and methylamine.

¹H NMR (400 MHz, DMSO-d₆): δ ppm 0.85-0.89 (t, 3H), 2.63-2.76 (m, 8H), 3.79-3.95 (m, 2H), 6.38-6.44 (m, 2H), 6.69 (br s, 1H), 6.91 (br s, 1H), 7.18-7.29 (m, 3H), 7.43-7.45 (m, 3H), 7.62 (br s, 1H). MS m/z 381 [M+H]⁺

### Preparation 13B

### N-methyl-N³-{6-(methylamino)-3-oxo-4-[(1S)-1-phenylpropyl]-3,4-dihydropyrido[2,3-b]pyrazin-2-yl}-beta-alaninamide

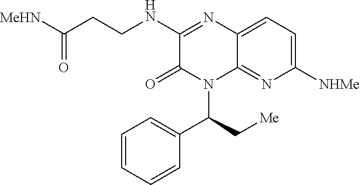 [SMILES: CC[C@@H](c1ccccc1)n1c(=O)c(NCCC(=O)NC)nc2ccc(NC)nc21]

The title compound may be prepared according to the method described for Example 1 Step 1 using N³-{6-chloro-3-oxo-4-[(1S)-1-phenylpropyl]-3,4-dihydropyrido[2,3-b]pyrazin-2-yl}-N-methyl-beta-alaninamide (Preparation 12C) and methylamine and taken directly on to the next step.

### Preparation 13C

### N-methyl-N²-{6-(methylamino)-4-[(1S)-2-methyl-1-(pyridin-2-yl)propyl]-3-oxo-3,4-dihydropyrido[2,3-b]pyrazin-2-yl}glycinamide

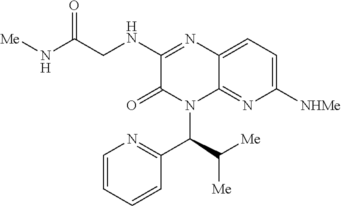 [SMILES: CNC(=O)CNc1nc2ccc(NC)nc2n([C@H](c2ccccn2)C(C)C)c1=O]

The title compound was prepared according to the method described for Example 1 Step 1 using N²-{6-chloro-4-[(1S)-2-methyl-1-(pyridin-2-yl)propyl]-3-oxo-3,4-dihydropyrido[2,3-b]pyrazin-2-yl}-N-methylglycinamide (Preparation 12D) and methylamine.

¹H NMR (400 MHz, DMSO-de): δ ppm 0.78 (d, 3H), 1.22 (d, 3H), 2.65 (d, 3H), 2.77 (d, 3H), 3.53-3.55 (m, 1H), 3.95 (d, 2H), 6.26-6.43 (m, 3H), 6.82 (br s, 1H), 7.15-7.18 (m, 1H), 7.42-7.68 (m, 4H), 8.43 (d, 1H). MS m/z 396 [M+H]⁺

### Preparation 13D

### tert-butyl N-{6-(methylamino)-4-[(1S)-2-methyl-1-(pyridin-2-yl)propyl]-3-oxo-3,4-dihydropyrido[2,3-b]pyrazin-2-yl}-beta-alaninate

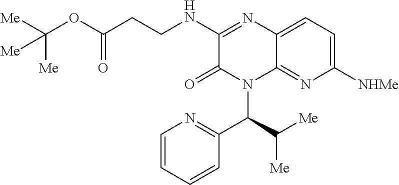 [SMILES: CNc1ccc2nc(NCCC(=O)OC(C)(C)C)c(=O)n([C@H](c3ccccn3)C(C)C)c2n1]

The title compound was prepared according to the method described for Example 1 Step 1 using tert-butyl N-{6-chloro-4-[(1S)-2-methyl-1-(pyridin-2-yl)propyl]-3-oxo-3,4-dihydropyrido[2,3-b]pyrazin-2-yl}-beta-alaninate (Preparation 12A) and methylamine.

¹H NMR (400 MHz, DMSO-d₆): δ ppm 0.76 (d, 3H), 1.20 (d, 3H), 1.36 (s, 9H), 2.55-2.57 (m, 3H), 2.76 (d, 3H), 3.51-3.62 (m, 2H), 6.23-6.25 (m, 1H), 6.35-6.42 (m, 2H), 6.66 (br s, 1H), 7.14-7.17 (m, 1H), 7.43-7.46 (m, 1H), 7.63-7.67 (m, 2H), 7.68-7.70 (m, 1H), 8.42-8.44 (m, 1H). MS m/z 453 [M+H]⁺

### Preparation 13E

### tert-butyl N-[4-(1-cyclopentylcyclopropyl)-6-(methylamino)-3-oxo-3,4-dihydropyrido[2,3-b]pyrazin-2-yl]-beta-alaninate

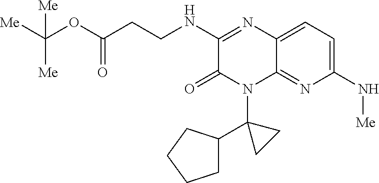 [SMILES: CNc1ccc2nc(NCCC(=O)OC(C)(C)C)c(=O)n(C3(C4CCCC4)CC3)c2n1]

The title compound was prepared according to the method described for Example 1 Step 1 using tert-butyl N-[6-chloro-4-(1-cyclopentylcyclopropyl)-3-oxo-3,4-dihydropyrido[2,3-b]pyrazin-2-yl]-beta-alaninate (Preparation 12F) and methylamine. MS m/z 428 [M+H]⁺

### Preparation 13F

### tert-butyl N-{4-[(1S)-1-(2-methoxyphenyl)ethyl]-3-oxo-6-(propan-2-ylamino)-3,4-dihydropyrido[2,3-b]pyrazin-2-yl}-beta-alaninate

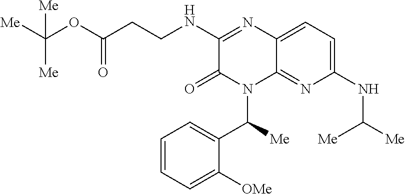 [SMILES: COc1ccccc1[C@H](C)n1c(=O)c(NCCC(=O)OC(C)(C)C)nc2ccc(NC(C)C)nc21]

The title compound was prepared according to the method described for Example 1 Step 1 using tert-butyl N-{6-chloro-4-[(1S)-1-(2-methoxyphenyl)ethyl]-3-oxo-3,4-dihydropyrido[2,3-b]pyrazin-2-yl}-beta-alaninate (Preparation 4) and isopropylamine. MS m/z 482 [M+H]⁺

### Example 22

### N-{6-[(dimethylcarbamoyl)(methyl)amino]-3-oxo-4-[(1S)-1-phenylpropyl]-3,4-dihydropyrido[2,3-b]pyrazin-2-yl}-beta-alanine

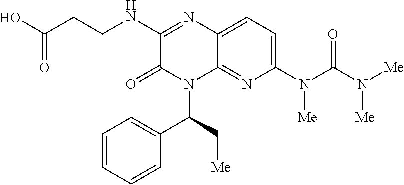 [SMILES: CC[C@@H](c1ccccc1)n1c(=O)c(NCCC(=O)O)nc2ccc(N(C)C(=O)N(C)C)nc21]

To a solution of tert-butyl N-{6-(methylamino)-3-oxo-4-[(1S)-1-phenylpropyl]-3,4-dihydropyrido[2,3-b]pyrazin-2-yl}-beta-alaninate (Preparation 13, 100 mg, 0.229 mmol) in toluene (2 mL) was added N,N-dimethyl carbamoyl chloride (36 mg, 0.343 mmol), triethylamine (0.095 mL, 0.686 mmol) and a catalytic amount of DMAP. The reaction was heated to reflux overnight. Further N,N-dimethyl carbamoyl chloride (1.5 eq) and triethylamine were added and the reaction continued for 4 hours. The reaction was cooled, concentrated in vacuo and partitioned between EtOAc and water. The organic layer was collected, dried over sodium sulfate and concentrated in vacuo. The residue was purified using preparative TLC eluting with 5% MeOH in DCM and then treated with TFA (1 mL) for 1 hour. The reaction was concentrated in vacuo and purified using preparative TLC eluting with 5% MeOH in DCM to afford the title compound as a white solid (12 mg, 65% over two steps). ¹H NMR (400 MHz, DMSO-d₆): δ ppm 0.85-0.89 (m, 3H), 2.55-2.67 (m, 4H), 2.78 (br s, 6H), 3.09 (s, 3H), 3.61-3.66 (m, 2H), 6.55-6.59 (m, 1H), 6.77-6.79 (m, 1H), 7.18-7.40 (m, 5H), 7.71-7.73 (m, 1H). MS m/z 453 [M+H]⁺

### Example 23

### N-{6-[methyl(propanol)amino]-4-[(1S)-2-methyl-1-(pyridin-2-yl)propyl]-3-oxo-3,4-dihydropyrido[2,3-b]pyrazin-2-yl}-beta-alanine

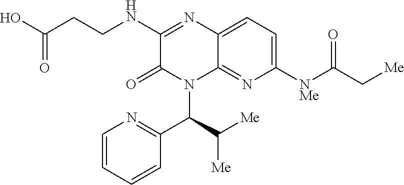 [SMILES: CCC(=O)N(C)c1ccc2nc(NCCC(=O)O)c(=O)n([C@H](c3ccccn3)C(C)C)c2n1]

The title compound was prepared according to the method described for Example 22 above using tert-butyl N-{6-(methylamino)-4-[(1S)-2-methyl-1-(pyridin-2-yl)propyl]-3-oxo-3,4-dihydropyrido[2,3-b]pyrazin-2-yl}-beta-alaninate (Preparation 13D) and propionyl chloride.

¹H NMR (400 MHz, DMSO-d₆): δ ppm 0.73 (d, 3H), 0.97 (t, 3H), 1.20 (d, 3H), 2.11-2.26 (m, 2H), 2.62-2.65 (m, 2H), 3.16 (s, 3H), 3.43-3.49 (m, 1H), 3.65-3.70 (m, 2H), 6.29-6.31 (m, 1H), 7.16-7.19 (m, 1H), 7.26-7.28 (m, 1H), 7.48-7.50 (m, 2H), 7.66-7.80 (m, 2H), 8.42-8.43 (m, 1H). MS m/z 453 [M+H]⁺

### Example 24

### N-{4-(1-cyclopentylcyclopropyl)-6-[methyl(propanoyl)amino]-3-oxo-3,4-dihdropyrido[2, 3-b]pyrazin-2-yl}-beta-alanine

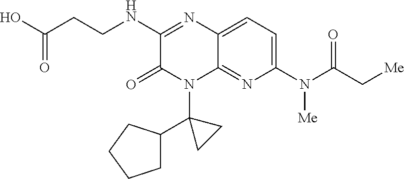 [SMILES: CCC(=O)N(C)c1ccc2nc(NCCC(=O)O)c(=O)n(C3(C4CCCC4)CC3)c2n1]

The title compound was prepared according to the method described for Example 22 above using tert-butyl 3-((4-(1-cyclopentylcyclopropyl)-6-(methylamino)-3-oxo-3,4-dihydropyrido[2,3-b]pyrazin-2-yl)amino)propanoate (Preparation 13E) and propionyl chloride. ¹H NMR (400 MHz, DMSO-d₆): δ ppm 0.98-1.54 (m, 16H), 2.32-2.39 (m, 2H), 2.56-2.66 (m, 2H), 3.31 (s, 3H), 3.56-3.59 (m, 2H), 7.35-7.37 (m, 1H), 7.74-7.76 (m, 1H). MS m/z 428 [M+H]⁺

### Example 25

### N-(6-{[(3,3-dimethylcyclobutyl)carbonyl](methyl)amino}-3-oxo-4-[(1S)-1-phenylpropyl]-3,4-dihydropyrido[2,3-b]pyrazin-2-yl)-beta-alanine

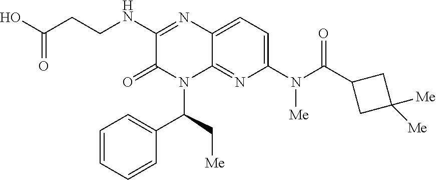 [SMILES: CC[C@@H](c1ccccc1)n1c(=O)c(NCCC(=O)O)nc2ccc(N(C)C(=O)C3CC(C)(C)C3)nc21]

To a solution of tert-butyl N-{6-(methylamino)-3-oxo-4-[(1S)-1-phenylpropyl]-3,4-dihydropyrido[2,3-b]pyrazin-2-yl}-beta-alaninate (Preparation 13, 50 mg, 0.11 mmol) and 3,3-dimethylcyclobutane-1-carbonyl chloride (33 mg, 0.229 mmol) in THF (2 mL) was added LiHMDS (1M in THF, 0.7 mL) slowly at 0° C. The resulting mixture was stirred at the same temperature for 2 hours then gradually warmed to room temperature and stirred for an additional 16 hours. The reaction was diluted with EtOAc, washed with water, brine, dried over sodium sulfate, and concentrated in vacuo. The residue was purified by silica gel column chromatography eluting with 20% EtOAc in hexanes and then dissolved in DCM (2 mL). The solution was treated with TFA (0.6 mL, 7.67 mmol) at 0° C. and the reaction was stirred at room temperature for 4 hours. The reaction was concentrated in vacuo and purified using preparative TLC eluting with 2% MeOH in DCM to afford the title compound as a yellow solid (9 mg, 9% over two steps).

¹H NMR (400 MHz, DMSO-d₆): δ ppm 0.86-0.89 (t, 3H), 0.99 (s, 6H), 1.26-1.30 (m, 2H), 1.55-1.60 (m, 2H), 1.80-1.90 (m, 2H), 2.62-2.67 (m, 3H), 3.14-3.17 (m, 3H), 3.66-3.71 (m, 2H), 6.53-6.57 (m, 1H), 7.20-7.49 (m, 6H), 7.79-7.81 (m, 2H). MS m/z 492 [M+H]⁺

### Example 26

### N-(6-{[(3,3-difluorocyclobutyl)carbonyl](methyl)amino}-3-oxo-4-[(1S)-1-phenylpropyl]-3,4-dihydropyrido[2,3-b]pyrazin-2-yl)-beta-alanine

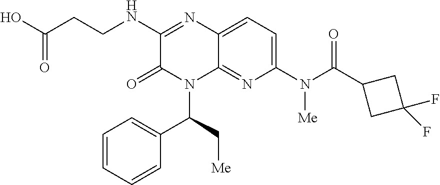 [SMILES: CC[C@@H](c1ccccc1)n1c(=O)c(NCCC(=O)O)nc2ccc(N(C)C(=O)C3CC(F)(F)C3)nc21]

The title compound was prepared according to the method described for Example 25 using 3,3-difluorocyclobutane-1-carbonyl chloride.

¹H NMR (400 MHz, DMSO-d₆): δ ppm 0.86-0.89 (t, 3H), 2.20-2.56 (m, 3H), 2.61-2.69 (m, 5H), 3.18-3.21 (m, 4H), 3.64-3.71 (m, 2H), 6.51-6.54 (m, 1H), 7.18-7.29 (m, 6H), 7.60-7.70 (m, 1H), 7.82-7.84 (m, 1H). MS m/z 498 [M−H].

### Example 27

### N-{6-[methyl(oxetan-3-ylcarbonyl)amino]-3-oxo-4-[(1S)-1-phenylpropyl]-3,4-dihydropyrido[2,3-b]pyrazin-2-yl}-beta-alanine

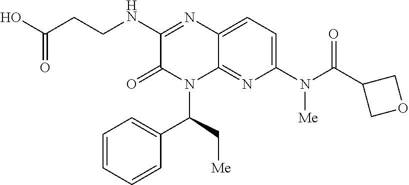 [SMILES: CC[C@@H](c1ccccc1)n1c(=O)c(NCCC(=O)O)nc2ccc(N(C)C(=O)C3COC3)nc21]

The title compound may be prepared according to the method described for Example 25 using oxetane-3-carbonyl chloride.

¹H NMR (400 MHz, DMSO-d₆): δ ppm 0.88 (t, 3H), 2.53-2.70 (m, 4H), 3.20 (s, 3H), 3.58-3.60 (m, 2H), 3.67-3.70 (m, 1H), 4.24-4.33 (m, 2H), 4.50-4.53 (m, 2H), 6.49-6.53 (m, 1H), 7.21-7.37 (m, 6H), 7.66 (br s, 1H), 7.81-7.83 (m, 1H). MS m/z 466 [M+H]⁺

### Example 28

### N-methyl-N-(2-{[3-(methylamino)-3-oxopropyl]amino}-3-oxo-4-[(1S)-1-phenylpropyl]-3,4-dihydropyrido[2,3-b]pyrazin-6-yl)oxetane-2-carboxamide

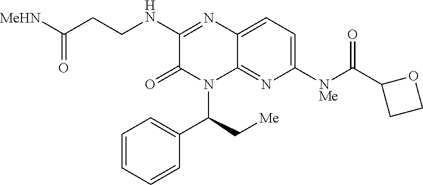 [SMILES: CC[C@@H](c1ccccc1)n1c(=O)c(NCCC(=O)NC)nc2ccc(N(C)C(=O)C3CCO3)nc21]

The title compound may be prepared according to the method described for Example 25 using N-methyl-N³-{6-(methylamino)-3-oxo-4-[(1S)-1-phenylpropyl]-3,4-dihydropyrido[2,3-b]pyrazin-2-yl}-beta-alaninamide (Preparation 13B) and oxetane-2-carbonyl chloride.

¹H NMR (400 MHz, DMSO-d₆): δ ppm: 7.82 (d, 3H), 7.25-7.40 (m, 5H), 7.22 (d, 1H), 6.55 (br s, 1H), 5.26 (br s, 1H), 4.22-4.41 (m, 2H), 3.52-3.69 (m, 2H), 3.30 (s, 3H), 2.61-2.72 (m, 2H), 2.57 (d, 3H), 2.39-2.47 (m, 4H), 0.82 (t, 3H) MS m/z 479 [M+H]⁺

### Example 29

### N-methyl-N-(2-{[2-(methylamino)-2-oxoethyl]amino}-3-oxo-4-[(1S)-1-phenylpropyl]-3,4-dihydropyrido[2,3-b]pyrazin-6-yl)propanamide

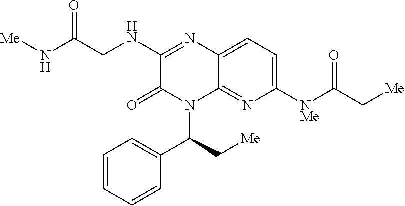 [SMILES: CCC(=O)N(C)c1ccc2nc(NCC(=O)NC)c(=O)n([C@@H](CC)c3ccccc3)c2n1]

To a solution of N-methyl-N²-{6-(methylamino)-3-oxo-4-[(1S)-1-phenylpropyl]-3,4-dihydropyrido[2,3-b]pyrazin-2-yl}glycinamide (Preparation 13A, 50 mg, 0.131 mmol) and TEA (0.051 mL, 0.394 mmol) in toluene (5 mL) was added propionyl chloride (0.018 mL, 0.197 mmol) and the reaction was stirred at room temperature for 4 hours. The reaction was concentrated in vacuo and purified using preparative TLC eluting with 5% MeOH in DCM to afford the title compound as a white solid (35 mg, 61%). ¹H NMR (400 MHz, DMSO-d₆): δ ppm 0.87-1.04 (m, 6H), 2.09-2.23 (m, 2H), 2.50-2.78 (m, 5H), 3.17 (s, 3H), 4.04-4.05 (m, 2H), 6.54-6.58 (m, 1H), 7.18-7.40 (m, 6H), 7.50-7.60 (m, 2H), 7.78-7.80 (m, 1H). MS m/z 437 [M+H]⁺

### Example 30

### N-methyl-N-(2-{[2-(methylamino)-2-oxoethyl]amino}-4-[(1S)-2-methyl-1-(pyridin-2-yl)propyl]-3-oxo-3,4-dihydropyrido[2, 3-b]pyrazin-6-yl)propanamide

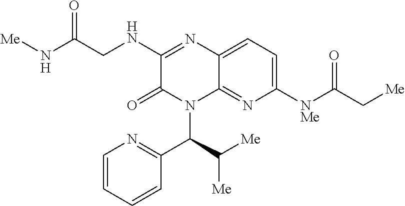 [SMILES: CCC(=O)N(C)c1ccc2nc(NCC(=O)NC)c(=O)n([C@H](c3ccccn3)C(C)C)c2n1]

The title compound was prepared according to the method described for Example 29 using N-methyl-N²-{6-(methylamino)-4-[(1S)-2-methyl-1-(pyridin-2-yl) propyl]-3-oxo-3,4-dihydropyrido[2,3-b]pyrazin-2-yl}glycinamide (Preparation 13C).

¹H NMR (400 MHz, DMSO-d₆): δ ppm 0.80 (d, 3H), 0.97 (t, 3H), 1.21 (d, 3H), 2.12-2.33 (m, 2H), 2.67 (d, 3H), 3.17 (s, 3H), 3.41-3.49 (m, 1H), 4.04-4.05 (m, 2H), 6.30-6.33 (m, 1H), 7.16-7.28 (m, 2H), 7.50-7.78 (m, 5H), 8.42-8.43 (m, 1H). MS m/z 452 [M+H]⁺

### Example 31

### N³-{4-[(1R)-2-methoxy-1-phenylethyl]-6-[methyl(propanoyl)amino]-3-oxo-3,4-dihydropyrido[2,3-b]pyrazin-2-yl}-N-methyl-beta-alaninamide

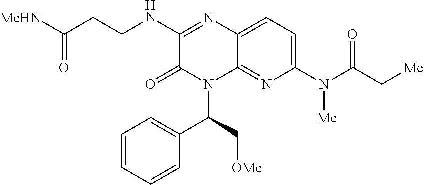 [SMILES: CCC(=O)N(C)c1ccc2nc(NCCC(=O)NC)c(=O)n([C@@H](COC)c3ccccc3)c2n1]

### Step 1

### N-{4-[(1R)-2-methoxy-1-phenylethyl]-6-[methyl(propanoyl)amino]-3-oxo-3,4-dihydropyrido[2,3-b]pyrazin-2-yl}-beta-alanine

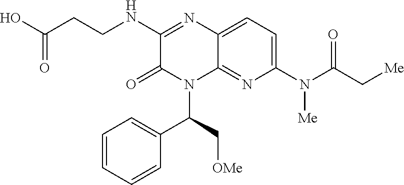 [SMILES: CCC(=O)N(C)c1ccc2nc(NCCC(=O)O)c(=O)n([C@@H](COC)c3ccccc3)c2n1]

The title compound was prepared according to the method described for Preparation 4 and Example 1 using 2,6-dichloro-4-[(1R)-2-methoxy-1-phenylethyl]pyrido[2,3-b]pyrazin-3(4H)-one (Preparation 11) and N-ethylacetamide with xantphos and cesium carbonate.

MS m/z 454 [M+H]⁺

### Step 2

To a stirring solution N³-{4-[(1R)-2-methoxy-1-phenylethyl]-6-[methyl(propanoyl)amino]-3-oxo-3,4-dihydropyrido[2,3-b]pyrazin-2-yl}-N-methyl-beta-alaninamide (31 mg, 0.068 mmol) in DMF (1 mL) at room temperature, MeNH₂—HCl (13.86 mg, 0.205 mmol), DIPEA (0.045 mL, 0.274 mmol), EDCl (19.68 mg, 0.103 mmol), HOBt (13.87 mg, 0.103 mmol) were added and the mixture allowed to stir overnight at rt. The reaction mixture was concentrated in vacuo and the residue partitioned between ethyl acetate and water. The organic extracts were dried on sodium sulfate, concentrated and purified by Preparative TLC to afford the title compound (23 mg, 72%) as an off white solid. ¹H NMR (400 MHz, DMSO-d₆): δ ppm 0.90-0.93 (m, 3H), 2.10-2.17 (m, 2H), 2.62-2.67 (m, 3H), 3.17 (s, 3H), 3.31 (s, 3H), 3.65-3.70 (m, 2H), 4.37-4.49 (m, 2H), 6.76-6.79 (m, 1H), 7.20-7.34 (m, 6H), 7.49 (br s, 2H), 7.80-7.83 (m, 1H). MS m/z 467 [M+H]⁺

### Example 32

### N-methyl-N³-{6-[methyl(propanoyl)amino]-3-oxo-4-[(1S)-1-phenylpropyl]-3,4-dihydropyrido[2, 3-b]pyrazin-2-yl}-beta-alaninamide

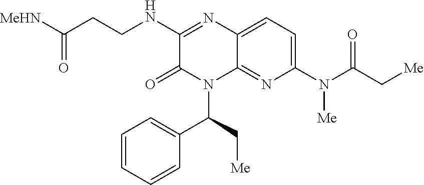 [SMILES: CCC(=O)N(C)c1ccc2nc(NCCC(=O)NC)c(=O)n([C@@H](CC)c3ccccc3)c2n1]

The title compound was prepared according to the method described for Example 31, Step 2, using N-{6-[methyl(propanoyl)amino]-3-oxo-4-[(1S)-1-phenylpropyl]-3,4-dihydropyrido[2,3-b]pyrazin-2-yl}-beta-alanine (Example 10) and methylamine hydrochloride.

¹H NMR (400 MHz, DMSO-d₆): δ ppm: 7.81 (d, 1H), 7.56 (brs, 2H), 7.36 (d, 2H), 7.25-7.33 (m, 3H), 7.21 (d, 1H), 6.46-6.61 (m, 1H), 3.66 (q, 3H), 3.17 (s, 4H), 2.67 (br s, 1H), 2.61 (d, 2H), 2.14 (br s, 2H), 0.92 (t, 3H), 0.86 (t, 3H). MS m/z 451 [M+H]⁺

### Example 33

### N-methyl-N³-{6-[methyl(propanoyl)amino]-3-oxo-4-[(1S)-1-(pyridin-2-yl)propyl]-3,4-dihydropyrido[2, 3-b]pyrazin-2-yl}-beta-alaninamide

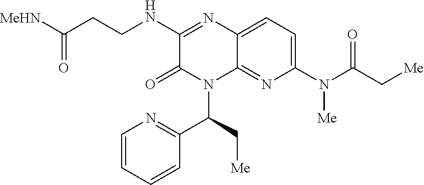 [SMILES: CCC(=O)N(C)c1ccc2nc(NCCC(=O)NC)c(=O)n([C@@H](CC)c3ccccn3)c2n1]

The title compound was prepared according to the method described for Example 31, Step 2, using N-{6-[methyl(propanoyl)amino]-3-oxo-4-[(1S)-1-(pyridin-2-yl)propyl]-3,4-dihydropyrido[2,3-b]pyrazin-2-yl}-beta-alanine (Example 11) and methylamine hydrochloride.

¹H NMR (400 MHz, DMSO-d₆): δ ppm 0.87-0.93 (m, 6H), 1.26-1.28 (m, 2H), 2.04-2.10 (m, 2H), 2.55-2.69 (m, 5H), 3.05 (s, 3H), 3.66-3.71 (m, 2H), 6.52-6.56 (m, 1H), 7.15-7.82 (m, 7H), 8.39-8.40 (m, 1H). MS m/z 452 [M+H]⁺

### Example 34

### N-methyl-N³-{6-[methyl(propanoyl)amino]-4-[(1S)-2-methyl-1-(pyridin-2-yl)propyl]-3-oxo-3,4-dihydropyrido[2, 3-b]pyrazin-2-yl}-beta-alaninamide

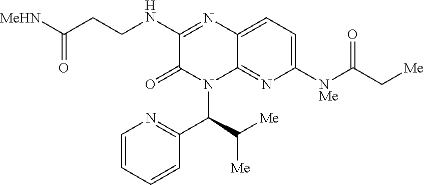 [SMILES: CCC(=O)N(C)c1ccc2nc(NCCC(=O)NC)c(=O)n([C@H](c3ccccn3)C(C)C)c2n1]

### Step 1

### N-{6-(methylamino)-4-[(1S)-2-methyl-1-(pyridin-2-yl)propyl]-3-oxo-3,4-dihydropyrido[2,3-b]pyrazin-2-yl}-beta-alanine

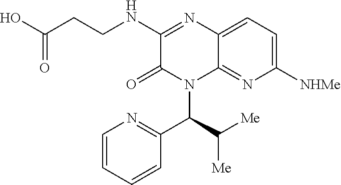 [SMILES: CNc1ccc2nc(NCCC(=O)O)c(=O)n([C@H](c3ccccn3)C(C)C)c2n1]

The title compound was prepared according to Example 1 step 2 using tert-butyl N-{6-chloro-4-[(1S)-2-methyl-1-(pyridin-2-yl) propyl]-3-oxo-3,4-dihydropyrido[2,3-b]pyrazin-2-yl}-beta-alaninate (Preparation 12A). MS m/z 397 [M+H]⁺

### Step 2

The title compound was prepared according to the methods described for Example 31, Step 2, using (S)-3-((4-(2-methyl-1-(pyridin-2-yl)propyl)-6-(methylamino)-3-oxo-3,4-dihydropyrido[2,3-b]pyrazin-2-yl)amino)propanoic acid with methylamine hydrochloride and propionyl chloride. HPLC Gemini NX-C18 (4.6×150 mm, 3 micron) operating at ambient temperature and flow rate of 1 mL/min. Mobile phase: (0.05% formic acid in water) in MeCN. Mobile phase from 90% [0.05% HCOOH in water] and 10% [MeCN] for 0.01 min, Mobile phase from 90% [0.05% HCOOH in water] and 10% [MeCN] for 0.5 min, 10% [0.05% HCOOH in water] and 90% [MeCN] for 3.5 min, held in this composition up to 8.0 min, then returned to initial composition in 8.5 min. Retention time 3.17 minutes. MS m/z 466 [M+H]⁺

### Example 35

### 6-(3,5-dimethyl-1,2-oxazol-4-yl)-4-[(1S)-1-(2-methoxyphenyl)ethyl]-2-{[2-(morpholin-4-yl)ethyl]amino}pyrido[2,3-b]pyrazin-3(4H)-one

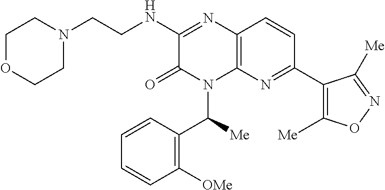 [SMILES: COc1ccccc1[C@H](C)n1c(=O)c(NCCN2CCOCC2)nc2ccc(-c3c(C)noc3C)nc21]

### Step 1

To a stirred solution of 2,6-dichloro-4-[(1S)-1-(2-methoxyphenyl)ethyl]pyrido[2,3-b]pyrazin-3(4H)-one (Preparation 3, 0.4 g, 1.14 mmol) in DCM (15 mL), was added Et₃N (0.48 mL, 3.43 mmol) followed by 4-(2-aminoethyl)morpholine (0.12 mL, 1.37 mmol). The resulting mixture was stirred at room temperature for 16 hours. After completion (TLC), the reaction mixture was diluted with water and extracted with ethyl acetate. Combined organic part was washed with 10% citric acid solution, water, and brine, dried over Na₂SO₄ and concentrated under reduced pressure. The crude mass was purified by column chromatography (1.5% Methanol-Dichloromethane) to afford 6-chloro-4-[(1S)-1-(2-methoxyphenyl)ethyl]-2-{[2-(morpholin-4-yl)ethyl]amino}pyrido[2,3-b]pyrazin-3(4H)-one (0.35 g, 69%) as yellow solid.

MS m/z 444 [M+H]⁺

### Step 2

To a stirred solution of 6-chloro-4-[(1S)-1-(2-methoxyphenyl)ethyl]-2-{[2-(morpholin-4-yl)ethyl]amino}pyrido[2,3-b]pyrazin-3(4H)-one (200 mg, 0.45 mmol) in THF (3 mL), were added (3,5-dimethylisoxazol-4-yl)boronic acid (190.6 mg, 1.35 mmol) and KF (78.4 mg, 1.35 mmol) at room temperature. After degassing the reaction mixture with argon for 20 min, Pd(OAc)₂ (1 mg, 0.005 mmol) and S-Phos (3.7 mg, 0.009) were added and the reaction mixture was heated at 100° C. for 16 h in a sealed tube. After completion (TLC), reaction mixture was diluted with water and extracted with ethyl acetate. Combined organic part was washed with water, brine, dried over Na₂SO₄ and concentrated under reduced pressure. Crude was purified by the preparative HPLC to afford the title compound (30 mg, 13%) as a white solid. ¹H NMR (400 MHz, DMSO-d₆): δ ppm 7.83 (d, 1H), 7.51 (m, 2H), 7.43 (d, 1H), 7.22 (t, 1H), 7.03 (m, 1H), 6.93 (t, 1H), 6.87 (d, 1H), 3.54 (m, 4H), 3.48 (m, 2H), 3.42 (s, 3H), 2.58 (s, 3H), 2.46 (m, 2H), 2.42 (m, 2H), 2.39 (s, 3H), 1.86 (d, 3H). MS m/z 505 [M+H]⁺

Purity: 91.46%, Rt=4.08 minutes. HPLC Zorbax SB C₁₈ (4.6×50 mm, 1.8 micron). Mobile phase: (0.05% TFA in water) in MeCN. 10 minute run.

### Example 36

### 6-(3,5-dimethyl-1,2-oxazol-4-yl)-4-(2-ethoxybenzyl)-2-{[2-(morpholin-4-yl)ethyl]amino}pyrido[2,3-b]pyrazin-3(4H)-one

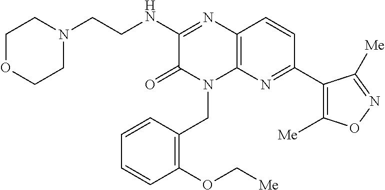 [SMILES: CCOc1ccccc1Cn1c(=O)c(NCCN2CCOCC2)nc2ccc(-c3c(C)noc3C)nc21]

The title compound was prepared according to the method described for Example 35 Step 1 using 2,6-dichloro-4-(2-ethoxybenzyl)pyrido[2,3-b]pyrazin-3(4H)-one (Preparation 11J) and 4-(2-aminoethyl)morpholine. The product of this step was then used following the method described for Example 35 Step 2 to prepare the title compound. ¹H NMR (400 MHz, DMSO-d₆): δ ppm: 7.86 (d, 1H), 7.76 (t, 1H), 7.41 (d, 1H), 7.18 (t, 1H), 7.01 (d, 1H), 6.74 (t, 1H), 6.57 (d, 1H), 4.11 (q, 2H), 3.54-3.63 (m, 5H), 3.28-3.34 (m, 4H), 2.60 (t, 2H), 2.46 (br s, 3H), 2.38 (s, 3H), 2.14 (s, 3H), 1.34 (t, 3H). MS m/z 505 [M+H]⁺

### Example 37

### 4-benzyl-6-(3,5-dimethyl-1,2-oxazol-4-yl)-2-{[2-(morpholin-4-yl)ethyl]amino}pyrido[2,3-b]pyrazin-3(4H)-one

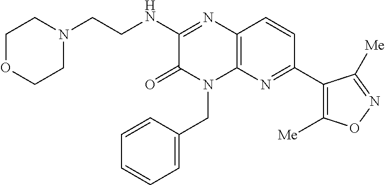 [SMILES: Cc1noc(C)c1-c1ccc2nc(NCCN3CCOCC3)c(=O)n(Cc3ccccc3)c2n1]

The title compound was prepared according to the method described for Example 35 Step 1 using 4-benzyl-2,6-dichloropyrido[2,3-b]pyrazin-3(4H)-one (Preparation 11K) and 4-(2-aminoethyl)morpholine. The product of this step was then used following the method described for Example 35 Step 2 to prepare the title compound.

¹H NMR (400 MHz, DMSO-d₆): δ ppm 7.86 (d, 1H), 7.77 (t, 1H), 7.42 (d, 1H), 7.30-7.19 (m, 5H), 5.60 (s, 2H), 3.58 (d, 6H), 2.58 (t, 2H), 2.44 (m, 7H), 2.23 (s, 3H). MS m/z 461 [M+H]⁺

Purity: 98.7%, Rt=3.81 minutes. HPLC Gemini NX-C₁₈ (4.6×150 mm, 3 micron). Mobile phase: (0.05% formic acid in water) in MeCN. 8 minute run.

### Example 38

### 6-(3,5-dimethyl-1,2-oxazol-4-yl)-2-{[2-(morpholin-4-yl)ethyl]amino}-4-[(1R)-1-phenylpropyl]pyrido[2,3-b]pyrazin-3(4H)-one

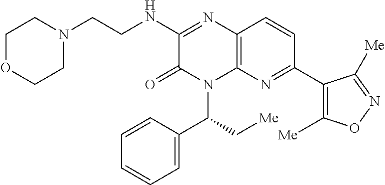 [SMILES: CC[C@H](c1ccccc1)n1c(=O)c(NCCN2CCOCC2)nc2ccc(-c3c(C)noc3C)nc21]

The title compound was prepared according to the method described for Example 35 Step 1 using 2,6-dichloro-4-[(1R)-1-phenylpropyl]pyrido[2,3-b]pyrazin-3(4H)-one (Preparation 11L) and 4-(2-aminoethyl)morpholine. The product of this step was then used following the method described for Example 35 Step 2 to prepare the title compound.

¹H NMR (400 MHz, CDCl₃): δ ppm 7.83 (d, 1H), 7.41 (m, 2H), 7.28 (m, 2H), 6.98 (m, 1H), 6.83 (m, 1H), 3.70 (bs, 4H), 3.59 (bs, 2H), 2.71 (m, 2H), 2.60-2.52 (m, 6H), 2.47 (s, 6H), 0.91 (t, 3H).

MS m/z 489 [M+H]⁺

Purity: 92.6%, Rt=4.13 minutes. HPLC Gemini NX-C₁₈ (4.6×150 mm, 3 micron). Mobile phase: (0.05% formic acid in water) in MeCN. 10 minute run.

### Example 39

### 6-(3,5-dimethyl-1,2-oxazol-4-yl)-2-{[2-(morpholin-4-yl)ethyl]amino}-4-[(1S)-1-phenylpropyl]pyrido[2,3-b]pyrazin-3(4H)-one

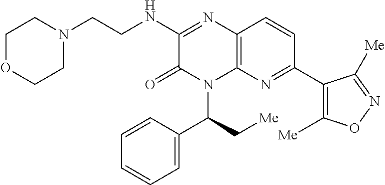 [SMILES: CC[C@@H](c1ccccc1)n1c(=O)c(NCCN2CCOCC2)nc2ccc(-c3c(C)noc3C)nc21]

The title compound was prepared according to the method described for Example 35 Step 1 using 2,6-dichloro-4-[(1S)-1-phenylpropyl]pyrido[2,3-b]pyrazin-3(4H)-one_(Preparation 6) and 4-(2-aminoethyl)morpholine. The product of this step was then used following the method described for Example 35 Step 2 to prepare the title compound.

¹H NMR (400 MHz, CDCl₃): δ 7.83 (d, 1H), 7.41 (m, 2H), 7.28 (m, 2H), 7.22 (m, 1H), 6.95 (m, 1H), 6.84 (m, 1H), 3.70 (bs, 4H), 3.59 (bs, 2H), 2.73 (m, 2H), 2.60-2.52 (m, 6H), 2.47 (s, 6H), 0.93 (t, 3H). MS m/z 489 [M+H]⁺

Purity: 95.70%, Rt=4.12 minutes. HPLC Gemini NX-C₁₈ (4.6×150 mm, 3 micron). Mobile phase: (0.05% formic acid in water) in MeCN. 10 minute run

### Preparation 14

### 6-(3,5-dimethyl-1,2-oxazol-4-yl)-3-nitro-N-[(1S)-1-phenylethyl]pyridin-2-amine

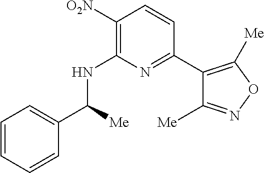 [SMILES: Cc1noc(C)c1-c1ccc([N+](=O)[O-])c(N[C@@H](C)c2ccccc2)n1]

To a stirred solution of 6-chloro-3-nitro-N-[(1S)-1-phenylethyl]pyridin-2-amine (Preparation 9M, 850 mg, 3.07 mmol) in water (0.5 mL) and dioxane (6 mL) in a sealed tube 3,5-dimethylisoxazole-4-boronic acid (1.03 g, 4.6 mmol) and Na₂CO₃ (975.7 mg, 9.21 mmol), were added. Reaction mixture was degassed with Argon for 10 min. Palladium acetate (34.44 mg, 0.153 mmol) and RuPhos (114.55 mg, 0.245 mmol) were added under inert atmosphere and again degassed for 5 min. It was heated at 80° C. for 2 h. After completion (TLC) the mixture was diluted with water and extracted with ethyl acetate. Combined organic part was washed with brine, dried over Na₂SO₄ and concentrated. Crude mass was purified by column chromatography (2-5% of ethyl acetate-hexanes) to afford the title compound as a yellow gum (850 mg, 82%). ¹H NMR (400 MHz, DMSO-d₆): δ ppm 8.61 (d, 1H), 8.50 (d, 1H), 7.41 (d, 2H), 7.33 (t, 2H), 7.23 (t, 1H), 6.96 (d, 1H), 5.48 (m, 1H), 2.47 (s, 3H), 2.25 (s, 3H), 1.60 (d, 3H). MS m/z 339 [M+H]⁺

### Preparation 14A

### 6-(3,5-dimethyl-1,2-oxazol-4-yl)-N-[(1S)-1-(2-methoxyphenyl)ethyl]-3-nitropyridin-2-amine

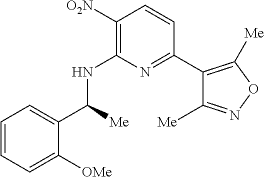 [SMILES: COc1ccccc1[C@H](C)Nc1nc(-c2c(C)noc2C)ccc1[N+](=O)[O-]]

The title compound was prepared according to the method described for Preparation 14 using 6-chloro-N-[(1S)-1-(2-methoxyphenyl)ethyl]-3-nitropyridin-2-amine (Preparation 9N).

¹H NMR (400 MHz, DMSO-d₆): δ ppm 8.95 (d, 1H), 8.48 (d, 1H), 7.29 (d, 1H), 7.24 (t, 1H), 7.03 (d, 1H), 6.94 (d, 1H), 6.89 (t, 1H), 5.71 (m, 1H), 3.87 (s, 3H), 2.53 (s, 3H), 2.31 (s, 3H), 1.52 (d, 3H). MS m/z 369 [M+H]⁺

### Preparation 14B

### 6-(3,5-dimethyl-1,2-oxazol-4-yl)-3-nitro-N-[(1S)-1-phenylpropyl]pyridin-2-amine

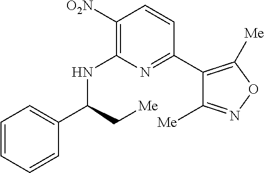 [SMILES: CC[C@H](Nc1nc(-c2c(C)noc2C)ccc1[N+](=O)[O-])c1ccccc1]

The title compound was prepared according to the method described for Preparation 14 using 6-chloro-3-nitro-N-[(1S)-1-phenylpropyl]pyridin-2-amine (Preparation 9O).

¹H NMR (400 MHz, DMSO-d₆): δ ppm 8.67 (d, 1H), 8.49 (d, 1H), 7.39 (d, 2H), 7.32 (t, 2H), 7.22 (t, 1H), 6.94 (d, 1H), 5.29 (dt, 1H), 2.48 (s, 3H), 2.26 (s, 3H), 2.00-1.90 (m, 2H), 0.92 (t, 3H). MS m/z 353 [M+H]⁺

### Preparation 14C

### 6-(3,5-dimethyl-1,2-oxazol-4-yl)-N-[(2S)-1-methoxybutan-2-yl]-3-nitropyridin-2-amine

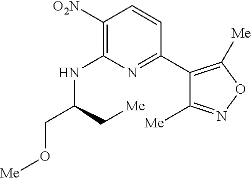 [SMILES: CC[C@@H](COC)Nc1nc(-c2c(C)noc2C)ccc1[N+](=O)[O-]]

The title compound was prepared according to the method described for Preparation 14 using 6-chloro-N-[(2S)-1-methoxybutan-2-yl]-3-nitropyridin-2-amine (Preparation 9P).

¹H NMR (400 MHz, DMSO-de): δ ppm 8.48 (d, 1H), 8.36 (d, 1H), 6.96 (d, 1H), 4.46 (m, 1H), 3.59-3.44 (m, 2H), 3.31 (s, 3H), 2.65 (s, 3H), 2.44 (s, 3H), 1.73-1.63 (m, 2H), 0.91 (t, 3H).

MS m/z 321 [M+H]⁺

### Preparation 14D

### 6-(3,5-dimethyl-1,2-oxazol-4-yl)-N-[(2R)-1-methoxybutan-2-yl]-3-nitropyridin-2-amine

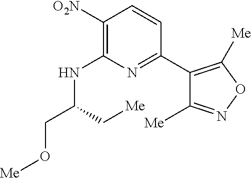 [SMILES: CC[C@H](COC)Nc1nc(-c2c(C)noc2C)ccc1[N+](=O)[O-]]

The title compound was prepared according to the method described for Preparation 14 using 6-chloro-N-[(2R)-1-methoxybutan-2-yl]-3-nitropyridin-2-amine (Preparation 9Q).

¹H NMR (400 MHz, DMSO-d₆): δ ppm 8.48 (d, 1H), 8.36 (d, 1H), 6.96 (d, 1H), 4.45 (m, 1H), 3.59-3.44 (m, 2H), 3.31 (s, 3H), 2.65 (s, 3H), 2.44 (s, 3H), 1.73-1.61 (m, 2H), 0.91 (t, 3H).

MS m/z 321 [M+H]⁺

### Preparation 14E

### N-(1,3-Dimethoxypropan-2-yl)-6-(3,5-dimethylisoxazol-4-yl)-3-nitropyridin-2-amine

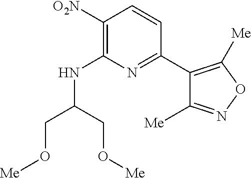 [SMILES: COCC(COC)Nc1nc(-c2c(C)noc2C)ccc1[N+](=O)[O-]]

The title compound was prepared according to the method described for Preparation 14 6-chloro-N-(1,3-dimethoxypropan-2-yl)-3-nitropyridin-2-amine (Preparation 9R).

¹H NMR (400 MHz, DMSO-d₆): δ ppm 8.49 (d, 1H), 8.43 (d, 1H), 7.00 (d, 1H), 4.64 (m, 1H), 3.61-3.49 (m, 4H), 3.31 (s, 6H), 2.65 (s, 3H), 2.44 (s, 3H). MS m/z 337 [M+H]⁺

### Preparation 14F

### 6-(3, 5-Dimethylisoxazol-4-yl)-3-nitro-N-(tetrahydro-2H-pyran-4-yl)pyridin-2-amine

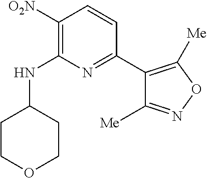 [SMILES: Cc1noc(C)c1-c1ccc([N+](=O)[O-])c(NC2CCOCC2)n1]

The title compound was prepared according to the method described for Preparation 14 using 6-chloro-3-nitro-N-(tetrahydro-2H-pyran-4-yl)pyridin-2-amine (Preparation 9S).

¹H NMR (400 MHz, DMSO-d₆): δ ppm 8.48 (d, 1H), 8.27 (d, 1H), 6.98 (d, 1H), 4.40 (m, 1H), 3.89 (d, 2H), 3.43 (t, 2H), 2.66 (s, 3H), 2.44 (s, 3H), 1.91 (d, 2H), 1.77-1.64 (m, 2H).

MS m/z 319 [M+H]⁺

### Preparation 14G

### 6-(3,5-dimethyl-1,2-oxazol-4-yl)-3-nitro-N-[(1S)-1-(pyrimidin-2-yl) propyl]pyridin-2-amine

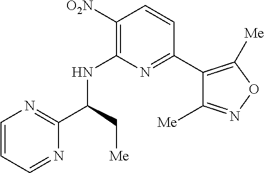 [SMILES: CC[C@H](Nc1nc(-c2c(C)noc2C)ccc1[N+](=O)[O-])c1ncccn1]

The title compound was prepared according to the method described for Preparation 14 using 6-chloro-3-nitro-N-[(1S)-1-(pyrimidin-2-yl)propyl]pyridin-2-amine (Preparation 9T).

¹H NMR (400 MHz, DMSO-d₆): δ ppm 9.24 (d, 1H), 8.86 (d, 2H), 8.52 (d, 1H), 7.46 (t, 1H), 7.00 (d, 1H), 5.53 (m, 1H), 2.61 (s, 3H), 2.39 (s, 3H), 2.10-2.04 (m, 2H), 0.81 (t, 3H).

MS m/z 355 [M+H]⁺

### Preparation 15

### 6-(3,5-dimethyl-1,2-oxazol-4-yl)-N²-[(1S)-1-phenylethyl]pyridine-2,3-diamine

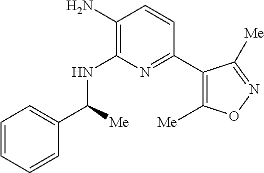

To a stirred solution of SnCl₂ (1.6 g, 8.4 mmol) in concentrated HCl (1.22 mL, 13.2 mmol) was added ethanol (8 mL). A solution of 6-(3,5-dimethyl-1,2-oxazol-4-yl)-3-nitro-N-[(1S)-1-phenylethyl]pyridin-2-amine (Preparation 14, 0.86 g, 2.54 mmol) in ethanol (4 mL) was added and stirred at 50° C. and for 2 hours. After completion (TLC); volatiles were removed under reduced pressure and the crude was basified with 2M KOH solutions until basic and extracted with EtOAc. Combined organic part was washed with brine, dried over Na₂SO₄ and concentrated. Crude compound was triturated with 5%-diethyl ether-pentane to afford the title compound (700 mg, 89%) as yellow solid. ¹H NMR (400 MHz, DMSO-d₆): δ ppm 7.34 (d, 2H), 7.27 (t, 2H), 7.15 (t, 1H), 6.78 (d, 1H), 6.47 (d, 1H), 6.05 (bs, 1H), 5.21 (m, 2H), 2.26 (s, 3H), 2.07 (s, 3H), 1.47 (d, 3H). MS m/z 309 [M+H]⁺

### Preparation 15A

### 6-(3,5-dimethyl-1, 2-oxazol-4-yl)-N²-[(1S)-1-(2-methoxyphenyl)ethyl]pyridine-2,3-diamine

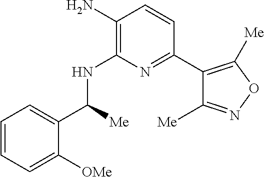

The title compound was prepared according to the method described for Preparation 15 using 6-(3,5-dimethyl-1,2-oxazol-4-yl)-N-[(1S)-1-(2-methoxyphenyl)ethyl]-3-nitropyridin-2-amine (Preparation 14A). ¹H NMR (400 MHz, DMSO-d₆): δ ppm 7.25 (d, 1H), 7.13 (t, 1H), 6.92 (d, 1H), 6.82 (t, 1H), 6.74 (d, 1H), 6.44 (d, 1H), 5.96 (d, 1H), 5.49 (m, 1H), 5.03 (bs, 2H), 3.81 (s, 3H), 2.25 (s, 3H), 2.07 (s, 3H), 1.39 (d, 3H). MS m/z 339 [M+H]⁺

### Preparation 15B

### 6-(3,5-dimethyl-1,2-oxazol-4-yl)-N²-[(1S)-1-phenylpropyl]pyridine-2,3-diamine

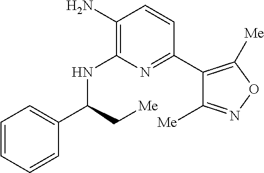 [SMILES: CC[C@H](Nc1nc(-c2c(C)noc2C)ccc1N)c1ccccc1]

The title compound was prepared according to the method described for Preparation 15 using 6-(3,5-dimethyl-1,2-oxazol-4-yl)-3-nitro-N-[(1S)-1-phenylpropyl]pyridin-2-amine (Preparation 14B). ¹H NMR (400 MHz, DMSO-d₆): δ ppm 7.33 (d, 2H), 7.26 (t, 2H), 7.15 (t, 1H), 6.73 (d, 1H), 6.44 (d, 1H), 5.94 (d, 1H), 5.02 (s, 2H), 5.00 (dt, 1H), 2.31 (s, 3H), 2.12 (s, 3H), 1.85-1.70 (m, 2H), 0.94 (t, 3H). MS m/z 323 [M+H]⁺

### Preparation 15C

### 6-(3,5-dimethyl-1,2-oxazol-4-yl)-N²-[(2S)-1-methoxybutan-2-yl]pyridine-2,3-diamine

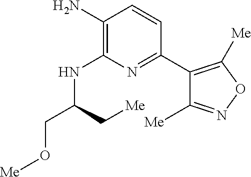 [SMILES: CC[C@@H](COC)Nc1nc(-c2c(C)noc2C)ccc1N]

The title compound was prepared according to the method described for Preparation 15 using 6-(3,5-dimethyl-1,2-oxazol-4-yl)-N-[(2S)-1-methoxybutan-2-yl]-3-nitropyridin-2-amine (Preparation 14C). ¹H NMR (400 MHz, DMSO-d₆): δ ppm 6.72 (d, 1H), 6.48 (d, 1H), 5.37 (d, 1H), 4.90 (s, 2H), 4.15 (m, 1H), 3.43 (m, 1H), 3.29 (m, 1H), 3.24 (s, 3H), 2.48 (s, 3H), 2.32 (s, 3H), 1.67 (m, 1H), 1.50 (m, 1H), 0.89 (t, 3H). MS m/z 291 [M+H]⁺

### Preparation 15D

### 6-(3,5-dimethyl-1,2-oxazol-4-yl)-N²-[(2R)-1-methoxybutan-2-yl]pyridine-2,3-diamine

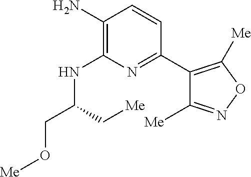 [SMILES: CC[C@H](COC)Nc1nc(-c2c(C)noc2C)ccc1N]

The title compound was prepared according to the method described for Preparation 15 using 6-(3,5-dimethyl-1,2-oxazol-4-yl)-N-[(2R)-1-methoxybutan-2-yl]-3-nitropyridin-2-amine (Preparation 14D). ¹H NMR (400 MHz, DMSO-d₆): δ ppm 6.73 (d, 1H), 6.48 (d, 1H), 5.36 (d, 1H), 4.89 (s, 2H), 4.15 (m, 1H), 3.44 (m, 1H), 3.27 (m, 1H), 3.24 (s, 3H), 2.49 (s, 3H), 2.32 (s, 3H), 1.69 (m, 1H), 1.50 (m, 1H), 0.90 (t, 3H). MS m/z 291 [M+H]⁺

### Preparation 15E

### N²-(1,3-dimethoxypropan-2-yl)-6-(3,5-dimethyl-1,2-oxazol-4-yl)pyridine-2,3-diamine

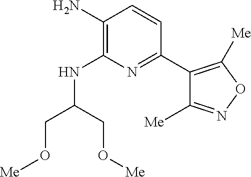 [SMILES: COCC(COC)Nc1nc(-c2c(C)noc2C)ccc1N]

The title compound was prepared according to the method described for Preparation 15 using N-(1,3-dimethoxypropan-2-yl)-6-(3,5-dimethylisoxazol-4-yl)-3-nitropyridin-2-amine (Preparation 14E). ¹H NMR (400 MHz, CDCl₃): δ ppm 6.87 (d, 1H), 6.56 (d, 1H), 4.59 (d, 1H), 4.44 (m, 1H), 3.65-3.51 (m, 4H), 3.36 (s, 6H), 3.25 (s, 2H), 2.53 (s, 3H), 2.41 (s, 3H). MS m/z 307 [M+H]⁺

### Preparation 15F

### 6-(3,5-dimethyl-1,2-oxazol-4-yl)-N²-(tetrahydro-2H-pyran-4-yl)pyridine-2,3-diamine

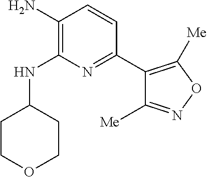 [SMILES: Cc1noc(C)c1-c1ccc(N)c(NC2CCOCC2)n1]

The title compound was prepared according to the method described for Preparation 15 using 6-(3,5-dimethylisoxazol-4-yl)-3-nitro-N-(tetrahydro-2H-pyran-4-yl)pyridin-2-amine (Preparation 14F). ¹H NMR (400 MHz, DMSO-d₆): δ ppm 6.74 (d, 1H), 6.51 (d, 1H), 5.53 (d, 1H), 4.88 (s, 2H), 4.05 (m, 1H), 3.89 (d, 2H), 3.39 (t, 2H), 2.49 (s, 3H), 2.32 (s, 3H), 1.92 (d, 2H), 1.51-1.41 (m, 2H). MS m/z 289 [M+H]⁺

### Preparation 15G

### 6-(3,5-dimethyl-1,2-oxazol-4-yl)-N²-[(1S)-1-(pyrimidin-2-yl)propyl]pyridine-2,3-diamine

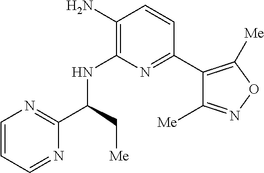 [SMILES: CC[C@H](Nc1nc(-c2c(C)noc2C)ccc1N)c1ncccn1]

The title compound was prepared according to the method described for Preparation 15 using 6-(3,5-dimethyl-1,2-oxazol-4-yl)-3-nitro-N-[(1S)-1-(pyrimidin-2-yl) propyl]pyridin-2-am ine (Preparation 14G). ¹H NMR (400 MHz, DMSO-d₆): δ ppm 8.73 (d, 1H), 7.32 (t, 1H), 6.74 (d, 1H), 6.46 (d, 1H), 6.02 (d, 1H), 5.10 (m, 1H), 5.04 (s, 2H), 4.03 (m, 1H), 2.30 (s, 3H), 2.09 (s, 3H), 1.91 (m, 2H), 0.95 (t, 3H). MS m/z 325 [M+H]⁺

### Preparation 16

### 2-chloro-6-(3,5-dimethyl-1,2-oxazol-4-yl)-4-[(1S)-1-phenylethyl]pyrido[2,3-b]pyrazin-3(4H)-one

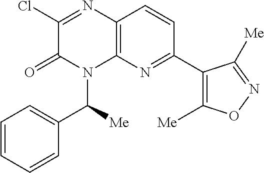 [SMILES: Cc1noc(C)c1-c1ccc2nc(Cl)c(=O)n([C@@H](C)c3ccccc3)c2n1]

A solution of oxalyl chloride (0.196 mL, 2.27 mmol) in 1,2-dichloro benzene (4 mL) was heated to 50° C. and a solution of 6-(3,5-dimethyl-1,2-oxazol-4-yl)-N²-[(1S)-1-phenylethyl]pyridine-2,3-diamine (Preparation 15, 700 mg, 2.27 mmol) in 1,2-dichloro benzene (8 mL) was added over a period of 10 min then stirred at 130° C. for 2 hours. The mixture was cooled to room temperature (TLC indicated a polar spot was formed) and oxalyl chloride (0.196 mL, 2.27 mmol) was added. It was again heated to 130° C. and continued for 1 hour. After completion (TLC); it was quenched with water and extracted with ethyl acetate. Organic part was washed with NaHCO₃ solution, brine, dried over Na₂SO₄ and concentrated. Crude mass was purified by column chromatography (5-10% EtOAc-hexanes) to afford the title compound (305 mg, 35%) as yellow solid. ¹H NMR (400 MHz, CDCl₃): δ ppm 8.17 (d, 1H), 7.41 (d, 1H), 7.36 (d, 2H), 7.29 (m, 2H), 7.11 (m 1H), 2.58 (s, 3H), 2.44 (s, 3H), 2.01 (d, 3H). MS m/z 381 [M+H]⁺

### Preparation 16A

### 2-chloro-6-(3,5-dimethyl-1,2-oxazol-4-yl)-4-[(1S)-1-(2-methoxyphenyl)ethyl]pyrido[2,3-b]pyrazin-3(4H)-one

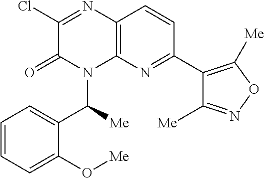 [SMILES: COc1ccccc1[C@H](C)n1c(=O)c(Cl)nc2ccc(-c3c(C)noc3C)nc21]

The title compound was prepared according to the method described for Preparation 16 using 6-(3,5-dimethyl-1,2-oxazol-4-yl)-N²-[(1S)-1-(2-methoxyphenyl)ethyl]pyridine-2,3-diamine (Preparation 15A). ¹H NMR (400 MHz, DMSO-de): δ ppm 8.31 (d, 1H), 7.67 (d, 1H), 7.51 (d, 1H), 7.24 (t, 1H), 7.09-7.00 (m, 1H), 6.95 (t, 1H), 6.88 (d, 1H), 3.41 (s, 3H), 2.64 (s, 3H), 2.43 (s, 3H), 1.86 (d, 3H). MS m/z 411 [M+H]⁺

### Preparation 16B

### 2-chloro-6-(3,5-dimethyl-1,2-oxazol-4-yl)-4-[(1S)-1-phenylpropyl]pyrido[2,3-b]pyrazin-3(4H)-one

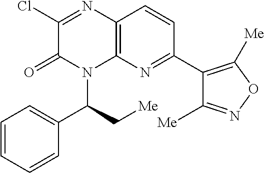 [SMILES: CC[C@@H](c1ccccc1)n1c(=O)c(Cl)nc2ccc(-c3c(C)noc3C)nc21]

The title compound was prepared according to the method described for Preparation 16 using 6-(3,5-dimethyl-1,2-oxazol-4-yl)-N²-[(1S)-1-phenylpropyl]pyridine-2,3-diamine (Preparation 15B). MS m/z 395 [M+H]⁺

### Preparation 16C

### 2-chloro-6-(3,5-dimethyl-1,2-oxazol-4-yl)-4-[(2S)-1-methoxybutan-2-yl]pyrido[2,3-b]pyrazin-3(4H)-one

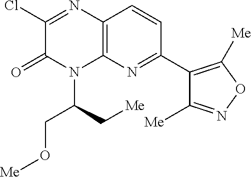 [SMILES: CC[C@@H](COC)n1c(=O)c(Cl)nc2ccc(-c3c(C)noc3C)nc21]

The title compound was prepared according to the method described for Preparation 16 using 6-(3,5-dimethyl-1,2-oxazol-4-yl)-N²-[(2S)-1-methoxybutan-2-yl]pyridine-2,3-diamine (Preparation 15C). ¹H NMR (400 MHz, CDCl₃): δ ppm 8.13 (d, 1H), 7.38 (d, 1H), 6.02 (m, 1H), 4.24 (t, 1H), 3.72 (dd, 1H), 3.24 (s, 3H), 2.67 (s, 3H), 2.52 (s, 3H), 2.22 (m, 1H), 1.99 (m, 1H), 0.87 (t, 3H). MS m/z 363 [M+H]⁺

### Preparation 16D

### 2-chloro-6-(3,5-dimethyl-1,2-oxazol-4-yl)-4-[(2R)-1-methoxybutan-2-yl]pyrido[2,3-b]pyrazin-3(4H)-one

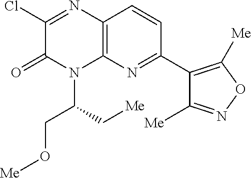 [SMILES: CC[C@H](COC)n1c(=O)c(Cl)nc2ccc(-c3c(C)noc3C)nc21]

The title compound was prepared according to the method described for Preparation 16 using 6-(3,5-dimethyl-1,2-oxazol-4-yl)-N²-[(2R)-1-methoxybutan-2-yl]pyridine-2,3-diamine (Preparation 15D). ¹H NMR (400 MHz, DMSO-d₆): δ ppm 8.31 (d, 1H), 7.66 (d, 1H), 5.89 (m, 1H), 4.04 (t, 1H), 3.74 (m, 1H), 3.16 (s, 3H), 2.67 (s, 3H), 2.48 (s, 3H), 2.11 (m, 1H), 1.93 (m, 1H), 0.83 (t, 3H). MS m/z 363 [M+H]⁺

### Preparation 16E

### 2-chloro-4-(1,3-dimethoxypropan-2-yl)-6-(3,5-dimethyl-1,2-oxazol-4-yl)pyrido[2,3-b]pyrazin-3(4H)-one

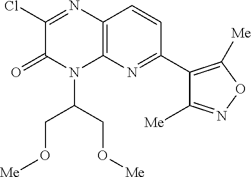 [SMILES: COCC(COC)n1c(=O)c(Cl)nc2ccc(-c3c(C)noc3C)nc21]

The title compound was prepared according to the method described for Preparation 16 using N²-(1,3-dimethoxypropan-2-yl)-6-(3,5-dimethyl-1,2-oxazol-4-yl)pyridine-2,3-diamine (Preparation 15E). ¹H NMR (400 MHz, CDCl₃): δ ppm 8.13 (d, 1H), 7.38 (d, 1H), 6.31 (m, 1H), 4.12 (m, 2H), 3.82 (m, 2H), 3.27 (s, 6H), 2.67 (s, 3H), 2.51 (s, 3H). MS m/z 379 [M+H]⁺

### Preparation 16F

### 2-chloro-6-(3,5-dimethyl-1,2-oxazol-4-yl)-4-(tetrahydro-2H-pyran-4-yl)pyrido[2,3-b]pyrazin-3(4H)-one

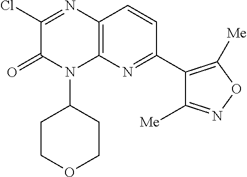 [SMILES: Cc1noc(C)c1-c1ccc2nc(Cl)c(=O)n(C3CCOCC3)c2n1]

The title compound was prepared according to the method described for Preparation 16 using 6-(3,5-dimethyl-1,2-oxazol-4-yl)-N²-(tetrahydro-2H-pyran-4-yl)pyridine-2,3-diamine (Preparation 15F). ¹H NMR (400 MHz, DMSO-d₆): δ ppm 8.29 (d, 1H), 7.66 (d, 1H), 5.79 (t, 1H), 4.01 (dd, 2H), 3.43 (t, 2H), 2.80-2.74 (m, 2H), 2.69 (s, 3H), 2.49 (s, 3H), 1.62 (d, 2H). MS m/z 361 [M+H]⁺

### Preparation 16G

### 2-chloro-6-(3,5-dimethyl-1,2-oxazol-4-yl)-4-[(1S)-1-(pyrimidin-2-yl)propyl]pyrido[2,3-b]pyrazin-3(4H)-one

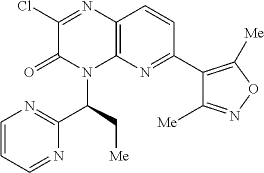 [SMILES: CC[C@@H](c1ncccn1)n1c(=O)c(Cl)nc2ccc(-c3c(C)noc3C)nc21]

The title compound was prepared according to the method described for Preparation 16 using 6-(3,5-dimethyl-1,2-oxazol-4-yl)-N²-[(1S)-1-(pyrimidin-2-yl)propyl]pyridine-2,3-diamine (Preparation 15G). MS m/z 397 [M+H]⁺

### Example 40

### 6-(3,5-dimethyl-1,2-oxazol-4-yl)-2-{[2-(morpholin-4-yl)ethyl]amino}-4-[(1S)-1-phenylethyl]pyrido[2,3-b]pyrazin-3(4H)-one

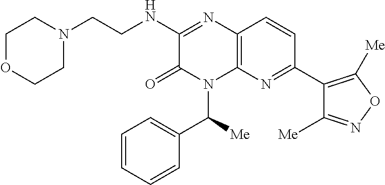 [SMILES: Cc1noc(C)c1-c1ccc2nc(NCCN3CCOCC3)c(=O)n([C@@H](C)c3ccccc3)c2n1]

To a stirred solution of 2-chloro-6-(3,5-dimethyl-1,2-oxazol-4-yl)-4-[(1S)-1-phenylethyl]pyrido[2,3-b]pyrazin-3(4H)-one (Preparation 16, 35 mg, 0.092 mmol) in DCM (2 mL) was added 2-(morpholin-4-yl)ethanamine (14.4 mg 0.11 mmol) and Et₃N (0.038 mL, 0.28 mmol) was added MeNH₂—HCl (67.52 mg, 0.111 mmol) and Et₃N (0.039 mL, 0.28 mmol). The resulting mixture was stirred at room temperature for 16 hours. After completion (TLC), reaction was diluted with DCM and was washed with water. Organic part was dried over Na₂SO₄ and concentrated. Crude mass was purified by preparative TLC (3% MeOH-DCM) to afford the title compound (12 mg, 27%) as a light brown solid.

¹H NMR (400 MHz, MeOH-d₄): δ ppm 7.88 (d, 1H), 7.38 (d, 1H), 7.30-7.24 (m, 4H), 7.20 (m, 1H), 7.07 (m, 1H), 3.70-3.64 (m, 6H), 2.66 (t, 2H), 2.55 (bs, 4H), 2.46 (s, 3H), 2.28 (s, 3H), 2.00 (d, 3H), 0.88 (m, 1H). MS m/z 475 [M+H]⁺

Purity: 95.5%, Rt=6.83 minutes. HPLC Gemini C₁₈ (4.6×100 mm, 5 micron). Mobile phase A: acetonitrile; mobile phase B: 10 mM NH₄OAc in water; 12 minute run; injection volume: 2 uL.

### Example 41

### N-{6-(3,5-dimethyl-1,2-oxazol-4-yl)-3-oxo-4-[(1S)-1-phenylethyl]-3,4-dihydropyrido[2,3-b]pyrazin-2-yl}glycine

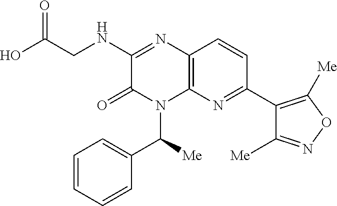 [SMILES: Cc1noc(C)c1-c1ccc2nc(NCC(=O)O)c(=O)n([C@@H](C)c3ccccc3)c2n1]

The title compound was prepared according to the method described for Example 40 using 2-chloro-6-(3,5-dimethyl-1,2-oxazol-4-yl)-4-[(1S)-1-phenylethyl]pyrido[2,3-b]pyrazin-3(4H)-one (Preparation 16) and glycine. ¹H NMR (400 MHz, MeOH-d₄): δ ppm 7.89 (d, 1H), 7.36 (d, 1H), 7.30-7.23 (m, 4H), 7.17 (d, 1H), 7.05 (m, 1H), 4.07 (bs, 1H), 2.43 (s, 3H), 2.25 (s, 3H), 2.00 (d, 3H), 0.89 (m, 2H). MS m/z 420 [M+H]⁺

Purity: 93.02%, Rt=4.64 minutes. HPLC Gemini C₁₈ (4.6×100 mm, 5 micron). Mobile phase A: acetonitrile; mobile phase B: 10 mM NH₄OAc in water; 12 minute run; injection volume: 2 uL.

### Example 42

### N-{6-(3,5-dimethyl-1,2-oxazol-4-yl)-3-oxo-4-[(1S)-1-phenylethyl]-3,4-dihydropyrido[2,3-b]pyrazin-2-yl}-beta-alanine

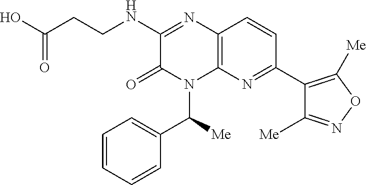 [SMILES: Cc1noc(C)c1-c1ccc2nc(NCCC(=O)O)c(=O)n([C@@H](C)c3ccccc3)c2n1]

The title compound was prepared according to the method described for Example 40 using 2-chloro-6-(3,5-dimethyl-1,2-oxazol-4-yl)-4-[(1S)-1-phenylethyl]pyrido[2,3-b]pyrazin-3(4H)-one (Preparation 16) and 3-aminopropanoic acid. ¹H NMR (400 MHz, MeOH-d₄): δ ppm 7.91 (d, 1H), 7.39 (d, 1H), 7.27-7.17 (m, 5H), 7.05 (m, 1H), 3.75 (t, 1H), 2.65 (bs, 2H), 2.45 (s, 3H), 2.28 (s, 3H), 1.99 (d, 3H), 0.86 (m, 2H). MS m/z 434 [M+H]⁺

Purity: 97.8%, Rt=4.20 minutes. HPLC Zorbax SB C₁₈ (4.6×50 mm, 1.8 micron). Mobile phase: (0.05% TFA in water) in acetonitrile. 8 minute run

### Example 43

### N-{6-(3,5-dimethyl-1,2-oxazol-4-yl)-4-[(1S)-1-(2-methoxyphenyl)ethyl]-3-oxo-3,4-dihydropyrido[2,3-b]pyrazin-2-yl}-beta-alanine

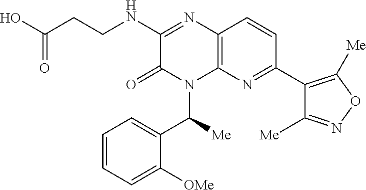 [SMILES: COc1ccccc1[C@H](C)n1c(=O)c(NCCC(=O)O)nc2ccc(-c3c(C)noc3C)nc21]

### Step 1

To a stirred solution of 2-chloro-6-(3,5-dimethyl-1,2-oxazol-4-yl)-4-[(1S)-1-(2-methoxyphenyl)ethyl]pyrido[2,3-b]pyrazin-3(4H)-one (Preparation 16A, 0.1 g, 0.24 mmol) in DCM (3.0 mL) in a pear shaped vial, Et₃N (0.1 mL, 0.73 mmol) and tert-butyl-3-aminopropanoate (95.9 mg, 0.73 mmol) was added and resulting mixture was stirred at room temperature for 48 hours. After completion (monitored by TLC), the mixture was quenched with water and extracted with DCM. Organic layer was washed with brine, dried over Na₂SO₄ and concentrated under reduced pressure. Crude mass was purified by column chromatography (22% ethyl acetate-hexanes) to afford tert-butyl (S)-3-((6-(3,5-dimethylisoxazol-4-yl)-4-(1-(2-methoxyphenyl)ethyl)-3-oxo-3,4-dihydropyrido[2,3-b]pyrazin-2-yl)amino)propanoate as a brown solid (90.0 mg, 73%).

### Step 2

To a stirred solution of tert-butyl (S)-3-((6-(3,5-dimethylisoxazol-4-yl)-4-(1-(2-methoxyphenyl)ethyl)-3-oxo-3,4-dihydropyrido[2,3-b]pyrazin-2-yl)amino)propanoate (50.0 mg, 0.1 mmol) in DCM (2.0 mL), TFA (0.52 mL, 6.73 mmol) was added and resulting mixture was stirred at room temperature for 5 h. After completion (monitored by TLC), the mixture was concentrated under reduced pressure and azeotroped with DCM three times. Purification by preparative TLC (5% MeOH-DCM) afforded the title compound as a brown solid (25 mg, 56%).

¹H NMR (400 MHz, DMSO-d₆): δ ppm 7.85 (d, 1H), 7.76 (m, 1H), 7.48 (d, 1H), 7.43 (d, 1H), 7.21 (t, 1H), 7.02 (m, 1H), 6.94-6.86 (m, 2H), 3.56 (m, 2H), 3.43 (s, 3H), 2.58 (s, 3H), 2.54 (m, 2H), 2.39 (s, 3H), 1.85 (d, 3H). MS m/z 462 [M−H]⁻

Purity: 95%, Rt=6.08 minutes. HPLC Gemini NX-C₁₈ (4.6×100 mm, 5 micron). Mobile phase A: acetonitrile; mobile phase B: 10 mM NH4OAc in water; 12 minute run; injection volume: 1 uL.

### Example 44

### N-{6-(3,5-dimethyl-1,2-oxazol-4-yl)-3-oxo-4-[(1S)-1-phenylpropyl]-3,4-dihydropyrido[2,3-b]pyrazin-2-yl}-beta-alanine

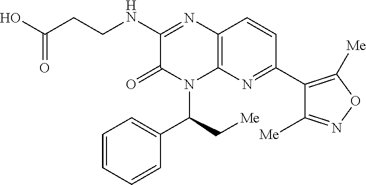 [SMILES: CC[C@@H](c1ccccc1)n1c(=O)c(NCCC(=O)O)nc2ccc(-c3c(C)noc3C)nc21]

The title compound was prepared according to the method described for Example 43 using 2-chloro-6-(3,5-dimethyl-1,2-oxazol-4-yl)-4-[(1S)-1-phenylpropyl]pyrido[2,3-b]pyrazin-3(4H)-one (Preparation 16B) and tert-butyl-3-aminopropanoate. ¹H NMR (400 MHz, MeOH-d₄): δ ppm 7.93-7.89 (m, 1H), 7.43-7.32 (m, 3H), 7.24 (t, 2H), 7.18 (m, 1H), 6.90 (bs, 1H), 3.76 (t, 2H), 2.80-2.68 (m, 4H), 2.57-2.50 (m, 4H), 2.40-2.30 (m, 3H), 0.89 (t, 3H). MS m/z 448 [M+H]⁺

Purity: 99.8%, Rt=5.21 minutes. HPLC Zorbax SB C₁₈ (4.6×50 mm, 5 micron). Mobile phase A: acetonitrile; mobile phase B: 10 mM NH4OAc in water; 12 minute run; injection volume: 2 uL.

### Example 45

### N-{6-(3,5-dimethyl-1,2-oxazol-4-yl)-3-oxo-4-[(1S)-1-(pyrimidin-2-yl)propyl]-3,4-dihydropyrido[2,3-b]pyrazin-2-yl}-beta-alanine

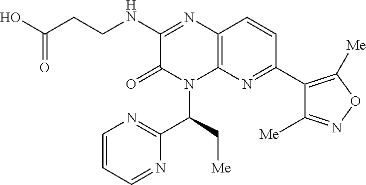 [SMILES: CC[C@@H](c1ncccn1)n1c(=O)c(NCCC(=O)O)nc2ccc(-c3c(C)noc3C)nc21]

The title compound was prepared according to the method described for Example 43 using 2-chloro-6-(3,5-dimethyl-1,2-oxazol-4-yl)-4-[(1S)-1-(pyrimidin-2-yl)propyl]pyrido[2,3-b]pyrazin-3(4H)-one (Preparation 16G) and tert-butyl-3-aminopropanoate. ¹H NMR (400 MHz, MeOH-d₄): δ ppm 8.63 (d, 2H), 7.91 (d, 1H), 7.34 (d, 1H), 7.25 (s, 1H), 6.83 (bs, 1H), 3.79 (bs, 2H), 2.88 (m, 1H), 2.71 (m, 2H), 2.60 (m, 1H), 2.42 (s, 3H), 2.23 (s, 3H), 0.96 (t, 3H). MS m/z 450 [M+H]^{+ Purity:} 97.2%, Rt=4.02 minutes. HPLC X-Bridge C₁₈ (4.6×50 mm, 5 micron). Mobile phase A: acetonitrile; mobile phase B: 0.1% TFA in water; 10 minute run; injection volume: 8 uL.

### Example 46

### N-{6-(3,5-dimethyl-1,2-oxazol-4-yl)-4-[(2S)-1-methoxybutan-2-yl]-3-oxo-3,4-dihydropyrido[2,3-b]pyrazin-2-yl}-beta-alanine

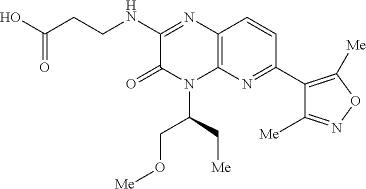 [SMILES: CC[C@@H](COC)n1c(=O)c(NCCC(=O)O)nc2ccc(-c3c(C)noc3C)nc21]

The title compound was prepared according to the method described for Example 43 using 2-chloro-6-(3,5-dimethyl-1,2-oxazol-4-yl)-4-[(2S)-1-methoxybutan-2-yl]pyrido[2,3-b]pyrazin-3(4H)-one (Preparation 16C) and tert-butyl-3-aminopropanoate. ¹H NMR (400 MHz, CDCl₃): δ ppm 7.81 (d, 1H), 7.21 (d, 1H), 6.81 (m, 1H), 6.00 (m, 1H), 4.19 (t, 1H), 3.81-3.74 (m, 3H), 3.25 (s, 3H), 2.62 (s, 2H), 2.49 (m, 1H), 2.48 (s, 3H), 2.38 (m, 1H), 2.20 (m, 1H), 1.96 (m, 1H), 0.85 (t, 3H). MS m/z 416 [M+H]⁺

Purity: 99.2%, Rt=6.57 minutes. HPLC Gemini NX-C₁₈ (4.6×50 mm, 3 micron). Mobile phase A: 0.05% HCOOH in water; mobile B: acetonitrile; 12 minute run; injection volume: 2 uL.

### Example 47

### N-{6-(3,5-dimethyl-1,2-oxazol-4-yl)-4-[(2R)-1-methoxybutan-2-yl]-3-oxo-3,4-dihydropyrido[2,3-b]pyrazin-2-yl}-beta-alanine

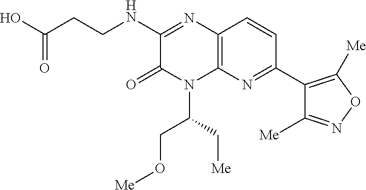 [SMILES: CC[C@H](COC)n1c(=O)c(NCCC(=O)O)nc2ccc(-c3c(C)noc3C)nc21]

The title compound was prepared according to the method described for Example 43 using 2-chloro-6-(3,5-dimethyl-1,2-oxazol-4-yl)-4-[(2R)-1-methoxybutan-2-yl]pyrido[2,3-b]pyrazin-3(4H)-one (Preparation 16D) and tert-butyl-3-aminopropanoate. ¹H NMR (400 MHz, DMSO-d₆): δ ppm 7.85 (d, 1H), 7.43 (d, 1H), 5.91 (m, 1H), 4.10 (t, 1H), 3.72 (m, 1H), 3.62 (m, 2H), 3.15 (s, 3H), 2.62 (m, 1H), 2.61 (s, 3H), 2.43 (s, 3H), 2.33 (m, 1H), 2.14 (m, 1H), 1.90 (m, 1H), 0.79 (t, 3H). MS m/z 416 [M+H]⁺

Purity: 98.7%, Rt=6.57 minutes. HPLC Gemini NX-C₁₈ (4.6×50 mm, 3 micron). Mobile phase A: 0.05% HCOOH in water; mobile phase B: acetonitrile; 12 minute run; injection volume: 2 uL.

### Example 48

### N-[4-(1,3-dimethoxypropan-2-yl)-6-(3,5-dimethyl-1,2-oxazol-4-yl)-3-oxo-3,4-dihydropyrido[2,3-b]pyrazin-2-yl]-beta-alanine

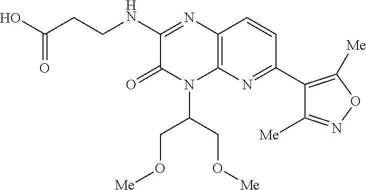 [SMILES: COCC(COC)n1c(=O)c(NCCC(=O)O)nc2ccc(-c3c(C)noc3C)nc21]

The title compound was prepared according to the method described for Example 43 using 2-chloro-4-(1,3-dimethoxypropan-2-yl)-6-(3,5-dimethyl-1,2-oxazol-4-yl)pyrido[2,3-b]pyrazin-3(4H)-one (Preparation 16E) and tert-butyl-3-aminopropanoate. ¹H NMR (400 MHz, DMSO-d₆): δ ppm 12.29 (s, 1H), 7.85 (d, 2H), 7.43 (d, 1H), 6.18 (m, 1H), 4.03 (m, 2H), 3.77 (m, 2H), 3.63 (m, 2H), 3.18 (s, 6H), 2.63 (m, 4H), 2.42 (s, 3H). MS m/z 432 [M+H]⁺

Purity: 96.2%, Rt=3.94 minutes. HPLC Zorbax SB C₁₈ (4.6×50 mm, 1.8 micron). Mobile phase A: 0.05% TFA in water; mobile phase B: acetonitrile; 10 minute run; injection volume: 2 uL.

### Example 49

### N-[6-(3,5-dimethyl-1,2-oxazol-4-yl)-3-oxo-4-(tetrahydro-2H-pyran-4-yl)-3,4-dihydropyrido[2,3-b]pyrazin-2-yl]-beta-alanine

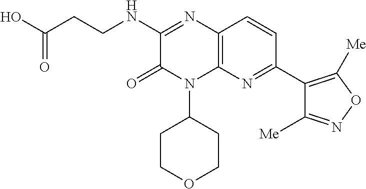 [SMILES: Cc1noc(C)c1-c1ccc2nc(NCCC(=O)O)c(=O)n(C3CCOCC3)c2n1]

The title compound was prepared according to the method described for Example 43 using 2-chloro-6-(3,5-dimethylisoxazol-4-yl)-4-(tetrahydro-2H-pyran-4-yl)pyrido[2,3-b]pyrazin-3(4H)-one (Preparation 16F) and tert-butyl-3-aminopropanoate. ¹H NMR (400 MHz, MeOH-d₄): δ ppm 7.92 (d, 1H), 7.44 (d, 1H), 5.90 (m, 1H), 4.09 (dd, 2H), 3.80 (t, 2H), 3.55 (t, 2H), 3.00 (m, 2H), 2.74 (t, 2H), 2.64 (s, 3H), 2.48 (s, 3H), 1.65 (d, 2H). MS m/z 414 [M+H]⁺

Purity: 96.9%, Rt=4.07 minutes. HPLC Zorbax SB C₁₈ (4.6×50 mm, 1.8 micron). Mobile phase A: 0.05% TFA in water; mobile phase B: acetonitrile; 10 minute run; injection volume: 2 uL.

### Example 50

### N³-{6-(3,5-dimethyl-1,2-oxazol-4-yl)-3-oxo-4-[(1S)-1-phenylethyl]-3,4-dihydropyrido[2,3-b]pyrazin-2-yl}-N-(methylsulfonyl)-beta-alaninamide

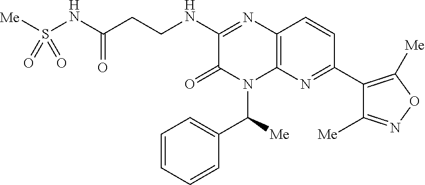

The title compound was prepared according to the method described for Example 40 using 2-chloro-6-(3,5-dimethyl-1,2-oxazol-4-yl)-4-[(1S)-1-phenylethyl]pyrido[2,3-b]pyrazin-3(4H)-one (Preparation 16) and 3-amino-N-(methylsulfonyl)propanamide. ¹H NMR (400 MHz, MeOH-d₄): δ ppm 7.92 (d, 1H), 7.39 (d, 1H), 7.30-7.23 (m, 4H), 7.19 (m, 1H), 7.06 (m, 1H), 3.79 (t, 2H), 3.16 (s, 3H), 2.69 (t, 2H), 2.46 (b s, 3H), 2.28 (b s, 3H), 1.99 (d, 3H). MS m/z 511 [M+H]⁺

Purity: 99.4%, Rt=4.80 minutes. HPLC Zorbax SB C₁₈ (4.6×50 mm, 1.8 micron). Mobile phase: (0.05% TFA in water) in acetonitrile. 10 minute run

### Example 51

### 6-(3,5-dimethyl-1,2-oxazol-4-yl)-4-[(1S)-1-phenylethyl]-2-{[2-(1H-tetrazol-5-yl)ethyl]amino}pyrido[2,3-b]pyrazin-3(4H)-one

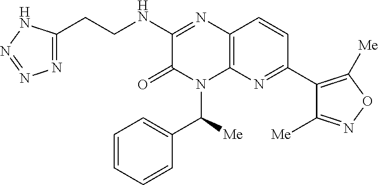

### Step 1

To a stirred solution of 2-chloro-6-(3,5-dimethyl-1,2-oxazol-4-yl)-4-[(1S)-1-phenylethyl]pyrido[2,3-b]pyrazin-3(4H)-one (Preparation 16, 100 mg, 0.26 mmol) in DCM (2 mL), 2-(1-trityl-1H-tetrazol-5-yl)ethan-1-amine (112 mg, 0.32 mmol) and DIPEA (0.13 mL, 0.79 mmol) was added at room temperature. Reaction mixture was stirred at room temperature for 16 hours. Additional 1.2 equivalent 2-(1-trityl-1H-tetrazol-5-yl)ethan-1-amine and 3 equivalents of DIPEA and stirred another 24 hours. After completion (TLC), reaction mixture was quenched with water and extracted with DCM, combined organic layer was washed with water, brine, dried over Na₂SO₄, filtered and concentrated under reduced pressure. Crude mass was purified by column chromatography (15-25% EA-Hexane) to afford (S)-6-(3,5-dimethylisoxazol-4-yl)-4-(1-phenyl ethyl)-2-((2-(1-trityl-1H-tetrazol-5-yl)ethyl)amino)pyrido[2,3-b]pyrazin-3(4H)-one as yellow solid (150 mg, 81%).

### Step 2

Ether in HCl (2M, 0.5 mL) was added to above compound under cooling condition. The resulting mixture was warmed to room temperature and stirred for 6 hours. After completion (TLC), ether layer was decanted. Solid thus obtained was washed with ether several times and dried under reduced pressure to afford the title compound as an off-white solid (70 mg, 71%). ¹H NMR (400 MHz, MeOH-d₄): δ ppm 8.00) d, 1H), 7.49 (d, 1H), 7.34 (d, 2H), 7.27 (t, 2H), 7.21 (d, 1H), 7.03 (m, 1H), 4.05 (t, 2H), 3.40 (t, 2H), 2.47 (s, 3H), 2.29 (s, 3H), 2.00 (d, 3H). MS m/z 458 [M+H]⁺

Purity: 95.8%, Rt=5.16 minutes. HPLC Zorbax Extend C₁₈ (4.6×50 mm, 5 micron). Mobile phase A: acetonitrile; mobile phase B: 10 mM NH₄OAc in water; 12 minute run; injection volume: 2 uL.

### Preparation 17

### 6-(3,5-dimethyl-1,2-oxazol-4-yl)-3-nitropyridin-2-amine

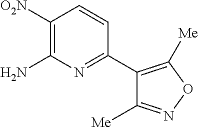 [SMILES: Cc1noc(C)c1-c1ccc([N+](=O)[O-])c(N)n1]

In a sealed tube, a solution of 6-chloro-3-nitropyridin-2-amine (25 g, 144.51 mmol), 3,5-dimethylisoxazole-4-boronic acid (30.56 g, 216.76 mmol), Cs₂CO₃ (140.9 g, 433.53 mmol) in dioxane-H₂O (2:1, 50 mL) was degassed with argon for 20 min. PdCl₂(dppf).DCM (11.79 g, 14.45 mmol) was added in it. The mixture was heated at 100° C. for 16 hours. After completion (TLC), reaction mixture was diluted with EtOAc and filtered on Celite bed and bed was washed with EtOAc. Organic layer was washed with brine dried over Na₂SO₄, filtered and concentrated under reduced pressure. Crude was purified by column chromatography (25% EA-Hexanes) to get the title compound as a yellow solid (26.5 g, 78%). ¹H NMR (400 MHz, DMSO-d₆): δ ppm 8.42 (d, 1H), 7.97 (bs, 2H), 6.91 (d, 1H), 2.64 (s, 3H), 2.43 (s, 3H). MS m/z 235 [M+H]⁺

### Preparation 18

### 2-chloro-6-(3, 5-dimethyl-1,2-oxazol-4-yl)-3-nitropyridine

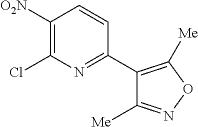 [SMILES: Cc1noc(C)c1-c1ccc([N+](=O)[O-])c(Cl)n1]

A solution of CuCl2 (36.5 g, 271.8 mmol), LiCl (9.6 g, 226.5 mmol) and t-butyl nitrite (43.1 mL, 362.4 mmol) in MeCN (530 mL) was heated at 65° C. 6-(3,5-dimethyl-1,2-oxazol-4-yl)-3-nitropyridin-2-amine (Preparation 17, 53 g, 226.5 mmol) was added portion wise under heating condition and heating was continued for 4 hours. The reaction was not completed and another 1 equivalent each of t-butyl nitrite, CuCl₂ and LiCl were added and continued heating for another 3 hours. After completion, reaction mixture was cooled to ambient temperature, quenched with 20% HCl solution and extracted with ethyl acetate. Combined organic layer was washed with brine, dried over Na₂SO₄ filtered and concentrated under reduced pressure. Crude mass was purified by column chromatography (15% EA-hexanes) to afford the title compound as yellow solid (39 g, 68%). ¹H NMR (400 MHz, DMSO-d₆): δ ppm 8.64 (d, 1H), 7.85 (d, 1H), 2.65 (s, 3H), 2.43 (s, 3H). MS m/z 254 [M+H]⁺

### Preparation 19

### 6-(3,5-dimethyl-1,2-oxazol-4-yl)-3-nitro-N-[(1S)-1-phenylbutyl]pyridin-2-amine

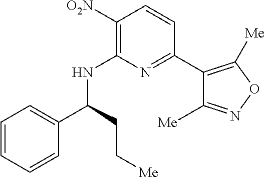 [SMILES: CCC[C@H](Nc1nc(-c2c(C)noc2C)ccc1[N+](=O)[O-])c1ccccc1]

The title compound was prepared according to the method described for Preparation 1 using 2-chloro-6-(3,5-dimethyl-1,2-oxazol-4-yl)-3-nitropyridine (Preparation 18) and (S)-1-phenylbutan-1-amine. ¹H NMR (400 MHz, CDCl₃): δ ppm 8.77 (d, 1H), 8.45 (d, 1H), 7.31 (s, 4H), 7.25 (s, 1H), 6.66 (d, 1H), 5.37 (dt, 1H), 2.44 (s, 3H), 2.30 (s, 3H), 1.92 (m, 2H), 1.50-1.38 (m, 2H), 0.99 (t, 3H). MS m/z 367 [M+H]⁺

### Preparation 19A

### 6-(3,5-dimethyl-1,2-oxazol-4-yl)-N-[(1S)-2-methyl-1-phenylpropyl]-3-nitropyridin-2-amine

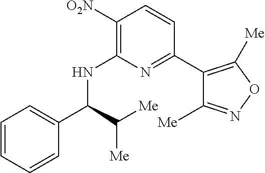 [SMILES: Cc1noc(C)c1-c1ccc([N+](=O)[O-])c(N[C@H](c2ccccc2)C(C)C)n1]

The title compound was prepared according to the method described for Preparation 19 using 4-(6-chloro-5-nitropyridin-2-yl)-3,5-dimethylisoxazole (Preparation 18) and (S)-2-methyl-1-phenylpropan-1-amine. 1H NMR (400 MHz, DMSO-d₆): δ ppm 8.83 (d, 1H), 8.50 (d, 1H), 7.33 (m, 4H), 7.22 (m, 1H), 6.94 (d, 1H), 5.24 (t, 1H), 2.47 (s, 3H), 2.25 (s, 3H), 0.93 (d, 6H). MS m/z 367 [M+H]⁺

### Preparation 19B

### 6-(3,5-dimethyl-1,2-oxazol-4-yl)-N-[(1S)-2-methyl-1-(pyridin-2-yl)propyl]-3-nitropyridin-2-amine

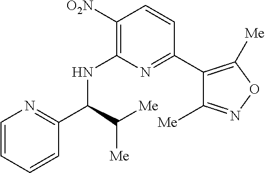 [SMILES: Cc1noc(C)c1-c1ccc([N+](=O)[O-])c(N[C@H](c2ccccn2)C(C)C)n1]

The title compound was prepared according to the method described for Preparation 1 using 4-(6-chloro-5-nitropyridin-2-yl)-3,5-dimethylisoxazole (Preparation 18) and (S)-1-(pyridin-2-yl)propan-1-amine. ¹H NMR (400 MHz, CDCl₃): δ ppm 9.25 (d, 1H), 8.61 (d, 1H), 8.47 (d, 1H), 7.60 (t, 1H), 7.16 (m, 2H), 6.68 (d, 1H), 5.38 (dd, 1H), 2.49 (s, 3H), 2.46 (m, 1H), 2.34 (s, 3H), 0.98 (d, 6H). MS m/z 368 [M+H]⁺

### Preparation 19C

### 6-(3,5-dimethyl-1,2-oxazol-4-yl)-3-nitro-N-(pentan-3-yl)pyridin-2-amine

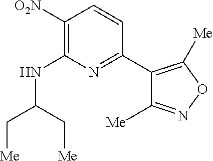 [SMILES: CCC(CC)Nc1nc(-c2c(C)noc2C)ccc1[N+](=O)[O-]]

The title compound was prepared according to the method described for Preparation 2 using 4-(6-chloro-5-nitropyridin-2-yl)-3,5-dimethylisoxazole (Preparation 18) and pentan-3-amine. ¹H NMR (400 MHz, DMSO-d₆): δ ppm 8.47 (d, 1H), 8.19 (d, 1H), 6.94 (d, 1H), 4.27 (m, 1H), 2.65 (s, 3H), 2.44 (s, 3H), 1.70-1.55 (m, 4H), 0.89 (t, 6H). MS m/z 305 [M+H]⁺

### Preparation 19D

### N-[(1S)-1-cyclohexylethyl]-6-(3,5-dimethyl-1,2-oxazol-4-yl)-3-nitropyridin-2-amine

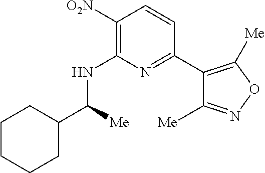 [SMILES: Cc1noc(C)c1-c1ccc([N+](=O)[O-])c(N[C@@H](C)C2CCCCC2)n1]

The title compound was prepared according to the method described for Preparation 1 using 4-(6-chloro-5-nitropyridin-2-yl)-3,5-dimethylisoxazole (Preparation 18) and (S)-1-cyclohexylethan-1-amine. ¹H NMR (400 MHz, DMSO-d₆): δ ppm 8.47 (d, 1H), 8.29 (d, 1H), 6.93 (d, 1H), 4.35-4.30 (m, 1H), 2.65 (s, 3H), 2.44 (s, 3H), 1.78 (d, 1H), 1.72 (d, 3H), 1.60 (m, 2H), 1.25-1.15 (m, 3H), 1.20 (d, 3H), 1.12-0.98 (m, 3H). MS m/z 345 [M+H]⁺

### Preparation 20

### 6-(3,5-dimethyl-1,2-oxazol-4-yl)-N²-[(1S)-1-phenylbutyl]pyridine-2,3-diamine

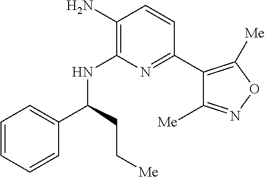 [SMILES: CCC[C@H](Nc1nc(-c2c(C)noc2C)ccc1N)c1ccccc1]

The title compound was prepared according to the method described for Preparation 2 using (S)-6-(3,5-dimethylisoxazol-4-yl)-3-nitro-N-(1-phenylbutyl)pyridin-2-amine (Preparation 19). ¹H NMR (400 MHz, DMSO-d₆): δ ppm 7.33 (d, 2H), 7.26 (t, 2H), 7.14 (t, 1H), 6.72 (d, 1H), 6.44 (d, 1H), 5.96 (d, 1H), 5.10 (dt, 1H), 5.02 (s, 2H), 2.31 (s, 3H), 2.12 (s, 3H), 1.85-1.65 (m, 2H), 1.50-1.30 (m, 2H), 0.89 (t, 3H). MS m/z 337 [M+H]⁺

### Preparation 20A

### 6-(3,5-dimethyl-1,2-oxazol-4-yl)-N²-[(1S)-2-methyl-1-phenylpropyl]pyridine-2,3-diamine

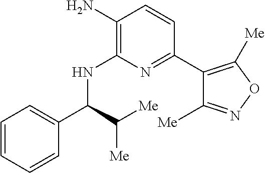 [SMILES: Cc1noc(C)c1-c1ccc(N)c(N[C@H](c2ccccc2)C(C)C)n1]

The title compound was prepared according to the method described for Preparation 2 using (S)-6-(3, 5-dimethylisoxazol-4-yl)-N-(2-methyl-1-phenylpropyl)-3-nitropyridin-2-amine (Preparation 18). ¹H NMR (400 MHz, DMSO-d₆): δ ppm 7.32 (d, 2H), 7.26 (t, 2H), 7.15 (t, 1H), 6.72 (d, 1H), 6.43 (d, 1H), 5.82 (d, 1H), 5.04 (s, 2H), 4.93 (t, 1H), 2.35 (s, 3H), 2.17 (s, 3H), 2.03 (m, 1H), 0.98 (d, 3H), 0.80 (d, 3H). MS m/z 337 [M+H]⁺

### Preparation 20B

### 6-(3,5-dimethyl-1,2-oxazol-4-yl)-N²-[(1S)-2-methyl-1-(pyridin-2-yl)propyl]pyridine-2,3-diamine

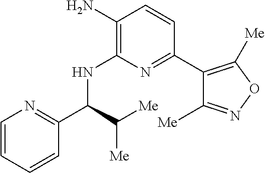 [SMILES: Cc1noc(C)c1-c1ccc(N)c(N[C@H](c2ccccn2)C(C)C)n1]

The title compound was prepared according to the method described for Preparation 2 using 6-(3,5-dimethyl-1,2-oxazol-4-yl)-N-[(1S)-2-methyl-1-(pyridin-2-yl)propyl]-3-nitropyridin-2-amine (Preparation 19B). ¹H NMR (400 MHz, DMSO-d₆): δ ppm 8.50 (d, 1H), 7.67 (t, 1H), 7.32 (d, 1H), 7.18 (t, 1h), 6.75 (d, 1H), 6.46 (d, 1H), 5.83 (d, 1H), 5.06 (m, 3H), 2.32 (s, 3H), 2.25 (m, 1H), 2.13 (s, 3H), 0.95 (d, 3H), 0.84 (d, 3H). MS m/z 338 [M+H]⁺

### Preparation 20C

### 6-(3,5-dimethyl-1,2-oxazol-4-yl)-N²-(pentan-3-yl)pyridine-2,3-diamine

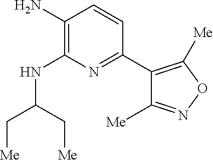 [SMILES: CCC(CC)Nc1nc(-c2c(C)noc2C)ccc1N]

The title compound was prepared according to the method described for Preparation 20 using 6-(3,5-dimethyl-1,2-oxazol-4-yl)-3-nitro-N-(pentan-3-yl)pyridin-2-amine (Preparation 19C). ¹H NMR (400 MHz, DMSO-d₆): δ ppm 6.71 (d, 1H), 6.45 (d, 1H), 5.26 (d, 1H), 4.87 (s, 2H), 3.93 (m, 1H), 2.49 (s, 3H), 2.32 (s, 3H), 1.57-1.47 (m, 4H), 0.87 (t, 6H). MS m/z 275 [M+H]⁺

### Preparation 20D

### N²-[(1S)-1-cyclohexylethyl]-6-(3, 5-dimethyl-1,2-oxazol-4-yl)pyridine-2, 3-diamine

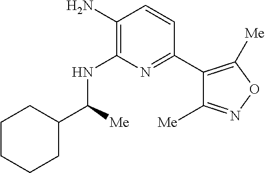 [SMILES: Cc1noc(C)c1-c1ccc(N)c(N[C@@H](C)C2CCCCC2)n1]

The title compound was prepared according to the method described for Preparation 2 using N-[(1S)-1-cyclohexylethyl]-6-(3,5-dimethyl-1,2-oxazol-4-yl)-3-nitropyridin-2-amine (Preparation 19D). MS m/z 315 [M+H]⁺

### Preparation 21

### methyl {[6-(3,5-dimethyl-1,2-oxazol-4-yl)-2-{[(1S)-1-phenylbutyl]amino}pyridin-3-yl]amino}(oxo)acetate

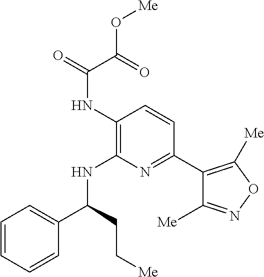 [SMILES: CCC[C@H](Nc1nc(-c2c(C)noc2C)ccc1NC(=O)C(=O)OC)c1ccccc1]

To a stirred solution of 6-(3,5-dimethyl-1,2-oxazol-4-yl)-N²-[(1S)-1-phenylbutyl]pyridine-2,3-diamine (Preparation 20, 210 mg, 0.62 mmol) in THF (5 mL) was added Na₂CO₃ (132.30 mg, 1.25 mmol) and methyl 2-chloro-2-oxoacetate (0.07 mL, 0.75 mmol) and resulting mixture was stirred at room temperature for 45 minutes. After completion (TLC), the reaction mass was diluted with EtOAc and separated. Organic part was then washed with water and brine dried over Na₂SO₄, filtered and concentrated under reduced pressure to afford the title compound as a yellow gum (260 mg, 98%) that was used in the next step without further purification. ¹H NMR (400 MHz, CDCl₃): δ ppm 8.70 (d, 1H), 8.09 (m, 1H), 7.74 (d, 1H), 7.65 (m, 1H), 7.55 (m, 1H), 6.65 (d, 1H), 5.15 (d, 1H), 4.01 (s, 3H), 2.39 (s, 3H), 2.23 (s, 3H), 1.12 (d, 3H), 0.90 (d, 3H). MS m/z 424 [M+H]⁺

### Preparation 21A

### Methyl {[6-(3,5-dimethyl-1,2-oxazol-4-yl)-2-{[(1S)-2-methyl-1-phenylpropyl]amino}pyridin-3-yl]amino}(oxo)acetic acid

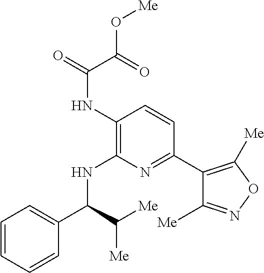 [SMILES: COC(=O)C(=O)Nc1ccc(-c2c(C)noc2C)nc1N[C@H](c1ccccc1)C(C)C]

The title compound was prepared according to the method described for Preparation 21 using methyl 6-(3,5-dimethyl-1,2-oxazol-4-yl)-N²-[(1S)-2-methyl-1-phenylpropyl]pyridine-2,3-diamine (Preparation 20A). ¹H NMR (400 MHz, DMSO-d₆): δ ppm 10.18 (s, 1H), 7.47 (d, 1H), 7.37 (d, 2H), 7.26 (t, 2H), 7.15 (t, 1H), 6.66 (d, 1H), 6.53 (d, 1H), 5.10 (m, 1H), 2.32 (s, 3H), 2.16 (s, 3H), 1.80 (m, 1H), 0.90 (dd, 6H). MS m/z 409 [M+H]⁺

### Preparation 21B

### Methyl {[6-(3,5-dimethyl-1,2-oxazol-4-yl)-2-{[(1S)-2-methyl-1-(pyridin-2-yl)propyl]amino}pyridin-3-yl]amino}(oxo)acetate

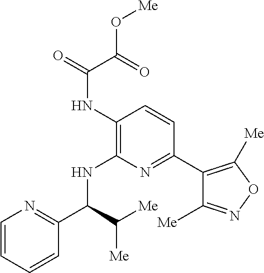 [SMILES: COC(=O)C(=O)Nc1ccc(-c2c(C)noc2C)nc1N[C@H](c1ccccn1)C(C)C]

The title compound was prepared according to the method described for Preparation 21 using 6-(3,5-dimethyl-1,2-oxazol-4-yl)-N²-[(1S)-2-methyl-1-(pyridin-2-yl)propyl]pyridine-2,3-diamine (Preparation 20B). ¹H NMR (400 MHz, CDCl₃): δ ppm 8.78 (m, 1H), 7.60 (d, 1H), 7.35-7.25 (m, 5H), 7.19 (m, 1H), 6.62 (d, 1H), 5.10 (t, 1H), 4.01 (s, 3H), 2.31 (s, 3H), 2.18 (s, 3H), 1.88-1.77 (m, 1H), 0.90 (dd, 6H). MS m/z 424 [M+H]⁺

### Preparation 21C

### Methyl {[6-(3,5-dimethyl-1,2-oxazol-4-yl)-2-(pentan-3-ylamino)pyridin-3-yl]amino}(oxo)acetate

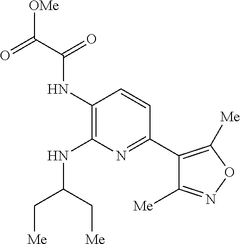 [SMILES: CCC(CC)Nc1nc(-c2c(C)noc2C)ccc1NC(=O)C(=O)OC]

The title compound was prepared according to the method described for Preparation 21 using 6-(3,5-dimethyl-1,2-oxazol-4-yl)-N²-(pentan-3-yl)pyridine-2,3-diamine (Preparation 20C). ¹H NMR (400 MHz, DMSO-de): δ ppm 10.13 (s, 1H), 7.40 (d, 1H), 6.66 (d, 1H), 5.80 (d, 1H), 4.00 (m, 1H), 3.85 (s, 3H), 2.56 (s, 3H), 2.38 (s, 3H), 1.57-1.43 (m, 4H), 0.86 (t, 6H).

### Preparation 22

### {[6-(3,5-dimethyl-1,2-oxazol-4-yl)-2-{[(1S)-1-phenylbutyl]amino}pyridin-3-yl]amino}(oxo)acetic acid

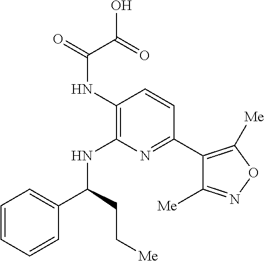 [SMILES: CCC[C@H](Nc1nc(-c2c(C)noc2C)ccc1NC(=O)C(=O)O)c1ccccc1]

To a stirred solution of methyl {[6-(3,5-dimethyl-1,2-oxazol-4-yl)-2-{[(1S)-1-phenylbutyl]amino}pyridin-3-yl]amino}(oxo)acetate (Preparation 21, 260 mg, 0.61 mmol) in THF (3 mL) was added 1N NaOH solution (1.5 mL) slowly at 00° C. and stirred at same temperature for 30 min. After completion (TLC); reaction mass was acidified with 1N HCl solution and was extracted with EtOAc. Organic part was then washed with water, brine, dried over Na₂SO₄ and concentrated under reduced pressure to afford the title compound as a brown solid (250 mg, 99%). It was used in the next step without further purification.

MS m/z 410 [M+H]⁺

### Preparation 22A

### {[6-(3,5-dimethyl-1,2-oxazol-4-yl)-2-{[(1S)-2-methyl-1-phenylpropyl]amino}pyridin-3-yl]amino}(oxo)acetate

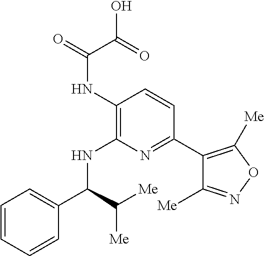 [SMILES: Cc1noc(C)c1-c1ccc(NC(=O)C(=O)O)c(N[C@H](c2ccccc2)C(C)C)n1]

The title compound was prepared according to the method described for Preparation 22 using {[6-(3,5-dimethyl-1,2-oxazol-4-yl)-2-{[(1S)-2-methyl-1-phenylpropyl]amino}pyridin-3-yl]amino}(oxo)acetic acid (Preparation 21A). ¹H NMR (400 MHz, DMSO-d₆): δ ppm 10.40 (s, 1H), 7.41 (d, 1H), 7.36 (d, 1H), 7.26 (t, 2H), 7.16 (t, 1H), 6.66 (d, 1H), 6.30 (d, 1H), 4.90 (t, 1H), 3.89 (s, 3H), 2.39 (s, 3H), 2.20 (s, 3H), 2.07 (m, 1H), 0.94 (d, 3H), 0.80 (d, 3H). MS m/z 409 [M+H]⁺

### Preparation 22B

### {[6-(3,5-dimethyl-1,2-oxazol-4-yl)-2-{[(1S)-2-methyl-1-(pyridin-2-yl)propyl]amino}pyridin-3-yl]amino}(oxo)acetic acid

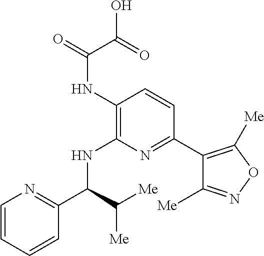 [SMILES: Cc1noc(C)c1-c1ccc(NC(=O)C(=O)O)c(N[C@H](c2ccccn2)C(C)C)n1]

The title compound was prepared according to the method described for Preparation 22 using methyl {[6-(3,5-dimethyl-1,2-oxazol-4-yl)-2-{[(1S)-2-methyl-1-(pyridin-2-yl)propyl]amino}pyridin-3-yl]amino}(oxo)acetate (Preparation 21B). ¹H NMR (400 MHz, DMSO-d₆): δ ppm 10.27 (s, 1H), 7.48 (d, 1h), 7.36 (d, 2H), 7.26 (t, 2H), 7.16 (m, 1H), 6.66 (d, 1H), 6.32 (d, 1H), 4.90 (t, 1H), 2.38 (s, 3H), 2.19 (s, 3H), 2.07 (m, 1H), 0.93 (d, 3H), 0.85 (d, 3H). MS m/z 410 [M+H]⁺

### Preparation 22C

### {[6-(3,5-dimethyl-1,2-oxazol-4-yl)-2-(pentan-3-ylamino)pyridin-3-yl]amino}(oxo)acetic acid

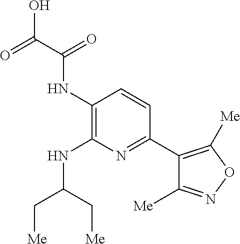 [SMILES: CCC(CC)Nc1nc(-c2c(C)noc2C)ccc1NC(=O)C(=O)O]

The title compound was prepared according to the method described for Preparation 22 using methyl {[6-(3,5-dimethyl-1,2-oxazol-4-yl)-2-(pentan-3-ylamino)pyridin-3-yl]amino}(oxo)acetate (Preparation 21C). ¹H NMR (400 MHz, DMSO-d₆): δ ppm 10.03 (s, 1H), 7.44 (d, 1H), 6.66 (d, 1H), 5.85 (m, 1H), 3.97 (m, 1H), 2.60 (s, 3H), 2.38 (s, 3H), 1.57-1.43 (m, 4H), 0.86 (t, 6H). MS m/z 347 [M+H]⁺

### Preparation 23

### 2-chloro-6-(3,5-dimethyl-1,2-oxazol-4-yl)-4-[(1S)-1-phenylbutyl]pyrido[2,3-b]pyrazin-3(4H)-one

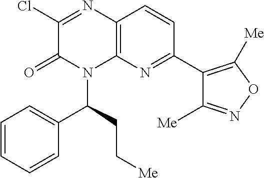 [SMILES: CCC[C@@H](c1ccccc1)n1c(=O)c(Cl)nc2ccc(-c3c(C)noc3C)nc21]

To a stirred solution of {[6-(3,5-dimethyl-1,2-oxazol-4-yl)-2-{[(1S)-1-phenylbutyl]amino}pyridin-3-yl]amino}(oxo)acetic acid (Preparation 22, 250 mg, 0.61 mmol) in THF (5 mL) was added oxalyl chloride (0.1 mL, 1.22 mmol), followed by DMF (catalytic amount) and the resulting mixture was stirred at 50° C. for 4 hours. After completion (TLC) volatiles were removed under reduced pressure and stripped with DCM three times to afford the title compound as a yellow gum (249 mg, 99%). It was used in the next step without further purification. MS m/z 409 [M+H]⁺

### Preparation 23A

### 2-chloro-6-(3,5-dimethyl-1,2-oxazol-4-yl)-4-[(1S)-2-methyl-1-phenylpropyl]pyrido[2,3-b]pyrazin-3(4H)-one

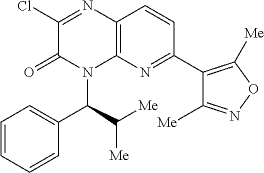 [SMILES: Cc1noc(C)c1-c1ccc2nc(Cl)c(=O)n([C@H](c3ccccc3)C(C)C)c2n1]

The title compound was prepared according to the method described for Preparation 23 using {[6-(3,5-dimethyl-1,2-oxazol-4-yl)-2-{[(1S)-2-methyl-1-phenylpropyl]amino}pyridin-3-yl]amino}(oxo)acetate (Preparation 22A). MS m/z 409 [M+H]⁺

### Preparation 23B

### 2-chloro-6-(3,5-dimethyl-1,2-oxazol-4-yl)-4-[(1S)-2-methyl-1-(pyridin-2-yl)propyl]pyrido[2,3-b]pyrazin-3(4H)-one

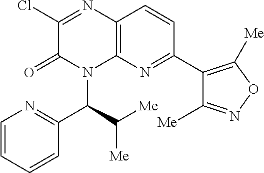 [SMILES: Cc1noc(C)c1-c1ccc2nc(Cl)c(=O)n([C@H](c3ccccn3)C(C)C)c2n1]

The title compound was prepared according to the method described for Preparation 23 using {[6-(3,5-dimethyl-1,2-oxazol-4-yl)-2-{[(1S)-2-methyl-1-(pyridin-2-yl)propyl]amino}pyridin-3-yl]amino}(oxo)acetic acid (Preparation 22B). MS m/z 410 [M+H]⁺

### Preparation 23C

### 2-chloro-6-(3,5-dimethyl-1,2-oxazol-4-yl)-4-(pentan-3-yl)pyrido[2,3-b]pyrazin-3(4H)-one

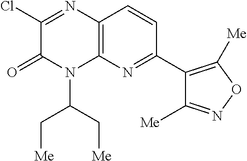 [SMILES: CCC(CC)n1c(=O)c(Cl)nc2ccc(-c3c(C)noc3C)nc21]

The title compound was prepared according to the method described for Preparation 22 using 2-((6-(3,5-dimethylisoxazol-4-yl)-2-(pentan-3-ylamino)pyridin-3-yl)amino)-2-oxoacetic acid (Preparation 21C). MS m/z 347 [M+H]⁺

### Preparation 23D

### 2-chloro-4-[(1S)-1-cyclohexylethyl]-6-(3,5-dimethyl-1,2-oxazol-4-yl)pyrido[2,3-b]pyrazin-3(4H)-one

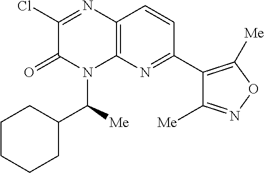 [SMILES: Cc1noc(C)c1-c1ccc2nc(Cl)c(=O)n([C@@H](C)C3CCCCC3)c2n1]

The title compound was prepared according to the method described for Preparation 16 using N²-[(1S)-1-cyclohexylethyl]-6-(3,5-dimethyl-1,2-oxazol-4-yl)pyridine-2,3-diamine (Preparation 20D). MS m/z 387 [M+H]⁺

### Example 52

### N-{6-(3,5-dimethyl-1,2-oxazol-4-yl)-3-oxo-4-[(1S)-1-phenylbutyl]-3,4-dihydropyrido[2,3-b]pyrazin-2-yl}-beta-alanine

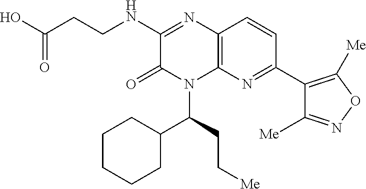 [SMILES: CCC[C@@H](C1CCCCC1)n1c(=O)c(NCCC(=O)O)nc2ccc(-c3c(C)noc3C)nc21]

The title compound was prepared according to the method described for Example 43 using 2-chloro-6-(3,5-dimethyl-1,2-oxazol-4-yl)-4-[(1S)-1-phenylbutyl]pyrido[2,3-b]pyrazin-3(4H)-one (Preparation 23) and tert-butyl-3-aminopropanoate. ¹H NMR (400 MHz, DMSO-d₆): δ ppm 7.89 (d, 1H), 7.80 (m, 1H), 7.47 (m, 1H), 7.34 (m, 2H), 7.27 (t, 2H), 7.21 (m, 1H), 6.93 (m, 1H), 3.60 (m, 2H), 2.60 (m, 5H), 2.41 (m, 3H), 1.23 (m, 3H), 0.87 (t, 3H). MS m/z 462 [M+H]⁺

Purity: 98.7%, Rt=5.18 minutes. HPLC Zorbax SB C₁₈ (4.6×50 mm, 1.8 micron). Mobile phase: (0.05% TFA in water) in acetonitrile. 10 minute run.

### Example 53

### N-{6-(3,5-dimethyl-1,2-oxazol-4-yl)-4-[(1S)-2-methyl-1-phenylpropyl]-3-oxo-3,4-dihydropyrido[2,3-b]pyrazin-2-yl}-beta-alanine

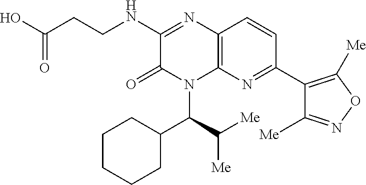 [SMILES: Cc1noc(C)c1-c1ccc2nc(NCCC(=O)O)c(=O)n([C@@H](C(C)C)C3CCCCC3)c2n1]

The title compound was prepared according to the method described for Example 43 using 2-chloro-6-(3,5-dimethyl-1, 2-oxazol-4-yl)-4-[(1S)-2-methyl-1-phenylpropyl]pyrido[2,3-b]pyrazin-3(4H)-one (Preparation 23A) and tert-butyl-3-aminopropanoate. ¹H NMR (400 MHz, DMSO-d₆): δ ppm 7.89 (m, 1H), 7.60 (d, 2H), 7.45 (m, 1H), 7.22 (m, 3H), 6.68 (m, 1H), 3.73 (t, 2H), 3.57 (m, 1H), 2.66 (m, 4H), 2.55 (br s, 3H), 1.28 (s, 2H), 1.03 (d, 3H), 0.86 (d, 3H). MS m/z 460 [M−H]⁻

Purity: 99.8%, Rt=4.65 minutes. HPLC Atlantis C₁₈ (4.6×50 mm, 3 micron). Mobile phase: (0.05% TFA in water) in acetonitrile. 10 minute run.

### Example 54

### N-{6-(3,5-dimethyl-1,2-oxazol-4-yl)-4-[(1S)-2-methyl-1-(pyridin-2-yl)propyl]-3-oxo-3,4-dihydropyrido[2,3-b]pyrazin-2-yl}-beta-alanine

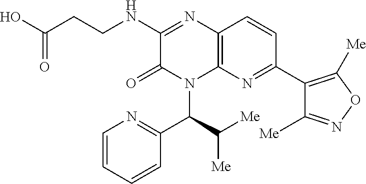 [SMILES: Cc1noc(C)c1-c1ccc2nc(NCCC(=O)O)c(=O)n([C@H](c3ccccn3)C(C)C)c2n1]

The title compound was prepared according to the method described for Example 43 using 2-chloro-6-(3,5-dimethyl-1,2-oxazol-4-yl)-4-[(1S)-2-methyl-1-(pyridin-2-yl)propyl]pyrido[2,3-b]pyrazin-3(4H)-one (Preparation 23B) and tert-butyl-3-aminopropanoate. ¹H NMR (400 MHz, CDCl₃-d): δ ppm: 8.48 (br s, 1H), 7.83 (br. s, 2H), 7.60 (br s, 2H), 7.12 (br s, 1H), 6.79 (br s, 2H), 3.77 (br s, 2H), 3.48 (br s, 2H), 2.60-2.80 (m, 4H), 2.54 (br s, 3H), 1.17 (d, 3H), 0.85 (d, 3H). MS m/z 463 [M−H]⁻

Purity: 98.5%, Rt=3.89 minutes. HPLC Zorbax SB C₁₈ (4.6×50 mm, 1.8 micron). Mobile phase: (0.05% TFA in water) in acetonitrile. 10 minute run.

### Example 55

### N-{4-[(1S)-1-cyclohexylethyl]-6-(3,5-dimethyl-1,2-oxazol-4-yl)-3-oxo-3,4-dihydropyrido[2,3-b]pyrazin-2-yl}-beta-alanine

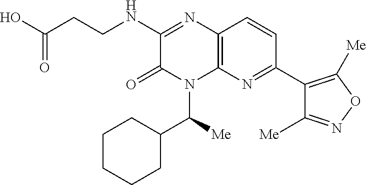 [SMILES: Cc1noc(C)c1-c1ccc2nc(NCCC(=O)O)c(=O)n([C@@H](C)C3CCCCC3)c2n1]

The title compound was prepared according to the method described for Example 43 using 2-chloro-4-[(1S)-1-cyclohexylethyl]-6-(3,5-dimethyl-1,2-oxazol-4-yl)pyrido[2,3-b]pyrazin-3(4H)-one (Preparation 23D) and tert-butyl-3-aminopropanoate.

¹H NMR (400 MHz, DMSO-d₆): δ ppm 7.84 (m, 2H), 7.42 (d, 1H), 5.53 (m, 1H), 3.62 (m, 2H), 2.64 (m, 3H, 2.43 (s, 3H),), 2.00 (m, 1H), 1.75 (m, 1H), 1.55 (m, 2H), 1.48 (d, 3H), 1.23 (m, 4H), 1.04-0.65 (m, 5H). MS m/z 440 [M−H]⁻.

Purity: 97.6%, Rt=5.31 minutes. HPLC Zorbax SB C₁₈ (4.6×50 mm, 1.8 micron). Mobile phase: (0.05% TFA in water) in acetonitrile. 10 minute run.

### Example 56

### N-[6-(3,5-dimethyl-1,2-oxazol-4-yl)-3-oxo-4-(pentan-3-yl)-3,4-dihydropyrido[2,3-b]pyrazin-2-yl]-beta-alanine

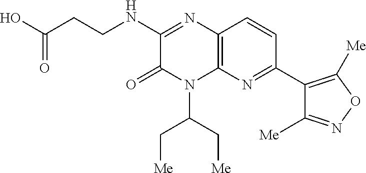 [SMILES: CCC(CC)n1c(=O)c(NCCC(=O)O)nc2ccc(-c3c(C)noc3C)nc21]

The title compound was prepared according to the method described for Example 43 using 2-chloro-6-(3,5-dimethyl-1,2-oxazol-4-yl)-4-(pentan-3-yl)pyrido[2,3-b]pyrazin-3(4H)-one (Preparation 23C) and tert-butyl-3-aminopropanoate. ¹H NMR (400 MHz, DMSO-d₆): δ ppm 7.85 (d, 1H), 7.59 (m, 1H), 7.39 (d, 1H), 5.56 (m, 1H), 3.68 (m, 2H), 2.64 (m, 1H), 2.60 (s, 3H), 2.42 (s, 3H), 2.24 (m, 2H), 1.94 (m, 2H), 0.78 (t, 6H). MS m/z 400 [M−H]⁻

Purity: 99.3%, Rt=4.18 minutes. HPLC Zorbax extended C₁₈ (4.6×50 mm, 5 micron). Mobile phase A: acetonitrile; mobile phase B: 10 mM NH₄OAc in water; 12 minute run; injection volume: 2 uL.

### Example 57

### N²-{6-(3, 5-dimethyl-1,2-oxazol-4-yl)-4-[(1S)-1-(2-methoxyphenyl)ethyl]-3-oxo-3,4-dihydropyrido[2,3-b]pyrazin-2-yl}-N-methylglycinamide

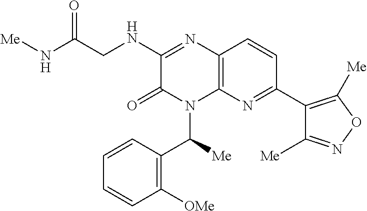 [SMILES: CNC(=O)CNc1nc2ccc(-c3c(C)noc3C)nc2n([C@@H](C)c2ccccc2OC)c1=O]

To a solution of N²-{6-chloro-4-[(1S)-1-(2-methoxyphenyl)ethyl]-3-oxo-3,4-dihydropyrido[2,3-b]pyrazin-2-yl}-N-methylglycinamide (Preparation 12N, 0.1 g, 0.25 mmol) in EtOH (5 mL), were added potassium 3,5-dimethylisoxazole-4-trifluoroborate (76 mg, 0.37 mmol) and K₂CO₃ (103.2 mg, 0.75 mmol) at room temperature. The mixture was degassed with argon for 20 min. and Pd(PPh₃)₄(28.8 mg, 0.025 mmol) was added under inert atmosphere. The resulting mixture was heated at 100° C. for 48 hours. It was cooled to ambient temperature, diluted with water and extracted with ethyl acetate. Organic layer was washed with brine, dried over Na₂SO₄ and concentrated under reduced pressure. Crude was purified by preparative HPLC to afford the title compound as an off-white solid (20 mg, 17%).

¹H NMR (400 MHz, MeOH-d₄): δ ppm 7.86 (d, 1H), 7.57 (d, 1H), 7.39 (d, 1H), 7.22-7.14 (m, 2H), 6.92 (t, 1H), 6.83 (d, 1H), 4.09 (d, 2H), 3.46 (s, 3H), 2.71 (s, 3H), 2.59 (s, 3H), 2.42 (s, 3H), 1.94 (d, 3H). MS m/z 463 [M+H]⁺

Purity: 95.4%, Rt=4.65 minutes. HPLC Zorbax SB C18 (4.6×50 mm, 1.8 micron). Mobile phase: (0.05% TFA in water) in acetonitrile. 9 minute run

### Preparation 24

### Ethyl [(6-chloro-2-{[(1S)-1-phenylpropyl]amino}pyridin-3-yl)amino](oxo)acetate

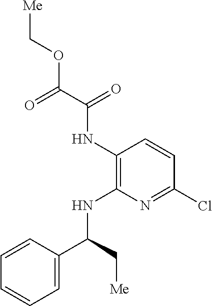 [SMILES: CCOC(=O)C(=O)Nc1ccc(Cl)nc1N[C@@H](CC)c1ccccc1]

To a solution of 6-chloro-N²-[(1S)-1-phenylpropyl]pyridine-2,3-diamine (Preparation 5, 187 g, 0.716 mol) in anhydrous THF (2 L) was added Na₂CO₃ (227 g, 2.148 mol) and ethyl chloro(oxo)acetate (112 g, 0.859 mol) at 20° C. The mixture was stirred at 20° C. for 18 hours.

The mixture was filtered through a pad of Celite. The filter cake was washed with ethyl acetate (5 L). The filtrate was concentrated in vacuo and purified by column chromatography on silica gel (petroleum ether/ethyl acetate (10:1 to 5:1)) to give the title compound as a grey solid (240 g, 92.9%). ¹H NMR (400 MHz, DMSO-d₆): δ ppm: 10.21 (s, 1H), 7.38 (d, 2H), 7.34 (d, 1H), 7.29 (t, 2H), 7.15-7.22 (m, 1H), 6.77 (d, 1H), 6.52 (d, 1H), 4.86-4.96 (m, 1H), 4.32 (q, 2H), 1.71-1.87 (m, 2H), 1.34 (t, 3H), 0.89 (t, 3H). HPLC Ultimate XB-C18.3 um, 3.0×50 mm, SN:111201514, Mobile phase:1.0% acetonitrile in water (0.1% TFA) to 5% ACN in water (0.1% TFA) in 1 minutes then from 5% acetonitrile in water (0.1% TFA) to 100% acetonitrile (0.1% TFA) in 5 minutes; hold at 100% acetonitrile (0.1% TFA) for 2 minutes then back to 1.0% acetonitrile in water (0.1% TFA) at 8.01 minutes, and hold two minutes. Flow rate: 1.2 mL/min, Retention time 4.97 minutes.

### Preparation 25

### [(6-chloro-2-{[(1S)-1-phenylpropyl]amino}pyridin-3-yl)amino](oxo)acetic acid

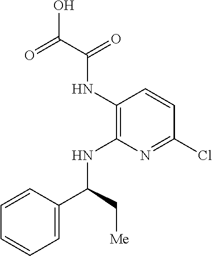 [SMILES: CC[C@H](Nc1nc(Cl)ccc1NC(=O)C(=O)O)c1ccccc1]

To a solution of ethyl [(6-chloro-2-{[(1S)-1-phenylpropyl]amino}pyridin-3-yl)amino](oxo)acetate (Preparation 24, 240 g, 0.665 mol) in THF (1.5 L) was added aq. NaOH (2M, 1 L, 2 mol) at 0° C.

Then the mixture was stirred at 0° C. for 1 hour. TLC (petroleum ether/ethyl acetate (2:1)) showed most of the starting material was consumed. The mixture was adjusted to pH 5 by adding aq. HCl (3 M) at 0° C. The mixture was extracted with ethyl acetate (3 L×2). The combined organic layers were concentrated in vacuo to give the title compound as a grey solid (220 g, 100%). ¹H NMR (400 MHz, DMSO-d₆): δ ppm: 10.05 (s, 1H), 7.51 (d, 1H), 7.40 (d, 2H), 7.28 (t, 2H), 7.14-7.21 (m, 1H), 6.91 (d, 1H), 6.52 (d, 1H), 4.87 (q, 1H), 1.81-1.90 (m, 1H), 1.75 (dt, 1H), 0.87 (t, 3H)

### Preparation 26

### 6-chloro-4-[(1S)-1-phenylpropyl]-1,4-dihydropyrido[2,3-b]pyrazine-2,3-dione

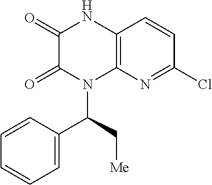 [SMILES: CC[C@@H](c1ccccc1)n1c(=O)c(=O)[nH]c2ccc(Cl)nc21]

To a solution of [(6-chloro-2-{[(1S)-1-phenylpropyl]amino}pyridin-3-yl)amino](oxo)acetic acid (Preparation 25, 220 g, 0.66 mol) in anhydrous THF (1.5 L) was added oxalyl chloride (60 mL, 0.695 mol) drop-wise slowly at 30° C. followed by the addition of DMF (3 mL). After addition, the mixture was warmed to 50° C. and stirred for 4 hours. The mixture was concentrated in vacuo and purified by column chromatography on silica gel (petroleum ether/ethyl acetate (10:1)) to give the title compound as a grey solid (220 g, 96.0%). ¹H NMR (400 MHz, DMSO-d₆): δ ppm: 12.27 (s, 1H), 7.53 (d, 1H), 7.43 (d, 2H), 7.26-7.32 (m, 3H), 7.18-7.24 (m, 1H), 6.25-6.32 (m, 1H), 2.41-2.61 (m, 2H), 0.87 (t, 3H). HPLC Chiralpak AS-H 250×4.6 mm I.D., 5 um. Mobile phase: A: CO₂ B: ethanol (0.05% DEA) Gradient: from 5% to 40% of B in 5.0 minutes and hold 40% for 2.5 minutes, then 5% of B for 2.5 minutes. Flow rate: 2.5 mL/min. Column temperature: 35° C. Retention time 5.989 minutes

### Preparation 27

### 2,6-dichloro-4-[(1S)-1-phenylpropyl]pyrido[2,3-b]pyrazin-3(4H)-one

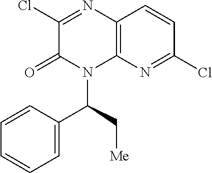 [SMILES: CC[C@@H](c1ccccc1)n1c(=O)c(Cl)nc2ccc(Cl)nc21]

To a solution of 6-chloro-4-[(1S)-1-phenylpropyl]-1,4-dihydropyrido[2,3-b]pyrazine-2,3-dione (Preparation 26, 200 g, 0.635 mol) in anhydrous THF (1.2 L) was added oxalyl chloride (200 mL, 2.318 mol) drop-wise slowly at 30° C. followed by the addition of DMF (5 mL). After addition, the mixture was warmed to 80° C. and stirred for 18 hours. TLC (DCM/MeOH (10:1)) indicated most of the starting material was consumed. The mixture was cooled to 40° C. and concentrated in vacuo. The residue was dissolved in ethyl acetate (3 L), poured into ice water (3 L). The mixture was separated. The organic layer was washed with water (1 L×2), aq. K₂CO₃ (1M, 1L), concentrated in vacuo and purified by column chromatography on silica gel (petroleum ether/ethyl acetate (5:1)) to give the title compound as a yellow oil (210 g, 74.4%). ¹H NMR (400 MHz, DMSO-d₆): δ ppm: 8.29 (d, 1H), 7.56 (d, 1H), 7.44 (s, 2H), 7.28-7.37 (m, 2H), 7.19-7.27 (m, 1H), 6.48 (br. s., 1H), 2.53-2.66 (m, 2H), 0.79-0.96 (m, 3H).

### Preparation 28

### tert-butyl N-{6-chloro-3-oxo-4-[(1S)-1-phenylpropyl]-3,4-dihydropyrido[2,3-b]pyrazin-2-yl}-beta-alaninate

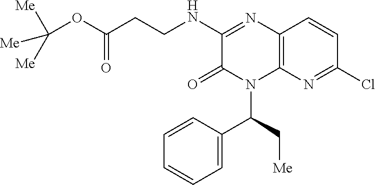 [SMILES: CC[C@@H](c1ccccc1)n1c(=O)c(NCCC(=O)OC(C)(C)C)nc2ccc(Cl)nc21]

To a solution of 2,6-dichloro-4-[(1S)-1-phenylpropyl]pyrido[2,3-b]pyrazin-3(4H)-one (Preparation 27, 210 g, 0.478 mol) in DCM (1.5 L) was added tert-butyl beta-alaninate (109 g, 0.602 mol), DIPEA (250 g, 1.937 mol) at 20° C. and stirred for 60 hours. TLC (petroleum ether/ethyl acetate (4:1)) showed most of the starting material was consumed. The mixture was washed with water (1 L). The organic layer was concentrated in vacuo and purified by column chromatography on silica gel (petroleum ether/ethyl acetate (100:1 to 10:1) to give a yellow oil.

The oil was dissolved in petroleum ether (600 mL) and stirred at 20° C. for 16 hours during which time solids precipitated. The mixture was filtered. The filter cake was washed with petroleum ether (100 mL) and dried in vacuo to give the title compound as a white solid (100 g, 43.6%). ¹H NMR (400 MHz, DMSO-d₆): δ ppm: 7.96 (br s, 1H), 7.79 (d, 1H), 7.39 (d, 2H), 7.29 (t, 3H), 7.18-7.24 (m, 1H), 6.49 (br s, 1H), 3.54-3.65 (m, 2H), 2.67 (br s, 1H), 2.58 (t, 2H), 2.42-2.49 (m, 1H), 1.35 (s, 9H), 0.82 (t, 3H).

### Preparation 29

### tert-butyl N-{6-[methyl(propanol)amino]-3-oxo-4-[(1S)-1-phenylpropyl]-3,4-dihydropyrido[2,3-b]pyrazin-2-yl}-beta-alaninate

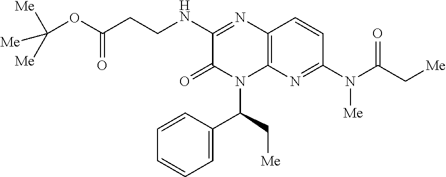 [SMILES: CCC(=O)N(C)c1ccc2nc(NCCC(=O)OC(C)(C)C)c(=O)n([C@@H](CC)c3ccccc3)c2n1]

To a mixture of tert-butyl N-{6-chloro-3-oxo-4-[(1S)-1-phenylpropyl]-3,4-dihydropyrido[2,3-b]pyrazin-2-yl}-beta-alaninate (Preparation 28, 54 g, 0.122 mol), N-methylpropanamide (12 g, 0.138 mol) and Cs₂CO₃ (48 g, 0.147 mol) in dioxane (300 mL) was degassed under vacuo and purged with N₂ two times. To the mixture was added Xantphos (8 g, 0.0138 mol) and Pd₂(dba)₃ (8 g, 8.7 mmol) at 20° C. The mixture was degassed under vacuo and purged with N₂ three times. The mixture was stirred at 100° C. for 18 hours. TLC (petroleum ether/ethyl acetate (2.5:1)) showed most of starting material was consumed. The mixture was cooled to 20° C. and combined with a previous crude batch of material synthesized following the same procedure above using 6.8 g of tert-butyl N-{6-chloro-3-oxo-4-[(1S)-1-phenylpropyl]-3,4-dihydropyrido[2,3-b]pyrazin-2-yl}-beta-alaninate. The combined mixture was filtered through a pad of Celite. The filter cake was washed with ethyl acetate (500 mL×2). The filtrate was concentrated in vacuo and purified by column chromatography on silica gel (petroleum ether/ethyl acetate (10:1 to 5:1)) to give the title compound as a brown gum (53 g, 88%). ¹H NMR (400 MHz, DMSO-d₆): δ ppm: 7.84 (br s, 2H), 7.23-7.43 (m, 5H), 7.20 (d, 1H), 6.56 (br s, 1H), 3.55-3.67 (m, 2H), 3.10-3.28 (m, 3H), 2.66 (br s, 2H), 2.59 (t, 2H), 2.41-2.48 (m, 2H), 1.36 (s, 9H), 0.87 (br s, 3H), 0.81 (t, 3H). MS m/z 494 [M+H]⁺

### Reference Compounds

Known BET inhibitors I-BET-762, I-BET151, JQ1(+) (active enantiomer) and JQ1(−) (inactive enantiomer) were purchased from Selleck Chemicals and resuspended in DMSO.

### SUMMARY OF BIOLOGICAL DATA

### BRD4 BD1 Fluorescence Polarization (FP) Binding Assay

Compound binding to BRD4 BD1 was assessed with a FP competition binding assay. His-tagged BRD4 BD1 (44-160) and PFI-411FP (Cy5-labeled FP probe) were prepared as previously described (Picaud S et al PFI-1, a highly selective protein interaction inhibitor, targeting BET Bromodomains. Cancer Res. (2013) 73: 3336-46 and Wu, J et al Design and chemoproteomic functional characterization of a chemical probe targeted to bromodomains of BET family proteins. Med. Chem. Commun. 2014, Advance Article DOI: 10.1039 respectively). All assay components were diluted in 50 mM HEPES pH 7.4 containing 0.08% bovine serum albumin (assay buffer). To start the assay, 8 μL of BRD4 BD1 was added to each well of a low volume 384-well black flat bottom microtiter plate (Corning 3820) containing 4 μl of various concentrations of test compound (each plate also contained positive and negative control wells to define the upper and lower limits of the assay signal). After addition of BRD4 BD1, the assay plate was incubated at room temperature (RT, 20° C.). After 15 minutes, 4 μL of PFI-411FP was added to each well and the assay plate was placed in the dark at RT. The final assay concentration (FAC) of PFI-411FP was 2 nM, the FAC of BRD4 BD1 was 40 nM, the FAC of test compound ranged from 120 to 0.0012 μM and the FAC of DMSO was 0.4%. After 60 min, polarization values were measured with an Envision 2103 multilabel reader (Perkin Elmer) using a Cy5 dual enhanced mirror and excitation at 620 nm and emission at 688 nm. The percent (%) effect was calculated for each concentration of test compound and was relative to the amount of polarization signal produced by the positive and negative control wells contained within each assay plate. The concentrations and % effect values for test compounds were plotted versus each other with a proprietary curve fitting program using a four-parameter logistic dose response equation and the concentration of compound required for 50% effect (IC₅₀) was determined. The Ki values of competitive inhibitors were calculated using the equation described by Nikolovska-Coleska et al. (Development and optimization of a binding assay for the XIAP BIR3 domain using fluorescence polarization. Analytical Biochemistry (2004) 332: 261-273).

### IL-6 Human Whole Blood (HWB) Assay

Compound effect on IL-6 production was determined using lipopolysaccharide (LPS)-stimulated human whole blood and an HTRF (Homogeneous Time-Resolved Fluorescence) IL-6 detection kit (Human IL6 HTRF Assay CisBio 62IL6PEC). HWB was collected by venous puncture from healthy donors, transferred to a 50 ml polypropylene tube containing sodium heparin (14.3 units per mL of HWB) and then placed in a 37° C. water bath until use. To start the assay, 80 μL of heparin-treated HWB containing 1 ng/ml of LPS (equivalent to LPS ˜EC₈₀ with regard to stimulation of IL-6 production in HWB under these assay conditions) was added to each well of a 384-well, sterile, endotoxin-free, polypropylene plate (Greiner 781281) containing 160 nL of various concentrations of test compound (each plate also contained positive and negative control wells to define the upper and lower limits of the assay signal). The plate was then placed in a thermoshaker incubator set to 370C (Boekel 270440). The final assay concentration (FAC) of test compound ranged from 60 to 0.0006 μM and the FAC of DMSO was 0.2%. After 4 hours, the assay plate was removed from the incubator and centrifuged at 700×g for 10 minutes. 5 μL of the resulting upper plasma layer was removed from each well and diluted 1:2 with 1×Dulbecco's phosphate buffered saline (DPBS). 5 μL of this diluted plasma was then transferred to a white 384-well low volume assay plate (Greiner 784080) containing 5 μL of combined anti-IL-6-Cryptate and anti-IL-6-XL665 detection antibodies (per manufacturer's protocol). The plate was then sealed with a Top Seal (Perkin Elmer 6005185) and incubated at room temperature (RT, 20° C.). After 16-18 hrs (overnight), the HTRF assay signal was read on an Envision 2103 plate reader using a Lance Delfia Dual/Bias mirror TRF laser excitation and emission was measured at 665 nM and 615 nm respectively. The percent (%) effect was calculated for each concentration of test compound and was relative to the amount of HTRF signal produced by the positive and negative control wells contained within each assay plate. The concentrations and % effect values for test compounds were plotted versus each other with a proprietary curve fitting program using a four-parameter logistic dose response equation and the concentration of compound required for 50% effect (IC₅₀) was determined.

### IC₅₀ Data

Certain compounds of the invention were tested in the Fluorescence Polarization assay and/or the IL-6 human whole blood assay described herein. The IC₅₀ data obtained is provided in the table below.

| | BRD4 | LPS-induced IL-6 |
|---|---|---|
| Example | binding | in whole blood |
| number | IC₅₀ (nM) | IC₅₀ (nM) |
| 1 | 730.3 | 38397.1 |
| 2 | 2710.3 | — |
| 3 | 117.3 | 2045.6 |
| 4 | 955.6 | 14125.7 |
| 5 | 652.6 | 1577.9 |
| 6 | 155.4 | 1418.8 |
| 7 | 129.6 | 2391.4 |
| 8 | 122.1 | 1804.0 |
| 9 | 150.4 | 3827.0 |
| 10 | 62.2 | 529.9 |
| 11 | 251.5 | 2683.4 |
| 12 | 804.9 | 18147.9 |
| 13 | 111.3 | 845.5 |
| 14 | 222.8 | 4798.8 |
| 15 | 10166.3 | — |
| 16 | 3884.6 | — |
| 17 | 267.9 | 2432.6 |
| 18 | 4191.1 | — |
| 19 | 4031.3 | — |
| 20 | 1074.4 | 1460.0 |
| 21 | 207.3 | 743.0 |
| 22 | 912.7 | 22547.1 |
| 23 | 158.9 | 2298.2 |
| 24 | 1581.3 | — |
| 25 | 6372.7 | — |
| 26 | 7592.3 | — |
| 27 | 1500.6 | — |
| 28 | 148.9 | 366.1 |
| 29 | 101.9 | 238.7 |
| 30 | 348.0 | 721.6 |
| 31 | 109.9 | 163.5 |
| 32 | 64.8 | 265.8 |
| 33 | 362.9 | 451.6 |
| 34 | 167.0 | 426.1 |
| 35 | 194.7 | 746.5 |
| 36 | 2484.8 | — |
| 37 | 474.0 | — |
| 38 | 532.3 | — |
| 39 | 123.2 | 822.7 |
| 40 | 225.2 | 1390.4 |
| 41 | 585.1 | 35527.6 |
| 42 | 145.6 | 6156.6 |
| 43 | 576.3 | 8350.3 |
| 44 | 89.8 | 2559.7 |
| 45 | 324.8 | 8441.4 |
| 46 | 355.3 | 6215.2 |
| 47 | 194.0 | 6163.2 |
| 48 | 464.7 | 15069.1 |
| 49 | 4870.6 | — |
| 50 | 235.1 | 31994.0 |
| 51 | 294.4 | 63730.6 |
| 52 | 62.7 | 1087.4 |
| 53 | 124.6 | 9257.2 |
| 54 | 99.4 | 2638.7 |
| 55 | 130.5 | 11440.7 |
| 56 | 345.9 | 11924.3 |
| 57 | 158.5 | 919.3 |

### MM1.S and OPM-2 Cell Proliferation Assays

MM1.S (dexamethasone sensitive) and OPM-2 cells were purchased from ATCC and maintained in RPMI-1640 medium with 10% fetal bovine serum at 37 degrees, 5% CO2. Cells were seeded at 10,000 cells/well in 100 uL medium. The following day compounds or control vehicle (DMSO) were added at indicated concentrations in 10 ul volume. Compound treated OPM-2 cells and MM1.S cell were analyzed for cell density at 72h and 96h, respectively, using CellTiter-Glo® (CTG) reagent (Promega). The CTG assay measures the amount of ATP present, which indicates the number of viable cells in culture.

Results:

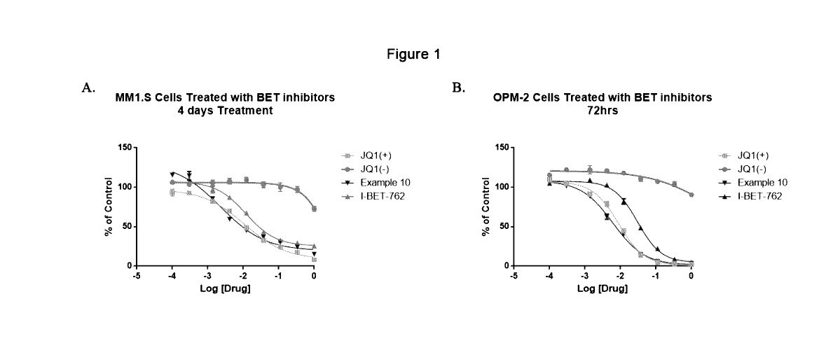

Compounds JQ1(+), IBET-762, Example 10 and Example 47 inhibited proliferation of MM1.S cells (FIG. 1.A) and OPM-2 cells (FIG. 1.B) in a dose dependent manner. JQ1(−) was used as a positive control. IC₅₀ values (nM) for test compounds are provided in the Table below.

| Cell | JQ1(+) | JQ1(−) | IBET-762 | Example 10 | Example 47 |
|---|---|---|---|---|---|
| Line | IC₅₀ (nM) | IC₅₀ (nM) | IC₅₀ (nM) | IC₅₀ (nM) | IC₅₀ (nM) |
| MM1.S | 11 | >1000 | 12 | 2 | 39 |
| OPM-2 | 9 | >1000 | 30 | 6 | 160 |

### Biomarker Analysis in MM1.S Cells

MM1.S cells (1,000,000 cells) were seeded in 10 cm² dishes in 10 mL medium. On the following day cells were treated with indicated compounds for 0, 2, 4, 6, 8, 16, and 24 hours. Cells were collected, washed in PBS, and split for western blot and RNA analysis. For western blot, cells were lysed in RIPA buffer and 30 ug protein was loaded on a 4-12% bis-tris gel under denaturing conditions and transferred to nitrocellulose. Blots were probed with primary antibodies for c-MYC (Cell Signaling #9402) and GAPDH (Cell Signaling #2118) diluted at 1:1000 and then secondary antibodies anti-mouse 680 (LiCor) and anti-rabbit 800 (Licor) and imaged on a LiCor reader. For gene expression analysis, total RNA was prepared using Qiagen total RNA kit (Qiagen) and cDNA was prepared from 100 ng RNA (high capacity cDNA kit; Applied Biosystems). Relative gene expression was analyzed on an Applied Biosystem 7900 qPCR thermocycler using gene expression assays for MYC (Hs00153408_m1), MYB (Hs00920556_m1), and GAPDH (Hs02758991_g1). Relative gene expression is calculated relative to DMSO and normalized to GAPDH expression.

Results:

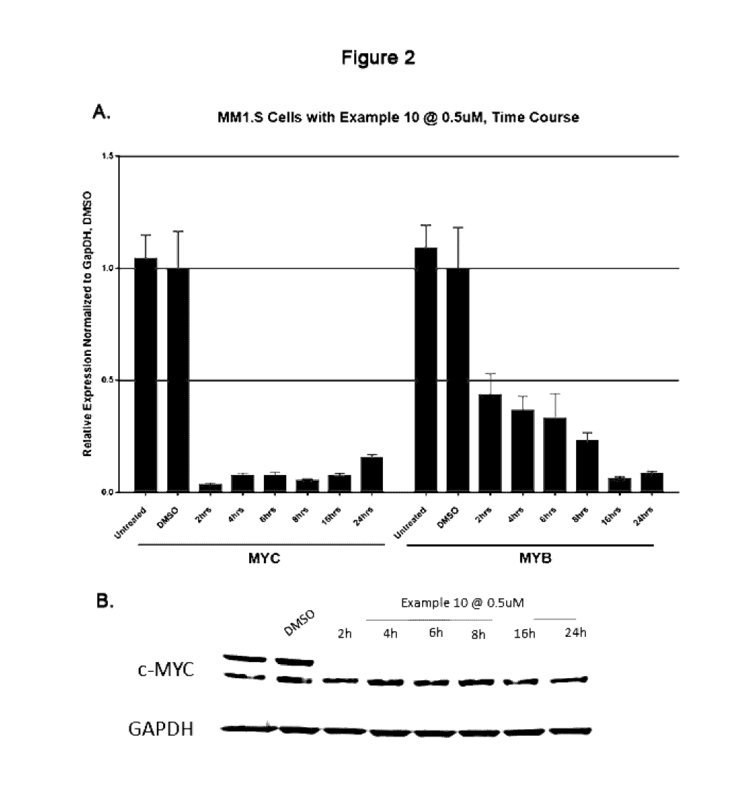

Treatment of MM1.S cells with Example 10 at a concentration of 0.5 uM strongly down-regulated the expression of c-MYC mRNA relative to GAPDH as measured by RT-PCR time-course. Down-regulation occurred rapidly and was sustained at 24 hours (FIG. 2.A). Relative expression of MYB mRNA was also down-regulated (FIG. 2.A). A Western blot showed that relative expression of c-MYC protein is also down-regulated at 2, 4, 6, 8, 16 and 24 hours (FIG. 2.B).

NMC HCC₂₄₂₉ Cell Proliferation Assay
HCC₂₄₂₉ cells were plated into a 96-well plate (10,000 cells/well). Compounds or control vehicle (DMSO) were added at indicated concentrations. Compound-treated HCC₂₄₂₉ cells were analyzed for cell density at 96h using CellTiter-Glo® (CTG) reagent (Promega).

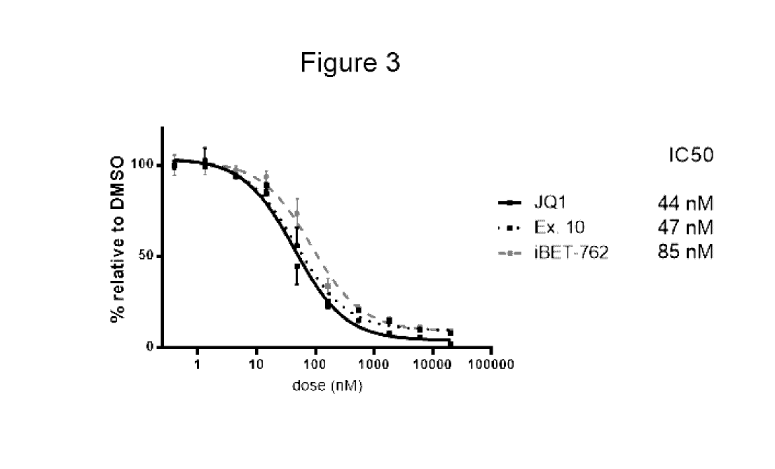

Results:
Example 10, JQ1(+) and IBET-762 inhibited cell proliferation in HCC₂₄₂₉ cells with IC₅₀ values of 47 nM, 44 nM and 85 nM, respectively (FIG. 3).

### Biomarker Analysis in HCC2429 Cells

HCC2429 cells were plated into a 6-well plate (250,000 cells/well). Cells were treated with compounds or control vehicle (DMSO) at the indicated concentrations for 72 hrs. Cells were collected, washed in PBS, and split for western blot and RNA analysis.

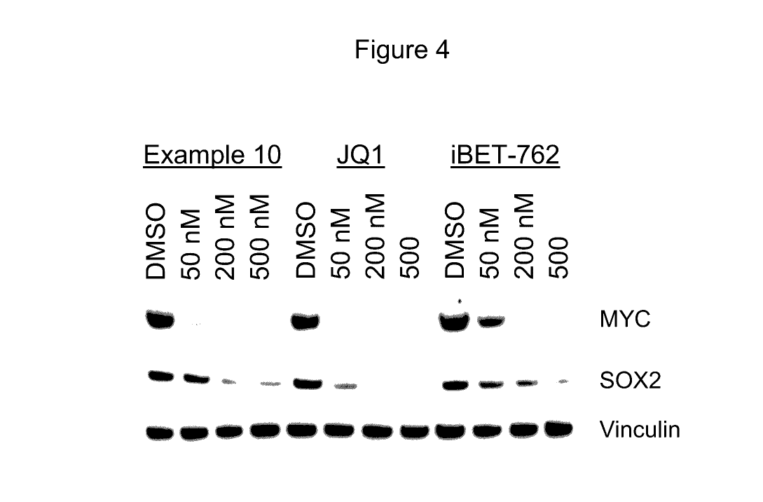

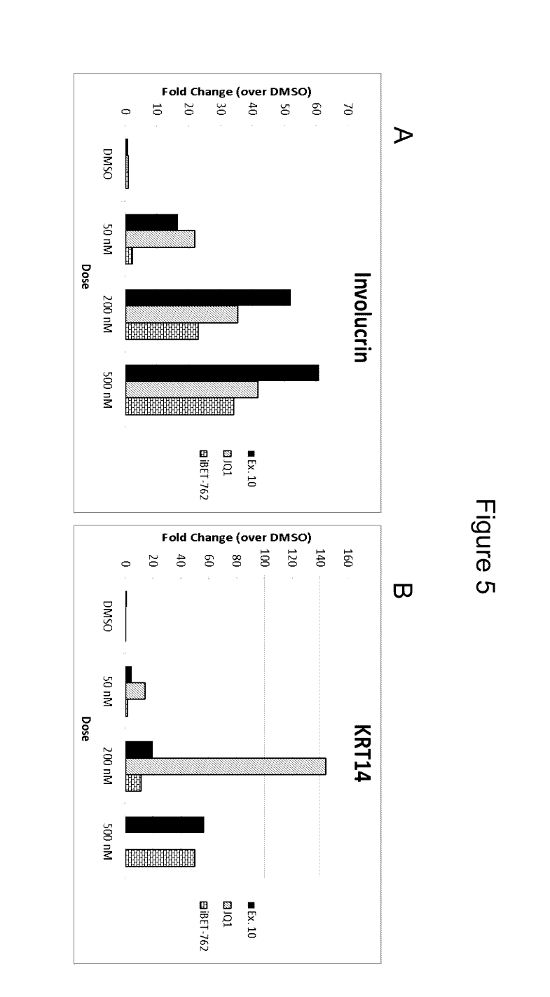

Results:
Modest down-regulation of MYC mRNA was observed (20-50%) in compound treated cells, but no down-regulation of SOX2 mRNA was observed (data not shown). Western blot showed that MYC and SOX2 protein levels were down-regulated by treatment with Example 10, JQ1(+) and IBET-762 for 72 hrs in a concentration-dependent manner (FIG. 4). Treatment with BET inhibitors was also observed to induce squamous cell differentiation in vitro as measured by RT-PCR after 72 hours. Treatment with Example 10, JQ1(+) and I-BET-762 at concentrations of 50 nM, 200 nM and 500 nM increased mRNA expression of involucrin in a dose dependent manner (FIG. 5.A). Keratin 14 (KRT14) mRNA expression was also induced in a dose dependent manner by treatment with BET inhibitors (FIG. 5.B). Altered cellular morphology was observed, suggesting a differentiation phenotype (data not shown).

Castrate-Resistant Prostate Cancer (CRPC) Cell Proliferation Assay
Cells were seeded into 96-well plates at 2,000-10,000 cells/well in 100 uL medium. Four AR(+) cell lines (LnCaP, C₄-2 (parental), C₄-2AR-WT and C₄-2AR—F876L) and one AR(−) cell line (DU-145) were studied. Compound treated cells were analyzed for cell density at 96h, using CellTiter-Glo® (CTG) reagent (Promega). Assay conditions were similar to those described by Asangani et al. (Nature (2014), 510:278-282).
Results:
BET bromodomain inhibition preferentially inhibited the growth of AR+CRPC cells in the CTG assay. Example 10 and IBET-151 BET had submicromolar IC₅₀ values in the AR(+) cell lines. The inhibitors maintained potency in C₄-2 cells engineered to express wild type (WT) AR receptor (C₄-2-AR-WT), as well as cells engineered to express the F876L mutation (C₄-2-AR-F876L) that confers resistance to androgen receptor (AR) antagonists. The AR antagonist MDV3100 showed weaker activity on cell proliferation in this short-term assay, but showed signs of differentiation between AR(+) and AR(−) cell lines. Treatment with BET inhibitors inhibited the expression of the target gene MYC in AR(+) cell lines. Down-regulation of MYC mRNA was observed in LNCaP, C₄-2, and the engineered cell lines C₄-2 AR and C₄-2 AR F876L, but no effect was observed in the AR(−) cell line DU145, which has low levels of endogenous MYC (data not shown). IC₅₀ values (LM) and maximal % inhibition are provided in the Table below.

| Cell | | | | | | | | |
|---|---|---|---|---|---|---|---|---|
| Line | | | | | | | MDV3100 (AR | |
| AR | Example 47 | | Example 10 | | i-BET151 | | antagonist) | |
| status: | IC₅₀ | Max % | IC₅₀ | Max % | IC₅₀ | Max % | IC₅₀ | Max % |
| (+/−) | (μM) | Inh | (μM) | Inh | (μM) | Inh | (μM) | Inh |
| LnCap (+) | >5 | 62 | 0.32 | 69 | 0.40 | 78 | 0.32 | 43 |
| C4-2 (+) | >5 | 80 | 0.50 | 87 | 0.49 | 90 | 1.06 | 33 |
| C4-2AR- | >5 | 82 | 0.50 | 85 | 0.43 | 92 | 0.84 | 39 |
| WT85 (+) | | | | | | | | |
| C4-2AR- | >5 | 81 | 0.56 | 85 | 0.47 | 90 | >10 | 44 |
| F876L (+) | | | | | | | | |
| DU 145 (−) | >5 | 43 | 1.88 | 55 | 1.13 | 74 | >10 | 23 |

Variations, modifications, and other implementations of what is described herein will occur to those skilled in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced therein.

Each of the printed publications, including but not limited to patents, patent applications, books, technical papers, trade publications and journal articles described or referenced in this specification are herein incorporated by reference in their entirety and for all purposes.

1. A compound of Formula I: 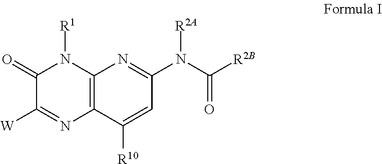
or a pharmaceutically acceptable salt thereof, wherein
R¹ is selected from the group consisting of:
(iv) —C₃-C₇cycloalkyl optionally substituted with one, two, three or four E;
(v) 4 to 7 membered heterocyclyl optionally substituted with one, two, three or four E, which said 4 to 7 membered heterocyclyl comprises one or two heteroatoms independently selected for each occurrence from the group consisting of N, O and S; and
(vi) 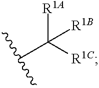
R^{1A} is selected from the group consisting of
(vi) —C₁-C₆alkyl optionally substituted with one, two, three, four, five or six E;
(vii) —C₃-C₇cycloalkyl optionally substituted with one, two, three, four or five E;
(viii) phenyl optionally substituted with one, two, three, four or five E;
(ix) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
(x) 5 to 6 membered heteroaryl optionally substituted with one, two, three, four or five E, which said 5 to 6 membered heteroaryl comprises one, two or three heteroatoms independently selected for each occurrence from the group consisting of N, O and S;
R^{1B} is selected from the group consisting of
(iii) —H; and
(iv) —C₁-C₆alkyl optionally substituted with one, two, three, four, five or six E;
R^{1C} is selected from the group consisting of
(vi) —H;
(vii) —CH₃ optionally substituted with one, two, or three J;
(viii) —CH₂CH₃ optionally substituted with one, two, three, four or five J;
(ix) —CH₂CH₂CH₃ optionally substituted with one, two, three, four, five, six or seven J; and
(x) —CH(CH₃)₂ optionally substituted with one, two, three, four, five, six or seven J;
R^{2A} is selected from the group consisting of
(v) —H;
(vi) —CH₃ optionally substituted with one, two or three J;
(vii) —CH₂CH₃ optionally substituted with one, two, three, four, or five J; and
(viii) cyclopropyl optionally substituted with one, two, three, four or five J;
R^{2B} is selected from the group consisting of
(viii) —C₁-C₆alkyl optionally substituted with one, two, three or four G;
(ix) —OC₁-C₆alkyl optionally substituted with one, two, three or four G;
(x) —NH₂;
(xi) —NH(C₁-C₆alkyl), which C₁-C₆alkyl is optionally substituted with one, two, three or four G;
(xii) —N(C₁-C₆alkyl)₂, which C₁-C₆alkyl is, independently for each occurrence, optionally substituted with one, two, three or four G;
(xiii) C₃-C₅cycloalkyl optionally substituted with one, two, three or four G; and
(xiv) 4 to 7 membered heterocyclyl optionally substituted with one, two, three or four G, which said 4 to 7 membered heterocyclyl comprises one or two heteroatoms independently selected for each occurrence from the group consisting of N, O and S;
W is selected from the group consisting of:
(vii) 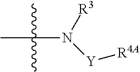
(viii) 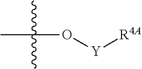
(ix) 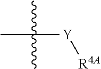
(x) 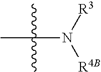
(xi) 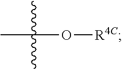
and
(xii) 4 to 7 membered heterocyclyl optionally substituted with one, two, three or four G, which said 4 to 7 membered heterocyclyl comprises one, two, three or four heteroatoms independently selected for each occurrence from the group consisting of N, O and S;
Y is selected from the group consisting of:
(v) —CH₂— optionally substituted with one or two J;
(vi) —(CH₂)₂— optionally substituted with one, two, three or four J;
(vii) —(CH₂)₃— optionally substituted with one, two, three, four, five or six J; and
(viii) —(CH₂)₄— optionally substituted with one, two, three, four, five, six, seven or eight J;
R³ is selected from the group consisting of:
(vi) —H;
(vii) —CH₃ optionally substituted with one, two, or three J;
(viii) —CH₂CH₃ optionally substituted with one, two, three, four or five J;
(ix) —CH₂CH₂CH₃ optionally substituted with one, two, three, four, five, six or seven J; and
(x) —CH(CH₃)₂ optionally substituted with one, two, three, four, five, six or seven J;
R^{4A} is selected from the group consisting of
(xxiii) —H;
(xxiv) —C₁-C₆alkyl optionally substituted with one, two, three or four G;
(xxv) —CO₂H;
(xxvi) —C(O)C₁-C₆alkyl optionally substituted with one, two, three or four G;
(xxvii) —C(O)OC₁-C₆alkyl optionally substituted with one, two, three or four G;
(xxviii) —C(O)NH₂;
(xxix) —C(O)NH(C₁-C₆alkyl) optionally substituted with one, two, three or four G;
(xxx) —C(O)N(C₁-C₆alkyl)₂ optionally substituted with one, two, three or four G;
(xxxi) —C(O)NHSO₂C₁-C₃alkyl optionally substituted with one, two, three or four G;
(xxxii) —NH(C₁-C₃alkyl) optionally substituted with one, two, three or four G;
(xxxiii) —N(C₁-C₃alkyl)₂ optionally substituted with one, two, three or four G;
(xxxiv) —NHC(O)C₁-C₃alkyl optionally substituted with one, two, three or four G;
(xxxv) —N(C₁-C₃alkyl)C(O)C₁-C₃alkyl optionally substituted with one, two, three or four G;
(xxxvi) —NHSO₂C₁-C₃alkyl optionally substituted with one, two, three or four G;
(xxxvii) —N(C₁-C₃alkyl)SO₂C₁-C₃alkyl optionally substituted with one, two, three or four G;
(xxxviii) —SO₂NH₂;
(xxxix) —SO₂NH(C₁-C₃alkyl) optionally substituted with one, two, three or four G;
(xl) —SO₂N(C₁-C₃alkyl)₂ optionally substituted with one, two, three or four G;
(xli) —C₃-C₇cycloalkyl optionally substituted with one, two, three or four G;
(xlii) phenyl optionally substituted with one, two, three or four G;
(xliii) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
(xliv) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4B} is selected from the group consisting of
(xiii) —H;
(xiv) —C₁-C₆alkyl optionally substituted with one, two, three or four G;
(xv) —C(O)C₁-C₆alkyl optionally substituted with one, two, three or four G;
(xvi) —C(O)OC₁-C₆alkyl optionally substituted with one, two, three or four G;
(xvii) —C(O)NH₂;
(xviii) —C(O)NH(C₁-C₆alkyl) optionally substituted with one, two, three or four G;
(xix) —C(O)N(C₁-C₆alkyl)₂ optionally substituted with one, two, three or four G;
(xx) —C(O)NHSO₂C₁-C₃alkyl optionally substituted with one, two, three or four G;
(xxi) —C₃-C₇cycloalkyl optionally substituted with one, two, three or four G;
(xxii) phenyl optionally substituted with one, two, three or four G;
(xxiii) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
(xxiv) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4C} is selected from the group consisting of
(vii) —H;
(viii) —C₁-C₆alkyl optionally substituted with one, two, three or four G;
(ix) —C₃-C₇cycloalkyl optionally substituted with one, two, three or four G;
(x) phenyl optionally substituted with one, two, three or four G;
(xi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
(xii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R¹⁰ is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂;
E is independently selected for each occurrence from the group consisting of:
(xxix) —OH;
(xxx) —CN;
(xxxi) —CO₂H;
(xxxii) —C(O)H;
(xxxiii) halo;
(xxxiv) —C₁-C₃alkyl optionally substituted with one, two, three or four J;
(xxxv) —C₁-C₃alkylCO₂H which —C₁-C₃alkyl is optionally substituted with one, two, three or four J;
(xxxvi) —C₃-C₇cycloalkyl optionally substituted with one, two, three, four, five or six J;
(xxxvii) —C₁-C₃alkylC₃-C₆cycloalkyl optionally substituted with one, two, three, four, five or six J;
(xxxviii) —OC₁-C₃alkyl, optionally substituted with one, two, three or four J;
(xxxix) —OC₃-C₇cycloalkyl optionally substituted with one, two, three, four, five or six J;
(xl) —OC₁-C₃alkylC₃-C₇cycloalkyl optionally substituted with one, two, three, four, five or six J;
(xli) —SC₁-C₃alkyl, optionally substituted with one, two, three or four J;
(xlii) —SC₃-C₇cycloalkyl optionally substituted with one, two, three, four, five or six J;
(xliii) —SC₁-C₃alkylC₃-C₇cycloalkyl optionally substituted with one, two, three, four, five or six J;
(xliv) —C(O)C₁-C₃alkyl, optionally substituted with one, two, three or four J;
(xlv) —C(O)OC₁-C₃alkyl, optionally substituted with one, two, three or four J;
(xlvi) —NH₂;
(xlvii) —NH(C₁-C₃alkyl) optionally substituted with one, two, three or four J;
(xlviii) —N(C₁-C₃alkyl)₂ which —C₁-C₃alkyl is, independently for each occurrence, optionally substituted with one, two, three or four J;
(xlix) —C(O)NH₂;
(l) —C(O)NHC₁-C₃alkyl, optionally substituted with one, two, three or four J;
(li) —C(O)N(C₁-C₃alkyl)₂, which —C₁-C₃alkyl is, independently for each occurrence, optionally substituted with one, two, three or four J;
(lii) —NHC(O)C₁-C₃alkyl, optionally substituted with one, two, three or four J;
(liii) —SO₂(C₁-C₃alkyl), optionally substituted with one, two, three or four J;
(liv) —SO₂NH(C₁-C₃alkyl), optionally substituted with one, two, three or four J;
(lv) —NHSO₂(C₁-C₃alkyl), optionally substituted with one, two, three or four J; and
(lvi) phenyl optionally substituted with one, two, three, or four J;
G is independently selected for each occurrence from the group consisting of
(xxv) —OH;
(xxvi) —CN;
(xxvii) —CO₂H;
(xxviii) —C(O)H;
(xxix) halo;
(xxx) —C₁-C₃alkyl, optionally substituted with one, two, three or four J;
(xxxi) —C₁-C₃alkylCO₂H, which —C₁-C₃alkyl is optionally substituted with one, two, three or four J;
(xxxii) —C₁-C₃alkylC₃-C₆cycloalkyl optionally substituted with one, two, three, four, five or six J;
(xxxiii) —OC₁-C₃alkyl, optionally substituted with one, two, three or four J;
(xxxiv) —OC₁-C₃alkylC₃-C₆cycloalkyl optionally substituted with one, two, three, four, five or six J;
(xxxv) —SC₁-C₃alkyl, optionally substituted with one, two, three or four J;
(xxxvi) —SC₁-C₃alkylC₃-C₆cycloalkyl optionally substituted with one, two, three, four, five or six J;
(xxxvii) —C(O)C₁-C₃alkyl, optionally substituted with one, two, three or four J;
(xxxviii) —C(O)OC₁-C₃alkyl, optionally substituted with one, two, three or four J;
(xxxix) —NH₂;
(xl) —NH(C₁-C₃alkyl), optionally substituted with one, two, three or four J;
(xli) —N(C₁-C₃alkyl)₂, which —C₁-C₃alkyl is, independently for each occurrence, optionally substituted with one, two, three or four J;
(xlii) —C(O)NH₂;
(xliii) —C(O)NHC₁-C₃alkyl, optionally substituted with one, two, three or four J;
(xliv) —C(O)N(C₁-C₃alkyl)₂, which —C₁-C₃alkyl is, independently for each occurrence, optionally substituted with one, two, three or four J;
(xlv) —NHC(O)C₁-C₃alkyl, optionally substituted with one, two, three or four J;
(xlvi) —SO₂(C₁-C₃alkyl), optionally substituted with one, two, three or four J;
(xlvii) —SO₂NH(C₁-C₃alkyl), optionally substituted with one, two, three or four J; and
(xlviii) —NHSO₂(C₁-C₃alkyl) optionally substituted with one, two, three or four J; and
J is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂.

2. A compound of Formula (I), according to claim 1, or a pharmaceutically acceptable salt thereof, which is a compound of Formula (I′)

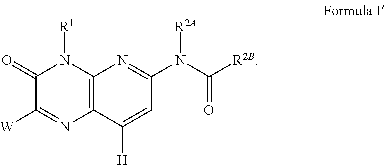

3. A compound of Formula (I), according to claim 1, or a pharmaceutically acceptable salt thereof, wherein R¹ is —C₃-C₇cycloalkyl optionally substituted with one, two, three or four E.

4. A compound of Formula (I), according to claim 3, or a pharmaceutically acceptable salt thereof, wherein R¹ is selected from the group consisting of cyclopropyl, cyclobutyl, cyclopentyl and cyclohexyl, which R¹ is optionally substituted with one, two, three or four E.

5. A compound of Formula (I), according to claim 1, or a pharmaceutically acceptable salt thereof, wherein R¹ is 4 to 7 membered heterocyclyl, which said 4 to 7 membered heterocyclyl comprises one or two heteroatoms independently selected for each occurrence from the group consisting of N, O and S, which R¹ is optionally substituted with one, two, three or four E.

6. A compound of Formula (I), according to claim 5, or a pharmaceutically acceptable salt thereof, wherein R¹ is selected from the group consisting of tetrahydrofuranyl and tetrahydropyranyl which R¹ is optionally substituted with one, two, three or four E.

7. A compound of Formula (I), according to claim 1, or a pharmaceutically acceptable salt thereof, wherein R¹ is

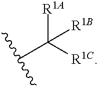

8. A compound of Formula (I), according to claim 7, or a pharmaceutically acceptable salt thereof, wherein R^{1A} is selected from the group consisting of methyl, ethyl, n-propyl, cyclohexyl, phenyl, pyridyl, pyridazinyl and pyrimidinyl which R^{1A} is optionally substituted with E as defined for a compound of Formula (I).

9. A compound of Formula (I), according to claim 8, or a pharmaceutically acceptable salt thereof, wherein R^{1A} is selected from the group consisting of methyl, phenyl, and pyridyl, which R^{1A} is optionally substituted with E as defined for a compound of Formula (I).

10. A compound of Formula (I), according to claim 7, or a pharmaceutically acceptable salt thereof, wherein R^{1A} is selected from the group consisting of —CH₂OCH₃; phenyl; methoxyphenyl; and pyridyl.

11. A compound of Formula (I) according to claim 7, or a pharmaceutically acceptable salt thereof, wherein R^{1B} is selected from the group consisting of —H, methyl, ethyl, n-propyl, and i-propyl, which R^{1B} is optionally substituted with E as defined for a compound of Formula (I).

12. A compound of Formula (I) according to claim 11, or a pharmaceutically acceptable salt thereof, wherein R^{1B} is selected from the group consisting of —H, methyl, ethyl, n-propyl, and i-propyl which R^{1B} is optionally substituted with E as defined for a compound of Formula (I), which E is independently selected for each occurrence from the group consisting of —OH; —F; —Cl; —CH₃; —OCH₃; and —CF₃.

13. A compound of Formula (I) according to claim 11, or a pharmaceutically acceptable salt thereof, wherein R^{1B} is selected from the group consisting of —H, methyl, ethyl, n-propyl, i-propyl, and —CH₂OCH₃.

14. A compound of Formula (I) according to claim 7, or a pharmaceutically acceptable salt thereof, wherein R^{1C} is selected from the group consisting of —CH₃; and —H.

15. A compound of Formula (I) according to claim 14, or a pharmaceutically acceptable salt thereof, wherein R^{1C} is —H.

16. A compound of Formula (I), according to claim 1, or a pharmaceutically acceptable salt thereof, which is a compound of Formula (Ia)

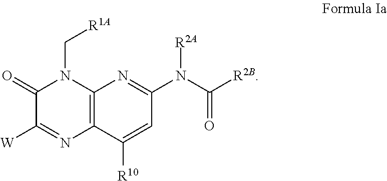

17. A compound of Formula (Ia), according to claim 16, or a pharmaceutically acceptable salt thereof, which is a compound of Formula (Ia′)

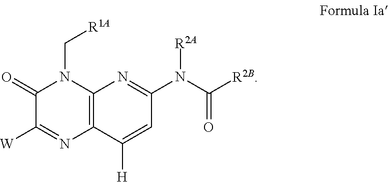

18. A compound of Formula (I), according to claim 1, or a pharmaceutically acceptable salt thereof, which is a compound of Formula (Ib)

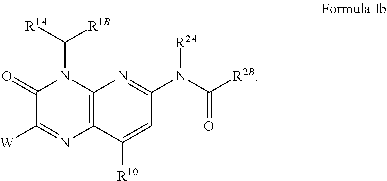

19. A compound of Formula (Ib), according to claim 18, or a pharmaceutically acceptable salt thereof, which is a compound of Formula (Ib′)

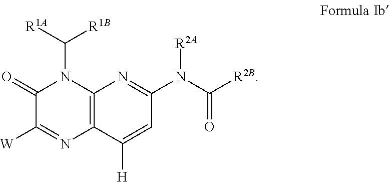

20. A compound of Formula (I) according to claim 1, selected from the group consisting of:
N-{6-[acetyl(methyl)amino]-4-[(1S)-1-(2-methoxyphenyl)ethyl]-3-oxo-3,4-dihydropyrido[2,3-b]pyrazin-2-yl}-beta-alanine;
N-{6-[acetyl(ethyl)amino]-4-[(1S)-1-(2-methoxyphenyl)ethyl]-3-oxo-3,4-dihydropyrido[2,3-b]pyrazin-2-yl}-beta-alanine;
N-{6-[acetyl(methyl)amino]-3-oxo-4-[(1S)-1-phenylpropyl]-3,4-dihydropyrido[2,3-b]pyrazin-2-yl}-beta-alanine;
N-{6-[(hydroxyacetyl)(methyl)amino]-3-oxo-4-[(1S)-1-phenylpropyl]-3,4-dihydropyrido[2, 3-b]pyrazin-2-yl}-beta-alanine;
N-{4-[(1S)-1-(2-methoxyphenyl)ethyl]-2-(methylamino)-3-oxo-3,4-dihydropyrido[2,3-b]pyrazin-6-yl}-N-methylacetamide;
N-{6-[methyl(2-methylpropanoyl)amino]-3-oxo-4-[(1S)-1-phenylpropyl]-3,4-dihydropyrido[2, 3-b]pyrazin-2-yl}-beta-alanine;
N-{6-[butanoyl(methyl)amino]-3-oxo-4-[(1S)-1-phenylpropyl]-3,4-dihydropyrido[2,3-b]pyrazin-2-yl}-beta-alanine;
N-{6-[(cyclobutylcarbonyl)(methyl)amino]-3-oxo-4-[(1S)-1-phenylpropyl]-3,4-dihydropyrido[2,3-b]pyrazin-2-yl}-beta-alanine;
N-{6-[methyl(methylcarbamoyl)amino]-3-oxo-4-[(1S)-1-phenylpropyl]-3,4-dihydropyrido[2,3-b]pyrazin-2-yl}-beta-alanine;
N-{6-[methyl(propanoyl)amino]-3-oxo-4-[(1S)-1-phenylpropyl]-3,4-dihydropyrido[2,3-b]pyrazin-2-yl}-beta-alanine;
N-{6-[methyl(propanoyl)amino]-3-oxo-4-[(1S)-1-(pyridin-2-yl)propyl]-3,4-dihydropyrido[2,3-b]pyrazin-2-yl}-beta-alanine;
N-{6-[methyl(propanoyl)amino]-3-oxo-4-(1-phenylcyclobutyl)-3,4-dihydropyrido[2,3-b]pyrazin-2-yl}-beta-alanine;
N-{4-(2,5-diethylcyclopentyl)-6-[methyl(propanoyl)amino]-3-oxo-3,4-dihydropyrido[2,3-b]pyrazin-2-yl}-beta-alanine;
N-{6-[acetyl(methyl)amino]-4-[(1R)-2-methoxy-1-phenylethyl]-3-oxo-3,4-dihydropyrido[2,3-b]pyrazin-2-yl}-beta-alanine;
N-{6-[methyl(propanoyl)amino]-3-oxo-4-[(3S,4S)-4-phenyltetrahydrofuran-3-yl]-3,4-dihydropyrido[2,3-b]pyrazin-2-yl}-beta-alanine;
N-{6-[acetyl(methyl)amino]-4-[(2R)-1-methoxybutan-2-yl]-3-oxo-3,4-dihydropyrido[2,3-b]pyrazin-2-yl}-beta-alanine;
N-{4-[(2R)-1-methoxypentan-2-yl]-6-[methyl(propanoyl)amino]-3-oxo-3,4-dihydropyrido[2, 3-b]pyrazin-2-yl}-beta-alanine;
N-(4-[(2R)-1-methoxybutan-2-yl]-2-{[2-(methylamino)-2-oxoethyl]amino}-3-oxo-3,4-dihydropyrido[2,3-b]pyrazin-6-yl)-N-methylpropanamide;
N-[4-(1,3-dimethoxypropan-2-yl)-2-{[2-(methylamino)-2-oxoethyl]amino}-3-oxo-3,4-dihydropyrido[2,3-b]pyrazin-6-yl]-N-methylpropanamide;
N-[4-(1,3-dimethoxypropan-2-yl)-2-(methylamino)-3-oxo-3,4-dihydropyrido[2,3-b]pyrazin-6-yl]-N-methylpropanamide;
N-{2-(acetylamino)-3-oxo-4-[(1S)-1-phenylpropyl]-3,4-dihydropyrido[2,3-b]pyrazin-6-yl}-N-methylacetamide;
N-{6-[(dimethylcarbamoyl)(methyl)amino]-3-oxo-4-[(1S)-1-phenylpropyl]-3,4-dihydropyrido[2,3-b]pyrazin-2-yl}-beta-alanine;
N-{6-[methyl(propanoyl)amino]-4-[(1S)-2-methyl-1-(pyridin-2-yl)propyl]-3-oxo-3,4-dihydropyrido[2,3-b]pyrazin-2-yl}-beta-alanine;
N-{4-(1-cyclopentylcyclopropyl)-6-[methyl(propanoyl)amino]-3-oxo-3,4-dihydropyrido[2,3-b]pyrazin-2-yl}-beta-alanine;
N-(6-{[(3,3-dimethylcyclobutyl)carbonyl](methyl)amino}-3-oxo-4-[(1S)-1-phenylpropyl]-3,4-dihydropyrido[2,3-b]pyrazin-2-yl)-beta-alanine;
N-(6-{[(3,3-difluorocyclobutyl)carbonyl](methyl)amino}-3-oxo-4-[(1S)-1-phenylpropyl]-3,4-dihydropyrido[2,3-b]pyrazin-2-yl)-beta-alanine;
N-{6-[methyl(oxetan-3-ylcarbonyl)amino]-3-oxo-4-[(1S)-1-phenylpropyl]-3,4-dihydropyrido[2,3-b]pyrazin-2-yl}-beta-alanine;
N-methyl-N-(2-{[3-(methylamino)-3-oxopropyl]amino}-3-oxo-4-[(1S)-1-phenylpropyl]-3,4-dihydropyrido[2, 3-b]pyrazin-6-yl)oxetane-2-carboxamide;
N-methyl-N-(2-{[2-(methylamino)-2-oxoethyl]amino}-3-oxo-4-[(1S)-1-phenylpropyl]-3,4-dihydropyrido[2,3-b]pyrazin-6-yl)propanamide;
N-methyl-N-(2-{[2-(methylamino)-2-oxoethyl]amino}-4-[(1S)-2-methyl-1-(pyridin-2-yl)propyl]-3-oxo-3,4-dihydropyrido[2,3-b]pyrazin-6-yl)propanamide;
N³-{4-[(1R)-2-methoxy-1-phenylethyl]-6-[methyl(propanoyl)amino]-3-oxo-3,4-dihydropyrido[2,3-b]pyrazin-2-yl}-N-methyl-beta-alaninamide;
N-methyl-N³-{6-[methyl(propanoyl)amino]-3-oxo-4-[(1S)-1-phenylpropyl]-3,4-dihydropyrido[2,3-b]pyrazin-2-yl}-beta-alaninamide;
N-methyl-N³-{6-[methyl(propanoyl)amino]-3-oxo-4-[(1S)-1-(pyridin-2-yl) propyl]-3,4-dihydropyrido[2,3-b]pyrazin-2-yl}-beta-alaninamide; and
N-methyl-N³-{6-[methyl(propanoyl)amino]-4-[(1S)-2-methyl-1-(pyridin-2-yl)propyl]-3-oxo-3,4-dihydropyrido[2,3-b]pyrazin-2-yl}-beta-alaninamide
or a pharmaceutically acceptable salt thereof.

21. A compound of Formula (I) according to claim 1, which is
N-{6-[methyl(propanoyl)amino]-3-oxo-4-[(1S)-1-phenylpropyl]-3,4-dihydropyrido[2,3-b]pyrazin-2-yl}-beta-alanine
or a pharmaceutically acceptable salt thereof.

22. A method of treating a disease or a disorder in a patient, comprising administering to a patient in need thereof a therapeutically effective amount of a compound of Formula (I) according to claim 1 comprising a therapeutically effective amount of a compound of Formula (I), or a pharmaceutically acceptable salt thereof.

23. A compound of Formula (II), having the structure: 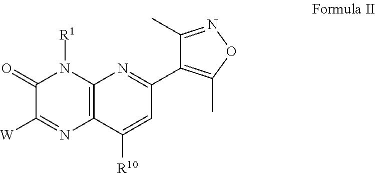
or a pharmaceutically acceptable salt thereof, wherein
R¹ is selected from the group consisting of:
(i) —C₃-C₇cycloalkyl optionally substituted with one, two, three or four E;
(ii) 4 to 7 membered heterocyclyl optionally substituted with one, two, three or four E, which said 4 to 7 membered heterocyclyl comprises one or two heteroatoms independently selected for each occurrence from the group consisting of N, O and S; and
(iii) 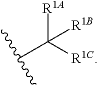
R^{1A} is selected from the group consisting of
(i) —C₁-C₆alkyl optionally substituted with one, two, three, four, five or six E;
(ii) —C₃-C₇cycloalkyl optionally substituted with one, two, three, four or five E;
(iii) phenyl optionally substituted with one, two, three, four or five E;
(iv) 4 to 7 membered heterocyclyl optionally substituted with one, two, three, four or five E, which said 4 to 7 membered heterocyclyl comprises one or two heteroatoms independently selected for each occurrence from the group consisting of N, O and S; and
(v) 5 to 6 membered heteroaryl optionally substituted with one, two, three, four or five E, which said 5 to 6 membered heteroaryl comprises one, two or three heteroatoms independently selected for each occurrence from the group consisting of N, O and S;
R^{1B} is selected from the group consisting of
(i) —H; and
(ii) —C₁-C₆alkyl optionally substituted with one, two, three, four, five or six E;
R^{1C} is selected from the group consisting of
(i) —H;
(ii) —CH₃ optionally substituted with one, two, or three J;
(iii) —CH₂CH₃ optionally substituted with one, two, three, four or five J;
(iv) —CH₂CH₂CH₃ optionally substituted with one, two, three, four, five, six or seven J; and
(v) —CH(CH₃)₂ optionally substituted with one, two, three, four, five, six or seven J;
W is selected from the group consisting of:
(i) 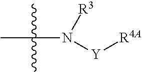
(ii) 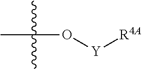
(iii) 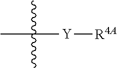
(iv) 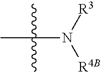
(v) 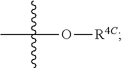
and
(vi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S;
Y is selected from the group consisting of:
(i) —CH₂— optionally substituted with one or two J;
(ii) —(CH₂)₂— optionally substituted with one, two, three or four J;
(iii) —(CH₂)₃— optionally substituted with one, two, three, four, five or six J; and
(iv) —(CH₂)₄— optionally substituted with one, two, three, four, five, six, seven or eight J;
R³ is selected from the group consisting of:
(i) —H;
(ii) —CH₃ optionally substituted with one, two, or three J;
(iii) —CH₂CH₃ optionally substituted with one, two, three, four or five J;
(iv) —CH₂CH₂CH₃ optionally substituted with one, two, three, four, five, six or seven J; and
(v) CH(CH₃)₂ optionally substituted with one, two, three, four, five, six or seven J;
R^{4A} is selected from the group consisting of
(i) —H;
(ii) —C₁-C₆alkyl optionally substituted with one, two, three or four G;
(iii) —CO₂H;
(iv) —C(O)C₁-C₆alkyl optionally substituted with one, two, three or four G;
(v) —C(O)OC₁-C₆alkyl optionally substituted with one, two, three or four G;
(vi) —C(O)NH₂;
(vii) —C(O)NH(C₁-C₆alkyl) optionally substituted with one, two, three or four G;
(viii) —C(O)N(C₁-C₆alkyl)₂ optionally substituted with one, two, three or four G;
(ix) —C(O)NHSO₂C₁-C₃alkyl optionally substituted with one, two, three or four G;
(x) —NH(C₁-C₃alkyl) optionally substituted with one, two, three or four G;
(xi) —N(C₁-C₃alkyl)₂ optionally substituted with one, two, three or four G;
(xii) —NHC(O)C₁-C₃alkyl optionally substituted with one, two, three or four G;
(xiii) —N(C₁-C₃alkyl)C(O)C₁-C₃alkyl optionally substituted with one, two, three or four G;
(xiv) —NHSO₂C₁-C₃alkyl optionally substituted with one, two, three or four G;
(xv) —N(C₁-C₃alkyl)SO₂C₁-C₃alkyl optionally substituted with one, two, three or four G;
(xvi) —SO₂NH₂;
(xvii) —SO₂NH(C₁-C₃alkyl) optionally substituted with one, two, three or four G;
(xviii) —SO₂N(C₁-C₃alkyl)₂ optionally substituted with one, two, three or four G;
(xix) —C₃-C₇cycloalkyl optionally substituted with one, two, three or four G;
(xx) phenyl optionally substituted with one, two, three or four G;
(xxi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
(xxii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4B} is selected from the group consisting of
(i) —H;
(ii) —C₁-C₆alkyl optionally substituted with one, two, three or four G;
(iii) —C(O)C₁-C₆alkyl optionally substituted with one, two, three or four G;
(iv) —C(O)OC₁-C₆alkyl optionally substituted with one, two, three or four G;
(v) —C(O)NH₂;
(vi) —C(O)NH(C₁-C₆alkyl) optionally substituted with one, two, three or four G;
(vii) —C(O)N(C₁-C₆alkyl)₂ optionally substituted with one, two, three or four G;
(viii) —C(O)NHSO₂C₁-C₃alkyl optionally substituted with one, two, three or four G;
(ix) —C₃-C₇cycloalkyl optionally substituted with one, two, three or four G;
(x) phenyl optionally substituted with one, two, three or four G;
(xi) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
(xii) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R^{4C} is selected from the group consisting of
(i) —H;
(ii) —C₁-C₆alkyl optionally substituted with one, two, three or four G;
(iii) —C₃-C₇cycloalkyl optionally substituted with one, two, three or four G;
(iv) phenyl optionally substituted with one, two, three or four G;
(v) 4 to 7 membered heterocyclyl optionally substituted with one, two, three or four G, which said 4 to 7 membered heterocyclyl comprises one, two, three or four heteroatoms independently selected for each occurrence from the group consisting of N, O and S; and
(vi) 5 to 6 membered heteroaryl optionally substituted with one, two, three or four G, which said 5 to 6 membered heteroaryl ring comprises one, two, three or four heteroatoms independently selected for each occurrence from the group consisting of N, O and S;
R¹⁰ is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂;
E is independently selected for each occurrence from the group consisting of:
(i) —OH;
(ii) —CN;
(iii) —CO₂H;
(iv) —C(O)H;
(v) halo;
(vi) —C₁-C₃alkyl optionally substituted with one, two, three or four J;
(vii) —C₁-C₃alkylCO₂H which —C₁-C₃alkyl is optionally substituted with one, two, three or four J;
(viii) —C₃-C₇cycloalkyl optionally substituted with one, two, three, four, five or six J;
(ix) —C₁-C₃alkylC₃-C₆cycloalkyl optionally substituted with one, two, three, four, five or six J;
(x) —OC₁-C₃alkyl, optionally substituted with one, two, three or four J;
(xi) —OC₃-C₇cycloalkyl optionally substituted with one, two, three, four, five or six J;
(xii) —OC₁-C₃alkylC₃-C₇cycloalkyl optionally substituted with one, two, three, four, five or six J;
(xiii) —SC₁-C₃alkyl, optionally substituted with one, two, three or four J;
(xiv) —SC₃-C₇cycloalkyl optionally substituted with one, two, three, four, five or six J;
(xv) —SC₁-C₃alkylC₃-C₇cycloalkyl optionally substituted with one, two, three, four, five or six J;
(xvi) —C(O)C₁-C₃alkyl, optionally substituted with one, two, three or four J;
(xvii) —C(O)OC₁-C₃alkyl, optionally substituted with one, two, three or four J;
(xviii) —NH₂;
(xix) —NH(C₁-C₃alkyl) optionally substituted with one, two, three or four J;
(xx) —N(C₁-C₃alkyl)₂ which —C₁-C₃alkyl is, independently for each occurrence, optionally substituted with one, two, three or four J;
(xxi) —C(O)NH₂;
(xxii) —C(O)NHC₁-C₃alkyl, optionally substituted with one, two, three or four J;
(xxiii) —C(O)N(C₁-C₃alkyl)₂, which —C₁-C₃alkyl is, independently for each occurrence, optionally substituted with one, two, three or four J;
(xxiv) —NHC(O)C₁-C₃alkyl, optionally substituted with one, two, three or four J;
(xxv) —SO₂(C₁-C₃alkyl), optionally substituted with one, two, three or four J;
(xxvi) —SO₂NH(C₁-C₃alkyl), optionally substituted with one, two, three or four J;
(xxvii) —NHSO₂(C₁-C₃alkyl), optionally substituted with one, two, three or four J; and
(xxviii) phenyl optionally substituted with one, two, three, or four J;
G is independently selected for each occurrence from the group consisting of
(i) —OH
(ii) —CN;
(iii) —CO₂H;
(iv) —C(O)H;
(v) halo;
(vi) —C₁-C₃alkyl, optionally substituted with one, two, three or four J;
(vii) —C₁-C₃alkylCO₂H, which —C₁-C₃alkyl is optionally substituted with one, two, three or four J;
(viii) —C₁-C₃alkylC₃-C₆cycloalkyl optionally substituted with one, two, three, four, five or six J;
(ix) —OC₁-C₃alkyl, optionally substituted with one, two, three or four J;
(x) —OC₁-C₃alkylC₃-C₆cycloalkyl optionally substituted with one, two, three, four, five or six J;
(xi) —SC₁-C₃alkyl, optionally substituted with one, two, three or four J;
(xii) —SC₁-C₃alkylC₃-C₆cycloalkyl optionally substituted with one, two, three, four, five or six J;
(xiii) —C(O)C₁-C₃alkyl, optionally substituted with one, two, three or four J;
(xiv) —C(O)OC₁-C₃alkyl, optionally substituted with one, two, three or four J;
(xv) —NH₂;
(xvi) —NH(C₁-C₃alkyl), optionally substituted with one, two, three or four J;
(xvii) —N(C₁-C₃alkyl)₂, which —C₁-C₃alkyl is, independently for each occurrence, optionally substituted with one, two, three or four J;
(xviii) —C(O)NH₂;
(xix) —C(O)NHC₁-C₃alkyl, optionally substituted with one, two, three or four J;
(xx) —C(O)N(C₁-C₃alkyl)₂, which —C₁-C₃alkyl is, independently for each occurrence, optionally substituted with one, two, three or four J;
(xxi) —NHC(O)C₁-C₃alkyl, optionally substituted with one, two, three or four J;
(xxii) —SO₂(C₁-C₃alkyl), optionally substituted with one, two, three or four J;
(xxiii) —SO₂NH(C₁-C₃alkyl), optionally substituted with one, two, three or four J; and
(xxiv) —NHSO₂(C₁-C₃alkyl) optionally substituted with one, two, three or four J; and
J is independently selected for each occurrence from the group consisting of —H, —F, —Cl, —OH, —CN, —CH₃, —CH₂CH₃, —CH₂F, —CHF₂, —CF₃, —CF₂CF₃, —CH₂OH, —OCH₃, —OCH₂F, —OCHF₂, —OCF₃, —SCH₃, —SCH₂F, —SCHF₂, —SCF₃—NH₂, —NH(CH₃), and —N(CH₃)₂.

24. The compound of claim 23, selected from the group consisting of:
6-(3,5-dimethyl-1,2-oxazol-4-yl)-4-[(1S)-1-(2-methoxyphenyl)ethyl]-2-{[2-(morpholin-4-yl)ethyl]amino}pyrido[2,3-b]pyrazin-3(4H)-one;
6-(3,5-dimethyl-1,2-oxazol-4-yl)-4-(2-ethoxybenzyl)-2-{[2-(morpholin-4-yl)ethyl]amino}pyrido[2,3-b]pyrazin-3(4H)-one;
4-benzyl-6-(3,5-dimethyl-1,2-oxazol-4-yl)-2-{[2-(morpholin-4-yl)ethyl]amino}pyrido[2,3-b]pyrazin-3(4H)-one;
6-(3,5-dimethyl-1,2-oxazol-4-yl)-2-{[2-(morpholin-4-yl)ethyl]amino}-4-[(1R)-1-phenylpropyl]pyrido[2, 3-b]pyrazin-3(4H)-one;
6-(3,5-dimethyl-1,2-oxazol-4-yl)-2-{[2-(morpholin-4-yl)ethyl]amino}-4-[(1S)-1-phenylpropyl]pyrido[2, 3-b]pyrazin-3(4H)-one;
6-(3,5-dimethyl-1,2-oxazol-4-yl)-2-{[2-(morpholin-4-yl)ethyl]amino}-4-[(1S)-1-phenylethyl]pyrido[2,3-b]pyrazin-3(4H)-one;
N-{6-(3,5-dimethyl-1,2-oxazol-4-yl)-3-oxo-4-[(1S)-1-phenylethyl]-3,4-dihydropyrido[2,3-b]pyrazin-2-yl}glycine;
N-{6-(3,5-dimethyl-1,2-oxazol-4-yl)-3-oxo-4-[(1S)-1-phenylethyl]-3,4-dihydropyrido[2,3-b]pyrazin-2-yl}-beta-alanine;
N-{6-(3,5-dimethyl-1,2-oxazol-4-yl)-4-[(1S)-1-(2-methoxyphenyl)ethyl]-3-oxo-3,4-dihydropyrido[2, 3-b]pyrazin-2-yl}-beta-alanine;
N-{6-(3,5-dimethyl-1,2-oxazol-4-yl)-3-oxo-4-[(1S)-1-phenylpropyl]-3,4-dihydropyrido[2,3-b]pyrazin-2-yl}-beta-alanine;
N-{6-(3,5-dimethyl-1,2-oxazol-4-yl)-3-oxo-4-[(1S)-1-(pyrimidin-2-yl)propyl]-3,4-dihydropyrido[2, 3-b]pyrazin-2-yl}-beta-alanine;
N-{6-(3,5-dimethyl-1,2-oxazol-4-yl)-4-[(2 S)-1-methoxybutan-2-yl]-3-oxo-3,4-dihydropyrido[2,3-b]pyrazin-2-yl}-beta-alanine;
N-{6-(3,5-dimethyl-1,2-oxazol-4-yl)-4-[(2R)-1-methoxybutan-2-yl]-3-oxo-3,4-dihydropyrido[2,3-b]pyrazin-2-yl}-beta-alanine;
N-[4-(1,3-dimethoxypropan-2-yl)-6-(3,5-dimethyl-1,2-oxazol-4-yl)-3-oxo-3,4-dihydropyrido[2,3-b]pyrazin-2-yl]-beta-alanine;
N-[6-(3,5-dimethyl-1,2-oxazol-4-yl)-3-oxo-4-(tetrahydro-2H-pyran-4-yl)-3,4-dihydropyrido[2, 3-b]pyrazin-2-yl]-beta-alanine;
N³-{6-(3,5-dimethyl-1,2-oxazol-4-yl)-3-oxo-4-[(1S)-1-phenylethyl]-3,4-dihydropyrido[2,3-b]pyrazin-2-yl}-N-(methylsulfonyl)-beta-alaninamide;
6-(3,5-dimethyl-1,2-oxazol-4-yl)-4-[(1S)-1-phenylethyl]-2-{[2-(1H-tetrazol-5-yl)ethyl]amino}pyrido[2,3-b]pyrazin-3(4H)-one;
N-{6-(3,5-dimethyl-1,2-oxazol-4-yl)-3-oxo-4-[(1S)-1-phenylbutyl]-3,4-dihydropyrido[2,3-b]pyrazin-2-yl}-beta-alanine;
N-{6-(3,5-dimethyl-1,2-oxazol-4-yl)-4-[(1S)-2-methyl-1-phenylpropyl]-3-oxo-3,4-dihydropyrido[2, 3-b]pyrazin-2-yl}-beta-alanine;
N-{6-(3,5-dimethyl-1,2-oxazol-4-yl)-4-[(1S)-2-methyl-1-(pyridin-2-yl)propyl]-3-oxo-3,4-dihydropyrido[2, 3-b]pyrazin-2-yl}-beta-alanine;
N-{4-[(1S)-1-cyclohexylethyl]-6-(3,5-dimethyl-1,2-oxazol-4-yl)-3-oxo-3,4-dihydropyrido[2, 3-b]pyrazin-2-yl}-beta-alanine;
N-[6-(3,5-dimethyl-1,2-oxazol-4-yl)-3-oxo-4-(pentan-3-yl)-3,4-dihydropyrido[2,3-b]pyrazin-2-yl]-beta-alanine; and
N²-{6-(3, 5-dimethyl-1,2-oxazol-4-yl)-4-[(1S)-1-(2-methoxyphenyl)ethyl]-3-oxo-3,4-dihydropyrido[2,3-b]pyrazin-2-yl}-N-methylglycinamide,
or a pharmaceutically acceptable salt thereof.

25. A compound of claim 23, which is:
N-{6-(3,5-dimethyl-1,2-oxazol-4-yl)-4-[(2R)-1-methoxybutan-2-yl]-3-oxo-3,4-dihydropyrido[2,3-b]pyrazin-2-yl}-beta-alanine,
or a pharmaceutically acceptable salt thereof.
